(19) United States
(12) Patent Application Publication
(10) Pub. No.: US 2026/0008775 A1
(43) Pub. Date: Jan. 8, 2026

(54) AZOLE MODULATORS OF CHOLESTEROL BIOSYNTHESIS AND THEIR USE FOR PROMOTING REMYELINATION

(71) Applicants: GENENTECH, INC., South San Francisco, CA (US); CONVELO THERAPEUTICS, INC., Cleveland, OH (US)

(72) Inventors: Marie-Gabrielle BRAUN, South San Francisco, CA (US); Georgette Marie CASTANEDO, South San Francisco, CA (US); Ruth DOREL, South San Francisco, CA (US); Man Un UNG, South San Francisco, CA (US); William VERNIER, South San Francisco, CA (US); Matthew VOLGRAF, South San Francisco, CA (US); Nick CARRUTHERS, Cleveland, OH (US)

(21) Appl. No.: 19/093,086

(22) Filed: Mar. 27, 2025

Related U.S. Application Data
(63) Continuation of application No. PCT/US2023/075511, filed on Sep. 29, 2023.
(60) Provisional application No. 63/377,821, filed on Sep. 30, 2022.

(51) Int. Cl. C07D 413/14; A61K 31/496; A61K 31/5377; A61K 31/5386; A61K 31/553; C07D 401/14; C07D 413/08; C07D 413/10; C07D 498/08

(52) U.S. Cl. CPC C07D 413/14; A61K 31/496; A61K 31/5377; A61K 31/5386; A61K 31/553; C07D 401/14; C07D 413/08; C07D 413/10; C07D 498/08

(57) ABSTRACT

The subject matter described herein is directed to myelin-promoting compounds of Formula I and pharmaceutical salts thereof, methods of preparing the compounds, pharmaceutical compositions comprising the compounds, and methods of administering the compounds for the treatment of disorders, such as myelin-related disorders.

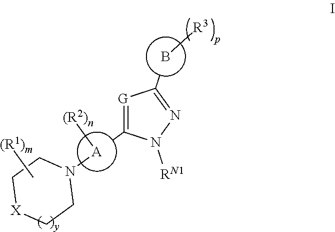

### CROSS-REFERENCE TO RELATED APPLICATIONS

The application claims the benefit of and priority to U.S. Provisional Patent Application No. 63/377,821, filed on Sep. 30, 2022, the entire contents of which are incorporated by reference herein.

### FIELD

The subject matter described herein is directed to myelin-promoting compounds of Formula I, methods of making the compounds, their pharmaceutical compositions, and their use in the treatment of myelin-related disorders.

### BACKGROUND

Myelin-related disorders are disorders that result in abnormalities of the myelin sheath (e.g., dysmyelination, demyelination and hypomyelination) in a subject's neural cells, e.g., CNS neurons including their axons. Loss or degradation of the myelin sheath in such disorders produces a slowing or cessation of nerve cell conduction. The resulting myelin related disorders are characterized by deficits in sensation, motor function, cognition, or other physiological functions. Myelin related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy, and radiation-induced demyelination.

MS is the most common myelin-related disorder affecting several million people globally and is estimated to result in about 18,000 deaths per year. MS is a complex neurological disease characterized by deterioration of central nervous system (CNS) myelin. Myelin, composed in its majority by lipids (70% lipids, 30% protein), protects axons and makes saltatory conduction possible, which speeds axonal electric impulse. Demyelination of axons in chronic MS can result in axon degeneration and neuronal cell death. Additionally, MS destroys oligodendrocytes, the highly specialized CNS cells that generate and maintain myelin. A repair process, called remyelination, takes place in early phases of the disease, but over time, the oligodendrocytes are unable to completely rebuild and restore the myelin sheath. Repeated attacks lead to successively less effective remyelination, until a scar-like plaque is built up around the damaged axons. These scars are the origin of the symptoms.

At present, there is no cure for myelin-related disorders, and only a handful of disease-modifying therapies are available. Accordingly, there is a need for new therapeutic approaches to the treatment of myelin-related disorders, including the promotion of remyelination. The subject matter described herein addresses this unmet need.

### BRIEF SUMMARY

In certain embodiments, the subject matter described herein is directed to a compound of Formulae I, Ia, Ia′, Ib, Ib′, Ia1, Ib1, Ia2, or Ib2, or a pharmaceutically acceptable salt thereof.

In certain embodiments, the subject matter described herein is directed to a pharmaceutical composition comprising a compound of Formulae I, Ia, Ia′, Ib, Ib′, Ia1, Ib1, Ia2, or Ib2, or a pharmaceutically acceptable salt thereof, and a pharmaceutically acceptable excipient.

In certain embodiments, the subject matter described herein is directed to methods of treating a disorder in a subject in need thereof, wherein the disorder is a myelin-related disorder, comprising administering to the subject an effective amount of a compound of Formula I, Ia, Ia′, Ib, Ib′, Ia1, Ib1, Ia2, or Ib2, or a pharmaceutically acceptable salt thereof; or a pharmaceutical composition comprising a compound of Formula I, Ia, Ia′, Ib, Ib′, Ia1, Ib1, Ia2, or Ib2, or a pharmaceutically acceptable salt thereof, and a pharmaceutically acceptable excipient.

In certain embodiments, the subject matter described herein is directed to a compound of Formula I, Ia, Ia′, Ib, Ib′, Ia1, Ib1, Ia2, or Ib2, or a pharmaceutically acceptable salt thereof, or a pharmaceutical composition comprising a pharmaceutically acceptable excipient and a compound of Formula I, Ia, Ia′, Ib, Ib′, Ia1, Ib1, Ia2, or Ib2, or a pharmaceutically acceptable salt thereof, for use in treating a myelin-related disorder.

In certain embodiments, the subject matter described herein is directed to methods of promoting myelination in a subject in need thereof, comprising administering to the subject a therapeutically effective amount of a compound of Formula I, Ia, Ia′, Ib, Ib′, Ia1, Ib1, Ia2, or Ib2, or a pharmaceutically acceptable salt thereof; or a pharmaceutical composition comprising a compound of Formula I, Ia, Ia′, Ib, Ib′, Ia1, Ib1, Ia2, or Ib2, or a pharmaceutically acceptable salt thereof, a pharmaceutically acceptable excipient.

In certain embodiments, the subject matter described herein is directed to the use of a compound of Formula I, Ia, Ia′, Ib, Ib′, Ia1, Ib1, Ia2, or Ib2, or a pharmaceutically acceptable salt thereof, or a pharmaceutical composition comprising a pharmaceutically acceptable excipient and a compound of Formula I, Ia, Ia′, Ib, Ib′, Ia1, Ib1, Ia2, or Ib2, or a pharmaceutically acceptable salt thereof, in the manufacture of a medicament for treating a myelin-related disorder.

In certain embodiments, the subject matter described herein is directed to methods of preparing compound of Formula I, Ia, Ia′, Ib, Ib′, Ia1, Ib1, Ia2, or Ib2, or a pharmaceutically acceptable salt thereof.

Other embodiments are also described.

### DETAILED DESCRIPTION

Described herein are compounds of Formulae I, Ia, Ia′, Ib, Ib′, Ia1, Ib1, Ia2, and Ib2, and pharmaceutically acceptable salts thereof, methods of making the compounds, their pharmaceutical compositions, and their use in the treatment of myelin-related disorders. In some embodiments, the compounds provided herein are myelin-promoting.

Without wishing to be bound by theory, the enhancement and/or inducement of the accumulation of Δ8,9-unsaturated sterol intermediates of the cholesterol biosynthesis pathway in oligodendrocyte progenitor cells (OPCs) can induce oligodendrocyte generation. Enhancement and/or inducement of the accumulation of Δ8,9-unsaturated sterol intermediates can be provided, for example, by modulating and/or inhibiting the enzymes within the OPC cholesterol biosynthesis pathway that inhibit Δ8,9-unsaturated sterol intermediate accumulation and/or for which the Δ8,9-unsaturated sterol intermediates are substrates, as well as directly and/or indirectly administering Δ8,9-unsaturated sterol intermediates to the OPCs. Enhancement and/or inducement of the accumulation of Δ8,9-unsaturated sterol intermediates may promote OPC differentiation, survival, proliferation, and/or maturation, and it is thought this might treat disease and/or disorders in subjects where myelination is beneficial to the subject.

As such, in some embodiments, an agent, such as a compound of Formula I, Ia, Ia′, Ib, Ib′, Ia1, Ib1, Ia2, or Ib2, or a pharmaceutically acceptable salt thereof, that can enhance and/or induce accumulation of Δ8,9-unsaturated sterol intermediates of the cholesterol biosynthesis pathway in OPCs can be administered to a subject, and/or to the OPCs, at an amount effective to promote and/or induce OPC differentiation, proliferation, and/or maturation, as well as oligodendrocyte generation. In certain embodiments, the agent, for example a compound of Formula I, Ia, Ia′, Ib, Ib′, Ia1, Ib1, Ia2, or Ib2, or a pharmaceutically acceptable salt thereof, is a compound that inhibits enzyme-mediated synthesis of one or more sterol intermediates in the cholesterol biosynthesis pathway of the OPCs, and/or promotes accumulation of Δ8,9-unsaturated sterol intermediates.

In certain embodiments, the compound of Formula I, Ia, Ia′, Ib, Ib′, Ia1, Ib1, Ia2, or Ib2, or a pharmaceutically acceptable salt thereof, can modulate and/or inhibit one or more enzyme-mediated conversion steps of the cholesterol biosynthesis pathway, such as in the pathway from lanosterol to cholesterol, for example, between lanosterol and/or lathosterol; modulating and/or inhibiting one or more of these steps in OPCs may promote and/or induce oligodendrocyte generation. For example, in some embodiments, a compound of Formula I, Ia, Ia′, Ib, Ib′, Ia1, Ib1, Ia2, or Ib2, or a pharmaceutically acceptable salt thereof can inhibit CYP51, sterol 14-reductase (TM7SF2 and/or LBR), SC4MOL, NSDHL, and/or emopamil binding protein (EBP) enzyme mediated synthesis of sterol intermediates in the cholesterol biosynthesis pathway. In certain embodiments, a compound of Formula I, Ia, Ia′, Ib, Ib′, Ia1, Ib1, Ia2, or Ib2, or a pharmaceutically acceptable salt thereof can inhibit CYP51, sterol 14-reductase and/or EBP. In certain embodiments, the compounds of Formula I, Ia, Ia′, Ib, Ib′, Ia1, Ib1, Ia2, or Ib2 or pharmaceutically acceptable salt thereof can inhibit EBP.

For example, in certain embodiments, a compound of Formula I, Ia, Ia′, Ib, Ib′, Ia1, Ib1, Ia2, or Ib2, or a pharmaceutically acceptable salt thereof, used in the methods described herein can inhibit enzyme mediated conversion of zymostenol to lathosterol through the inhibition of emopamil binding protein (EBP) isomerase enzyme activity. Alternatively, in certain embodiments, the compound of Formula I, Ia, Ia′, Ib, Ib′, Ia1, Ib1, Ia2, or Ib2, or a pharmaceutically acceptable salt thereof, used in the methods described herein can inhibit sterol C14 reductase enzyme activity or CYP51 enzyme activity in the cholesterol biosynthesis pathway.

Emopamil Binding Protein (EBP) is an enzyme responsible for one of the final steps in the production of cholesterol. Specifically, EBP converts zymostenol to lathosterol, where other enzymes then modify lathosterol to produce cholesterol. EBP is also referred to as Δ8-Δ7-sterol isomerase, 3-beta-hydroxysteroid-Delta(8),Delta(7)-isomerase, CDPX2, CHO2, CPX, or CPXD).

Without being bound by a particular theory, it is believed that compounds of Formula I, Ia, Ia′, Ib, Ib′, Ia1, Ib1, Ia2, or Ib2, or a pharmaceutically acceptable salt thereof can inhibit EBP mediated conversion of zymostenol to lathosterol in the cholesterol biosynthesis pathway of OPCs resulting in enhancement and/or inducement of the accumulation of Δ8,9-unsaturated sterol intermediates. In some embodiments, enhancement and/or inducement of the accumulation of Δ8,9-unsaturated sterol intermediates can promote OPC differentiation, survival, proliferation and/or maturation and treat disease and/or disorders in subjects where myelination or myelinization is beneficial to the subject. This mechanism of promoting myelination is distinct from the primary action of immunomodulatory agents that are often used to treat myelin-related disorders.

The presently disclosed subject matter will now be described more fully hereinafter. However, many modifications and other embodiments of the presently disclosed subject matter set forth herein may come to mind to one skilled in the art to which the presently disclosed subject matter pertains having the benefit of the teachings presented in the descriptions herein. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

### I. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±50%. In certain other embodiments, the term “about” includes the indicated amount±20%. In certain other embodiments, the term “about” includes the indicated amount±10%. In other embodiments, the term “about” includes the indicated amount±5%. In certain other embodiments, the term “about” includes the indicated amount±1%. In certain other embodiments, the term “about” includes the indicated amount±0.5% and in certain other embodiments, 0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl or aralkyl, the last-mentioned group contains the atom by which the moiety is attached to the rest of the molecule.

“Alkenyl” refers to an alkyl group containing at least one carbon-carbon double bond and, unless otherwise described, may have from 2 to 20 carbon atoms (i.e., C₂-C₂₀ alkenyl), 2 to 8 carbon atoms (i.e., C₂-C₈ alkenyl), 2 to 6 carbon atoms (i.e., C₂-C₆ alkenyl) or 2 to 4 carbon atoms (i.e., C₂-C₄ alkenyl). Examples of alkenyl groups include, e.g., ethenyl, propenyl, butadienyl (including 1,2-butadienyl and 1,3-butadienyl).

“Alkynyl” refers to an alkyl group containing at least one carbon-carbon triple bond, unless otherwise described, may have from 2 to 20 carbon atoms (i.e., C₂-C₂₀ alkynyl), 2 to 8 carbon atoms (i.e., C₂-C₈ alkynyl), 2 to 6 carbon atoms (i.e., C₂-C₆ alkynyl) or 2 to 4 carbon atoms (i.e., C₂-C₄ alkynyl). The term “alkynyl” also includes those groups having one triple bond and one double bond.

“Alkoxy” refers to the group “alkyl-O—” (e.g., C₁-C₃ alkoxy or C₁-C₆ alkoxy). Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Acyl” refers to a group —C(O)R^{y}, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of acyl include, e.g., formyl, acetyl, cyclohexylcarbonyl, cyclohexylmethyl-carbonyl and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein, or R^{y} and R^{z} are taken together to form a heterocyclyl; which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of the point of attachment. If one or more aryl groups are fused with a heterocyclyl, the resulting ring system is heterocyclyl regardless of the point of attachment.

“Arylalkyl” or “Aralkyl” refers to the group “aryl-alkyl-”, such as (C₆-C₁₀ aryl)-C₁-C₃ alkyl. A non-limiting example of arylalkyl is benzyl.

“Cycloalkyl” refers to a saturated or partially unsaturated cyclic alkyl group having a single ring or multiple rings which may include fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃-C₂₀ cycloalkyl), 3 to 12 ring carbon atoms (i.e., C₃-C₁₂ cycloalkyl), 3 to 10 ring carbon atoms (i.e., C₃-C₁₀ cycloalkyl), 3 to 8 ring carbon atoms (i.e., C₃-C₈ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any moiety comprising a non-aromatic alkyl ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

As used herein, “halocycloalkyl,” such as C₃-C₇ halocycloalkyl, refers to a C₃-C₇ cycloalkyl group that is substituted with one or more halogens.

“Cycloalkylalkyl” refers to the group “cycloalkyl-alkyl-”, such as (C₃-C₆ cycloalkyl)-C₁-C₃ alkyl.

“Halogen” or “halo” refers to atoms occupying group VIIA of the periodic table, such as fluoro (fluorine), chloro (chlorine), bromo (bromine) or iodo (iodine).

“Haloalkyl” refers to an unbranched or branched alkyl group as defined above, wherein one or more (e.g., 1 to 6, or 1 to 3) hydrogen atoms are replaced by a halogen. For example, halo-C₁-C₃ alkyl refers to an alkyl group of 1 to 3 carbons wherein at least one hydrogen atom is replaced by a halogen. Halo-C₁-C₆ alkyl refers to an alkyl group of 1 to 6 carbons wherein at least one hydrogen atom is replaced by a halogen. Where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6, or 1 to 3) hydrogen atoms are replaced by a halogen. For example, halo-C₁-C₃ alkoxy refers to an alkoxy group of 1 to 3 carbons wherein at least one hydrogen atom is replaced by a halogen. Halo-C₁-C₆ alkoxy refers to an alkoxy group of 1 to 6 carbons wherein at least one hydrogen atom is replaced by a halogen. Non-limiting examples of haloalkoxy are —OCH₂CF₃, —OCF₂H, and —OCF₃. [42]“Hydroxyalkyl” refers to an alkyl group as defined above, wherein one or more (e.g., 1 to 6, or 1 to 3) hydrogen atoms are replaced by a hydroxy group (e.g., hydroxy-C₁-C₃-alkyl, hydroxy-C₁-C₆-alkyl). The term “hydroxy-C₁-C₃ alkyl” refers to a one to three carbon alkyl chain where one or more hydrogens on any carbon is replaced by a hydroxy group, in particular, one hydrogen on one carbon of the chain is replaced by a hydroxy group. The term “hydroxy-C₁-C₆ alkyl” refers to a one to six carbon alkyl chain where one or more hydrogens on any carbon is replaced by a hydroxy group, in particular, one hydrogen on one carbon of the chain is replaced by a hydroxy group. Non-limiting examples of hydroxyalkyl include —CH₂OH, —CH₂CH₂OH, and —C(CH₃)₂CH₂OH.

“Heteroalkyl” refers to an alkyl group in which one or more of the carbon atoms (and any associated hydrogen atoms) are each independently replaced with the same or different heteroatomic group, provided the point of attachment to the remainder of the molecule is through a carbon atom. In certain embodiments, the heteroalkyl can have 1 to 3 carbon atoms (e.g., C₁-C₃ heteroalkyl) or 1 to 6 carbon atoms (e.g., C₁-C₆ heteroalkyl), and one or more (e.g., 1, 2, or 3) heteroatoms or heteroatomic groups. The term “heteroalkyl” includes unbranched or branched saturated chain having carbon and heteroatoms. By way of example, 1, 2, or 3 carbon atoms of the alkyl group in the “heteroalkyl” may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.) and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein. In certain embodiments, heteroalkyl can have 1 to 20 carbon atoms, 1 to 15 carbon atoms, 1 to 12 carbon atoms, 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems (i.e., 9-10 membered heteroaryl), 5-10 membered ring systems (i.e., 5-10 membered heteroaryl), 5-7 membered ring systems (i.e., 5-7 membered heteroaryl), 5-6 membered ring systems (i.e., 5-6 membered heteroaryl), or 4-6 membered ring systems (i.e., 4-6 membered heteroaryl), each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic group, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”, such as (5- to 10-membered monocyclic heteroaryl)-C₁-C₃ alkyl.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro. Any non-aromatic ring containing at least one heteroatom is considered a heterocyclyl, regardless of the attachment (i.e., can be bound through a carbon atom or a heteroatom). Further, the term heterocyclyl is intended to encompass a moiety comprising any non-aromatic ring containing at least one heteroatom, which ring may be fused to an aryl or heteroaryl ring, regardless of the attachment to the remainder of the molecule. The term heterocyclyl is also intended to encompass a moiety comprising a cycloalkyl ring which is fused to a heteroaryl ring, regardless of the attachment to the remainder of the molecule. Additionally, the term heterocyclyl is intended to encompass a moiety comprising a cycloalkyl ring which is fused to a heterocyclyl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₈ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocyclyl ring contains 4- to 6-ring atoms, it is also referred to herein as a 4- to 6-membered heterocyclyl. Also disclosed herein are 5- or 6-membered heterocyclyls, having 5 or 6 ring atoms, respectively, and 5- to 10-membered heterocyclyls, having 5 to 10 ring atoms. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, tetrahydropyranyl, thiomorpholinyl, thiamorpholinyl, 1-oxo-thiomorpholinyl and 1,1-dioxo-thiomorpholinyl. In certain embodiments, the term “heterocyclyl” can include “spiroheterocyclyl” when there are two positions for substitution on the same carbon atom, wherein at least one ring of the spiro system comprises at least one heteroatom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“Heterocyclylalkyl” refers to the group “heterocyclyl-alkyl-.”

“Oxo” refers to the group (═O).

“Cyano” refers to the group (—CN).

“Sulfonyl” refers to the group —S(O)₂R^{y}, where R^{y} is hydrogen, alkyl, alkenyl, alkynyl, cycloalkyl, heterocyclyl, aryl, heteroalkyl or heteroaryl; each of which may be optionally substituted, as defined herein. A non-limiting example of a sulfonyl group is —SO₂(C₁-C₆ alkyl), which is herein referred to as alkylsulfonyl. Examples of sulfonyl are methylsulfonyl, ethylsulfonyl, phenylsulfonyl and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfinyl are methylsulfinyl, ethylsulfinyl, phenylsulfinyl and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, 1 to 4, or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5, 1 to 4, or 1 to 3) hydrogen atom is replaced by a bond to a non-hydrogen moiety. Unless otherwise described, such non-hydrogen moieties may include, but are not limited to, alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanidino, halo, haloalkyl, haloalkoxy, hydroxyalkyl, heteroalkyl, heteroaryl, heteroarylalkyl, heterocyclyl, heterocyclylalkyl, —NHNH₂, ═NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, 1 to 4, or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NRS(═O)₁₋₂R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂₀R^{g}, —NR^{g}S(═O)₁₋₂NR^{g}R^{h}, ═NSO₂R^{g}, ═NOR^{g}, —S(═O)₁₋₂NR^{g}R^{h}, —SF₅, —SCF₃ or —OCF₃. In certain embodiments, “substituted” also means any of the above groups in which one or more (e.g., 1 to 5, 1 to 4, or 1 to 3) hydrogen atoms are replaced with —C(═O)R^{g}, —C(═O)OR^{g}, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5, 1 to 4, or 1 to 3) hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} and Rⁱ are taken together with the atoms to which they are attached to form a heterocyclyl ring optionally substituted with oxo, halo or alkyl optionally substituted with oxo, halo, amino, hydroxyl, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl)substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to four. In certain embodiments, as used herein, the phrase “one or more” refers to one to three.

Any compound or structure given herein, is intended to represent unlabeled forms as well as isotopically labeled forms (isotopologues) of the compounds. These forms of compounds may also be referred to as and include “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure include, for example, those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium. Further, in some embodiments, the corresponding deuterated analog is provided.

In many cases, the compounds of this disclosure are capable of forming acid and/or base salts by virtue of the presence of amino and/or carboxyl groups or groups similar thereto.

Provided also are a pharmaceutically acceptable salt, isotopically enriched analog, deuterated analog, isomer (such as a stereoisomer), and mixture of isomers (such as a mixture of stereoisomers), of the compounds described herein.

“Pharmaceutically acceptable” or “physiologically acceptable” refer to compounds, salts, compositions, dosage forms and other materials which are useful in preparing a pharmaceutical composition that is suitable for veterinary or human pharmaceutical use. Generally, such a material is not biologically or otherwise undesirable, e.g., the material may be incorporated into a pharmaceutical composition administered to a patient without causing any significant undesirable biological effects or interacting in a deleterious manner with any of the other components of the composition in which it is contained.

The term “pharmaceutically acceptable salt” of a given compound includes salts which are generally safe and not biologically or otherwise undesirable, and includes those which are acceptable for veterinary use as well as human pharmaceutical use. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The term “hydrate” refers to the complex formed by the combining of a compound described herein and water. A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine. Solvates include hydrates. Any compound or structure given herein is intended to encompass hydrates and/or solvates of the compound.

The compounds described herein, or their pharmaceutically acceptable salts, may include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC). When the compounds described herein contain olefinic double bonds or other centres of geometric asymmetry, and unless specified otherwise, it is intended that the compounds include both E and Z geometric isomers. In some embodiments, the flat structures shown herein include all possible stereochemistry. In certain embodiments of the compounds of Formula I, or pharmaceutically acceptable salts thereof, such as compounds of Formula Ia, Ia′, Ia1, or Ia2, or pharmaceutically acceptable salts of any of the foregoing, wherein Ring A is

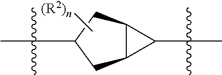

### and Ring C is

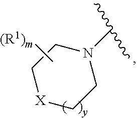

the combination of Ring A and Ring C may comprise any one of the following stereoisomers:

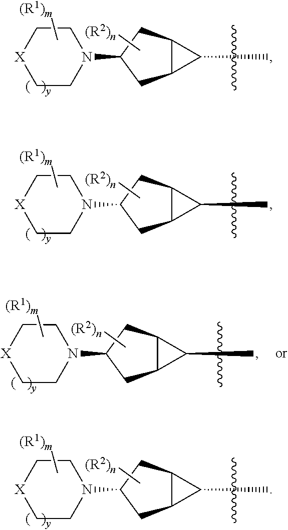

In certain embodiments of the compounds of Formula I, or pharmaceutically acceptable salts thereof, such as compounds of Formula Ib, Ib′, Ib1, or Ib2, or pharmaceutically acceptable salts of any of the foregoing, wherein Ring A is

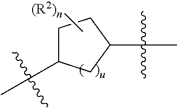

### and Ring C is

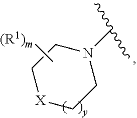

the combination of Ring A and Ring C may comprise any one of the following stereoisomers:

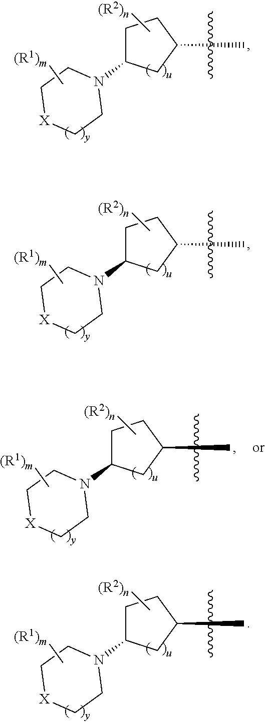

The compounds described herein can contain any combination of stereocenters and any combination of stereochemistry at each stereocenter.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Treatment” or “treating” is an approach for obtaining beneficial or desired results including but not limited to clinical results. Beneficial or desired results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease or condition, that is, causing the regression of clinical symptoms (e.g., ameliorating the disease state, providing partial or total remission of the disease or condition, enhancing effect of another medication, delaying the progression of the disease, increasing the quality of life, and/or prolonging survival). Also encompassed by “treatment” or “treating” is a reduction of pathological consequence of demyelination.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt thereof means an amount sufficient to effect treatment when administered to a subject, to provide a therapeutic benefit such as amelioration of symptoms or slowing of disease progression. The therapeutically effective amount may vary depending on the subject, and disease or condition being treated, the weight and age of the subject, the severity of the disease or condition, and the manner of administering, which can readily be determined by one of ordinary skill in the art. The effective amount of a compound of the disclosure in such a therapeutic method is, for example, from about 0.01 mg/kg/day to about 1000 mg/kg/day.

The term “excipient” as used herein refers to an inert or inactive substance that may be used in the production of a drug or pharmaceutical composition, such as a tablet containing a compound as described herein (or pharmaceutically acceptable salt) as an active ingredient. Various substances may be embraced by the term excipient, including without limitation any substance used as a diluent, filler or extender, binder, disintegrant, humectant, coating, emulsifier or dispersing agent, compression/encapsulation aid, cream or lotion, lubricant, solution for parenteral administration, material for chewable tablets, sweetener or flavoring, suspending/gelling agent, or wet granulation agent. Binders may include, e.g., carbomers, povidone, xanthan gum, etc.; coatings may include, e.g., cellulose acetate phthalate, ethylcellulose, gellan gum, maltodextrin, enteric coatings, etc.; compression/encapsulation aids include e.g. calcium carbonate, dextrose, fructose dc (dc—“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g. dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc. In some cases, the term “excipient” encompasses pharmaceutically acceptable carriers.

Additional definitions may also be provided below as appropriate.

### II. Compounds

In certain embodiments, the subject matter described herein is directed to compounds of Formula I:

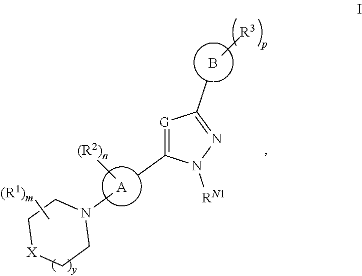

or a pharmaceutically acceptable salt thereof,
wherein:
n is 0 or 1;
p is 1 or 2;
Ring A is a saturated 5- or 6-membered monocyclic or bicyclic carbocyclyl;
Ring B is phenyl, 6-membered heteroaryl, or 6-membered saturated or partially saturated cycloalkyl, wherein the heteroaryl contains one or two heteroatoms;
R³, in each instance, is independently selected from the group consisting of C₁-C₆ alkyl, halo-C₁-C₆ alkyl, hydroxy, C₁-C₆ alkoxy, halo-C₁-C₆ alkoxy, halo, and cyano;
G is N or CR^{G}, wherein R^{G} is selected from the group consisting of hydrogen, C₁-C₆ alkyl, and halo-C₁-C₆ alkyl;
R^{N1} is selected from the group consisting of hydrogen, C₁-C₆ alkyl, halo-C₁-C₆ alkyl, and C₃-C₅ cycloalkyl;
R² is selected from the group consisting of C₁-C₆ alkyl, halo, and halo-C₁-C₆ alkyl;
y is 1 or 2;
m is 0, 1, 2, or 3;
X is O or NR^{H};
   R^{H} is selected from the group consisting of hydrogen, C₁-C₆ alkyl, halo-C₁-C₆ alkyl, C₃-C₅ cycloalkyl, hydroxy-C₁-C₆ alkyl, and C₁-C₆ alkoxy-C₁-C₆ alkyl;
      and,
R¹ in each instance, is selected from the group consisting of C₁-C₆ alkyl, halo-C₁-C₆ alkyl, C₁-C₆ alkoxy, hydroxy, NR^{E}R^{F}, halo-C₁-C₆ alkoxy, hydroxy-C₁-C₆ alkyl, and C₁-C₆ alkoxy-C₁-C₆ alkyl; or, two R¹ groups come together to form a —CH₂— or —CH₂CH₂— bridge
   R^{E} and R^{F} are each independently selected from the group consisting of hydrogen, C₁-C₆ alkyl, halo-C₁-C₆ alkyl, C₃-C₅ cycloalkyl, hydroxy-C₁-C₆ alkyl, and C₁-C₆ alkoxy-C₁-C₆ alkyl.

The ring system depicted in Formula I, and various subgenera as described herein, having the structure:

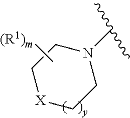

may also be referred to herein as “Ring C”.

The ring system depicted in Formula I, and various subgenera as described herein, having the structure:

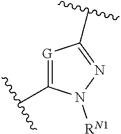

may also be referred to herein as “Ring D”.

In certain embodiments, compounds include those of Formula I, or pharmaceutically acceptable salts thereof, where Ring A is selected from the group consisting of cyclopentyl, cyclohexyl, and bicyclo[3.1.0]hexanyl.

In certain embodiments, compounds include those of Formula I, or pharmaceutically acceptable salts thereof, where Ring B is selected from the group consisting of cyclohexyl, phenyl, pyridinyl, pyrimidinyl, and pyrazinyl.

In certain embodiments, compounds of Formula I, or pharmaceutically acceptable salts thereof, include compounds of Formula Ia or Formula 1a′, or pharmaceutically acceptable salts thereof:

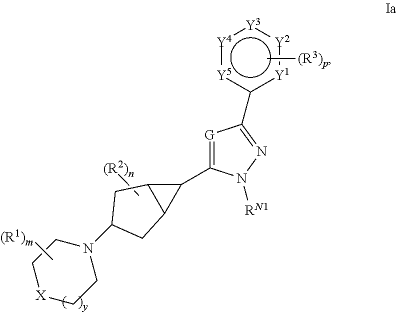

wherein,
Y¹, Y², Y³, Y⁴, and Y⁵ are each independently N, C or CH, provided that only one or two of Y¹, Y², Y³, Y⁴, and Y⁵ can be N; or,

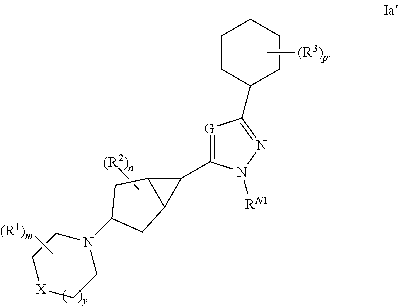

In certain embodiments, compounds of Formula I, or pharmaceutically acceptable salts thereof, include compounds of Formula Ib, or pharmaceutically acceptable salts thereof:

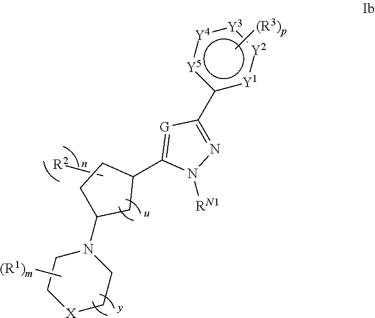

or a pharmaceutically acceptable salt thereof,
wherein:
Y¹, Y², Y³, Y⁴, and Y⁵ are each independently N, C or CH, provided that only one or two of Y¹, Y², Y³, Y⁴, and Y⁵ can be N; and
u is 1 or 2.

In certain embodiments, compounds include those of formula Ib, or pharmaceutically acceptable salts thereof, where u is 1. In certain embodiments, compounds include those of formula Ib, or pharmaceutically acceptable salts thereof, where u is 2.

In certain embodiments, compounds include those of Formula Ia or Ib, or pharmaceutically acceptable salts thereof, where Y¹ is CH, Y² is CR³, Y³ is CH, Y⁴ is N, and Y⁵ is CH. In certain embodiments, compounds include those of Formula Ia or Ib, or pharmaceutically acceptable salts thereof, where Y¹ is CH, Y² is N, Y³ is CR³, Y⁴ is CH, and Y⁵ is CH. In certain embodiments, compounds include those of Formula Ia or Ib, or pharmaceutically acceptable salts thereof, where Y¹ is N, Y² is CR³, Y³ is CH, Y⁴ is CH, and Y⁵ is CH. In certain embodiments, compounds include those of Formula Ia or Ib, or pharmaceutically acceptable salts thereof, where Y¹ is CH, Y² is CR³, Y³ is CH, Y⁴ is CH, and Y⁵ is CH. In certain embodiments, compounds include those of Formula Ia or Ib, or pharmaceutically acceptable salts thereof, where Y¹ is CH, Y² is CR³, Y³ is CH, Y⁴ is CH, and Y⁵ is N. In certain embodiments, compounds include those of Formula Ia or Ib, or pharmaceutically acceptable salts thereof, where Y¹ is CH, Y² is N, Y³ is CR³, Y⁴ is N, and Y⁵ is CH. In certain embodiments, compounds include those of Formula Ia or Ib, or pharmaceutically acceptable salts thereof, where Y¹ is N, Y² is CR³, Y³ is N, Y⁴ is CH, and Y⁵ is CH. In certain embodiments, compounds include those of Formula Ia or Ib, or pharmaceutically acceptable salts thereof, where Y¹ is CH, Y² is CR³, Y³ is N, Y⁴ is CH, and Y⁵ is N. In certain embodiments, compounds include those of Formula Ia or Ib, or pharmaceutically acceptable salts thereof, where Y¹ is N, Y² is CR³, Y³ is CH, Y⁴ is N, and Y⁵ is CH. In certain embodiments, compounds include those of Formula Ia or Ib, or pharmaceutically acceptable salts thereof, where Y¹ is CH, Y² is CR³, Y³ is CR³, Y⁴ is CH, and Y⁵ is CH. In certain embodiments, compounds include those of Formula Ia or Ib, or pharmaceutically acceptable salts thereof, where Y¹ is CH, Y² is N, Y³ is CR³, Y⁴ is CR³, and Y⁵ is CH. In certain embodiments, compounds include those of Formula Ia or Ib, or pharmaceutically acceptable salts thereof, where Y¹ is CH, Y² is CR³, Y³ is N, Y⁴ is CH, and Y⁵ is CH. In certain embodiments, compounds include those of Formula Ia or Ib, or pharmaceutically acceptable salts thereof, where Y¹ is CH, Y² is CR³, Y³ is N, Y⁴ is CH, and Y⁵ is CH. In certain embodiments, compounds include those of Formula Ia or Ib, or pharmaceutically acceptable salts thereof, where Y¹ is CH, Y² is CH, Y³ is CR³, Y⁴ is CH, and Y⁵ is CH. In certain embodiments, compounds include those of Formula Ia or Ib, or pharmaceutically acceptable salts thereof, where Y¹ is N, Y² is CH, Y³ is CR³, Y⁴ is CH, and Y⁵ is CH. In certain embodiments, compounds include those of Formula Ia or Ib, or pharmaceutically acceptable salts thereof, where Y¹ is CR³, Y² is CH, Y³ is CH, Y⁴ is CH, and Y⁵ is CH.

In certain embodiments, compounds of Formula I, or pharmaceutically acceptable salts thereof, include compounds of Formula Ib′:

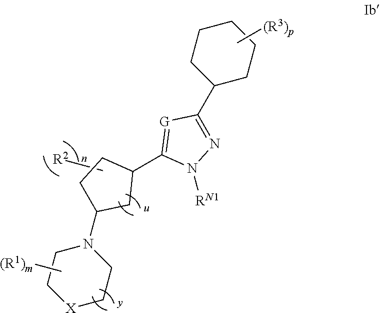

or pharmaceutically acceptable salts thereof.

In certain embodiments, compounds include those of formula Ib′, or pharmaceutically acceptable salts thereof, where u is 1. In certain embodiments, compounds include those of formula Ib, or pharmaceutically acceptable salts thereof, where u is 2.

In certain embodiments, compounds include those of Formula I, Ia, 1a, Ib, or 1b′, or pharmaceutically acceptable salts thereof, where R³ is selected from the group consisting of halo-C₁-C₆ alkyl, halo, cyano, C₁-C₆ alkyl, and halo-C₁-C₆ alkoxy. In certain embodiments, compounds include those of Formula I, Ia, 1a, Ib, or 1b′, or pharmaceutically acceptable salts thereof, where R³ is selected from the group consisting of —CF₃, —CHF₂, methyl, fluoro, chloro, cyano, —OCF₃, and —OCHF₂. In certain embodiments, compounds include those of Formula I, Ia, 1a, Ib, or 1b′, or pharmaceutically acceptable salts thereof, where R³ is —CF₃.

In certain embodiments, compounds include those of Formula I, Ia, 1a, Ib, or 1b′, or pharmaceutically acceptable salts thereof, where at least one R³ is halo. In certain embodiments, compounds include those of Formula I, Ia, 1a, Ib, or 1b′, or pharmaceutically acceptable salts thereof, where at least one of R³ is fluoro.

In certain embodiments, compounds include those of Formula I, Ia, 1a, Ib, or 1b′, or pharmaceutically acceptable salts thereof, where G is N. In certain embodiments, compounds include those of Formula I, Ia, 1a′, Ib, or 1b′, or pharmaceutically acceptable salts thereof, where G is CH.

In certain embodiments, compounds include those of Formula I, Ia, 1a, Ib, or 1b′, or pharmaceutically acceptable salts thereof, where R^{N1} is selected from the group consisting of C₁-C₆ alkyl and C₃-C₅ cycloalkyl. In certain embodiments, compounds include those of Formula I, Ia, 1a′, Ib, or 1b′, or pharmaceutically acceptable salts thereof, where R^{N1} is selected from the group consisting of methyl, ethyl, propyl, butyl, and cyclopropyl. In certain embodiments, compounds include those of Formula I, Ia, or Ib, or pharmaceutically acceptable salts thereof, where R^{N1} is propyl. In certain embodiments, compounds include those of Formula I, Ia, 1a′, Ib, or 1b′, or pharmaceutically acceptable salts thereof, where R^{N1} is isopropyl.

In certain embodiments, compounds include those of Formula I, Ia, 1a′, Ib, or 1b′, or pharmaceutically acceptable salts thereof, where n is 0.

In certain embodiments, compounds include those of Formula I, Ia, 1a′, Ib, or 1b′, or pharmaceutically acceptable salts thereof, where X is O. In certain embodiments, compounds include those of Formula I, Ia, 1a′, Ib, or 1b′, or pharmaceutically acceptable salts thereof, where y is 1. In certain embodiments, compounds include those of Formula I, Ia, 1a′, Ib, or 1b′, or pharmaceutically acceptable salts thereof, where y is 2. In certain embodiments, compounds include those of Formula I, Ia, 1a′, Ib, or 1b′, or pharmaceutically acceptable salts thereof, where R¹ in each instance is selected from the group consisting of C₁-C₆ alkyl and halo-C₁-C₆ alkyl. In certain embodiments, compounds include those of Formula I, Ia, 1a′, Ib, or 1b′, or pharmaceutically acceptable salts thereof, where R¹ in each instance is selected from the group consisting of methyl, ethyl, and —CH₂F. In certain embodiments, compounds include those of Formula I, Ia, 1a′, Ib, or 1b′, or pharmaceutically acceptable salts thereof, where R¹ in each instance is methyl. In certain embodiments, compounds include those of Formula I, Ia, 1a′, Ib, or Ib′, or pharmaceutically acceptable salts thereof, where two R¹ groups come together to form a —CH₂— or —CH₂CH₂— bridge. In certain embodiments, compounds include those of Formula I, Ia, 1a′, Ib, or 1b′, or pharmaceutically acceptable salts thereof, where m is 0. In certain embodiments, compounds include those of Formula I, Ia, 1a′, Ib, or 1b′, or pharmaceutically acceptable salts thereof, where m is 1. In certain embodiments, compounds include those of Formula I, Ia, 1a′, Ib, or 1b′, or pharmaceutically acceptable salts thereof, where m is 2. In certain embodiments, compounds include those of Formula I, Ia, 1a′, Ib, or 1b′, or pharmaceutically acceptable salts thereof, wherein Ring C is any one of:

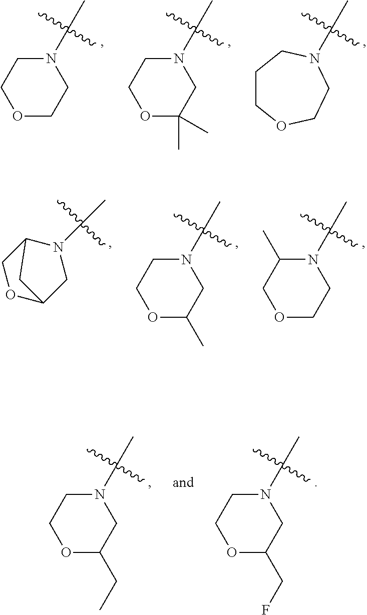

In certain embodiments, compounds include those of Formula I, Ia, 1a′, Ib, or 1b′, or pharmaceutically acceptable salts thereof, where X is NR^{H}. In certain embodiments, compounds include those of Formula I, Ia, 1a′, Ib, or 1b′, or pharmaceutically acceptable salts thereof, where R^{H} is selected from the group consisting of C₃-C₅ cycloalkyl, hydroxy-C₁-C₆ alkyl, and C₁-C₆ alkoxy-C₁-C₆ alkyl. In certain embodiments, compounds include those of Formula I, Ia, 1a′, Ib, or 1b′, or pharmaceutically acceptable salts thereof, where R^{H} is selected from the group consisting of —CH₂CH₂OCH₃, cyclobutyl, and —CH₂CH₂OH. In certain embodiments, compounds include those of Formula I, Ia, 1a′, Ib, or 1b′, or pharmaceutically acceptable salts thereof, where m is 0. In certain embodiments, compounds include those of Formula I, Ia, 1a′, Ib, or 1b′, or pharmaceutically acceptable salts thereof, wherein Ring C is any one of:

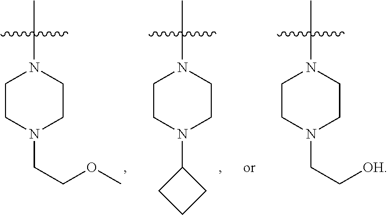

In certain embodiments, compounds of Formula I, or pharmaceutically acceptable salts thereof, include compounds of Formula Ia1 or Formula Ib1, or pharmaceutically acceptable salts thereof:

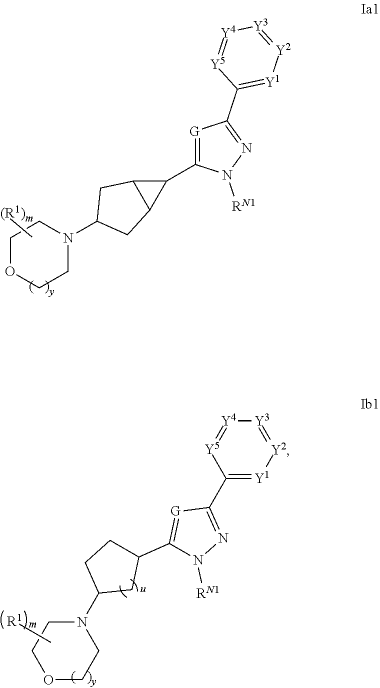

wherein:
u is 1 or 2;
m is 0, 1, or 2;
Y¹, Y², Y³, Y⁴, and Y⁵ are each independently CH, CR³, or N, provided that only one or two of Y¹, Y², Y³, Y⁴, and Y⁵ can be N;
G is N or CH; and
R¹, if present, in each instance, is independently selected from the group consisting of C₁-C₆ alkyl and halo-C₁-C₆ alkyl; or wherein two R¹ groups come together to form a —CH₂— or —CH₂CH₂— bridge.

In certain embodiments, compounds include those of Formula Ib, 1b′, or Ib1, or pharmaceutically acceptable salts thereof, where u is 1. In certain embodiments, compounds include those of Formula Ib, Ib′, or Ib1, or pharmaceutically acceptable salts thereof, where u is 2.

In certain embodiments, compounds include those of Formula I, Ia, 1a′, Ib, 1b′, Ia1, or Ib1, or pharmaceutically acceptable salts thereof, where R^{N1} is selected from the group consisting of C₁-C₆ alkyl and C₃-C₅ cycloalkyl. In certain embodiments, compounds include those of Formula I, Ia, 1a′, Ib, 1b′, Ia1, or Ib1, or pharmaceutically acceptable salts thereof, where R^{N1} is selected from the group consisting of methyl, ethyl, propyl, butyl, and cyclopropyl. In certain embodiments, compounds include those of Formula I, Ia, 1a′, Ib, 1b′, Ia1, or Ib1, or pharmaceutically acceptable salts thereof, where R^{N1} is propyl. In certain embodiments, compounds include those of Formula I, Ia, 1a′, Ib, 1b′, Ia1, or Ib1, or pharmaceutically acceptable salts thereof, where R^{N1} is isopropyl.

In certain embodiments, compounds include those of Formula I, Ia, 1a′, Ib, 1b′, Ia1, or Ib1, or pharmaceutically acceptable salts thereof, where y is 1. In certain embodiments, compounds include those of Formula I, Ia, 1a′, Ib, 1b′, Ia1, or Ib1, or pharmaceutically acceptable salts thereof, where y is 2.

In certain embodiments, compounds include those of Formula I, Ia, 1a′, Ib, 1b′, Ia1, or Ib1, or pharmaceutically acceptable salts thereof, where R¹ in each instance is selected from the group consisting of methyl, ethyl, and —CH₂F.

In certain embodiments, compounds include those of Formula I, Ia, 1a′, Ib, 1b′, Ia1, or Ib1, or pharmaceutically acceptable salts thereof, where m is 0. In certain embodiments, compounds include those of Formula I, Ia, 1a′, Ib, 1b′, Ia1, or Ib1, or pharmaceutically acceptable salts thereof, where m is 1.

In certain embodiments, compounds include those of Formula I, Ia, Ib, Ia1, or Ib1, or pharmaceutically acceptable salts thereof, where Y¹ is CH, Y² is CR³, Y³ is CH, Y⁴ is N, and Y⁵ is CH; Y¹ is CH, Y² is N, Y³ is CR³, Y⁴ is CH, and Y⁵ is CH; Y¹ is N, Y² is CR³, Y³ is CH, Y⁴ is CH, and Y⁵ is CH; Y¹ is CH, Y² is CR³, Y³ is CH, Y⁴ is CH, and Y⁵ is CH; Y¹ is CH, Y² is CR³, Y³ is CH, Y⁴ is CH, and Y⁵ is N; Y¹ is CH, Y² is N, Y³ is CR³, Y⁴ is N, and Y⁵ is CH; Y¹ is N, Y² is CR³, Y³ is N, Y⁴ is CH, and Y⁵ is CH; Y¹ is CH, Y² is CR³, Y³ is N, Y⁴ is CH, and Y⁵ is N; Y¹ is N, Y² is CR³, Y³ is CH, Y⁴ is N, and Y⁵ is CH; Y¹ is CH, Y² is CR³, Y³ is CR³, Y⁴ is CH, and Y⁵ is CH; Y¹ is CH, Y² is N, Y³ is CR³, Y⁴ is CR³, and Y⁵ is CH; Y¹ is CH, Y² is CR³, Y³ is N, Y⁴ is CH, and Y⁵ is CH; Y¹ is CH, Y² is CH, Y³ is CR³, Y⁴ is CH, and Y⁵ is CH; Y¹ is N, Y² is CH, Y³ is CR³, Y⁴ is CH, and Y⁵ is CH; or, Y¹ is CR³, Y² is CH, Y³ is CH, Y⁴ is CH, and Y⁵ is CH.

In certain embodiments, compounds include those of Formula I, Ia, 1a′, Ib, 1b′, Ia1, or Ib1, or pharmaceutically acceptable salts thereof, where R³ is selected from the group consisting of halo-C₁-C₆ alkyl, halo, cyano, C₁-C₆ alkyl, and halo-C₁-C₆ alkoxy. In certain embodiments, compounds include those of Formula I, Ia, 1a′, Ib, 1b′, Ia1, or Ib1, or pharmaceutically acceptable salts thereof, where R³ is selected from the group consisting of —CF₃, —CHF₂, methyl, fluoro, chloro, cyano, —OCF₃, and —OCHF₂. In certain embodiments, compounds include those of Formula I, Ia, 1a′, Ib, 1b′, Ia1, or Ib1, or pharmaceutically acceptable salts thereof, where R³ is —CF₃.

In certain embodiments, compounds include those of Formula I, Ia, 1a′, Ib, 1b′, Ia1, or Ib1, or pharmaceutically acceptable salts thereof, where at least one R³ is halo. In certain embodiments, compounds include those of Formula I, Ia, 1a′, Ib, 1b′, Ia1, or Ib1, or pharmaceutically acceptable salts thereof, where at least one of R³ is fluoro.

In certain embodiments, compounds of Formula I, or pharmaceutically acceptable salts thereof, include compounds of Formula Ia2 or Formula 1b2, or pharmaceutically acceptable salts thereof

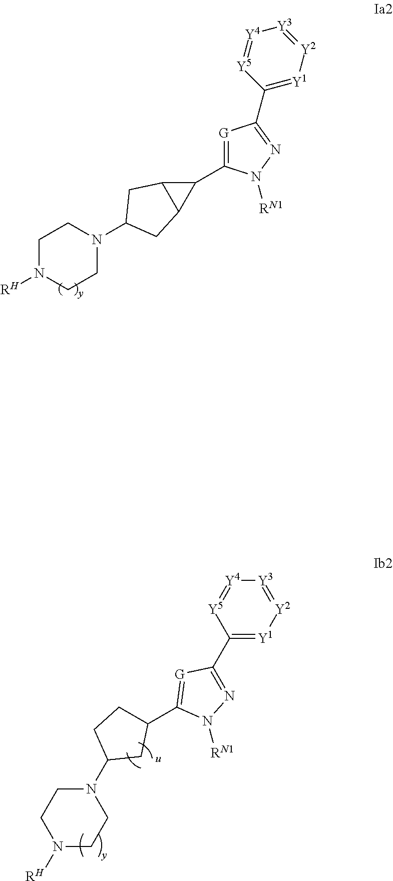

wherein:
u is 1 or 2;
Y¹, Y², Y³, Y⁴, and Y⁵ are each independently CH, CR³, or N, provided that only one or two of Y¹, Y², Y³, Y⁴, and Y⁵ can be N; and
G is N or CH.

In certain embodiments, compounds include those of Formula Ib, 1b′, Ib1, or Ib2, or pharmaceutically acceptable salts thereof, where u is 1.

In certain embodiments, compounds include those of Formula I, Ia, 1a′, Ia1, Ia2, Ib, 1b′, Ib1, or Ib2, or pharmaceutically acceptable salts thereof, where y is 1.

In certain embodiments, compounds include those of Formula I, Ia, 1a′, Ia1, Ia2, Ib, 1b′, Ib1, or Ib2, or pharmaceutically acceptable salts thereof, where R^{H} is selected from the group consisting of C₃-C₅ cycloalkyl, hydroxy-C₁-C₆ alkyl, and C₁-C₆ alkoxy-C₁-C₆ alkyl. In certain embodiments, compounds include those of Formula I, Ia, Ia2, Ib, or Ib2, or pharmaceutically acceptable salts thereof, where R^{H} is selected from the group consisting of —CH₂CH₂OCH₃, cyclobutyl, and —CH₂CH₂OH.

In certain embodiments, compounds include those of Formula I, Ia, 1a′, Ia1, Ia2, Ib, 1b′, Ib1, or Ib2, or pharmaceutically acceptable salts thereof, where R^{N1} is C₁-C₆ alkyl. In certain embodiments, compounds include those of Formula I, Ia, 1a′, Ia1, Ia2, Ib, 1b′, Ib1, or Ib2, or pharmaceutically acceptable salts thereof, where R^{N1} is selected from the group consisting of methyl, ethyl, propyl, and butyl. In certain embodiments, compounds include those of Formula I, Ia, 1a′, Ia1, Ia2, Ib, 1b′, Ib1, or Ib2, or pharmaceutically acceptable salts thereof, where R^{N1} is propyl. In certain embodiments, compounds include those of Formula I, Ia, 1a′, Ia1, Ia2, Ib, 1b′, Ib1, or Ib2, or pharmaceutically acceptable salts thereof, where R^{N1} is isopropyl.

In certain embodiments, compounds include those of Formula I, Ia, Ia1, Ia2, Ib, Ib1, or Ib2, or pharmaceutically acceptable salts thereof, where Y¹ is CH, Y² is N, Y³ is CR³, Y⁴ is CH, and Y⁵ is CH; or, Y¹ is N, Y² is CR³, Y³ is CH, Y⁴ is CH, and Y⁵ is CH. In certain embodiments, compounds include those of Formula I, Ia, Ia1, Ia2, Ib, Ib1, or Ib2, or pharmaceutically acceptable salts thereof, where R³ is halo-C₁-C₆ alkyl. In certain embodiments, compounds include those of Formula I, Ia, Ia1, Ia2, Ib, Ib1, or Ib2, or pharmaceutically acceptable salts thereof, where R³ is —CF₃.

In certain embodiments, compounds include those of Formula I, Ia, Ia′, Ia1, or Ia2, or pharmaceutically acceptable salts thereof, where Ring A is a bicyclo[3.1.0]hexanyl having the following structure:

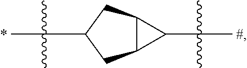

wherein # is the point of attachment to the C on Ring D, and * is the point of attachment to the N on Ring C.

In certain embodiments, compounds include those of Formula I, Ia, Ia′, Ia1, or Ia2, or pharmaceutically acceptable salts thereof, where Ring A is a bicyclo[3.1.0]hexanyl having the following structure:

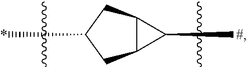

wherein # is the point of attachment to the C on Ring D, and * is the point of attachment to the N on Ring C.

In certain embodiments, compounds include those of Formula I, Ia, Ia′, Ia1, or Ia2, or pharmaceutically acceptable salts thereof, where Ring A is a bicyclo[3.1.0]hexanyl having the following structure:

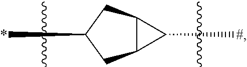

wherein # is the point of attachment to the C on Ring D, and * is the point of attachment to the N on Ring C.

In certain embodiments, compounds include those of Formula I, Ia, Ia′, Ia1, or Ia2, or pharmaceutically acceptable salts thereof, where Ring A is a bicyclo[3.1.0]hexanyl having the following structure:

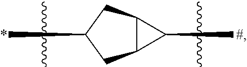

wherein # is the point of attachment to the C on Ring D, and * is the point of attachment to the N on Ring C.

In certain embodiments, compounds include those of Formula I, Ia, Ia′, Ia1, or Ia2, or pharmaceutically acceptable salts thereof, where Ring A is a bicyclo[3.1.0]hexanyl having the following structure:

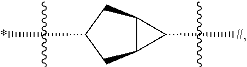

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclohexyl having the following structure:

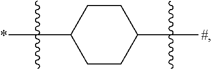

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclohexyl having the following structure:

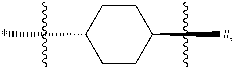

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclohexyl having the following structure:

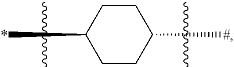

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclohexyl having the following structure:

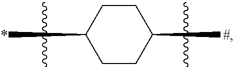

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclohexyl having the following structure:

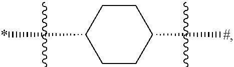

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclopentyl having the following structure:

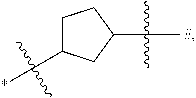

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclopentyl having the following structure:

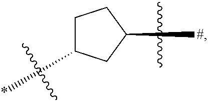

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclopentyl having the following structure:

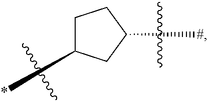

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclopentyl having the following structure:

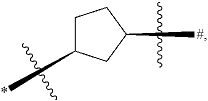

wherein # is the point of attachment to the C on Ring D, and * is the point of attachment to the N on Ring C.

In certain embodiments, compounds include those of Formula I, Ib, Ib′, Ib1, or Ib2, or pharmaceutically acceptable salts thereof, where Ring A is a cyclopentyl having the following structure:

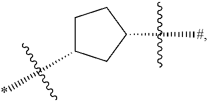

wherein # is the point of attachment to the C on Ring D, and * is the point of attachment to the N on Ring C.

In certain embodiments, the compounds include those of the following formulae:

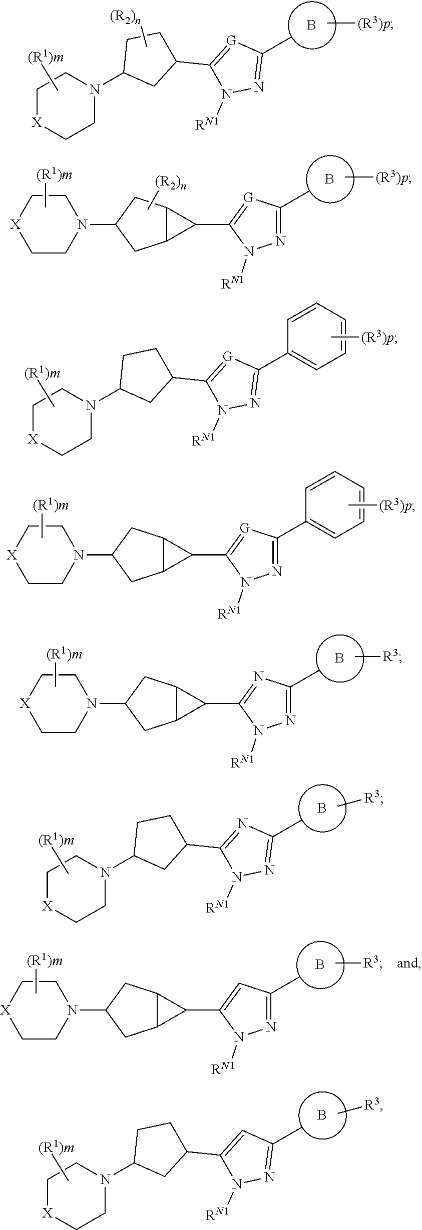

wherein, the variable are as described herein.

The subject matter described herein includes the following compounds in Table 1, or pharmaceutically acceptable salts thereof. In Table 1, the asterix (*) indicates an isolated isomer or isolated group of isomers, but that the stereochemistry has been arbitrarily assigned. Individual enantiomers and diastereomers are included in the table below by compound name, and their corresponding structures can be readily determined therefrom. In some instances, the enantiomers or diastereomers of the present disclosure may be identified by their respective properties, for example, retention times by chiral HPLC, NMR peaks, and/or biological activities (e.g., as described further in the Examples), and the absolute stereo configurations of one or more chiral centers are arbitrarily assigned (e.g., stereochemistry of all chiral centers is arbitrarily assigned, or stereochemistry of one chiral center is known and remaining chiral centers arbitrarily assigned, etc.).

TABLE 1

| Cmpd No. | Structure | Name |
|---|---|---|
| 1* | 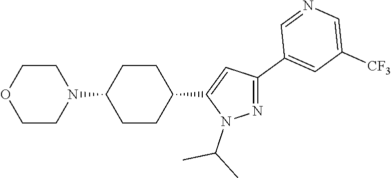 [SMILES: CC(C)n1nc(-c2cncc(C(F)(F)F)c2)cc1[C@H]1CC[C@@H](N2CCOCC2)CC1] | 4-((1s,4s)-4-(1-isopropyl-3- (5-(trifluoromethyl)pyridin- 3-yl)-1H-pyrazol-5-yl)cyclo- hexyl)morpholine |
| 2* | 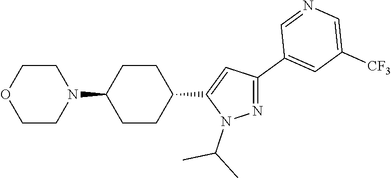 [SMILES: CC(C)n1nc(-c2cncc(C(F)(F)F)c2)cc1[C@H]1CC[C@H](N2CCOCC2)CC1] | 4-((1r,4r)-4-(1-isopropyl-3- (5-(trifluoromethyl)pyridin- 3-yl)-1H-pyrazol-5-yl)cyclo- hexyl)morpholine |
| 3 | 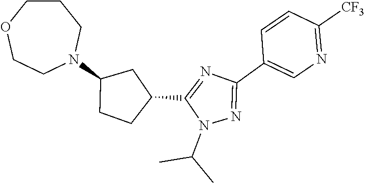 [SMILES: CC(C)n1nc(-c2ccc(C(F)(F)F)nc2)nc1[C@@H]1CC[C@@H](N2CCCOCC2)C1] | 4-((1R,3R)-3-(1-isopropyl-3- (6-(trifluoromethyl)pyridin- 3-yl)-1H-1,2,4-triazol-5-yl)- cyclopentyl)-1,4-oxazepane |
| 4 | 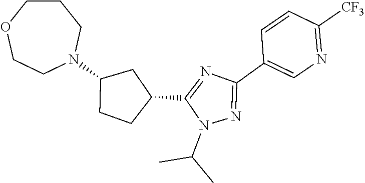 [SMILES: CC(C)n1nc(-c2ccc(C(F)(F)F)nc2)nc1[C@@H]1CC[C@H](N2CCCOCC2)C1] | 4-((1S,3R)-3-(1-isopropyl-3- (6-(trifluoromethyl)pyridin- 3-yl)-1H-1,2,4-triazol-5-yl)- cyclopentyl)-1,4-oxazepane |
| 5 | 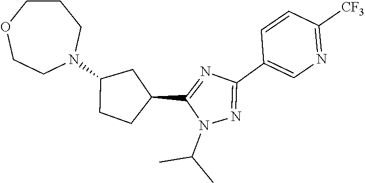 [SMILES: CC(C)n1nc(-c2ccc(C(F)(F)F)nc2)nc1[C@H]1CC[C@H](N2CCCOCC2)C1] | 4-((1S,3S)-3-(1-isopropyl-3- (6-(trifluoromethyl)pyridin- 3-yl)-1H-1,2,4-triazol-5-yl)- cyclopentyl)-1,4-oxazepane |
| 6 | 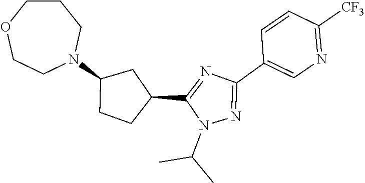 [SMILES: CC(C)n1nc(-c2ccc(C(F)(F)F)nc2)nc1[C@H]1CC[C@@H](N2CCCOCC2)C1] | 4-((1R,3S)-3-(1-isopropyl-3- (6-(trifluoromethyl)pyridin- 3-yl)-1H-1,2,4-triazol-5-yl)- cyclopentyl)-1,4-oxazepane |
| 7* | 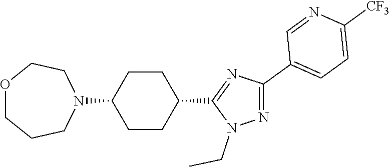 [SMILES: CCn1nc(-c2ccc(C(F)(F)F)nc2)nc1[C@H]1CC[C@@H](N2CCCOCC2)CC1] | 4-((1s,4s)-4-(1-ethyl-3-(6- (trifluoromethyl)pyridin-3- yl)-1H-1,2,4-triazol-5-yl)- cyclohexyl)-1,4-oxazepane |
| 8* | 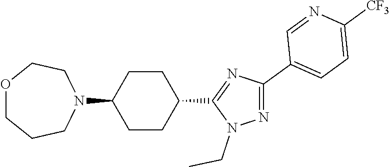 [SMILES: CCn1nc(-c2ccc(C(F)(F)F)nc2)nc1[C@H]1CC[C@H](N2CCCOCC2)CC1] | 4-((1r,4r)-4-(1-ethyl-3-(6- (trifluoromethyl)pyridin-3- yl)-1H-1,2,4-triazol-5-yl)- cyclohexyl)-1,4-oxazepane |
| 9* | 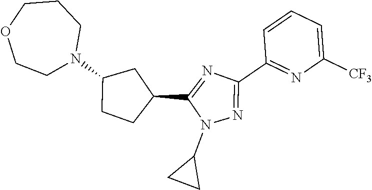 [SMILES: FC(F)(F)c1cccc(-c2nc([C@H]3CC[C@H](N4CCCOCC4)C3)n(C3CC3)n2)n1] | 4-((1S,3S)-3-(1-cycloprop- yl-3-(6-(trifluoromethyl)- pyridin-2-yl)-1H-1,2,4-tri- azol-5-yl)cyclopentyl)-1,4- oxazepane |
| 10* | 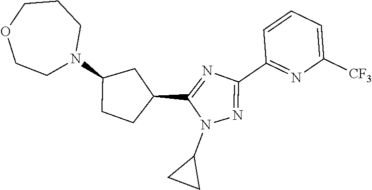 [SMILES: FC(F)(F)c1cccc(-c2nc([C@H]3CC[C@@H](N4CCCOCC4)C3)n(C3CC3)n2)n1] | 4-((1R,3S)-3-(1-cycloprop- yl-3-(6-(trifluoromethyl)- pyridin-2-yl)-1H-1,2,4-tri- azol-5-yl)cyclopentyl)-1,4- oxazepane |
| 11* | 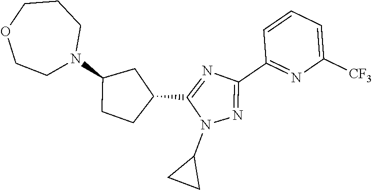 [SMILES: FC(F)(F)c1cccc(-c2nc([C@@H]3CC[C@@H](N4CCCOCC4)C3)n(C3CC3)n2)n1] | 4-((1R,3R)-3-(1-cyclopropyl- 3-(6-(trifluoromethyl)pyridin- 2-yl)-1H-1,2,4-triazol-5-yl)- cyclopentyl)-1,4-oxazepane |
| 12* | 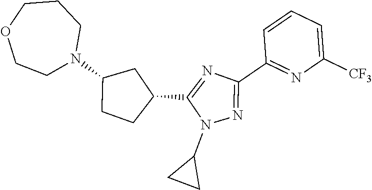 [SMILES: FC(F)(F)c1cccc(-c2nc([C@@H]3CC[C@H](N4CCCOCC4)C3)n(C3CC3)n2)n1] | 4-((1S,3R)-3-(1-cyclopropyl- 3-(6-(trifluoromethyl)pyridin- 2-yl)-1H-1,2,4-triazol-5-yl)- cyclopentyl)-1,4-oxazepane |
| 13 | 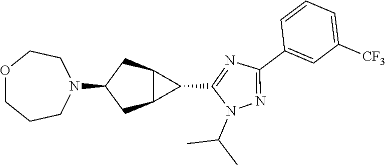 [SMILES: CC(C)n1nc(-c2cccc(C(F)(F)F)c2)nc1[C@H]1[C@@H]2C[C@H](N3CCCOCC3)C[C@@H]21] | 4-((1R,3r,5S,6r)-6-(1-iso- propyl-3-(3-(trifluorometh- yl)phenyl)-1H-1,2,4-triazol- 5-yl)bicyclo[3.1.0]hexan-3- yl)-1,4-oxazepane |
| 14 | 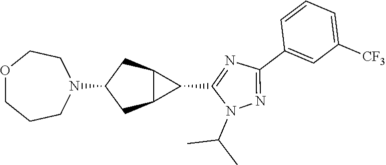 [SMILES: CC(C)n1nc(-c2cccc(C(F)(F)F)c2)nc1[C@H]1[C@@H]2C[C@@H](N3CCCOCC3)C[C@@H]21] | 4-((1R,3s,5S,6r)-6-(1-iso- propyl-3-(3-(trifluorometh- yl)phenyl)-1H-1,2,4-triazol- 5-yl)bicyclo[3.1.0]hexan-3- yl)-1,4-oxazepane |
| 15 | 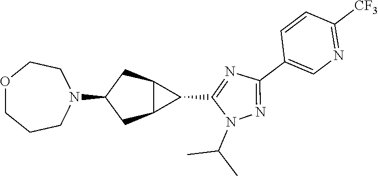 [SMILES: CC(C)n1nc(-c2ccc(C(F)(F)F)nc2)nc1[C@H]1[C@@H]2C[C@H](N3CCCOCC3)C[C@@H]21] | 4-((1R,3r,5S,6r)-6-(1-iso- propyl-3-(6-(trifluorometh- yl)pyridin-3-yl)-1H-1,2,4- triazol-5-yl)bicyclo[3.1.0] hexan-3-yl)-1,4-oxazepane |
| 16 | 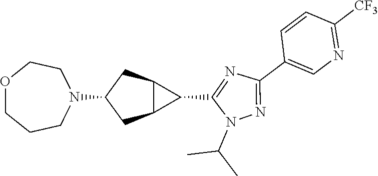 [SMILES: CC(C)n1nc(-c2ccc(C(F)(F)F)nc2)nc1[C@H]1[C@@H]2C[C@@H](N3CCCOCC3)C[C@@H]21] | 4-((1R,3s,5S,6r)-6-(1-iso- propyl-3-(6-(trifluorometh- yl)pyridin-3-yl)-1H-1,2,4- triazol-5-yl)bicyclo[3.1.0] hexan-3-yl)-1,4-oxazepane |
| 17 | 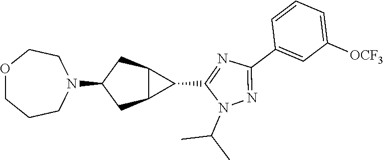 [SMILES: CC(C)n1nc(-c2cccc(OC(F)(F)F)c2)nc1[C@H]1[C@@H]2C[C@H](N3CCCOCC3)C[C@@H]21] | 4-((1R,3r,5S,6r)-6-(1-iso- propyl-3-(3-(trifluorometh- oxy)phenyl)-1H-1,2,4-tri- azol-5-yl)bicyclo[3.1.0] hexan-3-yl)-1,4-oxazepane |
| 18 | 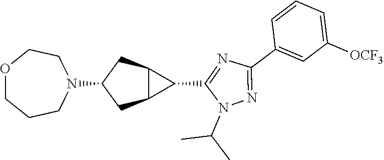 [SMILES: CC(C)n1nc(-c2cccc(OC(F)(F)F)c2)nc1[C@H]1[C@@H]2C[C@@H](N3CCCOCC3)C[C@@H]21] | 4-((1R,3s,5S,6r)-6-(1-iso- propyl-3-(3-(trifluorometh- oxy)phenyl)-1H-1,2,4-tri- azol-5-yl)bicyclo[3.1.0] hexan-3-yl)-1,4-oxazepane |
| 19* | 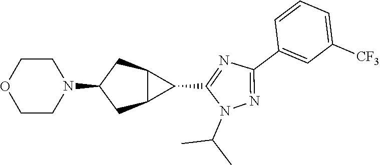 [SMILES: CC(C)n1nc(-c2cccc(C(F)(F)F)c2)nc1[C@H]1[C@@H]2C[C@H](N3CCOCC3)C[C@@H]21] | 4-((1R,3r,5S,6r)-6-(1-iso- propyl-3-(3-(trifluorometh- yl)phenyl)-1H-1,2,4-triazol- 5-yl)bicyclo[3.1.0]hexan-3- yl)morpholine |
| 20* | 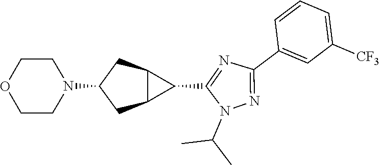 [SMILES: CC(C)n1nc(-c2cccc(C(F)(F)F)c2)nc1[C@H]1[C@@H]2C[C@@H](N3CCOCC3)C[C@@H]21] | 4-((1R,3s,5S,6r)-6-(1-iso- propyl-3-(3-(trifluorometh- yl)phenyl)-1H-1,2,4-triazol- 5-yl)bicyclo[3.1.0]hexan-3- yl)morpholine |
| 21* | 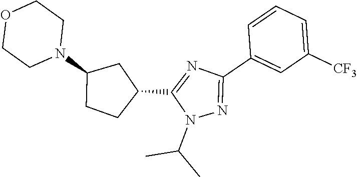 [SMILES: CC(C)n1nc(-c2cccc(C(F)(F)F)c2)nc1[C@@H]1CC[C@@H](N2CCOCC2)C1] | 4-((1R,3R)-3-(1-isopropyl-3- (3-(trifluoromethyl)phenyl)- 1H-1,2,4-triazol-5-yl)cyclo- pentyl)morpholine |
| 22* | 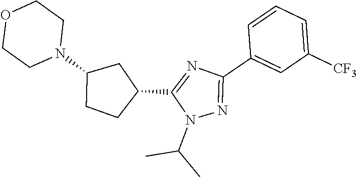 [SMILES: CC(C)n1nc(-c2cccc(C(F)(F)F)c2)nc1[C@@H]1CC[C@H](N2CCOCC2)C1] | 4-((1S,3R)-3-(1-isopropyl-3- (3-(trifluoromethyl)phenyl)- 1H-1,2,4-triazol-5-yl)cyclo- pentyl)morpholine |
| 23* | 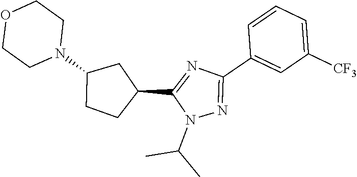 [SMILES: CC(C)n1nc(-c2cccc(C(F)(F)F)c2)nc1[C@H]1CC[C@H](N2CCOCC2)C1] | 4-((1S,3S)-3-(1-isopropyl-3- (3-(trifluoromethyl)phenyl)- 1H-1,2,4-triazol-5-yl)cyclo- pentyl)morpholine |
| 24* | 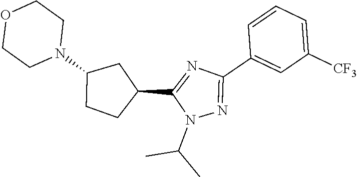 [SMILES: CC(C)n1nc(-c2cccc(C(F)(F)F)c2)nc1[C@H]1CC[C@H](N2CCOCC2)C1] | 4-((1R,3S)-3-(1-isopropyl-3- (3-(trifluoromethyl)phenyl)- 1H-1,2,4-triazol-5-yl)cyclo- pentyl)morpholine |
| 25* | 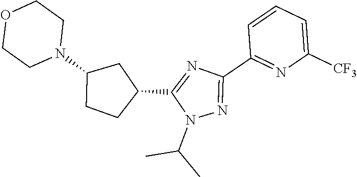 [SMILES: CC(C)n1nc(-c2cccc(C(F)(F)F)n2)nc1[C@@H]1CC[C@H](N2CCOCC2)C1] | 4-((1R,3R)-3-(1-isopropyl-3- (6-(trifluoromethyl)pyridin- 2-yl)-1H-1,2,4-triazol-5-yl)- cyclopentyl)morpholine |
| 26* | 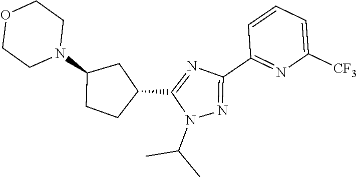 [SMILES: CC(C)n1nc(-c2cccc(C(F)(F)F)n2)nc1[C@@H]1CC[C@@H](N2CCOCC2)C1] | 4-((1S,3R)-3-(1-isopropyl-3- (6-(trifluoromethyl)pyridin- 2-yl)-1H-1,2,4-triazol-5-yl)- cyclopentyl)morpholine |
| 27* | 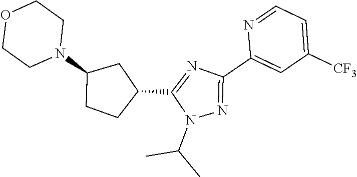 [SMILES: CC(C)n1nc(-c2cc(C(F)(F)F)ccn2)nc1[C@@H]1CC[C@@H](N2CCOCC2)C1] | 4-((1R,3R)-3-(1-isopropyl-3- (4-(trifluoromethyl)pyridin- 2-yl)-1H-1,2,4-triazol-5-yl)- cyclopentyl)morpholine |
| 28* | 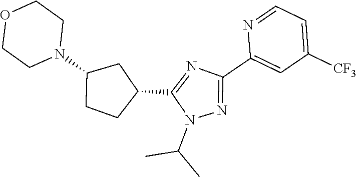 [SMILES: CC(C)n1nc(-c2cc(C(F)(F)F)ccn2)nc1[C@@H]1CC[C@H](N2CCOCC2)C1] | 4-((1S,3R)-3-(1-isopropyl-3- (4-(trifluoromethyl)pyridin- 2-yl)-1H-1,2,4-triazol-5-yl)- cyclopentyl)morpholine |
| 29* | 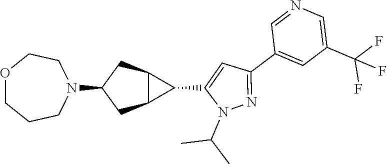 [SMILES: CC(C)n1nc(-c2cncc(C(F)(F)F)c2)cc1[C@H]1[C@@H]2C[C@H](N3CCCOCC3)C[C@@H]21] | 4-((1R,3r,5S,6r)-6-(1-iso- propyl-3-(5-(trifluorometh- yl)pyridin-3-yl)-1H-pyrazol- 5-yl)bicyclo[3.1.0]hexan-3- yl)-1,4-oxazepane |
| 30* | 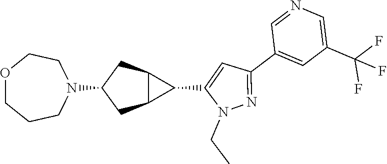 [SMILES: CCn1nc(-c2cncc(C(F)(F)F)c2)cc1[C@H]1[C@@H]2C[C@@H](N3CCCOCC3)C[C@@H]21] | 4-((1R,3s,5S,6r)-6-(1-iso- propyl-3-(5-(trifluorometh- yl)pyridin-3-yl)-1H-pyrazol- 5-yl)bicyclo[3.1.0]hexan-3- yl)-1,4-oxazepane |
| 31 | 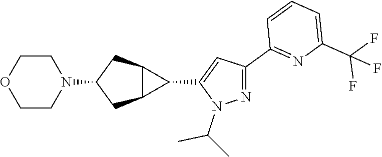 [SMILES: CC(C)n1nc(-c2cccc(C(F)(F)F)n2)cc1[C@H]1[C@@H]2C[C@@H](N3CCOCC3)C[C@@H]21] | 4-((1R,3s,5S,6r)-6-(1-iso- propyl-3-(6-(trifluorometh- yl)pyridin-2-yl)-1H-pyrazol- 5-yl)bicyclo[3.1.0]hexan-3- yl)morpholine |
| 32 | 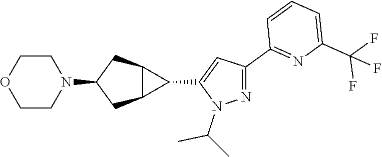 [SMILES: CC(C)n1nc(-c2cccc(C(F)(F)F)n2)cc1[C@H]1[C@@H]2C[C@H](N3CCOCC3)C[C@@H]21] | 4-((1R,3r,5S,6r)-6-(1-iso- propyl-3-(6-(trifluorometh- yl)pyridin-2-yl)-1H-pyrazol- 5-yl)bicyclo[3.1.0]hexan-3- yl)morpholine |
| 33 | 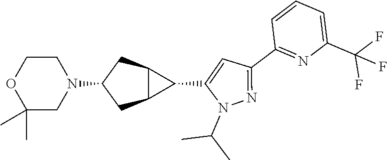 [SMILES: CC(C)n1nc(-c2cccc(C(F)(F)F)n2)cc1[C@H]1[C@@H]2C[C@@H](N3CCOC(C)(C)C3)C[C@@H]21] | 4-((1R,3s,5S,6r)-6-(1-iso- propyl-3-(5-(trifluorometh- yl)pyridin-3-yl)-1H-pyrazol- 5-yl)bicyclo[3.1.0]hexan-3- yl)-2,2-dimethylmorpholine |
| 34 | 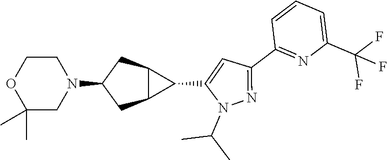 [SMILES: CC(C)n1nc(-c2cccc(C(F)(F)F)n2)cc1[C@H]1[C@@H]2C[C@H](N3CCOC(C)(C)C3)C[C@@H]21] | 4-((1R,3r,5S,6r)-6-(1-iso- propyl-3-(5-(trifluorometh- yl)pyridin-3-yl)-1H-pyrazol- 5-yl)bicyclo[3.1.0]hexan-3- yl)-2,2-dimethylmorpholine |
| 35* | 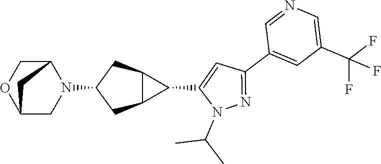 [SMILES: CC(C)n1nc(-c2cncc(C(F)(F)F)c2)cc1[C@H]1[C@@H]2C[C@@H](N3C[C@H]4C[C@@H]3CO4)C[C@@H]21] | (1R,4R)-5-((1R,3s,5S,6R)-6- (1-isopropyl-3-(5-(trifluoro- methyl)pyridin-3-yl)-1H- pyrazol-5-yl)bicyclo[3.1.0] hexan-3-yl)-2-oxa-5-azabi- cyclo[2.2.1]heptane |
| 36* | 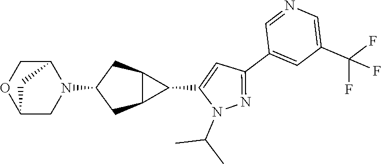 [SMILES: CC(C)n1nc(-c2cncc(C(F)(F)F)c2)cc1[C@H]1[C@@H]2C[C@@H](N3C[C@@H]4C[C@H]3CO4)C[C@@H]21] | (1S,4R)-5-((1R,3s,5S,6R)-6- (1-isopropyl-3-(5-(trifluoro- methyl)pyridin-3-yl)-1H- pyrazol-5-yl)bicyclo[3.1.0] hexan-3-yl)-2-oxa-5-azabi- cyclo[2.2.1]heptane |
| 37* | 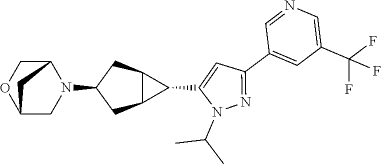 [SMILES: CC(C)n1nc(-c2cncc(C(F)(F)F)c2)cc1[C@H]1[C@@H]2C[C@H](N3C[C@H]4C[C@@H]3CO4)C[C@@H]21] | (1R,4R)-5-((1R,3r,5S,6R)-6- (1-isopropyl-3-(5-(trifluoro- methyl)pyridin-3-yl)-1H- pyrazol-5-yl)bicyclo[3.1.0] hexan-3-yl)-2-oxa-5-azabi- cyclo[2.2.1]heptane |
| 38* | 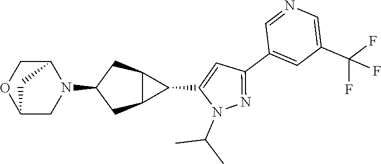 [SMILES: CC(C)n1nc(-c2cncc(C(F)(F)F)c2)cc1[C@H]1[C@@H]2C[C@H](N3C[C@@H]4C[C@H]3CO4)C[C@@H]21] | (1S,4R)-5-((1R,3r,5S,6R)-6- (1-isopropyl-3-(5-(trifluoro- methyl)pyridin-3-yl)-1H- pyrazol-5-yl)bicyclo[3.1.0] hexan-3-yl)-2-oxa-5-azabi- cyclo[2.2.1]heptane |
| 39* | 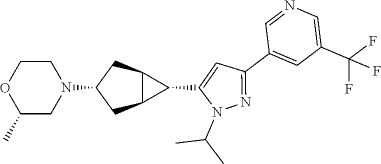 [SMILES: CC(C)n1nc(-c2cncc(C(F)(F)F)c2)cc1[C@H]1[C@@H]2C[C@@H](N3CCO[C@@H](C)C3)C[C@@H]21] | (S)-4-((1R,3s,5S,6S)-6-(1- isopropyl-3-(5-(trifluoro- methyl)pyridin-3-yl)-1H- pyrazol-5-yl)bicyclo[3.1.0] hexan-3-yl)-2-methylmor- pholine |
| 40* | 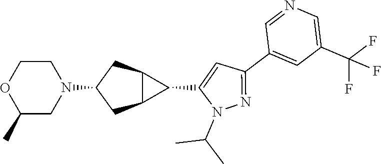 [SMILES: CC(C)n1nc(-c2cncc(C(F)(F)F)c2)cc1[C@H]1[C@@H]2C[C@@H](N3CCO[C@H](C)C3)C[C@@H]21] | (R)-4-((1R,3s,5S,6R)-6-(1- isopropyl-3-(5-(trifluoro- methyl)pyridin-3-yl)-1H- pyrazol-5-yl)bicyclo[3.1.0] hexan-3-yl)-2-methylmor- pholine |
| 41* | 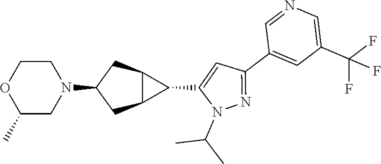 [SMILES: CC(C)n1nc(-c2cncc(C(F)(F)F)c2)cc1[C@H]1[C@@H]2C[C@H](N3CCO[C@@H](C)C3)C[C@@H]21] | (S)-4-((1R,3r,5S,6S)-6-(1- isopropyl-3-(5-(trifluoro- methyl)pyridin-3-yl)-1H- pyrazol-5-yl)bicyclo[3.1.0] hexan-3-yl)-2-methylmor- pholine |
| 42* | 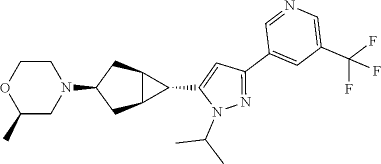 [SMILES: CC(C)n1nc(-c2cncc(C(F)(F)F)c2)cc1[C@H]1[C@@H]2C[C@H](N3CCO[C@H](C)C3)C[C@@H]21] | (R)-4-((1R,3r,5S,6R)-6-(1- isopropyl-3-(5-(trifluoro- methyl)pyridin-3-yl)-1H- pyrazol-5-yl)bicyclo[3.1.0] hexan-3-yl)-2-methylmor- pholine |
| 43* | 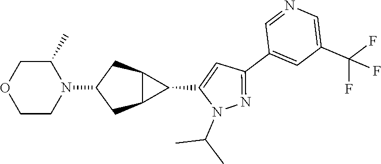 | (S)-4-((1R,3s,5S,6S)-6-(1- isopropyl-3-(5-(trifluoro- methyl)pyridin-3-yl)-1H- pyrazol-5-yl)bicyclo[3.1.0] hexan-3-yl)-3-methylmor- pholine |
| 44* | 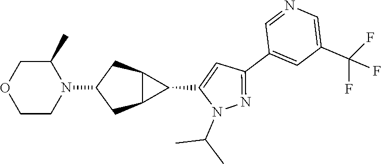 | (R)-4-((1R,3s,5S,6R)-6-(1- isopropyl-3-(5-(trifluorometh- yl)pyridin-3-yl)-1H-pyrazol- 5-yl)bicyclo[3.1.0]hexan-3- yl)-3-methylmorpholine |
| 45* | 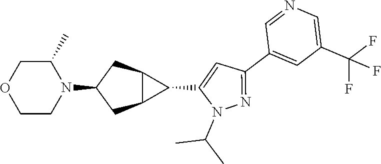 [SMILES: CC(C)n1nc(-c2cncc(C(F)(F)F)c2)cc1[C@H]1[C@@H]2C[C@H](N3CCOC[C@@H]3C)C[C@@H]21] | (S)-4-((1R,3r,5S,6S)-6-(1- isopropyl-3-(5-(trifluorometh- yl)pyridin-3-yl)-1H-pyrazol- 5-yl)bicyclo[3.1.0]hexan-3- yl)-3-methylmorpholine |
| 46* | 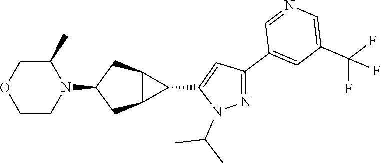 [SMILES: CC(C)n1nc(-c2cncc(C(F)(F)F)c2)cc1[C@H]1[C@@H]2C[C@H](N3CCOC[C@H]3C)C[C@@H]21] | (R)-4-((1R,3r,5S,6R)-6-(1- isopropyl-3-(5-(trifluorometh- yl)pyridin-3-yl)-1H-pyrazol- 5-yl)bicyclo[3.1.0]hexan-3- yl)-3-methylmorpholine |
| 47 | 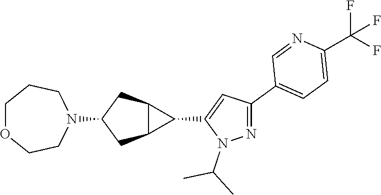 [SMILES: CC(C)n1nc(-c2ccc(C(F)(F)F)nc2)cc1[C@H]1[C@@H]2C[C@@H](N3CCCOCC3)C[C@@H]21] | 4-((1R,3S,5S,6R)-6-(1-iso- propyl-3-(6-(trifluorometh- yl)pyridin-3-yl)-1H-pyrazol- 5-yl)bicyclo[3.1.0]hexan-3- yl)-1,4-oxazepane |
| 48 | 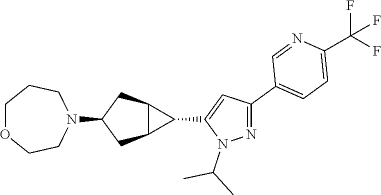 [SMILES: CC(C)n1nc(-c2ccc(C(F)(F)F)nc2)cc1[C@H]1[C@@H]2C[C@H](N3CCCOCC3)C[C@@H]21] | 4-((1R,3R,5S,6R)-6-(1-iso- propyl-3-(6-(trifluorometh- yl)pyridin-3-yl)-1H-pyrazol- 5-yl)bicyclo[3.1.0]hexan-3- yl)-1,4-oxazepane |
| 49 | 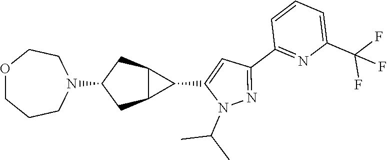 [SMILES: CC(C)n1nc(-c2cccc(C(F)(F)F)n2)cc1[C@H]1[C@@H]2C[C@@H](N3CCCOCC3)C[C@@H]21] | 4-((1R,3s,5S,6r)-6-(1-iso- propyl-3-(6-(trifluorometh- yl)pyridin-2-yl)-1H-pyrazol- 5-yl)bicyclo[3.1.0]hexan-3- yl)-1,4-oxazepane |
| 50 | 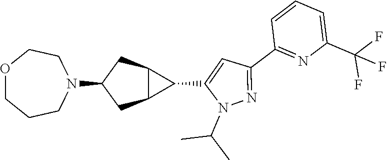 [SMILES: CC(C)n1nc(-c2cccc(C(F)(F)F)n2)cc1[C@H]1[C@@H]2C[C@H](N3CCCOCC3)C[C@@H]21] | 4-((1R,3r,5S,6r)-6-(1-iso- propyl-3-(6-(trifluorometh- yl)pyridin-2-yl)-1H-pyrazol- 5-yl)bicyclo[3.1.0]hexan-3- yl)-1,4-oxazepane |
| 51 | 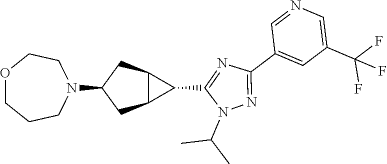 [SMILES: CC(C)n1nc(-c2cncc(C(F)(F)F)c2)nc1[C@H]1[C@@H]2C[C@H](N3CCCOCC3)C[C@@H]21] | 4-((1R,3R,5S,6R)-6-(1-iso- propyl-3-(5-(trifluorometh- yl)pyridin-3-yl)-1H-1,2,4- triazol-5-yl)bicyclo[3.1.0] hexan-3-yl)-1,4-oxazepane |
| 52 | 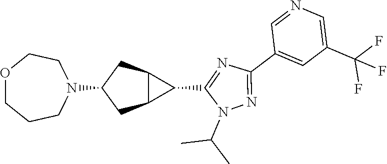 [SMILES: CC(C)n1nc(-c2cncc(C(F)(F)F)c2)nc1[C@H]1[C@@H]2C[C@@H](N3CCCOCC3)C[C@@H]21] | 4-((1R,3S,5S,6R)-6-(1-iso- propyl-3-(5-(trifluorometh- yl)pyridin-3-yl)-1H-1,2,4- triazol-5-yl)bicyclo[3.1.0] hexan-3-yl)-1,4-oxazepane |
| 53 | 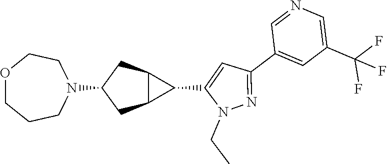 [SMILES: CCn1nc(-c2cncc(C(F)(F)F)c2)cc1[C@H]1[C@@H]2C[C@@H](N3CCCOCC3)C[C@@H]21] | 4-((1R,3s,5S,6r)-6-(1-ethyl- 3-(5-(trifluoromethyl)pyridin- 3-yl)-1H-pyrazol-5-yl)bicyclo [3.1.0]hexan-3-yl)-1,4- oxazepane |
| 54 | 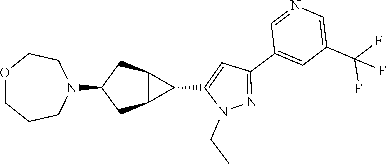 [SMILES: CCn1nc(-c2cncc(C(F)(F)F)c2)cc1[C@H]1[C@@H]2C[C@H](N3CCCOCC3)C[C@@H]21] | 4-((1R,3r,5S,6r)-6-(1-ethyl- 3-(5-(trifluoromethyl)pyr- idin-3-yl)-1H-pyrazol-5-yl)- bicyclo[3.1.0]hexan-3-yl)- 1,4-oxazepane |
| 58* | 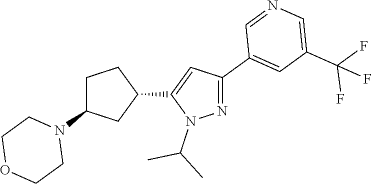 [SMILES: CC(C)n1nc(-c2cncc(C(F)(F)F)c2)cc1[C@H]1CC[C@H](N2CCOCC2)C1] | 4-((1S,3S)-3-(1-isopropyl-3- (5-(trifluoromethyl)pyridin- 3-yl)-1H-pyrazol-5-yl)cyclo- pentyl)morpholine |
| 59* | 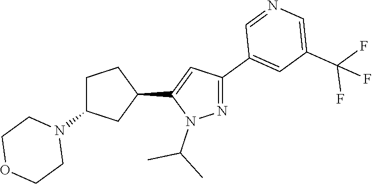 [SMILES: CC(C)n1nc(-c2cncc(C(F)(F)F)c2)cc1[C@@H]1CC[C@@H](N2CCOCC2)C1] | 4-((1R,3R)-3-(1-isopropyl-3- (5-(trifluoromethyl)pyridin- 3-yl)-1H-pyrazol-5-yl)cyclo- pentyl)morpholine |
| 60* | 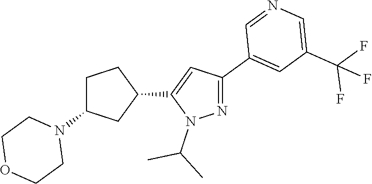 [SMILES: CC(C)n1nc(-c2cncc(C(F)(F)F)c2)cc1[C@H]1CC[C@@H](N2CCOCC2)C1] | 4-((1R,3S)-3-(1-isopropyl-3- (5-(trifluoromethyl)pyridin- 3-yl)-1H-pyrazol-5-yl)cyclo- pentyl)morpholine |
| 61* | 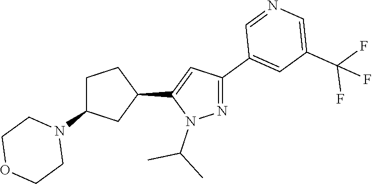 [SMILES: CC(C)n1nc(-c2cncc(C(F)(F)F)c2)cc1[C@@H]1CC[C@H](N2CCOCC2)C1] | 4-((1S,3R)-3-(1-isopropyl-3- (5-(trifluoromethyl)pyridin- 3-yl)-1H-pyrazol-5-yl)cyclo- pentyl)morpholine |
| 62* | 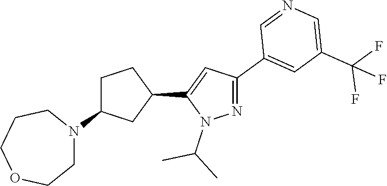 [SMILES: CC(C)n1nc(-c2cncc(C(F)(F)F)c2)cc1[C@@H]1CC[C@H](N2CCCOCC2)C1] | 4-((1S,3S)-3-(1-isopropyl-3- (5-(trifluoromethyl)pyridin- 3-yl)-1H-pyrazol-5-yl)cyclo- pentyl)-1,4-oxazepane |
| 63* | 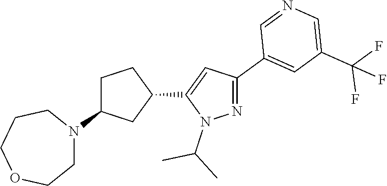 [SMILES: CC(C)n1nc(-c2cncc(C(F)(F)F)c2)cc1[C@H]1CC[C@H](N2CCCOCC2)C1] | 4-((1R,3R)-3-(1-isopropyl-3- (5-(trifluoromethyl)pyridin- 3-yl)-1H-pyrazol-5-yl)cyclo- pentyl)-1,4-oxazepane |
| 64* | 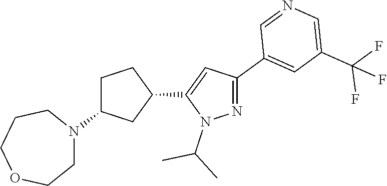 [SMILES: CC(C)n1nc(-c2cncc(C(F)(F)F)c2)cc1[C@H]1CC[C@@H](N2CCCOCC2)C1] | 4-((1R,3S)-3-(1-isopropyl-3- (5-(trifluoromethyl)pyridin- 3-yl)-1H-pyrazol-5-yl)cyclo- pentyl)-1,4-oxazepane |
| 65* | 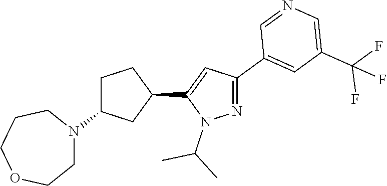 [SMILES: CC(C)n1nc(-c2cncc(C(F)(F)F)c2)cc1[C@@H]1CC[C@@H](N2CCCOCC2)C1] | 4-((1S,3R)-3-(1-isopropyl-3- (5-(trifluoromethyl)pyridin- 3-yl)-1H-pyrazol-5-yl)cyclo- pentyl)-1,4-oxazepane |
| 66 | 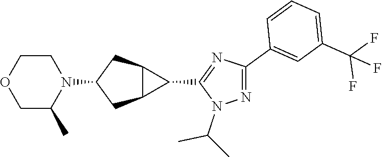 [SMILES: CC(C)n1nc(-c2cccc(C(F)(F)F)c2)nc1[C@H]1[C@@H]2C[C@@H](N3CCOC[C@@H]3C)C[C@@H]21] | (S)-4-((1R,3r,5S,6S)-6-(1- isopropyl-3-(3-(trifluoro- methyl)phenyl)-1H-1,2,4- triazol-5-yl)bicyclo[3.1.0] hexan-3-yl)-3-methylmor- pholine |
| 67 | 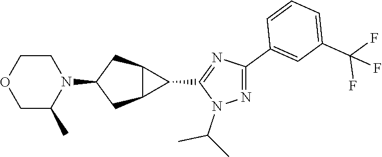 [SMILES: CC(C)n1nc(-c2cccc(C(F)(F)F)c2)nc1[C@H]1[C@@H]2C[C@H](N3CCOC[C@@H]3C)C[C@@H]21] | (S)-4-((1R,3s,5S,6S)-6-(1- isopropyl-3-(3-(trifluoro- methyl)phenyl)-1H-1,2,4- triazol-5-yl)bicyclo[3.1.0] hexan-3-yl)-3-methylmor- pholine |
| 68 | 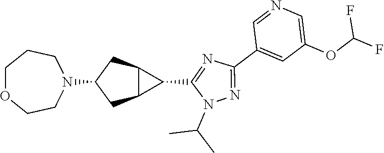 [SMILES: CC(C)n1nc(-c2cncc(OC(F)F)c2)nc1[C@H]1[C@@H]2C[C@@H](N3CCCOCC3)C[C@@H]21] | 4-((1R,3s,5S,6r)-6-(3-(5- (difluoromethoxy)pyridin-3- yl)-1-isopropyl-1H-1,2,4- triazol-5-yl)bicyclo[3.1.0] hexan-3-yl)-1,4-oxazepane |
| 69 | 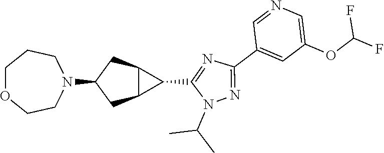 [SMILES: CC(C)n1nc(-c2cncc(OC(F)F)c2)nc1[C@H]1[C@@H]2C[C@H](N3CCCOCC3)C[C@@H]21] | 4-((1R,3r,5S,6r)-6-(3-(5- (difluoromethoxy)pyridin-3- yl)-1-isopropyl-1H-1,2,4- triazol-5-yl)bicyclo[3.1.0] hexan-3-yl)-1,4-oxazepane |
| 70* | 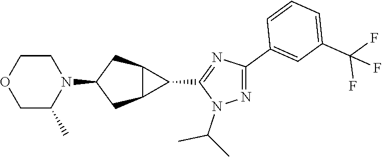 [SMILES: CC(C)n1nc(-c2cccc(C(F)(F)F)c2)nc1[C@H]1[C@@H]2C[C@H](N3CCOC[C@H]3C)C[C@@H]21] | (R)-4-((1R,3r,5S,6S)-6-(1- isopropyl-3-(3-(trifluoro- methyl)phenyl)-1H-1,2,4- triazol-5-yl)bicyclo[3.1.0] hexan-3-yl)-3-methylmor- pholine |
| 71* | 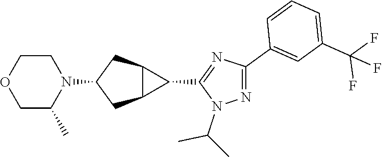 [SMILES: CC(C)n1nc(-c2cccc(C(F)(F)F)c2)nc1[C@H]1[C@@H]2C[C@@H](N3CCOC[C@H]3C)C[C@@H]21] | (R)-4-((1R,3s,5S,6S)-6-(1- isopropyl-3-(3-(trifluoro- methyl)phenyl)-1H-1,2,4- triazol-5-yl)bicyclo[3.1.0] hexan-3-yl)-3-methylmor- pholine |
| 72 | 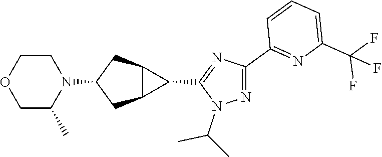 [SMILES: CC(C)n1nc(-c2cccc(C(F)(F)F)n2)nc1[C@H]1[C@@H]2C[C@@H](N3CCOC[C@H]3C)C[C@@H]21] | (R)-4-((1R,3s,5S,6R)-6-(1- isopropyl-3-(6-(trifluoro- methyl)pyridin-2-yl)-1H- 1,2,4-triazol-5-yl)bicyclo [3.1.0]hexan-3-yl)-3-methyl- morpholine |
| 73 | 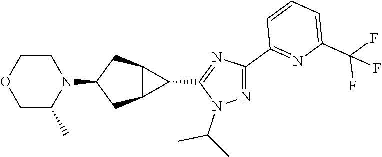 [SMILES: CC(C)n1nc(-c2cccc(C(F)(F)F)n2)nc1[C@H]1[C@@H]2C[C@H](N3CCOC[C@H]3C)C[C@@H]21] | (R)-4-((1R,3r,5S,6R)-6-(1- isopropyl-3-(6-(trifluoro- methyl)pyridin-2-yl)-1H- 1,2,4-triazol-5-yl)bicyclo [3.1.0]hexan-3-yl)-3-methyl- morpholine |
| 74 | 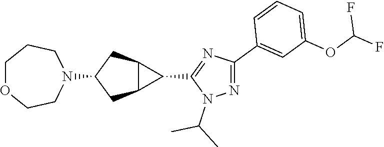 [SMILES: CC(C)n1nc(-c2cccc(OC(F)F)c2)nc1[C@H]1[C@@H]2C[C@@H](N3CCCOCC3)C[C@@H]21] | 4-((1R,3s,5S,6r)-6-(3-(3- (difluoromethoxy)phenyl)-1- isopropyl-1H-1,2,4-triazol-5- yl)bicyclo[3.1.0]hexan-3-yl)- 1,4-oxazepane |
| 75 | 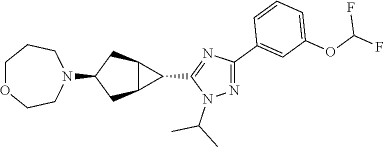 [SMILES: CC(C)n1nc(-c2cccc(OC(F)F)c2)nc1[C@H]1[C@@H]2C[C@H](N3CCCOCC3)C[C@@H]21] | 4-((1R,3r,5S,6r)-6-(3-(3- (difluoromethoxy)phenyl)-1- isopropyl-1H-1,2,4-triazol-5- yl)bicyclo[3.1.0]hexan-3-yl)- 1,4-oxazepane |
| 76 | 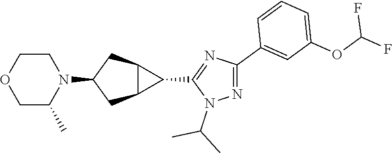 [SMILES: CC(C)n1nc(-c2cccc(OC(F)F)c2)nc1[C@H]1[C@@H]2C[C@H](N3CCOC[C@H]3C)C[C@@H]21] | (R)-4-((1R,3r,5S,6R)-6-(3- (3-(difluoromethoxy)phenyl)- 1-isopropyl-1H-1,2,4-triazol- 5-yl)bicyclo[3.1.0]hexan-3- yl)-3-methylmorpholine |
| 77 | 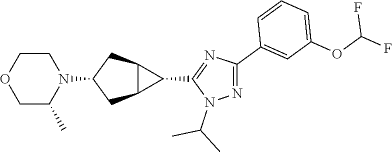 [SMILES: CC(C)n1nc(-c2cccc(OC(F)F)c2)nc1[C@H]1[C@@H]2C[C@@H](N3CCOC[C@H]3C)C[C@@H]21] | (R)-4-((1R,3s,5S,6R)-6-(3- (3-(difluoromethoxy)phenyl)- 1-isopropyl-1H-1,2,4-triazol- 5-yl)bicyclo[3.1.0]hexan-3- yl)-3-methylmorpholine |
| 78* | 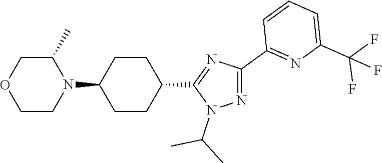 [SMILES: CC(C)n1nc(-c2cccc(C(F)(F)F)n2)nc1[C@H]1CC[C@H](N2CCOC[C@@H]2C)CC1] | (S)-4-((1R,4S)-4-(1-isoprop- yl-3-(6-(trifluoromethyl)- pyridin-2-yl)-1H-1,2,4-tri- azol-5-yl)cyclohexyl)-3- methylmorpholine |
| 79* | 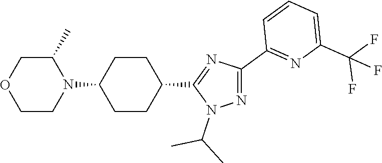 [SMILES: CC(C)n1nc(-c2cccc(C(F)(F)F)n2)nc1[C@H]1CC[C@@H](N2CCOC[C@@H]2C)CC1] | (S)-4-((1s,4R)-4-(1-isoprop- yl-3-(6-(trifluoromethyl)pyr- idin-2-yl)-1H-1,2,4-triazol- 5-yl)cyclohexyl)-3-methyl- morpholine |
| 80* | 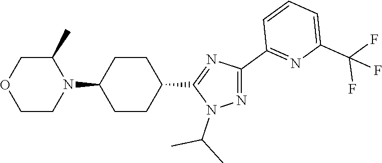 [SMILES: CC(C)n1nc(-c2cccc(C(F)(F)F)n2)nc1[C@H]1CC[C@H](N2CCOC[C@H]2C)CC1] | (R)-4-((1R,4R)-4-(1-isoprop- yl-3-(6-(trifluoromethyl)pyr- idin-2-yl)-1H-1,2,4-triazol-5- yl)cyclohexyl)-3-methylmor- pholine |
| 81* | 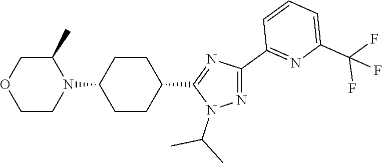 [SMILES: CC(C)n1nc(-c2cccc(C(F)(F)F)n2)nc1[C@H]1CC[C@@H](N2CCOC[C@H]2C)CC1] | (R)-4-((1s,4S)-4-(1-isoprop- yl-3-(6-(trifluoromethyl)pyr- idin-2-yl)-1H-1,2,4-triazol-5- yl)cyclohexyl)-3-methylmor- pholine |
| 82 | 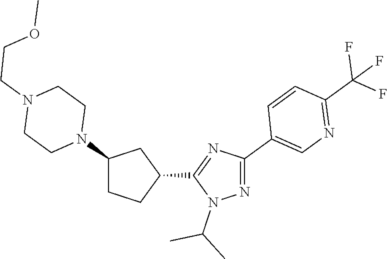 [SMILES: COCCN1CCN([C@@H]2CC[C@@H](c3nc(-c4ccc(C(F)(F)F)nc4)nn3C(C)C)C2)CC1] | 1-((1R,3R)-3-(1-isopropyl- 3-(6-(trifluoromethyl)pyr- idin-3-yl)-1H-1,2,4-triazol- 5-yl)cyclopentyl)-4-(2-meth- oxyethyl)piperazine |
| 83 | 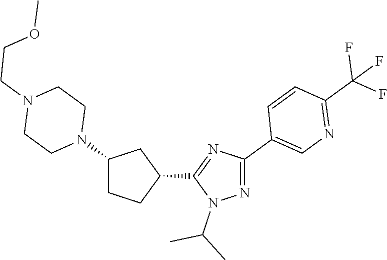 [SMILES: COCCN1CCN([C@H]2CC[C@@H](c3nc(-c4ccc(C(F)(F)F)nc4)nn3C(C)C)C2)CC1] | 1-((1S,3R)-3-(1-isopropyl-3- (6-(trifluoromethyl)pyridin- 3-yl)-1H-1,2,4-triazol-5-yl) cyclopentyl)-4-(2-methoxy- ethyl)piperazine |
| 84 | 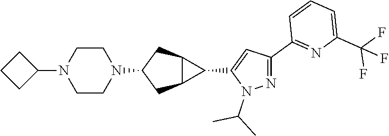 [SMILES: CC(C)n1nc(-c2cccc(C(F)(F)F)n2)cc1[C@H]1[C@@H]2C[C@@H](N3CCN(C4CCC4)CC3)C[C@@H]21] | 1-cyclobutyl-4-((1R,3s,5S, 6r)-6-(1-isopropyl-3-(6-(tri- fluoromethyl)pyridin-2-yl)- 1H-pyrazol-5-yl)bicyclo [3.1.0]hexan-3-ylpiperazine |
| 85 | 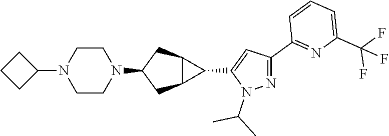 [SMILES: CC(C)n1nc(-c2cccc(C(F)(F)F)n2)cc1[C@H]1[C@@H]2C[C@H](N3CCN(C4CCC4)CC3)C[C@@H]21] | 1-cyclobutyl-4-((1R,3r,5S, 6r)-6-(1-isopropyl-3-(6-(tri- fluoromethyl)pyridin-2-yl)- 1H-pyrazol-5-yl)bicyclo [3.1.0]hexan-3-yl)piperazine |
| 86* | 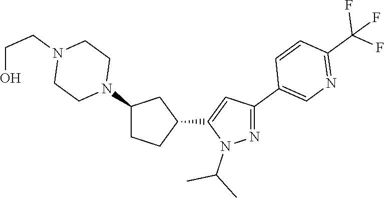 [SMILES: CC(C)n1nc(-c2ccc(C(F)(F)F)nc2)cc1[C@@H]1CC[C@@H](N2CCN(CCO)CC2)C1] | 2-(4-((1R,3R)-3-(1-isoprop- yl-3-(6-(trifluoromethyl)pyr- idin-3-yl)-1H-pyrazol-5-yl)- cyclopentyl)piperazin-1-yl)- ethanol |
| 87* | 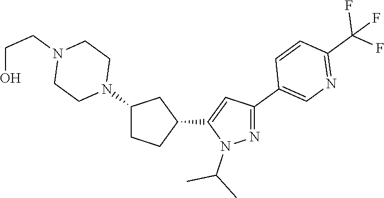 [SMILES: CC(C)n1nc(-c2ccc(C(F)(F)F)nc2)cc1[C@@H]1CC[C@H](N2CCN(CCO)CC2)C1] | 2-(4-((1S,3R)-3-(1-isoprop- yl-3-(6-(trifluoromethyl)pyr- idin-3-yl)-1H-pyrazol-5-yl) cyclopentyl)piperazin-1-yl)- ethanol |
| 88* | 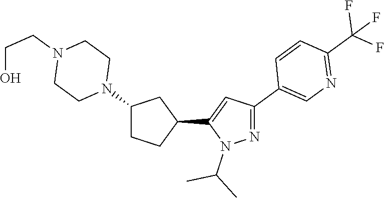 [SMILES: CC(C)n1nc(-c2ccc(C(F)(F)F)nc2)cc1[C@H]1CC[C@H](N2CCN(CCO)CC2)C1] | 2-(4-((1S,3S)-3-(1-isoprop- yl-3-(6-(trifluoromethyl)pyr- idin-3-yl)-1H-pyrazol-5-yl)- cyclopentyl)piperazin-1-yl)- ethanol |
| 89* | 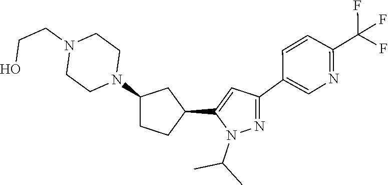 [SMILES: CC(C)n1nc(-c2ccc(C(F)(F)F)nc2)cc1[C@H]1CC[C@@H](N2CCN(CCO)CC2)C1] | 2-(4-((1R,3S)-3-(1-isoprop- yl-3-(6-(trifluoromethyl)pyr- idin-3-yl)-1H-pyrazol-5-yl) cyclopentyl)piperazin-1-yl)- ethanol |
| 90* | 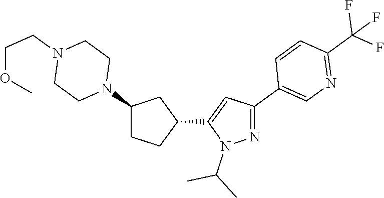 [SMILES: COCCN1CCN([C@@H]2CC[C@@H](c3cc(-c4ccc(C(F)(F)F)nc4)nn3C(C)C)C2)CC1] | 1-((1R,3R)-3-(1-isopropyl-3- (6-(trifluoromethyl)pyridin- 3-yl)-1H-pyrazol-5-yl)cyclo- pentyl)-4-(2-methoxyethyl)- piperazine |
| 91* | 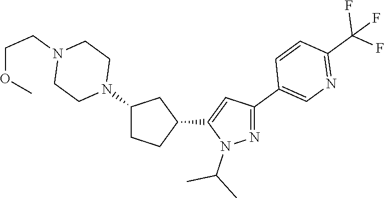 [SMILES: COCCN1CCN([C@H]2CC[C@@H](c3cc(-c4ccc(C(F)(F)F)nc4)nn3C(C)C)C2)CC1] | 1-((1S,3R)-3-(1-isopropyl-3- (6-(trifluoromethyl)pyridin- 3-yl)-1H-pyrazol-5-yl)cyclo- pentyl)-4-(2-methoxyethyl) piperazine |
| 92* | 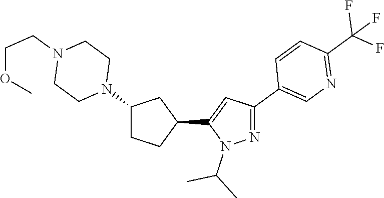 [SMILES: COCCN1CCN([C@H]2CC[C@H](c3cc(-c4ccc(C(F)(F)F)nc4)nn3C(C)C)C2)CC1] | 1-((1S,3S)-3-(1-isopropyl-3- (6-(trifluoromethyl)pyridin- 3-yl)-1H-pyrazol-5-yl)cyclo- pentyl)-4-(2-methoxyethyl)- piperazine |
| 93* | 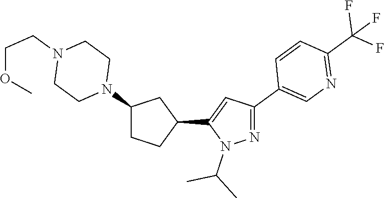 [SMILES: COCCN1CCN([C@@H]2CC[C@H](c3cc(-c4ccc(C(F)(F)F)nc4)nn3C(C)C)C2)CC1] | 1-((1R,3S)-3-(1-isopropyl-3- (6-(trifluoromethyl)pyridin- 3-yl)-1H-pyrazol-5-yl)cyclo- pentyl)-4-(2-methoxyethyl)- piperazine |
| 94 | 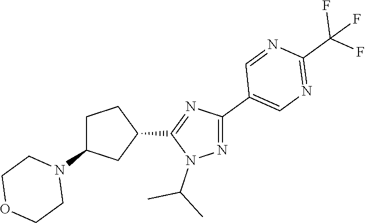 [SMILES: CC(C)n1nc(-c2cnc(C(F)(F)F)nc2)nc1[C@H]1CC[C@H](N2CCOCC2)C1] | 4-((1S,3S)-3-(1-isopropyl-3- (2-(trifluoromethyl)pyrim- idin-5-yl)-1H-1,2,4-triazol-5- yl)cyclopentyl)morpholine |
| 95 | 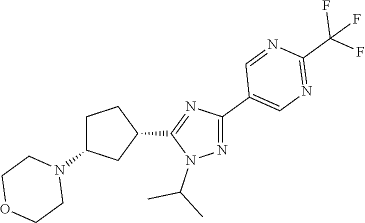 [SMILES: CC(C)n1nc(-c2cnc(C(F)(F)F)nc2)nc1[C@H]1CC[C@@H](N2CCOCC2)C1] | 4-((1R,3S)-3-(1-isopropyl-3- (2-(trifluoromethyl)pyrim- idin-5-yl)-1H-1,2,4-triazol-5- yl)cyclopentyl)morpholine |
| 96 | 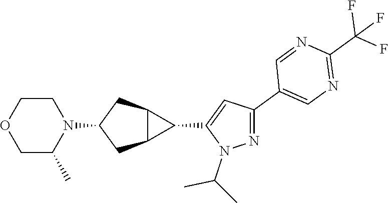 [SMILES: CC(C)n1nc(-c2cnc(C(F)(F)F)nc2)cc1[C@H]1[C@@H]2C[C@@H](N3CCOC[C@H]3C)C[C@@H]21] | (R)-4-((1R,3s,5S,6R)-6-(1- isopropyl-3-(2-(trifluoro- methyl)pyrimidin-5-yl)-1H- pyrazol-5-yl)bicyclo[3.1.0] hexan-3-yl)-3-methylmor- pholine |
| 97 | 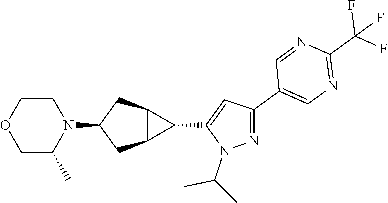 [SMILES: CC(C)n1nc(-c2cnc(C(F)(F)F)nc2)cc1[C@H]1[C@@H]2C[C@H](N3CCOC[C@H]3C)C[C@@H]21] | (R)-4-((1R,3r,5S,6R)-6-(1- isopropyl-3-(2-(trifluoro- methyl)pyrimidin-5-yl)-1H- pyrazol-5-yl)bicyclo[3.1.0] hexan-3-yl)-3-methylmor- pholine |
| 98 | 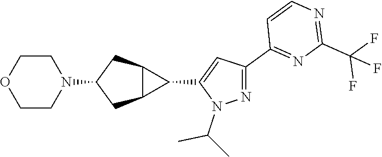 [SMILES: CC(C)n1nc(-c2ccnc(C(F)(F)F)n2)cc1[C@H]1[C@@H]2C[C@@H](N3CCOCC3)C[C@@H]21] | 4-((1R,3s,5S,6r)-6-(1-iso- propyl-3-(2-(trifluorometh- yl)pyrimidin-4-yl)-1H-pyr- azol-5-yl)bicyclo[3.1.0] hexan-3-yl)morpholine |
| 99 | 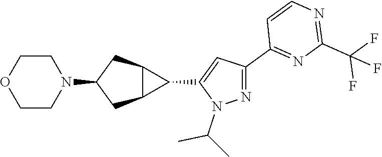 [SMILES: CC(C)n1nc(-c2ccnc(C(F)(F)F)n2)cc1[C@H]1[C@@H]2C[C@H](N3CCOCC3)C[C@@H]21] | 4-((1R,3r,5S,6r)-6-(1-iso- propyl-3-(2-(trifluorometh- yl)pyrimidin-4-yl)-1H-pyr- azol-5-yl)bicyclo[3.1.0] hexan-3-yl)morpholine |
| 100 | 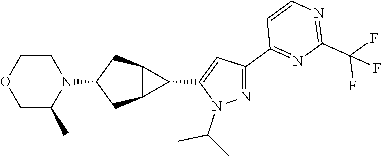 [SMILES: CC(C)n1nc(-c2ccnc(C(F)(F)F)n2)cc1[C@H]1[C@@H]2C[C@@H](N3CCOC[C@@H]3C)C[C@@H]21] | (S)-4-((1R,3s,5S,6S)-6-(1- isopropyl-3-(2-(trifluoro- methyl)pyrimidin-4-yl)-1H- pyrazol-5-yl)bicyclo[3.1.0] hexan-3-yl)-3-methylmor- pholine |
| 101 | 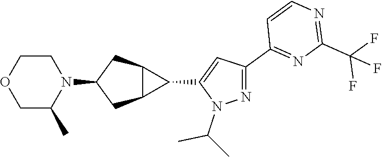 [SMILES: CC(C)n1nc(-c2ccnc(C(F)(F)F)n2)cc1[C@H]1[C@@H]2C[C@H](N3CCOC[C@@H]3C)C[C@@H]21] | (S)-4-((1R,3r,5S,6S)-6-(1- isopropyl-3-(2-(trifluoro- methyl)pyrimidin-4-yl)-1H- pyrazol-5-yl)bicyclo[3.1.0] hexan-3-yl)-3-methylmor- pholine |
| 102 | 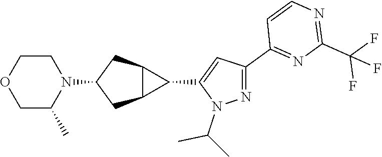 [SMILES: CC(C)n1nc(-c2ccnc(C(F)(F)F)n2)cc1[C@H]1[C@@H]2C[C@@H](N3CCOC[C@H]3C)C[C@@H]21] | (R)-4-((1R,3s,5S,6S)-6-(1- isopropyl-3-(2-(trifluoro- methyl)pyrimidin-4-yl)-1H- pyrazol-5-yl)bicyclo[3.1.0] hexan-3-yl)-3-methylmor- pholine |
| 103 | 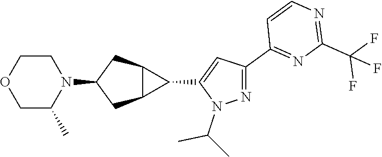 [SMILES: CC(C)n1nc(-c2ccnc(C(F)(F)F)n2)cc1[C@H]1[C@@H]2C[C@H](N3CCOC[C@H]3C)C[C@@H]21] | (R)-4-((1R,3r,5S,6S)-6-(1- isopropyl-3-(2-(trifluoro- methyl)pyrimidin-4-yl)-1H- pyrazol-5-yl)bicyclo[3.1.0] hexan-3-yl)-3-methylmor- pholine |
| 104 | 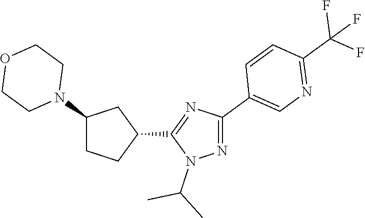 [SMILES: CC(C)n1nc(-c2ccc(C(F)(F)F)nc2)nc1[C@@H]1CC[C@@H](N2CCOCC2)C1] | 4-((1R,3R)-3-(1-isopropyl-3- (6-(trifluoromethyl)pyridin- 3-yl)-1H-1,2,4-triazol-5-yl)- cyclopentyl)morpholine |
| 105 | 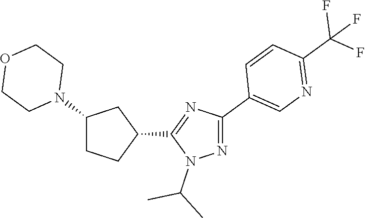 [SMILES: CC(C)n1nc(-c2ccc(C(F)(F)F)nc2)nc1[C@@H]1CC[C@H](N2CCOCC2)C1] | 4-((1S,3R)-3-(1-isopropyl-3- (6-(trifluoromethyl)pyridin-3- yl)-1H-1,2,4-triazol-5-yl)- cyclopentyl)morpholine |
| 202 | 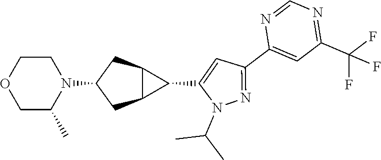 [SMILES: CC(C)n1nc(-c2cc(C(F)(F)F)ncn2)cc1[C@H]1[C@@H]2C[C@@H](N3CCOC[C@H]3C)C[C@@H]21] | (R)-4-((1R,3s,5S,6R)-6-(1- isopropyl-3-(6-(trifluoro- methyl)pyrimidin-4-yl)-1H- pyrazol-5-yl)bicyclo[3.1.0] hexan-3-yl)-3-methylmor- pholine |
| 106 | 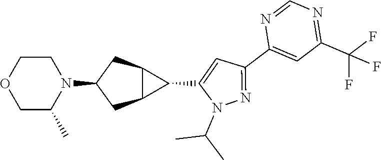 [SMILES: CC(C)n1nc(-c2cc(C(F)(F)F)ncn2)cc1[C@H]1[C@@H]2C[C@H](N3CCOC[C@H]3C)C[C@@H]21] | (R)-4-((1R,3r,5S,6R)-6-(1- isopropyl-3-(6-(trifluoro- methyl)pyrimidin-4-yl)-1H- pyrazol-5-yl)bicyclo[3.1.0] hexan-3-yl)-3-methylmor- pholine |
| 107 | 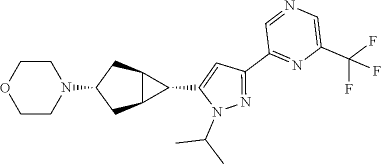 [SMILES: CC(C)n1nc(-c2cncc(C(F)(F)F)n2)cc1[C@H]1[C@@H]2C[C@@H](N3CCOCC3)C[C@@H]21] | 4-((1R,3s,5S,6r)-6-(1-iso- propyl-3-(6-(trifluorometh- yl)pyrazin-2-yl)-1H-pyrazol- 5-yl)bicyclo[3.1.0]hexan-3- yl)morpholine |
| 108 | 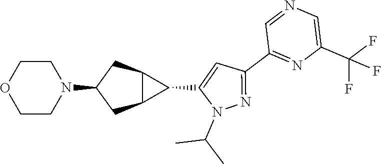 [SMILES: CC(C)n1nc(-c2cncc(C(F)(F)F)n2)cc1[C@H]1[C@@H]2C[C@H](N3CCOCC3)C[C@@H]21] | 4-((1R,3r,5S,6r)-6-(1-iso- propyl-3-(6-(trifluorometh- yl)pyrazin-2-yl)-1H-pyrazol- 5-yl)bicyclo[3.1.0]hexan-3- yl)morpholine |
| 109* | 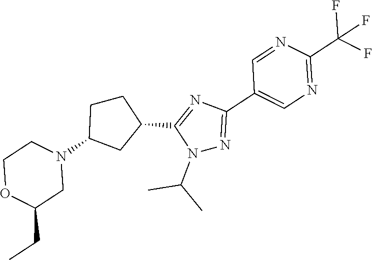 [SMILES: CC[C@@H]1CN([C@@H]2CC[C@H](c3nc(-c4cnc(C(F)(F)F)nc4)nn3C(C)C)C2)CCO1] | (R)-2-ethyl-4-((1R,3S)-3-(1- isopropyl-3-(2-(trifluoro- methyl)pyrimidin-5-yl)-1H- 1,2,4-triazol-5-yl)cyclopent- yl)morpholine |
| 110* | 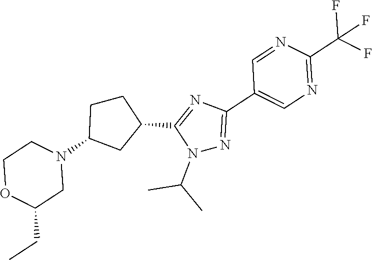 [SMILES: CC[C@H]1CN([C@@H]2CC[C@H](c3nc(-c4cnc(C(F)(F)F)nc4)nn3C(C)C)C2)CCO1] | (S)-2-ethyl-4-((1R,3S)-3-(1- isopropyl-3-(2-(trifluoro- methyl)pyrimidin-5-yl)-1H- 1,2,4-triazol-5-yl)cyclopent- yl)morpholine |
| 111* | 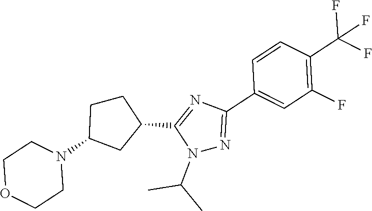 [SMILES: CC(C)n1nc(-c2ccc(C(F)(F)F)c(F)c2)nc1[C@H]1CC[C@@H](N2CCOCC2)C1] | 4-((1R,3S)-3-(3-(3-fluoro-4- (trifluoromethyl)phenyl)-1- isopropyl-1H-1,2,4-triazol-5- yl)cyclopentyl)morpholine |
| 112* | 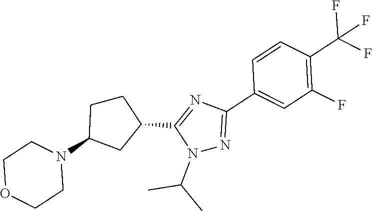 [SMILES: CC(C)n1nc(-c2ccc(C(F)(F)F)c(F)c2)nc1[C@H]1CC[C@H](N2CCOCC2)C1] | 4-((1S,3S)-3-(3-(3-fluoro-4- (trifluoromethyl)phenyl)-1- isopropyl-1H-1,2,4-triazol-5- yl)cyclopentyl)morpholine |
| 113* | 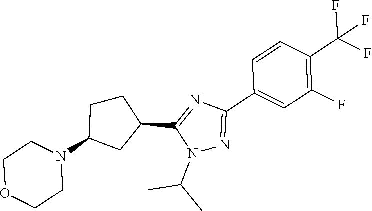 [SMILES: CC(C)n1nc(-c2ccc(C(F)(F)F)c(F)c2)nc1[C@@H]1CC[C@H](N2CCOCC2)C1] | 4-((1S,3R)-3-(3-(3-fluoro-4- (trifluoromethyl)phenyl)-1- isopropyl-1H-1,2,4-triazol-5- yl)cyclopentyl)morpholine |
| 114* | 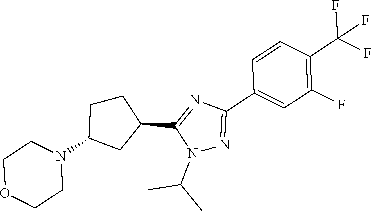 [SMILES: CC(C)n1nc(-c2ccc(C(F)(F)F)c(F)c2)nc1[C@@H]1CC[C@@H](N2CCOCC2)C1] | 4-((1R,3R)-3-(3-(3-fluoro-4- (trifluoromethyl)phenyl)-1- isopropyl-1H-1,2,4-triazol-5- yl)cyclopentyl)morpholine |
| 115* | 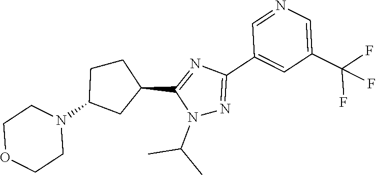 [SMILES: CC(C)n1nc(-c2cncc(C(F)(F)F)c2)nc1[C@@H]1CC[C@@H](N2CCOCC2)C1] | 4-((1R,3R)-3-(1-isopropyl-3- (5-(trifluoromethyl)pyridin- 3-yl)-1H-1,2,4-triazol-5-yl)- cyclopentyl)morpholine |
| 116* | 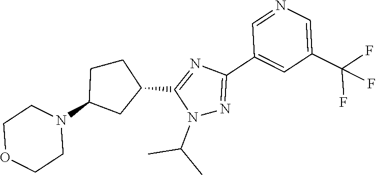 [SMILES: CC(C)n1nc(-c2cncc(C(F)(F)F)c2)nc1[C@H]1CC[C@H](N2CCOCC2)C1] | 4-((1S,3S)-3-(1-isopropyl-3- (5-(trifluoromethyl)pyridin-3- yl)-1H-1,2,4-triazol-5-yl)- cyclopentyl)morpholine |
| 117* | 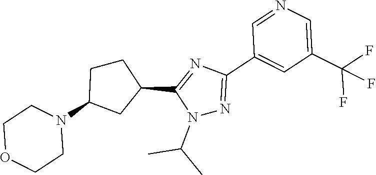 [SMILES: CC(C)n1nc(-c2cncc(C(F)(F)F)c2)nc1[C@@H]1CC[C@H](N2CCOCC2)C1] | 4-((1S,3R)-3-(1-isopropyl-3- (5-(trifluoromethyl)pyridin- 3-yl)-1H-1,2,4-triazol-5-yl)- cyclopentyl)morpholine |
| 118* | 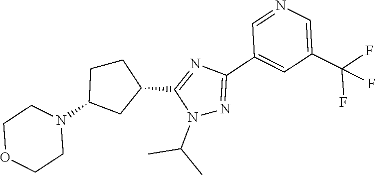 [SMILES: CC(C)n1nc(-c2cncc(C(F)(F)F)c2)nc1[C@H]1CC[C@@H](N2CCOCC2)C1] | 4-((1R,3S)-3-(1-isopropyl-3- (5-(trifluoromethyl)pyridin- 3-yl)-1H-1,2,4-triazol-5-yl)- cyclopentyl)morpholine |
| 119 | 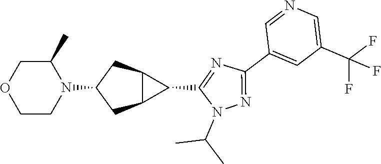 [SMILES: CC(C)n1nc(-c2cncc(C(F)(F)F)c2)nc1[C@H]1[C@@H]2C[C@@H](N3CCOC[C@H]3C)C[C@@H]21] | (R)-4-((1R,3s,5S,6R)-6-(1- isopropyl-3-(5-(trifluoro- methyl)pyridin-3-yl)-1H- 1,2,4-triazol-5-l)bicyclo [3.1.0]hexan-3-yl)-3-methyl- morpholine |
| 120 | 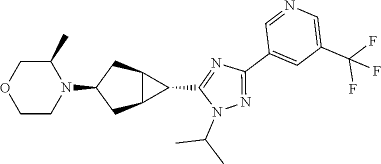 [SMILES: CC(C)n1nc(-c2cncc(C(F)(F)F)c2)nc1[C@H]1[C@@H]2C[C@H](N3CCOC[C@H]3C)C[C@@H]21] | (R)-4-((1R,3r,5S,6R)-6-(1- isopropyl-3-(5-(trifluoro- methyl)pyridin-3-yl)-1H- 1,2,4-triazol-5-l)bicyclo [3.1.0]hexan-3-yl)-3-methyl- morpholine |
| 121 | 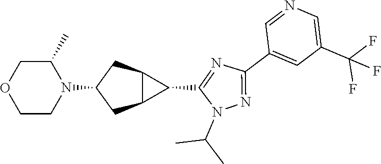 [SMILES: CC(C)n1nc(-c2cncc(C(F)(F)F)c2)nc1[C@H]1[C@@H]2C[C@@H](N3CCOC[C@@H]3C)C[C@@H]21] | (S)-4-((1R,3s,5S,6R)-6-(1- isopropyl-3-(5-(trifluoro- methyl)pyridin-3-yl)-1H- 1,2,4-triazol-5-yl)bicyclo [3.1.0]hexan-3-yl)-3-methyl- morpholine |
| 122 | 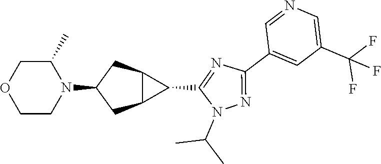 [SMILES: CC(C)n1nc(-c2cncc(C(F)(F)F)c2)nc1[C@H]1[C@@H]2C[C@H](N3CCOC[C@@H]3C)C[C@@H]21] | (S)-4-((1R,3r,5S,6R)-6-(1- isopropyl-3-(5-(trifluoro- methyl)pyridin-3-yl)-1H- 1,2,4-triazol-5-yl)bicyclo [3.1.0]hexan-3-yl)-3-methyl- morpholine |
| 123 | 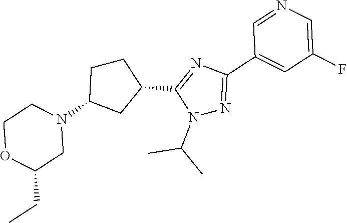 [SMILES: CC[C@H]1CN([C@@H]2CC[C@H](c3nc(-c4cncc(F)c4)nn3C(C)C)C2)CCO1] | (S)-2-ethyl-4-((1R,3S)-3-(3- (5-fluoropyridin-3-yl)-1- isopropyl-1H-1,2,4-triazol-5- yl)cyclopentyl)morpholine |
| 124 | 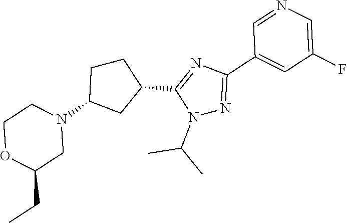 [SMILES: CC[C@@H]1CN([C@@H]2CC[C@H](c3nc(-c4cncc(F)c4)nn3C(C)C)C2)CCO1] | (R)-2-ethyl-4-((1R,3S)-3-(3- (5-fluoropyridin-3-yl)-1- isopropyl-1H-1,2,4-triazol- 5-yl)cyclopentyl)morpholine |
| 125 | 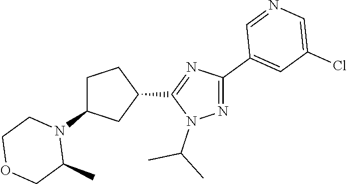 [SMILES: CC(C)n1nc(-c2cncc(Cl)c2)nc1[C@H]1CC[C@H](N2CCOC[C@@H]2C)C1] | (S)-4-((1S,3S)-3-(3-(5- chloropyridin-3-yl)-1-iso- propyl-1H-1,2,4-triazol-5- yl)cyclopentyl)-3-methyl- morpholine |
| 126 | 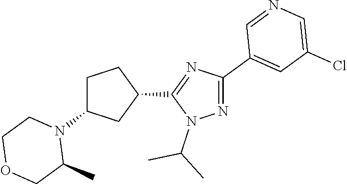 [SMILES: CC(C)n1nc(-c2cncc(Cl)c2)nc1[C@H]1CC[C@@H](N2CCOC[C@@H]2C)C1] | (S)-4-((1R,3S)-3-(3-(5- chloropyridin-3-yl)-1-iso- propyl-1H-1,2,4-triazol-5- yl)cyclopentyl)-3-methyl- morpholine |
| 127 | 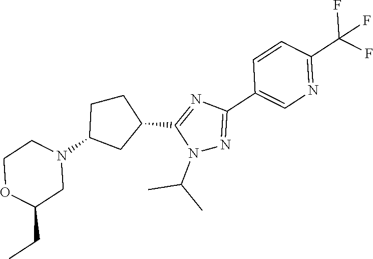 [SMILES: CC[C@@H]1CN([C@@H]2CC[C@H](c3nc(-c4ccc(C(F)(F)F)nc4)nn3C(C)C)C2)CCO1] | (R)-2-ethyl-4-((1R,3S)-3-(1- isopropyl-3-(6-(trifluoro- methyl)pyridin-3-yl)-1H- 1,2,4-triazol-5-yl)cyclopent- yl)morpholine |
| 128 | 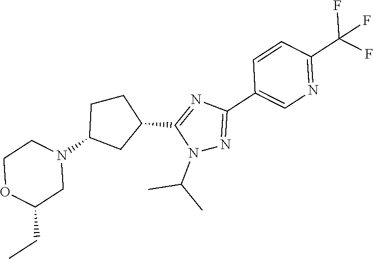 [SMILES: CC[C@H]1CN([C@@H]2CC[C@H](c3nc(-c4ccc(C(F)(F)F)nc4)nn3C(C)C)C2)CCO1] | (S)-2-ethyl-4-((1R,3S)-3-(1- isopropyl-3-(6-(trifluoro- methyl)pyridin-3-yl)-1H- 1,2,4-triazol-5-yl)cyclopent- yl)morpholine |
| 129 | 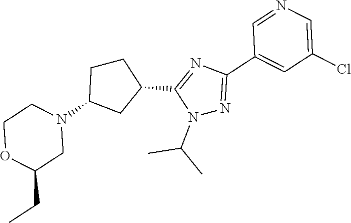 [SMILES: CC[C@@H]1CN([C@@H]2CC[C@H](c3nc(-c4cncc(Cl)c4)nn3C(C)C)C2)CCO1] | (R)-4-((1R,3S)-3-(3-(5- chloropyridin-3-yl)-1-iso- propyl-1H-1,2,4-triazol-5- yl)cyclopentyl)-2-ethylmor- pholine |
| 130* | 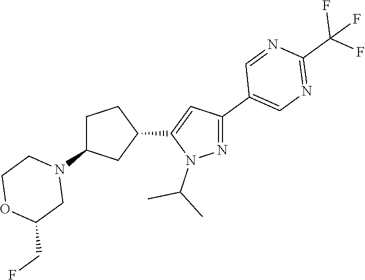 [SMILES: CC(C)n1nc(-c2cnc(C(F)(F)F)nc2)cc1[C@H]1CC[C@H](N2CCO[C@@H](CF)C2)C1] | (R)-2-(fluoromethyl)-4- ((1S,3S)-3-(1-isopropyl-3- (2-(trifluoromethyl)pyr- imidin-5-yl)-1H-pyrazol-5- yl)cyclopentyl)morpholine |
| 131* | 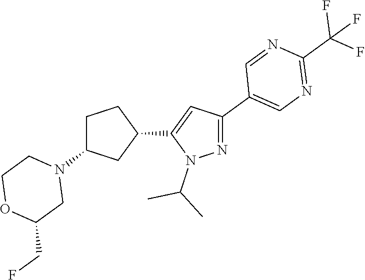 [SMILES: CC(C)n1nc(-c2cnc(C(F)(F)F)nc2)cc1[C@H]1CC[C@@H](N2CCO[C@@H](CF)C2)C1] | (R)-2-(fluoromethyl)-4- ((1R,3S)-3-(1-isopropyl-3- (2-(trifluoromethyl)pyr- imidin-5-yl)-1H-pyrazol-5- yl)cyclopentyl)morpholine |
| 132* | 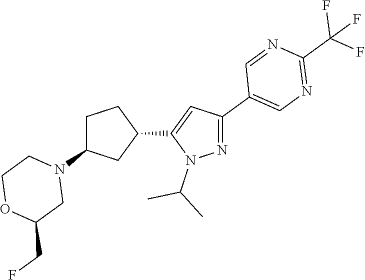 [SMILES: CC(C)n1nc(-c2cnc(C(F)(F)F)nc2)cc1[C@H]1CC[C@H](N2CCO[C@H](CF)C2)C1] | (S)-2-(fluoromethyl)-4- ((1S,3S)-3-(1-isopropyl-3- (2-(trifluoromethyl)pyr- imidin-5-yl)-1H-pyrazol-5- yl)cyclopentyl)morpholine |
| 133* | 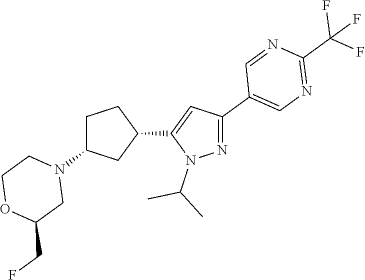 [SMILES: CC(C)n1nc(-c2cnc(C(F)(F)F)nc2)cc1[C@H]1CC[C@@H](N2CCO[C@H](CF)C2)C1] | (S)-2-(fluoromethyl)-4- ((1R,3S)-3-(1-isopropyl-3- (2-(trifluoromethyl)pyrim- idin-5-yl)-1H-pyrazol-5- yl)cyclopentyl)morpholine |
| 144 | 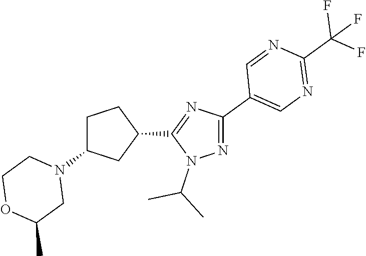 [SMILES: CC(C)n1nc(-c2cnc(C(F)(F)F)nc2)nc1[C@H]1CC[C@@H](N2CCO[C@H](C)C2)C1] | (R)-4-((1R,3S)-3-(1-iso- propyl-3-(2-(trifluorometh- yl)pyrimidin-5-yl)-1H-1,2,4- triazol-5-yl)cyclopentyl)-2- methylmorpholine |
| 145 | 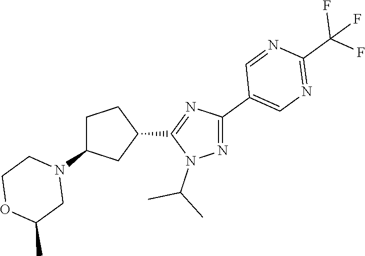 [SMILES: CC(C)n1nc(-c2cnc(C(F)(F)F)nc2)nc1[C@H]1CC[C@H](N2CCO[C@H](C)C2)C1] | (R)-4-((1S,3S)-3-(1-iso- propyl-3-(2-(trifluorometh- yl)pyrimidin-5-yl)-1H-1,2,4- triazol-5-yl)cyclopentyl)-2- methylmorpholine |
| 146 | 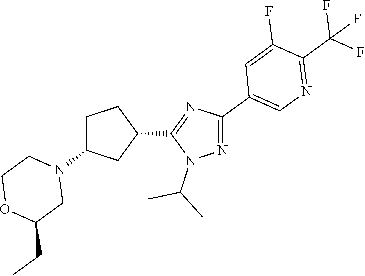 [SMILES: CC[C@@H]1CN([C@@H]2CC[C@H](c3nc(-c4cnc(C(F)(F)F)c(F)c4)nn3C(C)C)C2)CCO1] | (R)-2-ethyl-4-((1R,3S)-3-(3- (5-fluoro-6-(trifluoromethyl)- pyridin-3-yl)-1-isopropyl-1H- 1,2,4-triazol-5-yl)cyclopent- yl)morpholine |
| 147 | 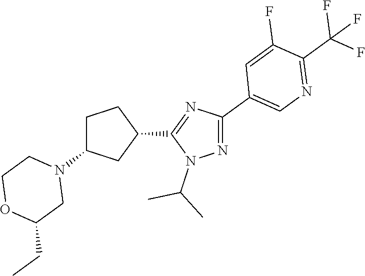 [SMILES: CC[C@H]1CN([C@@H]2CC[C@H](c3nc(-c4cnc(C(F)(F)F)c(F)c4)nn3C(C)C)C2)CCO1] | (S)-2-ethyl-4-((1R,3S)-3-(3- (5-fluoro-6-(trifluorometh- yl)pyridin-3-yl)-1-isopropyl- 1H-1,2,4-triazol-5-yl)cyclo- pentyl)morpholine |
| 148 | 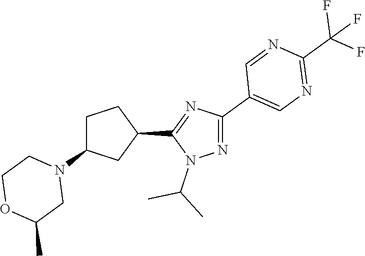 [SMILES: CC(C)n1nc(-c2cnc(C(F)(F)F)nc2)nc1[C@@H]1CC[C@H](N2CCO[C@H](C)C2)C1] | (R)-4-((1S,3R)-3-(1-isoprop- yl-3-(2-(trifluoromethyl)pyr- imidin-5-yl)-1H-1,2,4-tri- azol-5-yl)cyclopentyl)-2- methylmorpholine |
| 149 | 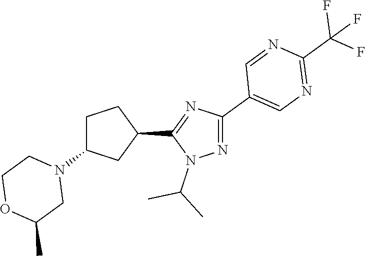 [SMILES: CC(C)n1nc(-c2cnc(C(F)(F)F)nc2)nc1[C@@H]1CC[C@@H](N2CCO[C@H](C)C2)C1] | (R)-4-((1R,3R)-3-(1-isoprop- yl-3-(2-(trifluoromethyl)pyr- imidin-5-yl)-1H-1,2,4-triazol- 5-yl)cyclopentyl)-2-methyl- morpholine |
| 150 | 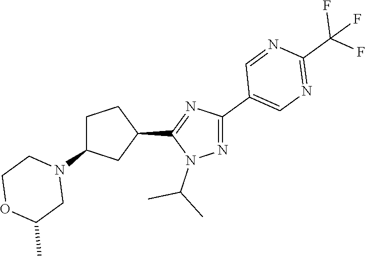 [SMILES: CC(C)n1nc(-c2cnc(C(F)(F)F)nc2)nc1[C@@H]1CC[C@H](N2CCO[C@@H](C)C2)C1] | (S)-4-((1S,3R)-3-(1-isoprop- yl-3-(2-(trifluoromethyl)- pyrimidin-5-yl)-1H-1,2,4- triazol-5-yl)cyclopentyl)-2- methylmorpholine |
| 151 | 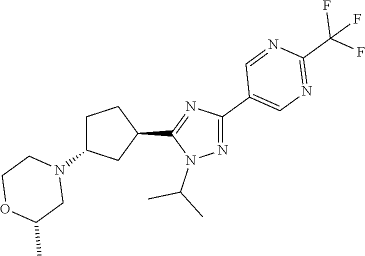 [SMILES: CC(C)n1nc(-c2cnc(C(F)(F)F)nc2)nc1[C@@H]1CC[C@@H](N2CCO[C@@H](C)C2)C1] | (S)-4-((1R,3R)-3-(1-isoprop- yl-3-(2-(trifluoromethyl)pyr- imidin-5-yl)-1H-1,2,4-triazol- 5-yl)cyclopentyl)-2-methyl- morpholine |
| 152 | 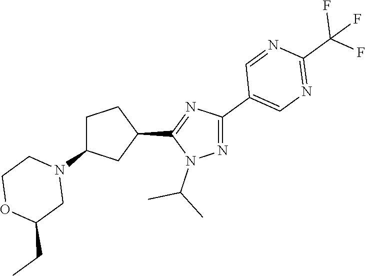 [SMILES: CC[C@@H]1CN([C@H]2CC[C@@H](c3nc(-c4cnc(C(F)(F)F)nc4)nn3C(C)C)C2)CCO1] | (R)-2-ethyl-4-((1S,3R)-3-(1- isopropyl-3-(2-(trifluoro- methyl)pyrimidin-5-yl)-1H- 1,2,4-triazol-5-yl)cyclopent- yl)morpholine |
| 153 | 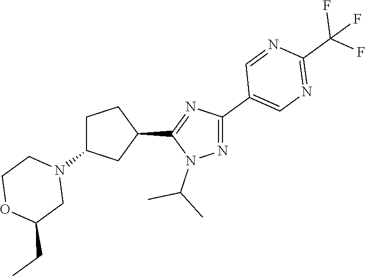 [SMILES: CC[C@@H]1CN([C@@H]2CC[C@@H](c3nc(-c4cnc(C(F)(F)F)nc4)nn3C(C)C)C2)CCO1] | (R)-2-ethyl-4-((1R,3R)-3-(1- isopropyl-3-(2-(trifluoro- methyl)pyrimidin-5-yl)-1H- 1,2,4-triazol-5-yl)cyclopent- yl)morpholine |
| 154 | 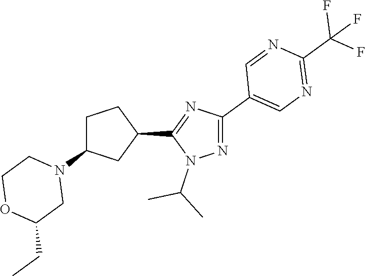 [SMILES: CC[C@H]1CN([C@H]2CC[C@@H](c3nc(-c4cnc(C(F)(F)F)nc4)nn3C(C)C)C2)CCO1] | (S)-2-ethyl-4-((1S,3R)-3-(1- isopropyl-3-(2-(trifluoro- methyl)pyrimidin-5-yl)-1H- 1,2,4-triazol-5-yl)cyclopent- yl)morpholine |
| 155 | 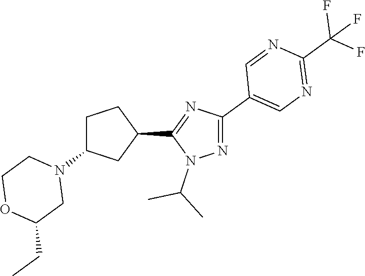 [SMILES: CC[C@H]1CN([C@@H]2CC[C@@H](c3nc(-c4cnc(C(F)(F)F)nc4)nn3C(C)C)C2)CCO1] | (S)-2-ethyl-4-((1R,3R)-3-(1- isopropyl-3-(2-(trifluoro- methyl)pyrimidin-5-yl)-1H- 1,2,4-triazol-5-yl)cyclopent- yl)morpholine |
| 156 | 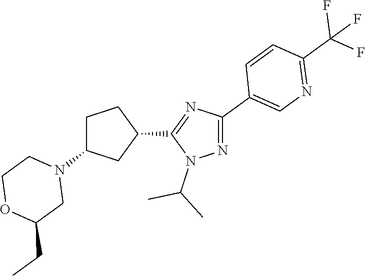 [SMILES: CC[C@@H]1CN([C@@H]2CC[C@H](c3nc(-c4ccc(C(F)(F)F)nc4)nn3C(C)C)C2)CCO1] | (R)-4-((1R,3S)-3-(3-(6- (difluoromethyl)pyridin-3- yl)-1-isopropyl-1H-1,2,4- triazol-5-yl)cyclopentyl)-2- ethylmorpholine |
| 157* | 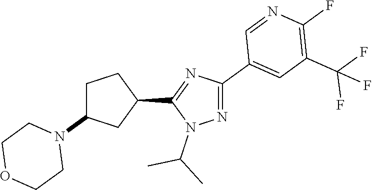 [SMILES: CC(C)n1nc(-c2cnc(F)c(C(F)(F)F)c2)nc1[C@@H]1CC[C@H](N2CCOCC2)C1] | 4-((1S,3R)-3-(3-(6-fluoro-5- (trifluoromethyl)pyridin-3- yl)-1-isopropyl-1H-1,2,4- triazol-5-yl)cyclopentyl)- morpholine |
| 158* | 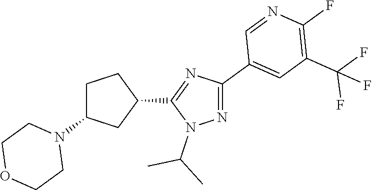 [SMILES: CC(C)n1nc(-c2cnc(F)c(C(F)(F)F)c2)nc1[C@H]1CC[C@@H](N2CCOCC2)C1] | 4-((1R,3S)-3-(3-(6-fluoro-5- (trifluoromethyl)pyridin-3- yl)-1-isopropyl-1H-1,2,4- triazol-5-yl)cyclopentyl)mor- pholine |
| 159* | 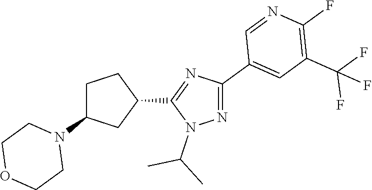 [SMILES: CC(C)n1nc(-c2cnc(F)c(C(F)(F)F)c2)nc1[C@H]1CC[C@H](N2CCOCC2)C1] | 4-((1S,3S)-3-(3-(6-fluoro-5- (trifluoromethyl)pyridin-3- yl)-1-isopropyl-1H-1,2,4- triazol-5-yl)cyclopentyl)- morpholine |
| 160 | 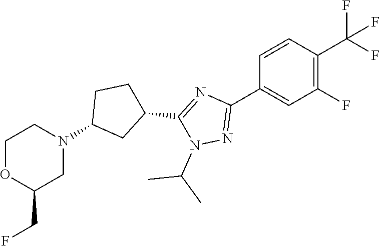 [SMILES: CC(C)n1nc(-c2ccc(C(F)(F)F)c(F)c2)nc1[C@H]1CC[C@@H](N2CCO[C@H](CF)C2)C1] | (S)-4-((1R,3S)-3-(3-(3- fluoro-4-(trifluoromethyl)- phenyl)-1-isopropyl-1H- 1,2,4-triazol-5-yl)cyclo- pentyl)-2-(fluoromethyl) morpholine |
| 161 | 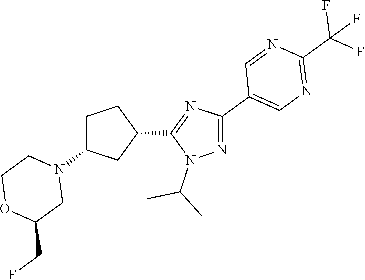 [SMILES: CC(C)n1nc(-c2cnc(C(F)(F)F)nc2)nc1[C@H]1CC[C@@H](N2CCO[C@H](CF)C2)C1] | (S)-2-(fluoromethyl)-4- ((1R,3S)-3-(1-isopropyl-3- (2-(trifluoromethyl)pyrim- idin-5-yl)-1H-1,2,4-triazol-5- yl)cyclopentyl)morpholine |
| 162 | 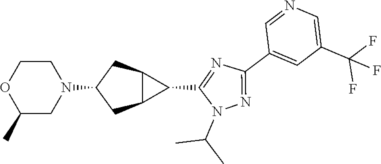 [SMILES: CC(C)n1nc(-c2cncc(C(F)(F)F)c2)nc1[C@H]1[C@@H]2C[C@@H](N3CCO[C@H](C)C3)C[C@@H]21] | (R)-4-((1R,3s,5S,6R)-6-(1- isopropyl-3-(5-(trifluoro- methyl)pyridin-3-yl)-1H- 1,2,4-triazol-5-yl)bicyclo [3.1.0]hexan-3-yl)-2-meth- ylmorpholine |
| 163 | 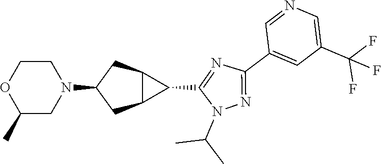 [SMILES: CC(C)n1nc(-c2cncc(C(F)(F)F)c2)nc1[C@H]1[C@@H]2C[C@H](N3CCO[C@H](C)C3)C[C@@H]21] | (R)-4-((1R,3r,5S,6R)-6-(1- isopropyl-3-(5-(trifluoro- methyl)pyridin-3-yl)-1H- 1,2,4-triazol-5-yl)bicyclo [3.1.0]hexan-3-yl)-2-meth- ylmorpholine |
| 164* | 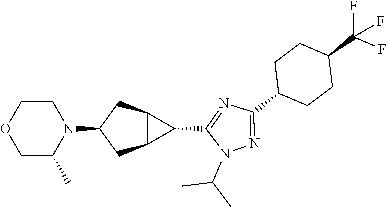 [SMILES: CC(C)n1nc([C@H]2CC[C@H](C(F)(F)F)CC2)nc1[C@H]1[C@@H]2C[C@H](N3CCOC[C@H]3C)C[C@@H]21] | (R)-4-((1R,3R,5S,6R)-6-(1- isopropyl-3-((1R,4R)-4- (trifluoromethyl)cyclohex- yl)-1H-1,2,4-triazol-5-yl)- bicyclo[3.1.0]hexan-3-yl)- 3-methylmorpholine |
| 165* | 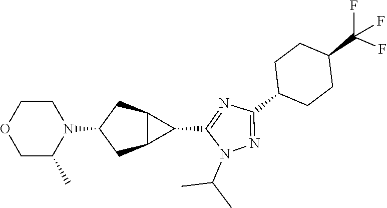 [SMILES: CC(C)n1nc([C@H]2CC[C@H](C(F)(F)F)CC2)nc1[C@H]1[C@@H]2C[C@@H](N3CCOC[C@H]3C)C[C@@H]21] | (R)-4-((1R,3S,5S,6R)-6-(1- isopropyl-3-((1R,4R)-4- (trifluoromethyl)cyclohexyl)- 1H-1,2,4-triazol-5-yl)bicyclo- [3.1.0]hexan-3-yl)-3-methyl- morpholine |
| 166* | 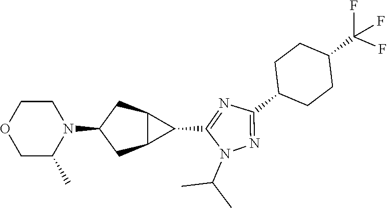 [SMILES: CC(C)n1nc([C@H]2CC[C@@H](C(F)(F)F)CC2)nc1[C@H]1[C@@H]2C[C@H](N3CCOC[C@H]3C)C[C@@H]21] | (R)-4-((1R,3R,5S,6R)-6-(1- isopropyl-3-((1s,4S)-4- (trifluoromethyl)cyclohexyl)- 1H-1,2,4-triazol-5-yl)bicyclo [3.1.0]hexan-3-yl)-3-methyl- morpholine |
| 167* | 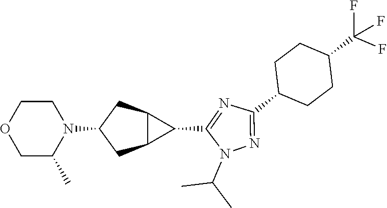 [SMILES: CC(C)n1nc([C@H]2CC[C@@H](C(F)(F)F)CC2)nc1[C@H]1[C@@H]2C[C@@H](N3CCOC[C@H]3C)C[C@@H]21] | (R)-4-((1R,3S,5S,6R)-6-(1- isopropyl-3-((1s,4S)-4- (trifluoromethyl)cyclohexyl)- 1H-1,2,4-triazol-5-yl)bicyclo [3.1.0]hexan-3-yl)-3-meth- ylmorpholine |
| 168 | 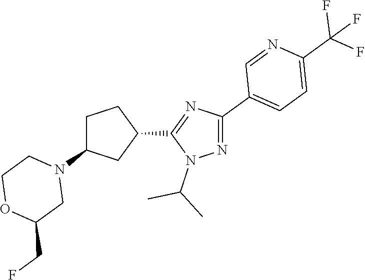 [SMILES: CC(C)n1nc(-c2ccc(C(F)(F)F)nc2)nc1[C@H]1CC[C@H](N2CCO[C@H](CF)C2)C1] | (S)-2-(fluoromethyl)-4- ((1S,3S)-3-(1-isopropyl-3-(6- (trifluoromethyl)pyridin-3- yl)-1H-1,2,4-triazol-5-yl)- cyclopentyl)morpholine |
| 169 | 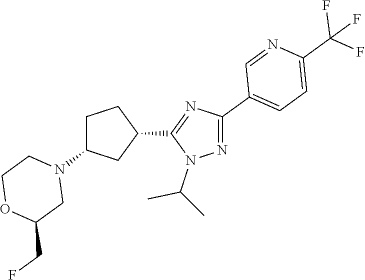 [SMILES: CC(C)n1nc(-c2ccc(C(F)(F)F)nc2)nc1[C@H]1CC[C@@H](N2CCO[C@H](CF)C2)C1] | (S)-2-(fluoromethyl)-4- ((1R,3S)-3-(1-isopropyl-3- (6-(trifluoromethyl)pyridin- 3-yl)-1H-1,2,4-triazol-5-yl)- cyclopentyl)morpholine |
| 170 | 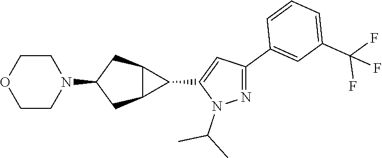 [SMILES: CC(C)n1nc(-c2cccc(C(F)(F)F)c2)cc1[C@H]1[C@@H]2C[C@H](N3CCOCC3)C[C@@H]21] | 4-((1R,3r,5S,6r)-6-(1-iso- propyl-3-(3-(trifluorometh- yl)phenyl)-1H-pyrazol-5- yl)bicyclo[3.1.0]hexan-3-yl)- morpholine |
| 171 | 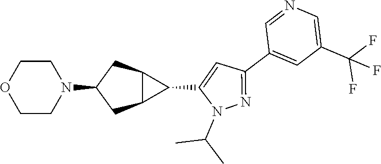 [SMILES: CC(C)n1nc(-c2cncc(C(F)(F)F)c2)cc1[C@H]1[C@@H]2C[C@H](N3CCOCC3)C[C@@H]21] | 4-((1R,3r,5S,6r)-6-(1-iso- propyl-3-(5-(trifluorometh- yl)pyridin-3-yl)-1H-pyrazol- 5-yl)bicyclo[3.1.0]hexan-3- yl)morpholine |
| 202 | 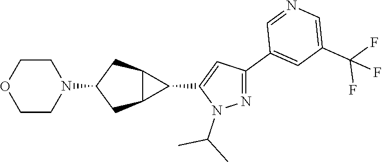 [SMILES: CC(C)n1nc(-c2cncc(C(F)(F)F)c2)cc1[C@H]1[C@@H]2C[C@@H](N3CCOCC3)C[C@@H]21] | 4-((1R,3s,5S,6r)-6-(1-iso- propyl-3-(5-trifluoromethyl)- pyridin-3-yl)-1H-pyrazol-5- yl)bicyclo[3.1.0]hexan-3-yl)- morpholine |
| 172 | 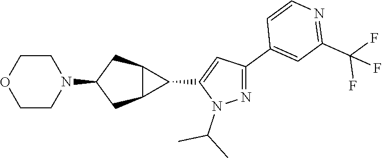 [SMILES: CC(C)n1nc(-c2ccnc(C(F)(F)F)c2)cc1[C@H]1[C@@H]2C[C@H](N3CCOCC3)C[C@@H]21] | 4-((1R,3r,5S,6r)-6-(1-iso- propyl-3-(2-(trifluorometh- yl)pyridin-4-yl)-1H-pyrazol- 5-yl)bicyclo[3.1.0]hexan-3- yl)morpholine |
| 173 | 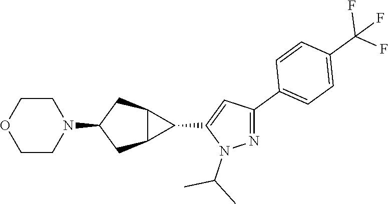 [SMILES: CC(C)n1nc(-c2ccc(C(F)(F)F)cc2)cc1[C@H]1[C@@H]2C[C@H](N3CCOCC3)C[C@@H]21] | 4-((1R,3r,5S,6r)-6-(1-iso- propyl-3-(4-(trifluorometh- yl)phenyl)-1H-5-yl)bicyclo [3.1.0]hexan-3-yl)morpholine |
| 174 | 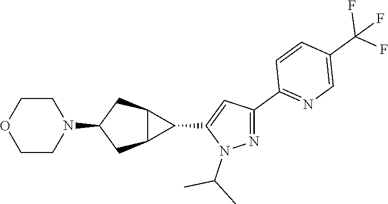 [SMILES: CC(C)n1nc(-c2ccc(C(F)(F)F)cn2)cc1[C@H]1[C@@H]2C[C@H](N3CCOCC3)C[C@@H]21] | 4-((1R,3r,5S,6r)-6-(1-iso- propyl-3-(5-(trifluorometh- yl)pyridin-2-yl)-1H-pyrazol- 5-yl)bicyclo[3.1.0]hexan-3- yl)morpholine |
| 175* | 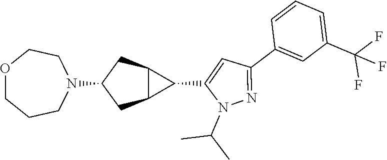 [SMILES: CC(C)n1nc(-c2cccc(C(F)(F)F)c2)cc1[C@H]1[C@@H]2C[C@@H](N3CCCOCC3)C[C@@H]21] | 4-((1R,3s,5S,6r)-6-(1-iso- propyl-3-(3-(trifluorometh- yl)phenyl)-1H-pyrazol-5-yl)- bicyclo[3.1.0]hexan-3-yl)- 1,4-oxazepane |
| 176* | 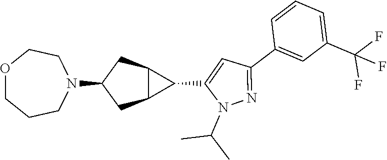 [SMILES: CC(C)n1nc(-c2cccc(C(F)(F)F)c2)cc1[C@H]1[C@@H]2C[C@H](N3CCCOCC3)C[C@@H]21] | 4-((1R,3r,5S,6r)-6-(1-iso- propyl-3-(3-(trifluorometh- yl)phenyl)-1H-pyrazol-5-yl)- bicyclo[3.1.0]hexan-3-yl)- 1,4-oxazepane |
| 177* | 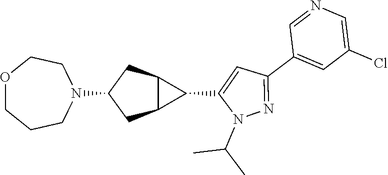 [SMILES: CC(C)n1nc(-c2cncc(Cl)c2)cc1[C@H]1[C@@H]2C[C@@H](N3CCCOCC3)C[C@@H]21] | 4-((1R,3s,5S,6r)-6-(3-(5- chloropyridin-3-yl)-1-iso- propyl-1H-pyrazol-5-yl)- bicyclo[3.1.0]hexan-3-yl)- 1,4-oxazepane |
| 178* | 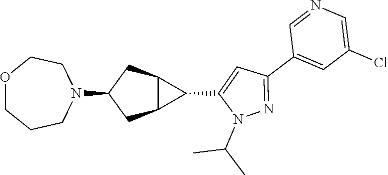 [SMILES: CC(C)n1nc(-c2cncc(Cl)c2)cc1[C@H]1[C@@H]2C[C@H](N3CCCOCC3)C[C@@H]21] | 4-((1R,3r,5S,6r)-6-(3-(5- chloropyridin-3-yl)-1-iso- propyl-1H-pyrazol-5-yl)- bicyclo[3.1.0]hexan-3-yl)- 1,4-oxazepane |
| 179* | 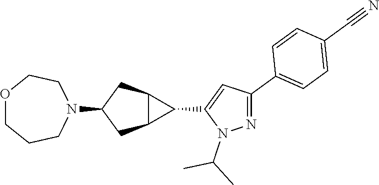 [SMILES: CC(C)n1nc(-c2ccc(C#N)cc2)cc1[C@H]1[C@@H]2C[C@H](N3CCCOCC3)C[C@@H]21] | 4-(5-((1R,3r,5S,6r)-3-(1,4- oxazepan-4-yl)bicyclo[3.1.0] hexan-6-yl)-1-isopropyl-1H- pyrazol-3-yl)benzonitrile |
| 180* | 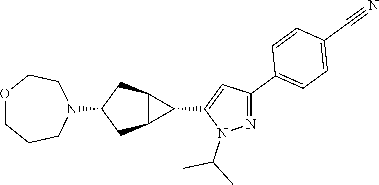 [SMILES: CC(C)n1nc(-c2ccc(C#N)cc2)cc1[C@H]1[C@@H]2C[C@@H](N3CCCOCC3)C[C@@H]21] | 4-(5-((1R,3s,5S,6r)-3-(1,4- oxazepan-4-yl)bicyclo[3.1.0] hexan-6-yl)-1-isopropyl-1H- pyrazol-3-yl)benzonitrile |
| 181* | 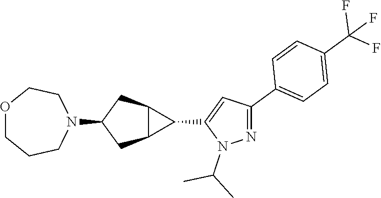 [SMILES: CC(C)n1nc(-c2ccc(C(F)(F)F)cc2)cc1[C@H]1[C@@H]2C[C@H](N3CCCOCC3)C[C@@H]21] | 4-((1R,3r,5S,6r)-6-(1-iso- propyl-3-(4-(trifluorometh- yl)phenyl)-1H-pyrazol-5-yl)- bicyclo[3.1.0]hexan-3-yl)- 1,4-oxazepane |
| 182* | 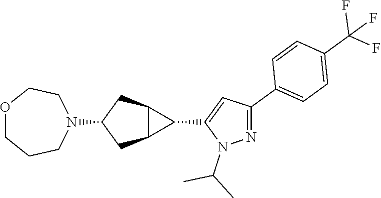 [SMILES: CC(C)n1nc(-c2ccc(C(F)(F)F)cc2)cc1[C@H]1[C@@H]2C[C@@H](N3CCCOCC3)C[C@@H]21] | 4-((1R,3s,5S,6r)-6-(1-iso- propyl-3-(4-(trifluorometh- yl)phenyl)-1H-pyrazol-5- yl)bicyclo[3.1.0]hexan-3- yl)-1,4-oxazepane |
| 183* | 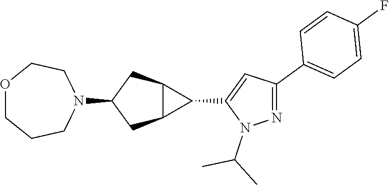 [SMILES: CC(C)n1nc(-c2ccc(F)cc2)cc1[C@H]1[C@@H]2C[C@H](N3CCCOCC3)C[C@@H]21] | 4-((1R,3r,5S,6r)-6-(3-(4- fluorophenyl)-1-isopropyl- 1H-pyrazol-5-yl)bicyclo [3.1.0]hexan-3-yl)-1,4- oxazepane |
| 184* | 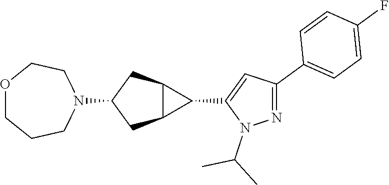 [SMILES: CC(C)n1nc(-c2ccc(F)cc2)cc1[C@H]1[C@@H]2C[C@@H](N3CCCOCC3)C[C@@H]21] | 4-((1R,3s,5S,6r)-6-(3-(4- fluorophenyl)-1-isopropyl- 1H-pyrazol-5-yl)bicyclo [3.1.0]hexan-3-yl)-1,4- oxazepane |
| 185* | 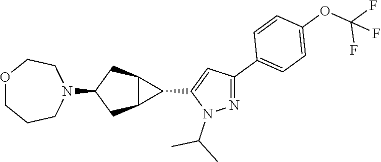 [SMILES: CC(C)n1nc(-c2ccc(OC(F)(F)F)cc2)cc1[C@H]1[C@@H]2C[C@H](N3CCCOCC3)C[C@@H]21] | 4-((1R,3r,5S,6r)-6-(1- isopropyl-3-(4-(trifluoro- methoxy)phenyl)-1H-pyr- azol-5-yl)bicyclo[3.1.0] hexan-3-yl)-1,4-oxazepane |
| 186* | 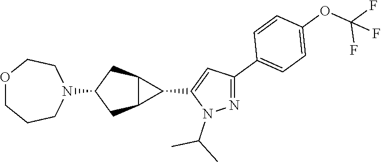 [SMILES: CC(C)n1nc(-c2ccc(OC(F)(F)F)cc2)cc1[C@H]1[C@@H]2C[C@@H](N3CCCOCC3)C[C@@H]21] | 4-((1R,3s,5S,6r)-6-(1-iso- propyl-3-(4-(trifluorometh- oxy)phenyl)-1H-pyrazol-5- yl)bicyclo[3.1.0]hexan-3- yl)-1,4-oxazepane |
| 187* | 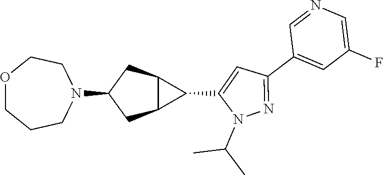 [SMILES: CC(C)n1nc(-c2cncc(F)c2)cc1[C@H]1[C@@H]2C[C@H](N3CCCOCC3)C[C@@H]21] | 4-((1R,3r,5S,6r)-6-(3-(5- fluoropyridin-3-yl)-1-iso- propyl-1H-pyrazol-5-yl)- bicyclo[3.1.0]hexan-3-yl)- 1,4-oxazepane |
| 188* | 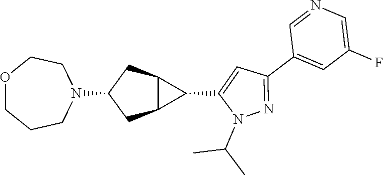 [SMILES: CC(C)n1nc(-c2cncc(F)c2)cc1[C@H]1[C@@H]2C[C@@H](N3CCCOCC3)C[C@@H]21] | 4-((1R,3s,5S,6r)-6-(3-(5- fluoropyridin-3-yl)-1-iso- propyl-1H-pyrazol-5-yl)- bicyclo[3.1.0]hexan-3-yl)- 1,4-oxazepane |
| 189* | 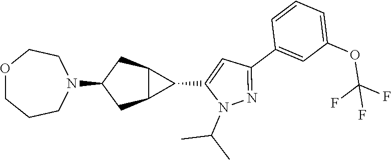 [SMILES: CC(C)n1nc(-c2cccc(OC(F)(F)F)c2)cc1[C@H]1[C@@H]2C[C@H](N3CCCOCC3)C[C@@H]21] | 4-((1R,3r,5S,6r)-6-(1-iso- propyl-3-(3-(trifluorometh- oxy)phenyl)-1H-pyrazol-5- yl)bicyclo[3.1.0]hexan-3-yl)- 1,4-oxazepane |
| 190* | 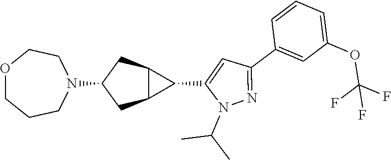 [SMILES: CC(C)n1nc(-c2cccc(OC(F)(F)F)c2)cc1[C@H]1[C@@H]2C[C@@H](N3CCCOCC3)C[C@@H]21] | 4-((1R,3s,5S,6r)-6-(1-iso- propyl-3-(3-(trifluorometh- oxy)phenyl)-1H-pyrazol-5- yl)bicyclo[3.1.0]hexan-3-yl)- 1,4-oxazepane |
| 191* | 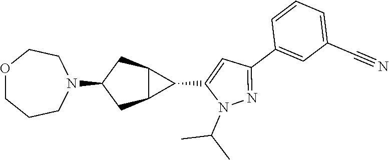 [SMILES: CC(C)n1nc(-c2cccc(C#N)c2)cc1[C@H]1[C@@H]2C[C@H](N3CCCOCC3)C[C@@H]21] | 3-(5-((1R,3r,5S,6r)-3-(1,4- oxazepan-4-yl)bicyclo[3.1.0] hexan-6-yl)-1-isopropyl-1H- pyrazol-3-yl)benzonitrile |
| 192* | 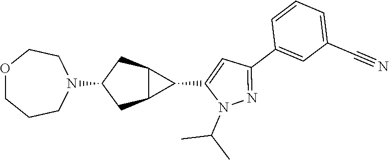 [SMILES: CC(C)n1nc(-c2cccc(C#N)c2)cc1[C@H]1[C@@H]2C[C@@H](N3CCCOCC3)C[C@@H]21] | 3-(5-((1R,3s,5S,6r)-3-(1,4- oxazepan-4-yl)bicyclo[3.1.0] hexan-6-yl)-1-isopropyl-1H- pyrazol-3-yl)benzonitrile |
| 193* | 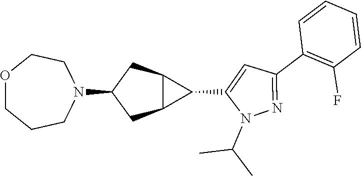 [SMILES: CC(C)n1nc(-c2ccccc2F)cc1[C@H]1[C@@H]2C[C@H](N3CCCOCC3)C[C@@H]21] | 4-((1R,3r,5S,6r)-6-(3-(2- fluorophenyl)-1-isopropyl- 1H-pyrazol-5-yl)bicyclo [3.1.0]hexan-3-yl)-1,4- oxazepane |
| 194* | 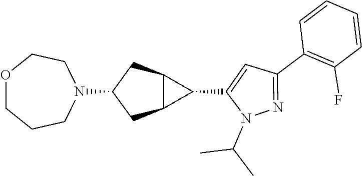 [SMILES: CC(C)n1nc(-c2ccccc2F)cc1[C@H]1[C@@H]2C[C@@H](N3CCCOCC3)C[C@@H]21] | 4-((1R,3s,5S,6r)-6-(3-(2- fluorophenyl)-1-isopropyl- 1H-pyrazol-5-yl)bicyclo [3.1.0]hexan-3-yl)-1,4- oxazepane |
| 195* | 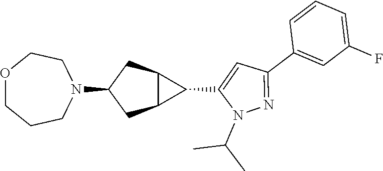 [SMILES: CC(C)n1nc(-c2cccc(F)c2)cc1[C@H]1[C@@H]2C[C@H](N3CCCOCC3)C[C@@H]21] | 4-((1R,3r,5S,6r)-6-(3-(3- fluorophenyl)-1-isopropyl- 1H-pyrazol-5-yl)bicyclo [3.1.0]hexan-3-yl)-1,4- oxazepane |
| 196* | 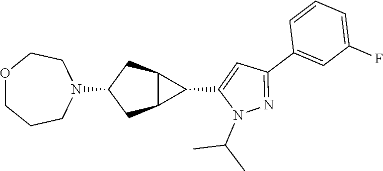 [SMILES: CC(C)n1nc(-c2cccc(F)c2)cc1[C@H]1[C@@H]2C[C@@H](N3CCCOCC3)C[C@@H]21] | 4-((1R,3s,5S,6r)-6-(3-(3- fluorophenyl)-1-isopropyl- 1H-pyrazol-5-yl)bicyclo [3.1.0]hexan-3-yl)-1,4- oxazepane |
| 197* | 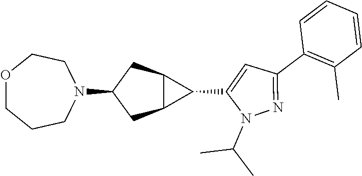 [SMILES: Cc1ccccc1-c1cc([C@H]2[C@@H]3C[C@H](N4CCCOCC4)C[C@@H]32)n(C(C)C)n1] | 4-((1R,3r,5S,6r)-6-(1-iso- propyl-3-(o-tolyl)-1H- pyrazol-5-yl)bicyclo[3.1.0] hexan-3-yl)-1,4-oxazepane |
| 198* | 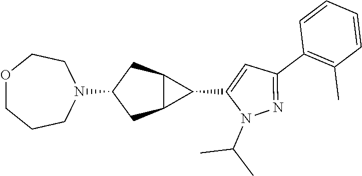 [SMILES: Cc1ccccc1-c1cc([C@H]2[C@@H]3C[C@@H](N4CCCOCC4)C[C@@H]32)n(C(C)C)n1] | 4-((1R,3s,5S,6r)-6-(1-iso- propyl-3-(o-tolyl)-1H- pyrazol-5-yl)bicyclo[3.1.0] hexan-3-yl)-1,4-oxazepane |
| 199* | 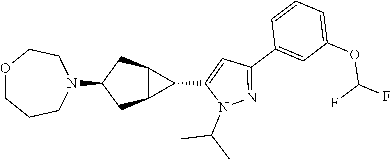 [SMILES: CC(C)n1nc(-c2cccc(OC(F)F)c2)cc1[C@H]1[C@@H]2C[C@H](N3CCCOCC3)C[C@@H]21] | 4-((1R,3r,5S,6r)-6-(3-(3- (difluoromethoxy)phenyl)-1- isopropyl-1H-pyrazol-5-yl)- bicyclo[3.1.0]hexan-3-yl)- 1,4-oxazepane |
| 200* | 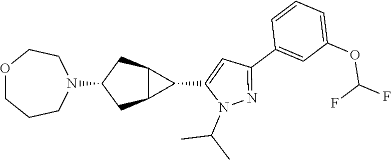 [SMILES: CC(C)n1nc(-c2cccc(OC(F)F)c2)cc1[C@H]1[C@@H]2C[C@@H](N3CCCOCC3)C[C@@H]21] | 4-((1R,3s,5S,6r)-6-(3-(3- (difluoromethoxy)phenyl)-1- isopropyl-1H-pyrazol-5-yl)- bicyclo[3.1.0]hexan-3-yl)- 1,4-oxazepane |
| 201* | 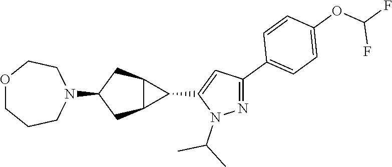 [SMILES: CC(C)n1nc(-c2ccc(OC(F)F)cc2)cc1[C@H]1[C@@H]2C[C@H](N3CCCOCC3)C[C@@H]21] | 4-((1R,3r,5S,6r)-6-(3-(4- (difluoromethoxy)phenyl)-1- isopropyl-1H-pyrazol-5-yl)- bicyclo[3.1.0]hexan-3-yl)- 1,4-oxazepane |
| 203 | 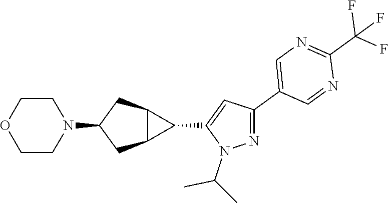 [SMILES: CC(C)n1nc(-c2cnc(C(F)(F)F)nc2)cc1[C@H]1[C@@H]2C[C@H](N3CCOCC3)C[C@@H]21] | 4-((1R,3r,5S,6r)-6-(1-iso- propyl-3-(2-(trifluorometh- yl)pyrimidin-5-yl)-1H- pyrazol-5-yl)bicyclo[3.1.0] hexan-3-yl)morpholine |
| 204 | 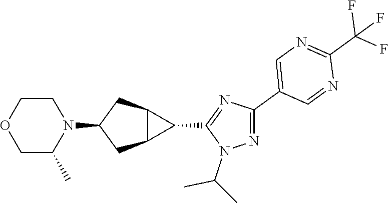 [SMILES: CC(C)n1nc(-c2cnc(C(F)(F)F)nc2)nc1[C@H]1[C@@H]2C[C@H](N3CCOC[C@H]3C)C[C@@H]21] | (R)-4-((1R,3r,5S,6r)-6-(1- isopropyl-3-(2-(trifluoro- methyl)pyrimidin-5-yl)-1H- 1,2,4-triazol-5-yl)bicyclo [3.1.0]hexan-3-yl)-3-methyl- morpholine |

### III. Pharmaceutical Compositions and Modes of Administration

Compounds provided herein are usually administered in the form of pharmaceutical compositions. Thus, provided herein are also pharmaceutical compositions that comprise one or more of the compounds described herein or a pharmaceutically acceptable salt, a stereoisomer, or a mixture of stereoisomers thereof and one or more pharmaceutically acceptable excipients. Suitable pharmaceutically acceptable excipients may include, for example, inert solid diluents and fillers, liquid diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In some embodiments, the pharmaceutical composition comprises a compound of Formula I, or a pharmaceutically acceptable salt thereof, and a pharmaceutically acceptable excipient. In some embodiments, the pharmaceutical composition comprises a compound of Formula Ia, or a pharmaceutically acceptable salt thereof, and a pharmaceutically acceptable excipient. In some embodiments, the compound of Formula I is a compound of Formula Ia′, or a pharmaceutically acceptable salt thereof, and a pharmaceutically acceptable excipient. In some embodiments, the pharmaceutical composition comprises a compound of Formula Ib, or a pharmaceutically acceptable salt thereof, and a pharmaceutically acceptable excipient. In some embodiments, the compound of Formula I is a compound of Formula Ib′, or a pharmaceutically acceptable salt thereof, and a pharmaceutically acceptable excipient. In some embodiments, the pharmaceutical composition comprises a compound of Formula Ia1, or a pharmaceutically acceptable salt thereof, and a pharmaceutically acceptable excipient. In some embodiments, the pharmaceutical composition comprises a compound of Formula Ib1, or a pharmaceutically acceptable salt thereof, and a pharmaceutically acceptable excipient. In some embodiments, the pharmaceutical composition comprises a compound of Formula Ia2, or a pharmaceutically acceptable salt thereof, and a pharmaceutically acceptable excipient. In some embodiments, the pharmaceutical composition comprises a compound of Formula Ib2, or a pharmaceutically acceptable salt thereof, and a pharmaceutically acceptable excipient. In some embodiments, the pharmaceutical composition comprises a compound of Table 1, or a pharmaceutically acceptable salt thereof, and a pharmaceutically acceptable excipient.

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or tablet, such as enteric coated tablets. In making the pharmaceutical compositions that include at least one compound described herein or a pharmaceutically acceptable salt, a stereoisomer, or a mixture of stereoisomers thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, a stereoisomer, or a mixture of stereoisomers thereof can be formulated so as to provide quick, sustained, or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, a stereoisomer, or a mixture of stereoisomers thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### IV. Methods of Treatment

Described herein are methods for promoting myelination of central nervous system neurons in a subject suffering from a myelin-related disorder, the method comprising administering to the subject a therapeutically effective amount of a compound of Formula I, Ia, Ia′, Ib, Ib′, Ia1, Ib1, Ia2, or Ib2, or a pharmaceutical composition comprising the same. In certain embodiments, the subject matter disclosed herein is directed to a compound of Formula I, Ia, Ia′, Ib, Ib′, Ia1, Ib1, Ia2, or Ib2, or a pharmaceutically acceptable salt thereof for promoting myelination of central nervous system neurons in a subject suffering from a myelin-related disorder. In another embodiment, the subject matter described herein is directed to the use of a compound of Formula I, Ia, Ia′, Ib, Ib′, Ia1, Ib1, Ia2, or Ib2, or a pharmaceutically acceptable salt thereof for the manufacture of a medicament for promoting myelination of central nervous system neurons in a subject suffering from a myelin-related disorder.

In certain embodiments, in the methods for promoting myelination of central nervous system neurons in a subject suffering from a myelin-related disorder, the compound of Formula I, Ia, Ia′, Ib, Ib′, Ia1, Ib1, Ia2, or Ib2, or a pharmaceutically acceptable salt thereof, or a pharmaceutical composition comprising the same, inhibits enzyme mediated synthesis of one or more sterol intermediates in the cholesterol biosynthesis pathway.

In certain embodiments, in the methods for promoting myelination of central nervous system neurons in a subject suffering from a myelin-related disorder, the compound of Formula I, Ia, Ia′, Ib, Ib′, Ia1, Ib1, Ia2, or Ib2, or a pharmaceutically acceptable salt thereof, or a pharmaceutical composition comprising the same, promotes accumulation of Δ8,9-unsaturated sterol intermediates in the cholesterol biosynthesis pathway.

In certain embodiments, in the methods for promoting myelination of central nervous system neurons in a subject suffering from a myelin-related disorder, the compound of Formula I, Ia, Ia′, Ib, Ib′, Ia1, Ib1, Ia2, or Ib2, or a pharmaceutically acceptable salt thereof, or a pharmaceutical composition comprising the same, inhibits one or more of CYP51, sterol-14-reductase, or EBP enzyme mediated synthesis of sterol intermediates in the cholesterol biosynthesis pathway.

In certain embodiments, in the methods for promoting myelination of central nervous system neurons in a subject suffering from a myelin-related disorder, the compound of Formula I, Ia, Ia′, Ib, Ib′, Ia1, Ib1, Ia2, or Ib2, or a pharmaceutically acceptable salt thereof, or a pharmaceutical composition comprising the same, induces, promotes, and/or modulates oligodendrocyte precursor cell (OPC) differentiation, proliferation and/or maturation. In certain embodiments, the induction of OPC differentiation is characterized by an increase in myelin basic protein (MBP) expression.

In certain embodiments, the subject matter described herein is directed to a method of treating a disorder in a subject in need thereof, the method comprising administering to the subject in need thereof a therapeutically effective amount of a compound of Formula I, Ia, Ia′, Ib, Ib′, Ia1, Ib1, Ia2, or Ib2, or a pharmaceutically acceptable salt thereof. In certain embodiments, the subject has a myelin-related disorder. In some embodiments, the compound of Formula I is a compound of Formula Ia, or a pharmaceutically acceptable salt thereof. In some embodiments, the compound of Formula I is a compound of Formula Ia′, or a pharmaceutically acceptable salt thereof. In other embodiments, the compound of Formula I is a compound of Formula Ib, or a pharmaceutically acceptable salt thereof. In some embodiments, the compound of Formula I is a compound of Formula Ib′, or a pharmaceutically acceptable salt thereof. In other embodiments, the compound of Formula I is a compound of Formula Ia1, or a pharmaceutically acceptable salt thereof. In other embodiments, the compound of Formula I is a compound of Formula Ib1, or a pharmaceutically acceptable salt thereof. In other embodiments, the compound of Formula I is a compound of Formula Ia2, or a pharmaceutically acceptable salt thereof. In other embodiments, the compound of Formula I is a compound of Formula Ib2, or a pharmaceutically acceptable salt thereof. In some embodiments, the compound of Formula I is a compound of Table 1, or a pharmaceutically acceptable salt thereof.

In certain embodiments, the subject matter disclosed herein is directed to a compound of Formula I, Ia, Ia′, Ib, Ib′, Ia1, Ib1, Ia2, or Ib2, or a pharmaceutically acceptable salt thereof, for use in treating a disorder in a subject in need thereof. In certain embodiments, the subject has a myelin-related disorder. In some embodiments, the compound of Formula I is a compound of Formula Ia, or a pharmaceutically acceptable salt thereof. In some embodiments, the compound of Formula I is a compound of Formula Ia′, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ib′, or a pharmaceutically acceptable salt thereof. In other embodiments, the compound of Formula I is a compound of Formula Ia1, or a pharmaceutically acceptable salt thereof. In other embodiments, the compound of Formula I is a compound of Formula Ib1, or a pharmaceutically acceptable salt thereof. In other embodiments, the compound of Formula I is a compound of Formula Ia2, or a pharmaceutically acceptable salt thereof. In other embodiments, the compound of Formula I is a compound of Formula Ib2, or a pharmaceutically acceptable salt thereof. In some embodiments, the compound of Formula I is a compound of Table 1, or a pharmaceutically acceptable salt thereof.

In certain embodiments, the subject matter disclosed herein is directed to the use of a compound of Formula I, Ia, Ia′, Ib, Ib′, Ia1, Ib1, Ia2, or Ib2, or a pharmaceutically acceptable salt thereof, in the manufacture of a medicament for treating a disorder in a subject in need thereof. In certain embodiments, the subject has a myelin-related disorder. In some embodiments, the compound of Formula I is a compound of Formula Ia, or a pharmaceutically acceptable salt thereof. In some embodiments, the compound of Formula I is a compound of Formula Ia′, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ib′, or a pharmaceutically acceptable salt thereof. In other embodiments, the compound of Formula I is a compound of Formula Ia1, or a pharmaceutically acceptable salt thereof. In other embodiments, the compound of Formula I is a compound of Formula Ib1, or a pharmaceutically acceptable salt thereof. In other embodiments, the compound of Formula I is a compound of Formula Ia2, or a pharmaceutically acceptable salt thereof. In other embodiments, the compound of Formula I is a compound of Formula Ib2, or a pharmaceutically acceptable salt thereof. In some embodiments, the compound of Formula I is a compound of Table 1, or a pharmaceutically acceptable salt thereof.

In certain embodiments, the subject matter disclosed herein is directed to a method of promoting myelination in a subject in need thereof, comprising administering to the subject a therapeutically effective amount of a compound of Formula I, Ia, Ia′, Ib, Ib′, Ia1, Ib1, Ia2, or Ib2, or a pharmaceutically acceptable salt thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ia1, or a pharmaceutically acceptable salt thereof. In other embodiments, the compound of Formula I is a compound of Formula Ib1, or a pharmaceutically acceptable salt thereof. In other embodiments, the compound of Formula I is a compound of Formula Ia2, or a pharmaceutically acceptable salt thereof. In other embodiments, the compound of Formula I is a compound of Formula Ib2, or a pharmaceutically acceptable salt thereof. In some embodiments, the compound of Formula I is a compound of Table 1, or a pharmaceutically acceptable salt thereof.

In certain embodiments, the subject matter disclosed herein is directed to a compound of Formula I, Ia, Ia′, Ib, Ib′, Ia1, Ib1, Ia2, or Ib2, or a pharmaceutically acceptable salt thereof, or a pharmaceutical composition comprising the compound, for use in promoting myelination in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ia1, or a pharmaceutically acceptable salt thereof. In other embodiments, the compound of Formula I is a compound of Formula Ib1, or a pharmaceutically acceptable salt thereof. In other embodiments, the compound of Formula I is a compound of Formula Ia2, or a pharmaceutically acceptable salt thereof. In other embodiments, the compound of Formula I is a compound of Formula Ib2, or a pharmaceutically acceptable salt thereof. In some embodiments, the compound of Formula I is a compound of Table 1, or a pharmaceutically acceptable salt thereof.

In certain embodiments, the subject matter disclosed herein is directed to use of a compound of Formula I, Ia, Ia′, Ib, Ib′, Ia1, Ib1, Ia2, or Ib2, or a pharmaceutically acceptable salt thereof, or a pharmaceutical composition comprising the compound, in the manufacture of a medicament for promoting myelination in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ia1, or a pharmaceutically acceptable salt thereof. In other embodiments, the compound of Formula I is a compound of Formula Ib1, or a pharmaceutically acceptable salt thereof. In other embodiments, the compound of Formula I is a compound of Formula Ia2, or a pharmaceutically acceptable salt thereof. In other embodiments, the compound of Formula I is a compound of Formula Ib2, or a pharmaceutically acceptable salt thereof. In some embodiments, the compound of Formula I is a compound of Table 1, or a pharmaceutically acceptable salt thereof.

In certain embodiments, the subject matter disclosed herein is directed to a method of inducing endogenous oligodendrocyte precursor cell (OPC) differentiation in a subject in need thereof, the method comprising administering to the subject a therapeutically effective amount of a compound of Formula I, Ia, Ia′, Ib, Ib′, Ia1, Ib1, Ia2, or Ib2, or a pharmaceutically acceptable salt thereof, or a pharmaceutical composition comprising the same. In certain embodiments, the subject is suffering from a myelin-related disorder. In certain embodiments, the myelin-related disorder is multiple sclerosis.

Such myelin-related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy, and radiation-induced demyelination.

The compound of Formula I, Ia, Ia′, Ib, Ib′, Ia1, Ib1, Ia2, or Ib2, or a pharmaceutically acceptable salt thereof can be administered alone or in combination with another agent to a subject suffering from a myelin-related disorder to promote myelination of neurons (e.g., neuronal axons). A myelin-related disorder can include any disease, condition (e.g., those occurring from traumatic spinal cord injury and cerebral infarction), or disorder resulting in abnormalities of the myelin sheath. Abnormalities can be caused by loss of myelin referred to as demyelination, dysfunctional myelin referred to as dysmyelination, or failure to form enough myelin referred to as hypomyelination. A myelin related disorder as described herein can arise from a genetic disorder or from one or more of a variety of neurotoxic insults. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ia1, or a pharmaceutically acceptable salt thereof. In other embodiments, the compound of Formula I is a compound of Formula Ib1, or a pharmaceutically acceptable salt thereof. In other embodiments, the compound of Formula I is a compound of Formula Ia2, or a pharmaceutically acceptable salt thereof. In other embodiments, the compound of Formula I is a compound of Formula Ib2, or a pharmaceutically acceptable salt thereof. In some embodiments, the compound of Formula I is a compound of Table 1, or a pharmaceutically acceptable salt thereof.

“Demyelination” as used herein, refers to the act of demyelinating, or the damage or loss of part or all of the myelin sheath insulating the nerves, and is the hallmark of myelin-related disorders. In certain embodiments, demyelination refers to the damage or loss of part or all of the myelin sheath insulating a subset of nerves in an individual, such as, for example, one or more nerves localized in a particular area of the body (e.g., neurons in the brain or spinal cord, or both brain and spinal cord; or the optic nerve).

Myelination of neurons requires oligodendrocytes. The term “myelination”, as used herein, refers to the generation of the nerve's myelin sheath by replacing myelin producing cells or restoring their function. The neurons that undergo remyelination may be in the brain, spinal cord, or both the brain and spinal cord. Restoring the function of a myelin producing cell may include, for example, increasing the rate of myelin production in a cell (or cells) with a less-than-average production level. Such increase may encompass raising the rate of myelin production up to or exceeding average production level; but also may encompass raising the rate of myelin production to a level that is still less than average, but higher than the previous level.

“Promoting Myelination” as used herein refers to increasing the rate of myelin production rather than a mere net increase in the amount of myelin as compared to a baseline level of myelin production rate in a subject. An increase in the rate of myelin production can be determined using imaging techniques or functional measurements. In some embodiments, myelination is promoted by increasing the differentiation of OPCs, increasing the accumulation of 8,9-unsaturated sterol intermediates in the biosynthetic pathway, increasing the formation of OPCs, or any combinations thereof. Such activities may be evaluated, for example, using one or more in vitro assays, such as those described herein or known to one of skill in the art.

A “baseline level of myelin production rate” as used herein, refers to the rate of myelin production in subject being treated before the onset of treatment.

### V. Methods of Preparing Compounds of Formula I and Pharmaceutically Acceptable Salts Thereof

Compounds can be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g., Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources such as Aldrich Chemicals (Milwaukee, WI) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula I, or pharmaceutically acceptable salts thereof, may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or pharmaceutically acceptable salts thereof.

### EXAMPLES

The Examples provide exemplary methods for preparing compounds. Those skilled in the art will appreciate that other synthetic routes may be used to synthesize the compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art. The asterix (*) indicates an isolated isomer or isolated group of isomers, but that the stereochemistry has not been assigned.

### Example A: 4-((1s,4s)-4-(1-Isopropyl-3-(5-(trifluoromethyl)pyridin-3-yl)-1H-pyrazol-5-yl)cyclohexyl)morpholine and 4-((1r,4r)-4-(1-isopropyl-3-(5-(trifluoromethyl)pyridin-3-yl)-1H-pyrazol-5-yl)cyclohexyl)morpholine (Compounds 1* and 2*)

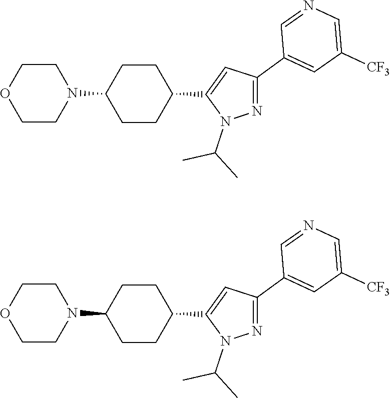 [SMILES: CC(C)n1nc(-c2cncc(C(F)(F)F)c2)cc1[C@H]1CC[C@@H](N2CCOCC2)CC1 ; CC(C)n1nc(-c2cncc(C(F)(F)F)c2)cc1[C@H]1CC[C@H](N2CCOCC2)CC1]

### Step 1: 3-Bromo-5-(1,4-dioxaspiro[4.5]dec-7-en-8-yl)-1-isopropyl-pyrazole

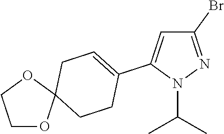 [SMILES: CC(C)n1nc(Br)cc1C1=CCC2(CC1)OCCO2]

A mixture of 3,5-dibromo-1-isopropyl-pyrazole (500 mg, 1.87 mmol), 4,4,5,5-tetramethyl-2-(1,4-dioxaspiro[4.5]dec-7-en-8-yl)-1,3,2-dioxaborolane (575 mg, 2.05 mmol), bis(triphenylphosphine)palladium(II) dichloride (65 mg, 0.09 mmol) and cesium carbonate (1.28 g, 3.73 mmol) in 1,4 dioxane (7.5 mL) and water (7.5 mL) was stirred at 75° C. for 18 h under N₂. The reaction mixture was cooled to rt, diluted with DCM, washed with brine, dried over anhydrous Mg₂SO₄ filtered and concentrated in vacuo. The residue was purified by silica flash chromatography (heptanes/isopropyl acetate) to give the title compound (380 mg, 62% yield). LCMS (ESI) [M+2H]⁺=328.1.

### Step 2: 3-[5-(1,4-Dioxaspiro[4.5]dec-7-en-8-yl)-1-isopropyl-pyrazol-3-yl]-5-(trifluoromethyl)pyridine

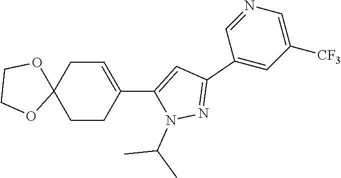 [SMILES: CC(C)n1nc(-c2cncc(C(F)(F)F)c2)cc1C1=CCC2(CC1)OCCO2]

A mixture of 3-bromo-5-(1,4-dioxaspiro[4.5]dec-7-en-8-yl)-1-isopropyl-pyrazole (380 mg, 1.16 mmol), 5-trifluoromethyl-pyridine-3-boronic acid (280 mg, 1.39 mmol), bis(triphenylphosphine)palladium(II) dichloride (41 mg, 0.06 mmol) and cesium carbonate (757 mg, 2.32 mmol) in 1,4 dioxane (4.6 mL) and water (4.6 mL) was stirred at 75° C. for 18 h under N₂. The reaction mixture was cooled to rt, diluted with DCM, washed with brine, dried over anhydrous Mg₂SO₄ filtered and concentrated in vacuo. The residue was purified by silica flash chromatography (heptanes/isopropylacetate) to give the title compound (80 mg, 18% yield). LCMS (ESI) [M+H]⁺=394.2.

### Step 3: 3-[5-(1,4-Dioxaspiro[4.5]decan-8-yl)-1-isopropyl-pyrazol-3-yl]-5-(trifluoromethyl)pyridine

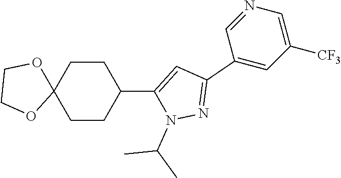 [SMILES: CC(C)n1nc(-c2cncc(C(F)(F)F)c2)cc1C1CCC2(CC1)OCCO2]

A solution of 3-[5-(1,4-dioxaspiro[4.5]dec-7-en-8-yl)-1-isopropyl-pyrazol-3-yl]-5-(trifluoromethyl)pyridine (80 mg, 0.20 mmol in ethanol (2 mL) was purged with nitrogen to remove oxygen and charged with 10% palladium on carbon (43 mg, 0.04 mmol) followed by a hydrogen balloon. The reaction mixture was stirred overnight at rt, filter through a celite cake, rinsed with ethanol 3 times and dried to afford the title compound as a crude intermediate which was taken into the next step without further purification. LCMS (ESI) [M+H]⁺=396.2.

### Step 4: 4-[2-Isopropyl-5-[5-(trifluoromethyl)-3-pyridyl]pyrazol-3-yl]cyclohexanone

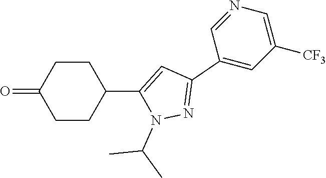 [SMILES: CC(C)n1nc(-c2cncc(C(F)(F)F)c2)cc1C1CCC(=O)CC1]

To a solution of 3-[5-(1,4-dioxaspiro[4.5]decan-8-yl)-1-isopropyl-pyrazol-3-yl]-5-(trifluoromethyl)pyridine (70 mg, 0.18 mmol) in acetone (0.4 mL) and water (0.2 mL) was added TFA (0.16 mL, 2.12 mmol) at 0° C., and the resulting mixture was stirred at room temperature for 18 h. The reaction solution was neutralized with a 1N NaOH solution. The resulting solution was extracted with saturated sodium bicarbonate solution and DCM. The organic layer was dried over anhydrous Mg₂SO₄, and concentrated under reduced pressure to afford the title compound (62 mg, quantitative yield) as a crude intermediate, which was taken into the next step without purification. LCMS (ESI) [M+H]⁺=352.2.

### Step 5: 4-((1s,4s)-4-(1-Isopropyl-3-(5-(trifluoromethyl)pyridin-3-yl)-1H-pyrazol-5-yl)cyclohexyl)morpholine and 4-((1r,4r)-4-(1-isopropyl-3-(5-(trifluoromethyl)pyridin-3-yl)-1H-pyrazol-5-yl)cyclohexyl)morpholine (Compounds 1* and 2*)

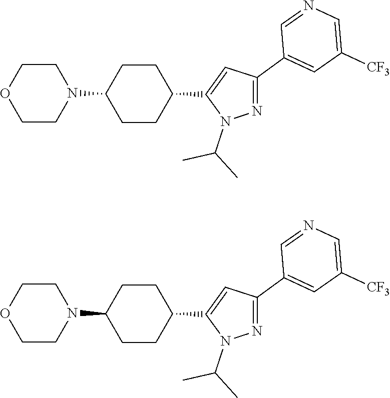 [SMILES: CC(C)n1nc(-c2cncc(C(F)(F)F)c2)cc1[C@H]1CC[C@@H](N2CCOCC2)CC1 ; CC(C)n1nc(-c2cncc(C(F)(F)F)c2)cc1[C@H]1CC[C@H](N2CCOCC2)CC1]

To a mixture of 4-[2-isopropyl-5-[5-(trifluoromethyl)-3-pyridyl]pyrazol-3-yl]cyclohexanone (62 mg, 0.18 mmol) and morpholine (0.19 mL, 0.22 mmol) in MeOH (2 mL) was added acetic acid (1 μL, 0.02 mmol) and sodium cyanoborohydride (25 mg, 0.40 mmol). The reaction was stirred at rt for 2, diluted with DCM, washed with a saturated sodium bicarbonate solution, dried over anhydrous Mg₂SO₄, filtered and concentrated in vacuo. The mixture was purified by reverse phase HPLC and the cis/trans isomers were then separated by chiral SFC [Chiralpak ID (150×21.2 mm, 5 μm); 15% isocratic 0.1% NH₄OH in MeOH @40° C.; 70 mL/min] to afford the titled compounds 1* (peak 1, 8.4 mg, 20% yield) and 2* (peak 2, 29.9 mg, 23% yield). LCMS (ESI) [M+H]⁺=423.20. The relative stereochemistry was arbitrarily assigned.

Compound 1*: ¹H NMR: (400 MHz, DMSO-d₆) δ 9.31 (d, J=1.9 Hz, 1H), 8.87-8.82 (m, 1H), 8.50-8.44 (m, 1H), 6.82 (s, 1H), 4.62 (hept, J=6.5 Hz, 1H), 3.65-3.59 (m, 4H), 2.95-2.85 (m, 1H), 2.43-2.39 (m, 3H), 2.21-2.15 (m, 1H), 2.02-1.93 (m, 2H), 1.86-1.74 (m, 2H), 1.65-1.51 (m, 4H), 1.44 (d, J=6.5 Hz, 6H).

Compound 2*: ¹H NMR: (400 MHz, DMSO-d₆) δ 9.27 (d, J=2.1 Hz, 1H), 8.89-8.83 (m, 1H), 8.44-8.38 (m, 1H), 6.80 (s, 1H), 4.62 (hept, J=6.5 Hz, 1H), 3.61-3.54 (m, 4H), 2.78-2.67 (m, 1H), 2.60-2.52 (m, 1H), 2.49-2.38 (m, 1H), 2.34-2.24 (m, 1H), 2.02-1.87 (m, 4H), 1.52-1.34 (m, 10H).

### Example B: 4-((1R,3R)-3-(1-Isopropyl-3-(6-(trifluoromethyl)pyridin-3-yl)-1H-1,2,4-triazol-5-yl)cyclopentyl)-1,4-oxazepane and 4-((1S,3R)-3-(1-Isopropyl-3-(6-(trifluoromethyl)pyridin-3-yl)-1H-1,2,4-triazol-5-yl)cyclopentyl)-1,4-oxazepane (Compounds 3 and 4)

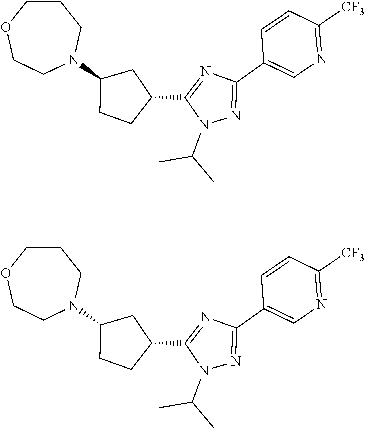 [SMILES: CC(C)n1nc(-c2ccc(C(F)(F)F)nc2)nc1[C@@H]1CC[C@@H](N2CCCOCC2)C1 ; CC(C)n1nc(-c2ccc(C(F)(F)F)nc2)nc1[C@@H]1CC[C@H](N2CCCOCC2)C1]

### Step 1: (R)-3-(1-Isopropyl-3-(6-(trifluoromethyl)pyridin-3-yl)-1H-1,2,4-triazol-5-yl)cyclopentanone

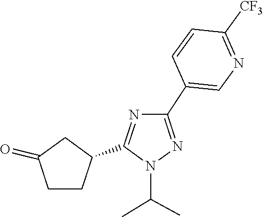 [SMILES: CC(C)n1nc(-c2ccc(C(F)(F)F)nc2)nc1[C@@H]1CCC(=O)C1]

To a solution of (1R)-3-oxocyclopentanecarboxylic acid (500.0 mg, 3.9 mmol) and 6-(trifluoromethyl)pyridine-3-amidine HCl (1300.0 mg, 5.76 mmol) in NN-dimethylformamide (4 mL)) were added N,N-diisopropylethylamine (2.65 mL, 15.61 mmol) and HATU (1632 mg, 4.29 mmol) and stirred at 20° C. for 1 h. Acetic acid (2.20 mL, 39 mmol) and isopropylhydrazine HCl (647.0 mg, 5.85 mmol) were then added and the mixture was heated to 80° C. and monitored by LCMS for consumption of acylamidine intermediate. After 2 h, the reaction mixture was cooled to room temperature. The mixture was diluted with ethyl acetate (20 mL), adjusted with NaHCO₃ (aq.) to pH=8 and extracted with ethyl acetate (100 mL×3). The combined organics were washed with brine (100 mL×3), dried over anhydrous sodium sulfate, filtered and concentrated in vacuo. The residue was dissolved in dioxane (10 mL) and HCl (12 M, 5 mL), and stirred for 2 h at 20° C. The mixture was adjusted with NaHCO₃ (aq.) to pH=8 and extracted with ethyl acetate (100 mL). The organic layer was dried over anhydrous sodium sulfate, filtered and concentrated in vacuo. The residue was purified by silica flash column chromatography (eluting with 0-50% ethyl acetate in petroleum ether) to provide the title compound (600 mg, 45% yield). LCMS (ESI), [M+H]⁺=339.1.

### Step 2: 4-((3R)-3-(1-Isopropyl-3-(6-(trifluoromethyl)pyridin-3-yl)-1H-1,2,4-triazol-5-yl)cyclopentyl)-1,4-oxazepane

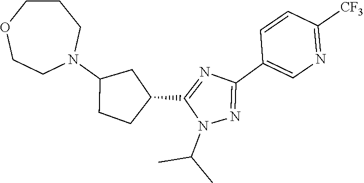 [SMILES: CC(C)n1nc(-c2ccc(C(F)(F)F)nc2)nc1[C@@H]1CCC(N2CCCOCC2)C1]

To a solution of homomorpholine HCl (366.0 mg, 2.66 mmol), (3R)-3-[2-isopropyl-5-[6-(trifluoromethyl)-3-pyridyl]-1,2,4-triazol-3-yl]cyclopentanone (600.0 mg, 1.77 mmol) and 4 Å molecular sieves in anhydrous 1,2-dichloroethane (3 mL) was added acetic acid (213.0 mg, 3.55 mmol). The reaction mixture stirred at 25° C. for 1 h. Sodium triacetoxyborohydride (1127 mg, 5.32 mmol) was then added and stirred at 25° C. for 16 h. The reaction was quenched with saturated sodium bicarbonate (10 mL) and extracted with DCM (50 mL×3). The combined organics were dried over anhydrous Na₂SO₄, filtered and concentrated in vacuo. The residue was purified by silica flash chromatography (0-10% MeOH in DCM) to provide the title compound (550 mg, 1.2858 mmol, 72.5% yield). LCMS (ESI), [M+H]⁺=424.2.

### Step 3: 4-((1R,3R)-3-(1-Isopropyl-3-(6-(trifluoromethyl)pyridin-3-yl)-1H-1,2,4-triazol-5-yl)cyclopentyl)-1,4-oxazepane and 4-((1S,3R)-3-(1-Isopropyl-3-(6-(trifluoromethyl)pyridin-3-yl)-1H-1,2,4-triazol-5-yl)cyclopentyl)-1,4-oxazepane (Compounds 3 and 4)

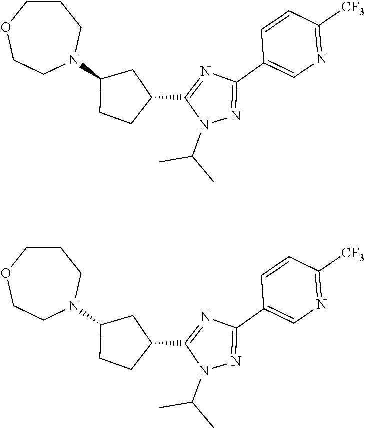 [SMILES: CC(C)n1nc(-c2ccc(C(F)(F)F)nc2)nc1[C@@H]1CC[C@@H](N2CCCOCC2)C1 ; CC(C)n1nc(-c2ccc(C(F)(F)F)nc2)nc1[C@@H]1CC[C@H](N2CCCOCC2)C1]

4-[Rac-(3R)-3-[2-isopropyl-5-[6-(trifluoromethyl)-3-pyridyl]-1,2,4-triazol-3-yl]cyclopentyl]-1,4-oxazepane (550.0 mg, 1.3 mmol) was separated using chiral SFC [Phenomenex-Cellulose-2 (250 mm*30 mm, 10 μm); 0.1% NH₃ in H₂O; MeOH 30/30; 70 mL/min] to provide the title compounds 3 (1^{st} peak on SFC, 26.8 mg, 4.7% yield) and 4 (2^{nd} peak on SFC, 454.2 mg, 81.8% yield). The relative stereochemistry was determined based on 2D-NMR analysis.

Compound 3: ¹H NMR (400 MHz, DMSO-d₆) δ 9.27 (s, 1H), 8.53 (dd, J=1.6, 8.0 Hz, 1H), 7.99 (d, J=8.4 Hz, 1H), 4.79-4.73 (m, 1H), 3.70-3.56 (m, 6H), 2.75 (brs, 4H), 2.13-2.03 (m, 4H), 1.91-1.78 (m, 3H), 1.70-1.54 (m, 1H), 1.45 (dd, J=4.0, 6.4 Hz, 6H).

Compound 4: ¹H NMR (400 MHz, DMSO-d₆) δ 9.27 (d, J=1.6 Hz, 1H), 8.54 (dd, J=1.6, 8.0 Hz, 1H), 7.98 (d, J=8.0 Hz, 1H), 4.79-4.71 (m, 1H), 3.67 (t, J=6.0 Hz, 2H), 3.63-3.61 (m, 2H), 3.46-3.42 (m, 1H), 3.16-3.07 (m, 1H), 2.78-2.62 (m, 4H), 2.30-2.25 (m, 1H), 2.09-2.02 (m, 1H), 1.97-1.87 (m, 2H), 1.84-1.75 (m, 3H), 1.68-1.58 (m, 1H), 1.45 (dd, J=3.2, 6.4 Hz, 6H).

### Example C: 4-((1S,3S)-3-(1-Isopropyl-3-(6-(trifluoromethyl)pyridin-3-yl)-1H-1,2,4-triazol-5-yl)cyclopentyl)-1,4-oxazepane and 4-((1R,3S)-3-(1-Isopropyl-3-(6-(trifluoromethyl)pyridin-3-yl)-1H-1,2,4-triazol-5-yl)cyclopentyl)-1,4-oxazepane (Compounds 5 and 6)

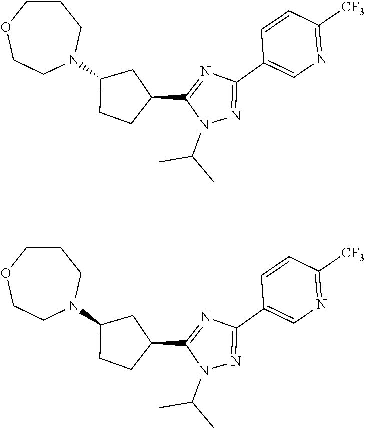 [SMILES: CC(C)n1nc(-c2ccc(C(F)(F)F)nc2)nc1[C@H]1CC[C@H](N2CCCOCC2)C1 ; CC(C)n1nc(-c2ccc(C(F)(F)F)nc2)nc1[C@H]1CC[C@@H](N2CCCOCC2)C1]

### Step 1: (S)-3-(1-Isopropyl-3-(6-(trifluoromethyl)pyridin-3-yl)-1H-1,2,4-triazol-5-yl)cyclopentanone

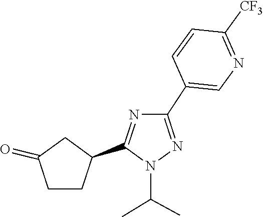 [SMILES: CC(C)n1nc(-c2ccc(C(F)(F)F)nc2)nc1[C@H]1CCC(=O)C1]

To a solution of (1S)-3-oxocyclopentanecarboxylic acid (500.0 mg, 3.9 mmol) and 6-(trifluoromethyl)pyridine-3-amidine HCl (1300.0 mg, 5.76 mmol) in N,N-dimethylformamide (4 mL)) were added N,N-diisopropylethylamine (2.65 mL, 15.61 mmol) and HATU (1632 mg, 4.29 mmol) and stirred at 20° C. for 1 h. Acetic acid (2.20 mL, 39 mmol) and isopropylhydrazine HCl (647.0 mg, 5.85 mmol) were then added. The reaction mixture was heated to 80° C. and monitored by LCMS for consumption of acylamidine intermediate. After 2 h, the reaction mixture was cooled to room temperature. The mixture was diluted with ethyl acetate (20 mL), adjusted with NaHCO₃ (aq.) to pH=8 and extracted with ethyl acetate (100 mL×3). The combined organics were washed with brine (100 mL×3), dried over anhydrous sodium sulfate, filtered and concentrated in vacuo. The residue was dissolved in dioxane (10 mL) and HCl (12 M, 5 mL), and stirred for 2 h at 20° C. The mixture was adjusted with NaHCO₃ (aq.) to pH=8 and extracted with ethyl acetate (100 mL). The organic layer was dried over sodium sulfate, filtered and concentrated in vacuo. The residue was purified by silica flash column chromatography (eluting with 0-50% ethyl acetate in petroleum ether) to provide the title compound (600 mg, 45% yield). LCMS (ESI), [M+H]⁺=339.1.

### Step 2: 4-((3S)-3-(1-Isopropyl-3-(6-(trifluoromethyl)pyridin-3-yl)-1H-1,2,4-triazol-5-yl)cyclopentyl)-1,4-oxazepane

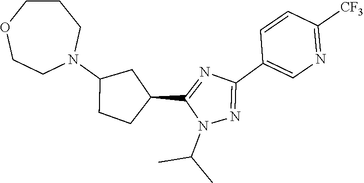 [SMILES: CC(C)n1nc(-c2ccc(C(F)(F)F)nc2)nc1[C@H]1CCC(N2CCCOCC2)C1]

To a solution of (3S)-3-[2-isopropyl-5-[6-(trifluoromethyl)-3-pyridyl]-1,2,4-triazol-3-yl]cyclopentanone (100.0 mg, 0.30 mmol) and homomorpholine HCl (122.0 mg, 0.89 mmol) in anhydrous methyl alcohol (5 mL) were added acetic acid (0.08 mL, 1.48 mmol) and sodium cyanoborohydride (93.0 mg, 1.48 mmol) at 20° C. The reaction stirred at 50° C. for 2 hours. The reaction was quenched with saturated sodium bicarbonate (10 mL) and extracted with dichloromethane (50 mL×3). The combined organics were dried over anhydrous Na₂SO₄, filtered and concentrated in vacuo. The residue was purified by silica flash chromatography (0-10% methanol in dichloromethane) to provide the title compounds (100 mg, 79.9% yield). LCMS (ESI), [M+H]⁺=424.2.

### Step 3: 4-((1S,3S)-3-(1-Isopropyl-3-(6-(trifluoromethyl)pyridin-3-yl)-1H-1,2,4-triazol-5-yl)cyclopentyl)-1,4-oxazepane and 4-((1R,3S)-3-(1-Isopropyl-3-(6-(trifluoromethyl)pyridin-3-yl)-1H-1,2,4-triazol-5-yl)cyclopentyl)-1,4-oxazepane (Compounds 5 and 6)

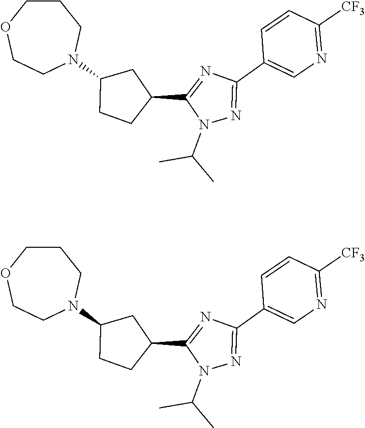 [SMILES: CC(C)n1nc(-c2ccc(C(F)(F)F)nc2)nc1[C@H]1CC[C@H](N2CCCOCC2)C1 ; CC(C)n1nc(-c2ccc(C(F)(F)F)nc2)nc1[C@H]1CC[C@@H](N2CCCOCC2)C1]

4-[rac-(3S)-3-[2-Isopropyl-5-[6-(trifluoromethyl)-3-pyridyl]-1,2,4-triazol-3-yl]cyclopentyl]-1,4-oxazepane (150.0 mg, 0.35 mmol) was separated using chiral SFC (phenomenex-cellulose-2 (250 mm*30 mm, 10 μm); 0.1% NH₃H₂O; MeOH; 25/25; 70 mL/min] to provide the title compounds 5 (1^{st} peak on SFC, 25.68 mg, 16.9% yield) and 6 (2^{nd} peak on SFC, 87.3 mg, 57.6% yield). The relative stereochemistry was determined by 2D-NMR.

Compound 5: ¹H NMR (400 MHz, DMSO-d₆) δ 9.26 (s, 1H), 8.53 (d, J=8.0 Hz, 1H), 7.98 (d, J=8.0 Hz, 1H), 4.79-4.72 (m, 1H), 3.70-3.56 (m, 6H), 2.82-2.61 (m, 4H), 2.13-2.02 (m, 4H), 1.89-1.75 (m, 3H), 1.59-1.49 (m, 1H), 1.45 (dd, J=4.0, 6.4 Hz, 6H).

Compound 6: ¹H NMR (400 MHz, DMSO-d₆) δ 9.26 (s, 1H), 8.54 (dd, J=1.2, 8.0 Hz 1H), 7.98 (d, J=8.0 Hz, 1H), 4.79-4.72 (m, 1H), 3.67 (t, J=6.0 Hz, 2H), 3.62-3.60 (m, 2H), 3.47-3.40 (m, 1H), 3.16-3.07 (m, 1H), 2.71-2.68 (m, 4H), 2.28-2.21 (m, 1H), 2.08-2.03 (m, 1H), 1.94-1.87 (m, 2H), 1.81-1.73 (m, 3H), 1.67-1.61 (m, 1H), 1.44 (dd, J=3.2, 6.4 Hz, 6H).

### Example D: 4-((1s,4s)-4-(1-Ethyl-3-(6-(trifluoromethyl)pyridin-3-yl)-1H-1,2,4-triazol-5-yl)cyclohexyl)-1,4-oxazepane and 4-((1r,4r)-4-(1-ethyl-3-(6-(trifluoromethyl)pyridin-3-yl)-1H-1,2,4-triazol-5-yl)cyclohexyl)-1,4-oxazepane (Compounds 7* and 8*)

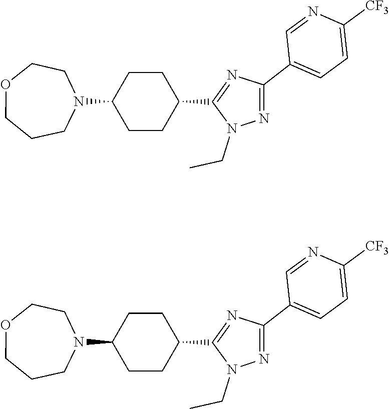 [SMILES: CCn1nc(-c2ccc(C(F)(F)F)nc2)nc1[C@H]1CC[C@@H](N2CCCOCC2)CC1 ; CCn1nc(-c2ccc(C(F)(F)F)nc2)nc1[C@H]1CC[C@H](N2CCCOCC2)CC1]

The title compounds were synthesized following a procedure similar to examples E and F, compounds 5 and 6, but instead using 4-oxocyclohexane-1-carboxylic acid, ethylhydrazine HCl and 6-(trifluoromethyl)nicotinimidamide HCl in step 1. The cis/trans mixture was separated by reverse phase HPLC to provide the title compounds 7* (1^{st} peak by LCMS, 26 mg, 13% yield) and 8* (2^{nd} peak by LCMS, 39 mg, 20% yield). LCMS (ESI) [M+H]⁺=424.20. The relative stereochemistry was arbitrarily assigned.

Compound 7*: ¹H NMR (400 MHz, DMSO-d₆) δ 9.26 (d, J=2.1 Hz, 1H), 8.56-8.49 (m, 1H), 8.04-7.98 (m, 1H), 4.27 (q, J=7.2 Hz, 2H), 3.93-3.64 (m, 4H), 3.56-3.33 (m, 6H), 3.09-3.00 (m, 1H), 2.17-2.11 (m, 2H), 2.07-1.98 (m, 3H), 1.80-1.68 (m, 4H), 1.43 (t, J=7.2 Hz, 3H).

Compound 8*: ¹H NMR (400 MHz, DMSO-d₆) δ 9.35 (d, J=2.0 Hz, 1H), 8.61-8.54 (m, 1H), 8.05-7.98 (m, 1H), 4.25 (q, J=7.2 Hz, 2H), 3.99-3.66 (m, 4H), 3.55-3.34 (m, 7H), 2.29-2.14 (m, 2H), 2.07-1.96 (m, 3H), 1.95-1.81 (m, 4H), 1.42 (t, J=7.2 Hz, 3H).

### Example E: 4-((1S,3S)-3-(1-Cyclopropyl-3-(6-(trifluoromethyl)pyridin-2-yl)-1H-1,2,4-triazol-5-yl)cyclopentyl)-1,4-oxazepane and 4-((1R,3S)-3-(1-cyclopropyl-3-(6-(trifluoromethyl)pyridin-2-yl)-1H-1,2,4-triazol-5-yl)cyclopentyl)-1,4-oxazepane (Compounds 9* and 10*)

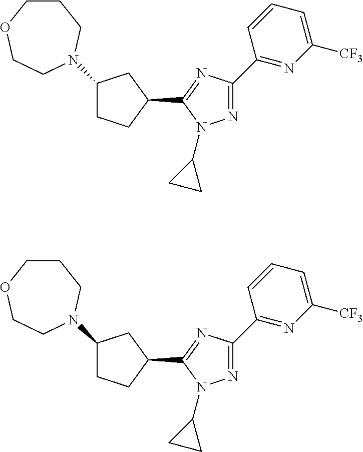 [SMILES: FC(F)(F)c1cccc(-c2nc([C@H]3CC[C@H](N4CCCOCC4)C3)n(C3CC3)n2)n1 ; FC(F)(F)c1cccc(-c2nc([C@H]3CC[C@@H](N4CCCOCC4)C3)n(C3CC3)n2)n1]

The title compounds were synthesized following a procedure similar to examples E and F, compounds 5 and 6, but instead using cyclopropylhydrazine HCl and 6-(trifluoromethyl)picolinimidamide HCl in step 1. The cis/trans mixture was separated by chiral SFC [Chiralpak ID (150×21.2 mm, 5 μm); 30% isocratic 0.1% NH₄OH in methanol; 40° C.; 70 mL/min] to provide the title compounds 9* (1^{st} peak on SFC, 10 mg, 9% yield) and compound 10* (2^{nd} peak on SFC, 47 mg, 42% yield). LCMS (ESI) [M+H]⁺=422.10. The relative stereochemistry was arbitrarily assigned.

Compound 9*: ¹H NMR (400 MHz, DMSO-d₆) δ 8.27 (d, J=8.0 Hz, 1H), 8.20-8.14 (m, 1H), 7.94-7.88 (m, 1H), 3.78-3.70 (m, 1H), 3.70-3.59 (m, 5H), 3.28-3.20 (m, 1H), 2.72-2.66 (m, 4H), 2.23-1.97 (m, 4H), 1.93-1.75 (m, 3H), 1.62-1.50 (m, 1H), 1.21-1.09 (m, 4H).

Compound 10*: ¹H NMR (400 MHz, DMSO-d₆) δ 8.28 (d, J=8.0 Hz, 1H), 8.21-8.13 (m, 1H), 7.94-7.88 (m, 1H), 3.79-3.59 (m, 5H), 3.59-3.48 (m, 1H), 3.20-3.08 (m, 1H), 2.75-2.67 (m, 4H), 2.37-2.27 (m, 1H), 2.17-2.05 (m, 1H), 2.01-1.86 (m, 2H), 1.85-1.73 (m, 3H), 1.73-1.59 (m, 1H), 1.26-1.06 (m, 4H).

### Example F: 4-((1S,3R)-3-(1-Cyclopropyl-3-(6-(trifluoromethyl)pyridin-2-yl)-1H-1,2,4 triazol-5-yl)cyclopentyl)-1,4-oxazepane and 4-((1R,3S)-3-(1-cyclopropyl-3-(6-(trifluoromethyl)pyridin-2-yl)-1H-1,2,4-triazol-5-yl)cyclopentyl)-1,4-oxazepane (Compounds 11* and 12*)

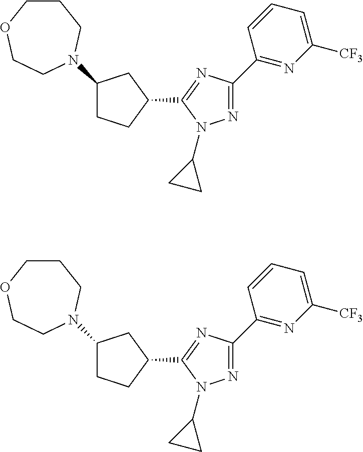 [SMILES: FC(F)(F)c1cccc(-c2nc([C@@H]3CC[C@@H](N4CCCOCC4)C3)n(C3CC3)n2)n1 ; FC(F)(F)c1cccc(-c2nc([C@@H]3CC[C@H](N4CCCOCC4)C3)n(C3CC3)n2)n1]

The title compounds were synthesized following a procedure similar to examples C and D, compounds 3 and 4, but instead using cyclopropylhydrazine HCl and 6-(trifluoromethyl)picolinimidamide HCl in step 1. The cis/trans mixture was separated by chiral SFC [Chiralpak IB—N (150×21.2 mm, 5 μm); 30% isocratic 0.1% NH₄OH in methanol, 30° C.; 70 mL/min] to provide the title compounds 11* (1^{st} peak on SFC, 6 mg, 5% yield) and 12* (2^{nd} peak on SFC, 41 mg, 33% yield). LCMS (ESI) [M+H]⁺=422.10. The relative stereochemistry was arbitrarily assigned.

Compound 11*: ¹H NMR (400 MHz, DMSO-d₆) δ 8.27 (d, J=7.9 Hz, 1H), 8.20-8.14 (m, 1H), 7.93-7.89 (m, 1H), 3.78-3.71 (m, 1H), 3.70-3.59 (m, 5H), 3.27-3.20 (m, 1H), 2.75-2.64 (m, 4H), 2.23-1.97 (m, 4H), 1.92-1.76 (m, 3H), 1.61-1.50 (m, 1H), 1.21-1.09 (m, 4H).

Compound 12*: ¹H NMR (400 MHz, DMSO-d₆) δ 8.28 (d, J=7.9 Hz, 1H), 8.20-8.14 (m, 1H), 7.93-7.89 (m, 1H), 3.77-3.65 (m, 3H), 3.65-3.59 (m, 2H), 3.59-3.49 (m, 1H), 3.19-3.09 (m, 1H), 2.75-2.66 (m, 4H), 2.37-2.27 (m, 1H), 2.17-2.06 (m, 1H), 1.99-1.88 (m, 2H), 1.85-1.73 (m, 3H), 1.73-1.59 (m, 1H), 1.23-1.08 (m, 4H).

### Example G: 4-((1R,3r,5S,6r)-6-(1-Isopropyl-3-(3-(trifluoromethyl)phenyl)-1H-1,2,4-triazol-5-yl)bicyclo[3.1.0]hexan-3-yl)-1,4-oxazepane and 4-((1R,3s,5S,6r)-6-(1-isopropyl-3-(3-(trifluoromethyl)phenyl)-1H-1,2,4-triazol-5-yl)bicyclo[3.1.0]hexan-3-yl)-1,4-oxazepane (Compounds 13 and 14)

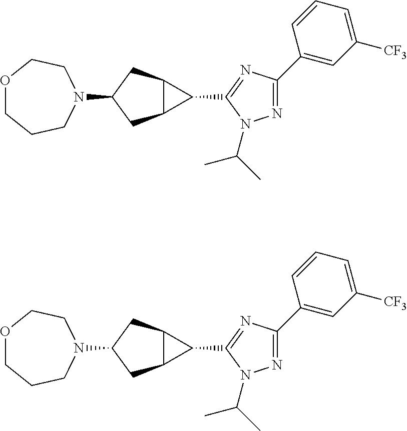 [SMILES: CC(C)n1nc(-c2cccc(C(F)(F)F)c2)nc1[C@H]1[C@@H]2C[C@H](N3CCCOCC3)C[C@@H]21 ; CC(C)n1nc(-c2cccc(C(F)(F)F)c2)nc1[C@H]1[C@@H]2C[C@@H](N3CCCOCC3)C[C@@H]21]

### Step 1: 5-((1R,5S)-3-((tert-Butyldiphenylsilyl)oxy)bicyclo[3.1.0]hexan-6-yl)-1-isopropyl-3-(3-(trifluoromethyl)phenyl)-1H-1,2,4-triazole

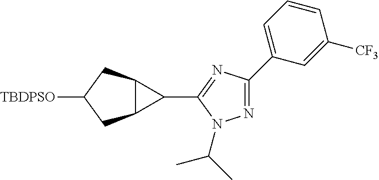

Following the general procedure in J. Org. Chem. 2011, 76, 1177, using (1R,5S)-3-((tert-butyldiphenylsilyl)oxy)bicyclo[3.1.0]hexane-6-carboxylic acid, isopropyl hydrazine HCl and 3-(trifluoromethyl)benzimidamide HCl, the title compound (1.31 g, 84.5% yield) was obtained following silica flash column chromatography (IprOAc/heptanes). LCMS (ESI) [M+H]⁺=590.3.

### Step 2: (1R,5S)-6-(1-Isopropyl-3-(3-(trifluoromethyl)phenyl)-1H-1,2,4-triazol-5-yl)bicyclo[3.1.0]hexan-3-ol

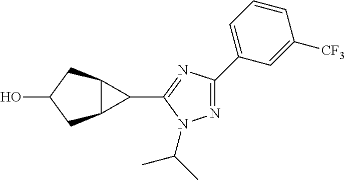

To a mixture of 5-((1R,5S)-3-((tert-butyldiphenylsilyl)oxy)bicyclo[3.1.0]hexan-6-yl)-1-isopropyl-3-(3-(trifluoromethyl)phenyl)-1H-1,2,4-triazole (1.31 g, 2.22 mmol) in THF (22 mL) was slowly added Et₃N·3HF (7.8 mL, 44.4 mmol). The reaction mixture was then heated to 70° C. for 14 h. The reaction mixture was cooled down to room temperature, quenched with saturated NaHCO₃ 200 mL) and then extracted with IprOAc (100 mL×2). The combined organic layers were washed with brine (100 mL), dried over anhydrous Mg₂SO₄, filtered, concentrated and purified by silica flash chromatography (0-100% heptanes/IprOAc) to provide the title compound (750 mg, 96% yield). LCMS (ESI) [M+H]⁺=352.2.

### Step 3: (1R,5S,6r)-6-(1-Isopropyl-3-(3-(trifluoromethyl)phenyl)-1H-1,2,4-triazol-5-yl)bicyclo[3.1.0]hexan-3-one

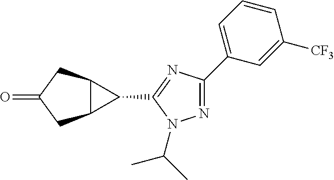 [SMILES: CC(C)n1nc(-c2cccc(C(F)(F)F)c2)nc1[C@H]1[C@@H]2CC(=O)C[C@@H]21]

To a stirred solution of (1R,5S)-6-(1-isopropyl-3-(3-(trifluoromethyl)phenyl)-1H-1,2,4-triazol-5-yl)bicyclo[3.1.0]hexan-3-ol (750 mg, 2.13 mmol) in DCM (21 mL) was added Dess-Martin reagent (1.36 g, 3.2 mmol). The reaction mixture was stirred at room temperature for 2 h. Saturated aqueous NaHCO₃ (100 mL) and sodium sulfite (100 mL) was slowly added to the reaction mixture and the resulting reaction mixture was stirred at room temperature for 0.5 h. The organic layer was separated, and the aqueous layer was extracted with DCM (100 mL×2). The combined organic layers were washed with brine, over anhydrous Mg₂SO₄, filtered, concentrated and purified by silica flash chromatography (IprOAc/Heptanes) to afford the title compound (710 mg, 2 mmol). LCMS (ESI) [M+H]⁺=350.1.

### Step 4: 4-((1R,3r,5S,6r)-6-(1-Isopropyl-3-(3-(trifluoromethyl)phenyl)-1H-1,2,4-triazol-5-yl)bicyclo[3.1.0]hexan-3-yl)-1,4-oxazepane and 4-((1R,3s,5S,6r)-6-(1-isopropyl-3-(3-(trifluoromethyl)phenyl)-1H-1,2,4-triazol-5-yl)bicyclo[3.1.0]hexan-3-yl)-1,4-oxazepane (Compounds 13 and 14)

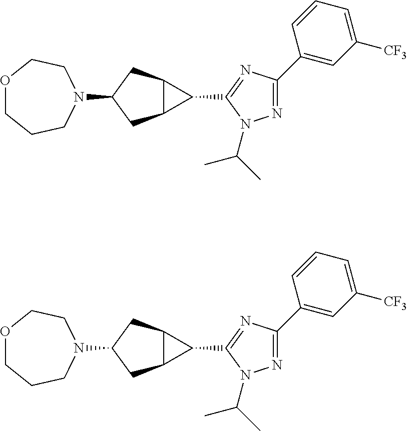 [SMILES: CC(C)n1nc(-c2cccc(C(F)(F)F)c2)nc1[C@H]1[C@@H]2C[C@H](N3CCCOCC3)C[C@@H]21 ; CC(C)n1nc(-c2cccc(C(F)(F)F)c2)nc1[C@H]1[C@@H]2C[C@@H](N3CCCOCC3)C[C@@H]21]

To a mixture of (1R,5S,6r)-6-(1-isopropyl-3-(3-(trifluoromethyl)phenyl)-1H-1,2,4-triazol-5-yl)bicyclo[3.1.0]hexan-3-one (142 mg, 0.41 mmol) and homomorpholine (65 mg, 0.61 mmol) in MeOH (4.1 mL) were added acetic acid (203 μL, 3.54 mmol) and sodium cyanoborohydride (51 mg, 0.81 mmol). The reaction mixture was stirred at 50° C. for 2 h, cooled then diluted with DCM, washed with an aq. saturated NaHCO₃ solution, dried over anhydrous Mg₂SO₄, filtered and concentrated in vacuo. The cis/trans mixture was separated by reverse phase HPLC (Interchim HPLC, 0.1% ammonium hydroxide in water, acetonitrile, XSelect CSH Prep C18, 50×30 mm×5 μm, 40-80% B over 10 min @60 mL/min) to give the titled compounds 13 (trans isomer, 27 mg, 15% yield) and 14 (cis isomer, 26 mg, 15% yield). LCMS (ESI) [M+H]⁺=435.2. The relative stereochemistry was determined by 2D-NMR analysis.

Compound 13: ¹H NMR (400 MHz, DMSO-d₆) δ 8.23-8.16 (m, 1H), 8.16-8.11 (m, 1H), 7.78-7.71 (m, 1H), 7.71-7.63 (m, 1H), 4.85 (hept, J=6.6 Hz, 1H), 3.66 (t, J=5.9 Hz, 2H), 3.63-3.56 (m, 2H), 3.30-3.21 (m, 1H), 2.68-2.60 (m, 4H), 2.21 (t, J=3.2 Hz, 1H), 2.20-2.12 (m, 2H), 1.92-1.85 (m, 2H), 1.82-1.71 (m, 2H), 1.67 (dd, J=13.6, 7.1 Hz, 2H), 1.48 (d, J=6.5 Hz, 6H).

Compound 14: ¹H NMR (400 MHz, DMSO-d₆) δ 8.23-8.17 (m, 1H), 8.15 (d, J=1.8 Hz, 1H), 7.78-7.71 (m, 1H), 7.71-7.63 (m, 1H), 4.88 (h, J=6.6 Hz, 1H), 3.66 (t, J=6.0 Hz, 2H), 3.63-3.56 (m, 2H), 3.00-2.87 (m, 1H), 2.66-2.57 (m, 4H), 2.15-2.05 (m, 3H), 1.95-1.86 (m, 2H), 1.83-1.67 (m, 4H), 1.45 (d, J=6.6 Hz, 6H).

### Example H: 4-((1R,3r,5S,6r)-6-(1-Isopropyl-3-(6-(trifluoromethyl)pyridin-3-yl)-1H-1,2,4-triazol-5-yl)bicyclo[3.1.0]hexan-3-yl)-1,4-oxazepane and 4-((1R,3s,5S,6r)-6-(1-isopropyl-3-(6-(trifluoromethyl)pyridin-3-yl)-1H-1,2,4-triazol-5-yl)bicyclo[3.1.0]hexan-3-yl)-1,4-oxazepane (Compounds 15 and 16)

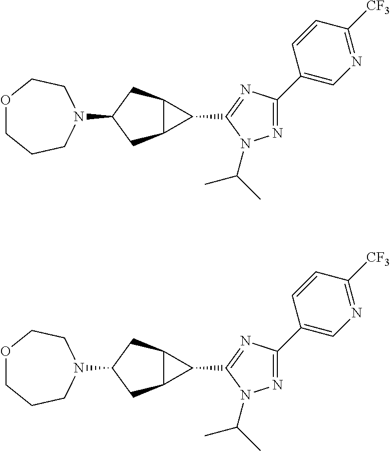 [SMILES: CC(C)n1nc(-c2ccc(C(F)(F)F)nc2)nc1[C@H]1[C@@H]2C[C@H](N3CCCOCC3)C[C@@H]21 ; CC(C)n1nc(-c2ccc(C(F)(F)F)nc2)nc1[C@H]1[C@@H]2C[C@@H](N3CCCOCC3)C[C@@H]21]

The title compounds were synthesized following a procedure similar to examples K and L, Compounds 13 and 14, but instead using 6-(trifluoromethyl)picolinimidamide HCl in step 1. The cis/trans mixture was separated by reverse phase HPLC (Interchim HPLC, 0.1% ammonium hydroxide in water, Solvent B: Acetonitrile, Column: XSelect CSH Prep C18, Column Dimension: 50×30 mm (5 μm), Column Temp: 25° C., Method: 30-70% B over 10 min @60 mL/min) to give the title compounds 15 (trans isomer; 17.5 mg, 11% yield) and 16 (cis isomer; 15 mg, 10% yield). LCMS (ESI) [M+H]⁺=436.2. The relative stereochemistry was determined by 2D-NMR.

Compound 15: ¹H NMR (400 MHz, DMSO-d₆) δ 9.24-9.18 (m, 1H), 8.51-8.44 (m, 1H), 7.99-7.92 (m, 1H), 4.88 (h, J=6.6 Hz, 1H), 3.66 (t, J=5.9 Hz, 2H), 3.63-3.56 (m, 2H), 3.30-3.19 (m, 1H), 2.68-2.60 (m, 4H), 2.28-2.23 (m, 1H), 2.23-2.12 (m, 2H), 1.93-1.85 (m, 2H), 1.76 (p, J=5.9 Hz, 2H), 1.72-1.63 (m, 2H), 1.49 (d, J=6.6 Hz, 6H).

Compound 16: ¹H NMR (400 MHz, DMSO-d₆) δ 9.24-9.19 (m, 1H), 8.52-8.44 (m, 1H), 8.00-7.92 (m, 1H), 4.93 (hept, J=6.6 Hz, 1H), 3.66 (t, J=6.0 Hz, 2H), 3.63-3.56 (m, 2H), 2.95 (p, J=8.4 Hz, 1H), 2.65-2.57 (m, 4H), 2.16-2.06 (m, 3H), 1.92 (tt, J=1.9, 1.0 Hz, 2H), 1.82-1.75 (m, 2H), 1.75-1.67 (m, 2H), 1.46 (d, J=6.6 Hz, 6H).

### Example I: 4-((1R,3r,5S,6r)-6-(1-Isopropyl-3-(3-(trifluoromethoxy)phenyl)-1H-1,2,4-triazol-5-yl)bicyclo[3.1.0]hexan-3-yl)-1,4-oxazepane and 4-((1R,3s,5S,6r)-6-(1-isopropyl-3-(3-(trifluoromethoxy)phenyl)-1H-1,2,4-triazol-5-yl)bicyclo[3.1.0]hexan-3-yl)-1,4-oxazepane (Compounds 17 and 18)

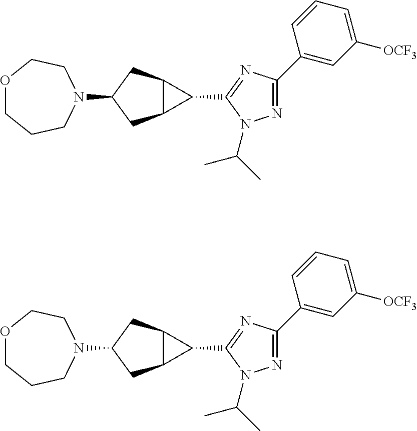 [SMILES: CC(C)n1nc(-c2cccc(OC(F)(F)F)c2)nc1[C@H]1[C@@H]2C[C@H](N3CCCOCC3)C[C@@H]21 ; CC(C)n1nc(-c2cccc(OC(F)(F)F)c2)nc1[C@H]1[C@@H]2C[C@@H](N3CCCOCC3)C[C@@H]21]

The title compounds were synthesized following a procedure similar to examples K and L, Compounds 13 and 14, but instead using 3-(trifluoromethoxy)benzimidamide in step 1. The cis/trans mixture was separated by reverse phase HPLC (Interchim HPLC, 0.1% ammonium hydroxide in water, acetonitrile, XSelect CSH Prep C18, 50×30 mm (5 μm), 25° C., 40-80% B over 10 min, 60 mL/min) to give the title compounds 17 (trans isomer; 31.8 mg, 14.4% yield) and 18 (cis isomer; 33.7 mg, 15% yield). LCMS (ESI) [M+H]⁺=451.2. The relative stereochemistry was determined by 2D-NMR analysis.

Compound 17: ¹H NMR (400 MHz, DMSO-d₆) δ 7.97-7.89 (m, 1H), 7.79-7.73 (m, 1H), 7.61-7.52 (m, 1H), 7.42-7.33 (m, 1H), 4.83 (h, J=6.6 Hz, 1H), 3.66 (t, J=5.9 Hz, 2H), 3.63-3.56 (m, 2H), 3.30-3.20 (m, 1H), 2.65-2.61 (m, 4H), 2.21-2.14 (m, 3H), 1.91-1.82 (m, 2H), 1.76 (p, J=5.9 Hz, 2H), 1.71-1.61 (m, 2H), 1.47 (d, J=6.6 Hz, 6H).

Compound 18: ¹H NMR (400 MHz, DMSO-d₆) δ 7.97-7.90 (m, 1H), 7.79-7.73 (m, 1H), 7.61-7.52 (m, 1H), 7.41-7.33 (m, 1H), 4.88 (hept, J=6.6 Hz, 1H), 3.66 (t, J=6.0 Hz, 2H), 3.62-3.56 (m, 2H), 3.00-2.87 (m, 1H), 2.66-2.57 (m, 4H), 2.15-2.07 (m, 2H), 2.07-2.04 (m, 1H), 1.94-1.84 (m, 2H), 1.82-1.75 (m, 2H), 1.75-1.66 (m, 2H), 1.44 (d, J=6.5 Hz, 6H).

### Example J: 4-((1R,3r,5S,6r)-6-(1-Isopropyl-3-(3-(trifluoromethyl)phenyl)-1H-1,2,4-triazol-5-yl)bicyclo[3.1.0]hexan-3-yl)morpholine and 4-((1R,3s,5S,6r)-6-(1-isopropyl-3-(3-(trifluoromethyl)phenyl)-1H-1,2,4-triazol-5-yl)bicyclo[3.1.0]hexan-3-yl)morpholine (Compounds 19* and 20*)

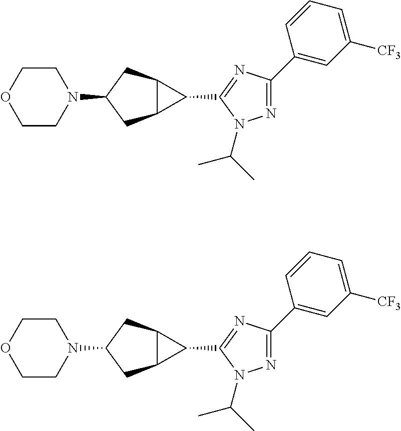 [SMILES: CC(C)n1nc(-c2cccc(C(F)(F)F)c2)nc1[C@H]1[C@@H]2C[C@H](N3CCOCC3)C[C@@H]21 ; CC(C)n1nc(-c2cccc(C(F)(F)F)c2)nc1[C@H]1[C@@H]2C[C@@H](N3CCOCC3)C[C@@H]21]

The title compounds were synthesized following a procedure similar to examples K and L, Compounds 13 and 14, but instead using morpholine in step 4. The cis/trans mixture was separated by reverse phase HPLC (Interchim HPLC; 0.1% ammonium hydroxide in water, acetonitrile, XSelect CSH Prep C18, 50×30 mm×5 μm), 25° C., 40-80% over 10 min @60 mL/min) to give the title compounds 19* (trans isomer, 31.9 mg, 17% yield) and 20* (cis isomer, 55.3 mg, 29% yield). LCMS (ESI) [M+H]⁺=421.1. The relative stereochemistry was arbitrarily assigned.

Compound 19*: ¹H NMR (400 MHz, DMSO-d₆) δ 8.23-8.17 (m, 1H), 8.17-8.11 (m, 1H), 7.78-7.71 (m, 1H), 7.71-7.63 (m, 1H), 4.81 (p, J=6.6 Hz, 1H), 3.57 (t, J=4.6 Hz, 4H), 2.85-2.74 (m, 1H), 2.38-2.34 (m, 4H), 2.34-2.28 (m, 1H), 2.16-2.05 (m, 2H), 1.96-1.86 (m, 2H), 1.85-1.75 (m, 2H), 1.48 (d, J=6.6 Hz, 6H).

Compound 20*: ¹H NMR (400 MHz, DMSO-d₆) δ 8.24-8.17 (m, 1H), 8.17-8.13 (m, 1H), 7.80-7.73 (m, 1H), 7.73-7.65 (m, 1H), 4.91-4.79 (m, 1H), 4.05-3.98 (m, 2H), 3.69-3.59 (m, 3H), 3.49-3.41 (m, 2H), 3.04-2.99 (m, 2H), 2.44-2.39 (m, 2H), 2.14-2.08 (m, 1H), 2.11-2.06 (m, 4H), 1.47 (d, J=6.6 Hz, 6H).

### Example K: 4-((1R,3R)-3-(1-Isopropyl-3-(3-(trifluoromethyl)phenyl)-1H-1,2,4-triazol-5-yl)cyclopentyl)morpholine and 4-((1S,3R)-3-(1-isopropyl-3-(3-(trifluoromethyl)phenyl)-1H-1,2,4-triazol-5-yl)cyclopentyl)morpholine (Compounds 21* and 22*)

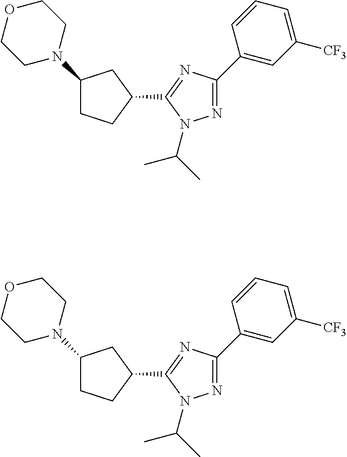 [SMILES: CC(C)n1nc(-c2cccc(C(F)(F)F)c2)nc1[C@@H]1CC[C@@H](N2CCOCC2)C1 ; CC(C)n1nc(-c2cccc(C(F)(F)F)c2)nc1[C@@H]1CC[C@H](N2CCOCC2)C1]

The title compounds were synthesized following a procedure similar to examples C and D, compounds 3 and 4, but instead using 3-(trifluoromethyl)benzimidamide HCl in step 1 and morpholine in step 2. The cis/trans mixture was separated by chiral SFC [PIC 200; Cellulose-2 (250×21.2 mm, 5 μm); 15% isocratic 0.1% NH₄OH in MeOH @40° C.; 70 mL/min] to provide the title compounds 21* (1^{st} peak on SFC, 27.6 mg, 13% yield) and 22* (2^{nd} peak on SFC, 75.4 mg, 36% yield). LCMS (ESI) [M+H]⁺=409.2. The relative stereochemistry was arbitrarily assigned.

Compound 21*: ¹H NMR (400 MHz, DMSO-d₆) δ 8.26 (d, J=7.7 Hz, 1H), 8.20 (s, 1H), 7.79 (d, J=7.8 Hz, 1H), 7.75-7.66 (m, 1H), 4.73 (hept, J=6.6 Hz, 1H), 4.05-3.99 (m, 1H), 3.93 (s, 1H), 3.60 (s, 5H), 3.19-3.13 (m, 1H), 2.79 (s, 1H), 2.45-2.39 (m, 2H), 2.30-2.24 (m, 2H), 2.14 (s, 1H), 2.05-1.99 (m, 1H), 1.91-1.82 (m, 1H), 1.49-1.41 (m, 6H)

Compound 22*: ¹H NMR (400 MHz, DMSO-d₆) δ 8.30-8.22 (m, 1H), 8.22-8.16 (m, 1H), 7.82-7.74 (m, 1H), 7.74-7.65 (m, 1H), 4.72 (h, J=6.6 Hz, 1H), 3.62-3.54 (m, 4H), 3.49-3.37 (m, 1H), 2.72-2.59 (m, 1H), 2.43 (s, 4H), 2.32-2.20 (m, 1H), 2.12-1.99 (m, 1H), 1.98-1.84 (m, 2H), 1.84-1.71 (m, 1H), 1.71-1.61 (m, 1H), 1.48-1.40 (m, 6H).

### Example L: 4-((1S,3S)-3-(1-Isopropyl-3-(3-(trifluoromethyl)phenyl)-1H-1,2,4-triazol-5-yl)cyclopentyl)morpholine and 4-((1R,3S)-3-(1-isopropyl-3-(3-(trifluoromethyl)phenyl)-1H-1,2,4-triazol-5-yl)cyclopentyl)morpholine (Compounds 23* and 24*)

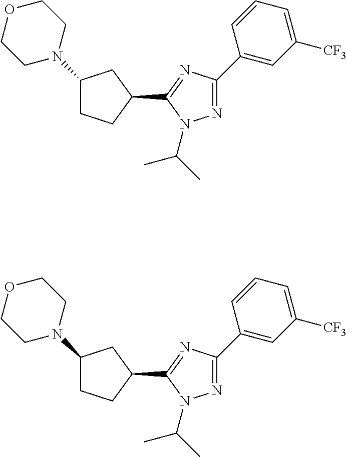 [SMILES: CC(C)n1nc(-c2cccc(C(F)(F)F)c2)nc1[C@H]1CC[C@H](N2CCOCC2)C1 ; CC(C)n1nc(-c2cccc(C(F)(F)F)c2)nc1[C@H]1CC[C@@H](N2CCOCC2)C1]

The title compounds were synthesized following a procedure similar to examples E and F, Compounds 5 and 6, but instead using 3-(trifluoromethyl)benzimidamide HCl in step 1 and morpholine in step 2. The cis/trans mixture was separated by chiral SFC [Cellulose-2 (250×21.2 mm×5 μm); 15% isocratic 0.1% NH₄OH in MeOH @40° C.; 70 mL/min] to provide the title compounds 23* (1^{st} peak on SFC, 22.9 mg, 11% yield) and 24* (2^{nd} peak on SFC, 68.9 mg, 32.4% yield). LCMS (ESI) [M+H]⁺=409.2. The relative stereochemistry was arbitrarily assigned.

Compound 23*: ¹H NMR (400 MHz, DMSO-d₆) δ 8.30-8.22 (m, 1H), 8.22-8.16 (m, 1H), 7.82-7.74 (m, 1H), 7.74-7.65 (m, 1H), 4.72 (h, J=6.5 Hz, 1H), 3.63-3.52 (m, 4H), 3.56-3.44 (m, 1H), 2.79 (p, J=7.6 Hz, 1H), 2.45-2.39 (m, 4H), 2.20-2.07 (m, 1H), 2.11-2.00 (m, 1H), 2.05-1.93 (m, 2H), 1.93-1.77 (m, 1H), 1.61-1.47 (m, 1H), 1.47-1.40 (m, 6H).

Compound 24*: ¹H NMR (400 MHz, DMSO-d₆) δ 8.30-8.22 (m, 1H), 8.22-8.16 (m, 1H), 7.82-7.74 (m, 1H), 7.74-7.65 (m, 1H), 4.73 (hept, J=6.5 Hz, 1H), 3.58 (t, J=4.6 Hz, 4H), 3.51-3.37 (m, 1H), 2.73-2.59 (m, 1H), 2.46-2.40 (m, 4H), 2.32-2.20 (m, 1H), 2.12-1.99 (m, 1H), 1.98-1.84 (m, 2H), 1.89-1.60 (m, 2H), 1.48-1.40 (m, 6H).

### Example M: 4-((1R,3R)-3-(1-Isopropyl-3-(6-(trifluoromethyl)pyridin-2-yl)-1H-1,2,4-triazol-5-yl)cyclopentyl)morpholine and 4-((1S,3R)-3-(1-isopropyl-3-(6-(trifluoromethyl)pyridin-2-yl)-1H-1,2,4-triazol-5-yl)cyclopentyl)morpholine (Compounds 25* and 26*)

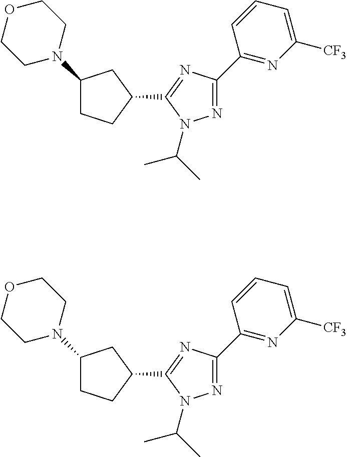 [SMILES: CC(C)n1nc(-c2cccc(C(F)(F)F)n2)nc1[C@@H]1CC[C@@H](N2CCOCC2)C1 ; CC(C)n1nc(-c2cccc(C(F)(F)F)n2)nc1[C@@H]1CC[C@H](N2CCOCC2)C1]

The title compound was synthesized following a procedure similar to examples C and D, Compounds 3 and 4, but instead using 6-(trifluoromethyl)picolinimidamide HCl in step 1 and morpholine in step 2. The cis/trans mixture was separated by chiral SFC [chiralcel OX (150×21.2 mm, 5 μm); 20% isocratic; 0.1% NH₄OH in MeOH @40° C.; 70 mL/min] to provide the title compounds 24* (1^{st} peak on SFC, 28.2 mg, 13.3% yield) and 26* (2^{nd} peak on SFC, 115.7 mg, 55% yield). LCMS (ESI) [M+H]⁺=410.2. The relative stereochemistry was arbitrarily assigned.

Compound 25*: ¹H NMR (400 MHz, DMSO-d₆) δ 8.30 (d, J=8.0 Hz, 1H), 8.22-8.13 (m, 1H), 7.92 (d, J=7.7 Hz, 1H), 4.76 (hept, J=6.5 Hz, 1H), 3.63-3.55 (m, 4H), 3.58-3.46 (m, 1H), 2.80 (p, J=7.5 Hz, 1H), 2.46-2.38 (m, 4H), 2.21-1.94 (m, 4H), 1.93-1.78 (m, 1H), 1.62-1.49 (m, 1H), 1.54-1.41 (m, 6H).

Compound 26*: ¹H NMR (400 MHz, DMSO-d₆) δ 8.31 (d, J=8.0 Hz, 1H), 8.22-8.13 (m, 1H), 7.96-7.88 (m, 1H), 4.76 (hept, J=6.6 Hz, 1H), 3.58 (t, J=4.6 Hz, 4H), 3.51-3.39 (m, 1H), 2.72-2.63 (m, 1H), 2.47-2.38 (m, 4H), 2.33-2.21 (m, 1H), 2.11-2.01 (m, 1H), 1.98-1.85 (m, 2H), 1.89-1.75 (m, 1H), 1.78-1.60 (m, 1H), 1.49-1.41 (m, 6H).

### Example N: 4-((1R,3R)-3-(1-Isopropyl-3-(4-(trifluoromethyl)pyridin-2-yl)-1H-1,2,4-triazol-5-yl)cyclopentyl)morpholine and 4-((1S,3R)-3-(1-isopropyl-3-(4-(trifluoromethyl)pyridin-2-yl)-1H-1,2,4-triazol-5-yl)cyclopentyl)morpholine and (Compounds 27* and 28*)

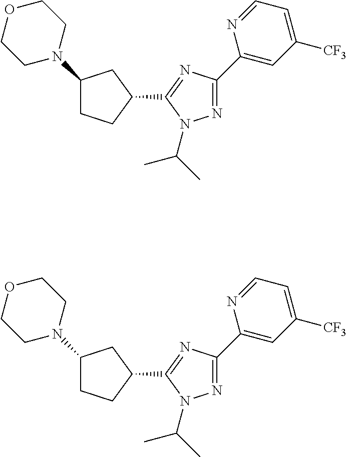 [SMILES: CC(C)n1nc(-c2cc(C(F)(F)F)ccn2)nc1[C@@H]1CC[C@@H](N2CCOCC2)C1 ; CC(C)n1nc(-c2cc(C(F)(F)F)ccn2)nc1[C@@H]1CC[C@H](N2CCOCC2)C1]

Title compounds were synthesized following a procedure similar to examples C and D, Compounds 3 and 4, but instead using 4-(trifluoromethyl)picolinimidamide HCl in step 1 and morpholine in step 2. The cis/trans mixture was separated by chiral SFC [Chiralpak IC (150×21.2 mm×5 μm); 20% isocratic 0.1% NH₄OH in MeOH; 40° C.; 70 mL/min] to provide the title compounds 27* (1^{st} peak on SFC, 1 mg, 3% yield) and 28* (2^{nd} peak on SFC, 4.1 mg, 19% yield). LCMS (ESI) [M+H]⁺=410.2. The relative stereochemistry was arbitrarily assigned.

Compound 27: not measured.

Compound 28: ¹H NMR (400 MHz, DMSO-d₆) δ 8.96-8.89 (m, 1H), 8.22-8.16 (m, 1H), 7.84-7.76 (m, 1H), 4.82-4.64 (m, 1H), 3.58 (t, J=4.6 Hz, 4H), 3.46 (p, J=8.1 Hz, 1H), 2.74-2.61 (m, 1H), 2.47-2.40 (m, 4H), 2.33-2.21 (m, 1H), 2.15-2.00 (m, 1H), 1.98-1.85 (m, 2H), 1.89-1.73 (m, 1H), 1.73-1.59 (m, 1H), 1.48-1.41 (m, 6H).

### Example O: 4-((1R,3r,5S,6r)-6-(1-Isopropyl-3-(5-(trifluoromethyl)pyridin-3-yl)-1H-pyrazol-5-yl)bicyclo[3.1.0]hexan-3-yl)-1,4-oxazepane and 4-((1R,3s,5S,6r)-6-(1-isopropyl-3-(5-(trifluoromethyl)pyridin-3-yl)-1H-pyrazol-5-yl)bicyclo[3.1.0]hexan-3-yl)-1,4-oxazepane (Compounds 29 and 30)

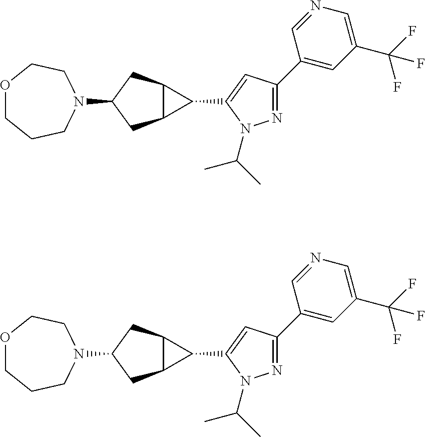 [SMILES: CC(C)n1nc(-c2cncc(C(F)(F)F)c2)cc1[C@H]1[C@@H]2C[C@H](N3CCCOCC3)C[C@@H]21 ; CC(C)n1nc(-c2cncc(C(F)(F)F)c2)cc1[C@H]1[C@@H]2C[C@@H](N3CCCOCC3)C[C@@H]21]

### Step 1: (1R,5S, 6r)-6-(1-Isopropyl-3-(5-(trifluoromethyl)pyridin-3-yl)-1H-pyrazol-5-yl)bicyclo [3.1.0]hexan-3-one

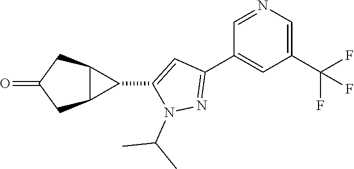 [SMILES: CC(C)n1nc(-c2cncc(C(F)(F)F)c2)cc1[C@H]1[C@@H]2CC(=O)C[C@@H]21]

To a solution of (1R,5S,6r)-6-(3-iodo-1-isopropyl-1H-pyrazol-5-yl)bicyclo[3.1.0]hexan-3-one (2.0 g, 6.1 mmol) and 3-(4,4,5,5-tetramethyl-1,3,2-dioxaborolan-2-yl)-5-(trifluoromethyl)pyridine (2.5 g, 9.2 mmol) in 1,4-dioxane (32 mL) and water (8 mL) were added Cs₂CO₃ (6 g, 18.4 mmol) and bis(di-tert-butyl(4-dimethylaminophenyl)phosphine)dichloropalladium(II) (460 mg, 0.65 mmol). The reaction mixture was then placed under nitrogen atmosphere and stirred at 100° C. for 4 hours. The reaction was quenched by water (30 mL) and extracted with ethyl acetate (100 mL×2). The combined organic layers were washed with brine (20 mL), dried over anhydrous Na₂SO₄, filtered and concentrated in vacuo. The residue was purified by silica flash chromatography (0-30% ethyl acetate in petroleum ether) to provide the title compound (2.1 g, 5.8 mmol, 96% yield). LCMS (ESI) [M+H]⁺=350.2.

### Step 2: 4-((1R,3s,5S,6r)-6-(1-Isopropyl-3-(5-(trifluoromethyl)pyridin-3-yl)-1H-pyrazol-5-yl)bicyclo [3.1.0]hexan-3-yl)-1,4-oxazepane & 4-((1R,3r,5S,6r)-6-(1-Isopropyl-3-(5-(trifluoromethyl) pyridin-3-yl)-1H-pyrazol-5-yl)bicyclo[3.1.0]hexan-3-yl)-1,4-oxazepane (Compounds 29* and 30*)

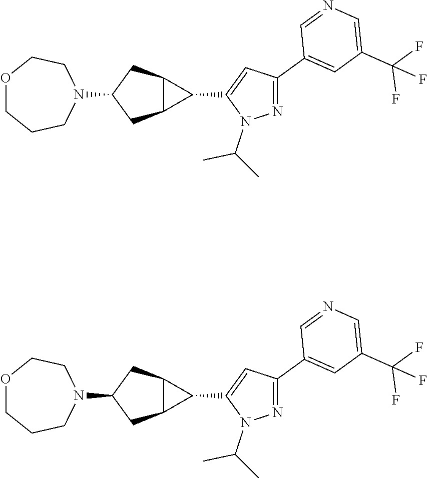 [SMILES: CC(C)n1nc(-c2cncc(C(F)(F)F)c2)cc1[C@H]1[C@@H]2C[C@@H](N3CCCOCC3)C[C@@H]21 ; CC(C)n1nc(-c2cncc(C(F)(F)F)c2)cc1[C@H]1[C@@H]2C[C@H](N3CCCOCC3)C[C@@H]21]

To a solution of (1R,5S,6r)-6-(1-isopropyl-3-(5-(trifluoromethyl)pyridin-3-yl)-1H-pyrazol-5-yl) bicyclo[3.1.0]hexan-3-one (100 mg, 0.28 mmol) and 1,4-oxazepane hydrochloride (60 mg, 0.44 mmol) in anhydrous methanol (4 mL) was added NaBH₃CN (90 mg, 1.44 mmol) at 20° C. The reaction mixture was then heated to 70° C. and stirred for 2 hours. The reaction was quenched by saturated NaHCO₃ solution (5 mL), and extracted with dichloromethane (30 mL×2). The combined organic phase was washed with brine, dried over anhydrous Na₂SO₄, filtered and concentrated under reduced pressure. The resulting residue was purified by prep-TLC (6% methanol in dichloromethane) to provide the mixture of diasteromeric compounds (90 mg, 72% yield), which were separated by chiral SFC (Daicel Chiralcel OJ-H (250 mm*30 mm, 5 μm); Supercritical CO₂/EtOH+NH₃·H₂O=90/10; 60 mL/min) to provide compound 29 (first peak on SFC, 14.8 mg, 16.3% yield) and compound 30 (second peak on SFC, 23.5 mg, 26% yield). LCMS (ESI) [M+H]⁺=435.4. The relative stereochemistry was assigned based on ¹H NMR.

Compound 29: ¹H NMR (400 MHz, CDCl₃) δ 9.11 (s, 1H), 8.74 (s, 1H), 8.29 (s, 1H), 6.13 (s, 1H), 4.80-4.60 (m, 1H), 3.80 (t, J=6.0 Hz, 2H), 3.76 (s, 2H), 3.39-3.33 (m, 1H), 2.74-2.67 (m, 4H), 2.39-2.32 (m, 2H), 1.95-1.88 (m, 2H), 1.82-1.79 (m, 1H), 1.56 (d, J=6.8 Hz, 6H), 1.26 (s, 4H).

Compound 30: ¹H NMR (400 MHz, CDCl₃) δ 9.11 (s, 1H), 8.74 (s, 1H), 8.29 (s, 1H), 6.17 (s, 1H), 4.76-4.69 (m, 1H), 3.79 (t, J=6.4 Hz, 2H), 3.74 (d, J=4.0 Hz, 2H), 2.90-2.62 (m, 5H), 2.33-2.13 (m, 2H), 1.98-1.91 (m, J=10.8 Hz, 4H), 1.70-1.65 (m, 3H), 1.55 (d, J=6.4 Hz, 6H).

### Example P: 4-((1R,3s,5S,6r)-6-(1-Isopropyl-3-(6-(trifluoromethyl)pyridin-2-yl)-1H-pyrazol-5-yl)bicyclo[3.1.0]hexan-3-yl)morpholine (Compound 31) and 4-((1R,3r,5S,6r)-6-(1-isopropyl-3-(6-(trifluoromethyl)pyridin-2-yl)-1H-pyrazol-5-yl)bicyclo[3.1.0]hexan-3-yl)morpholine (Compound 32)

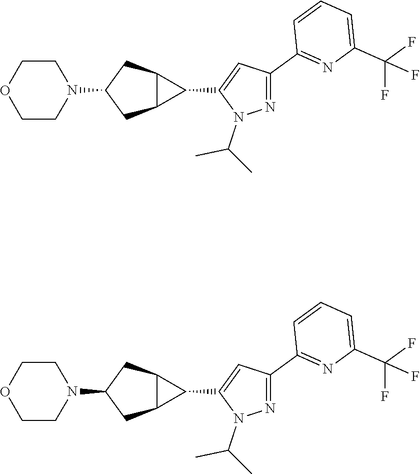 [SMILES: CC(C)n1nc(-c2cccc(C(F)(F)F)n2)cc1[C@H]1[C@@H]2C[C@@H](N3CCOCC3)C[C@@H]21 ; CC(C)n1nc(-c2cccc(C(F)(F)F)n2)cc1[C@H]1[C@@H]2C[C@H](N3CCOCC3)C[C@@H]21]

Title compounds were synthesized following a procedure similar to compound 29* using 2-(4,4,5,5-tetramethyl-1,3,2-dioxaborolan-2-yl)-6-(trifluoromethyl)pyridine in step 1. Purification of the crude mixture by silica flash chromatography (0-100% ethyl acetate in petroleum ether) to provide compound 31 (second peak on HPLC (basic), 19.2 mg, 23% yield) and compound 32 (first peak on HPLC (basic), 18.7 mg, 22% yield). LCMS (ESI) [M+H]⁺=421.1. The relative stereochemistry was determined by 2D-NMR analysis.

Compound 31: ¹H NMR (400 MHz, CD₃OD) δ 8.15 (d, J=8.0 Hz, 1H), 7.97 (t, J=7.6 Hz, 1H), 7.62 (d, J=7.6 Hz, 1H), 6.51 (s, 1H), 4.84-4.76 (m, 1H), 3.70 (t, J=4.4 Hz, 4H), 2.61-2.40 (m, 5H), 2.29 (dd, J=13.2, 7.2 Hz, 2H), 1.89-1.78 (m, 2H), 1.75 (s, 3H), 1.53 (d, J=6.4 Hz, 6H).

Compound 32: ¹H NMR (400 MHz, DMSO-d₆) δ 8.17-8.11 (m, 1H), 8.10-8.02 (m, 1H), 7.75 (d, J=7.6 Hz, 1H), 6.38 (s, 1H), 4.79-4.73 (m, 1H), 3.55 (t, J=4.4 Hz, 4H), 2.84-2.72 (m, 1H), 2.35-2.33 (m, 4H), 2.19-2.05 (m, 2H), 2.03-2.01 (m, 1H), 1.76-1.71 (m, 2H), 1.67 (brs, 2H), 1.47 (d, J=6.4 Hz, 6H).

### Example O: 4-((1R,3s,5S,6r)-6-(1-Isopropyl-3-(5-(trifluoromethyl)pyridin-3-yl)-1H-pyrazol-5-yl)bicyclo[3.1.0]hexan-3-yl)-2,2-dimethylmorpholine (Compound 33) and 4-((1R,3r,5S,6r)-6-(1-isopropyl-3-(5-(trifluoromethyl)pyridin-3-yl)-1H-pyrazol-5-yl)bicyclo[3.1.0]hexan-3-yl)-2,2-dimethylmorpholine (Compound 34)

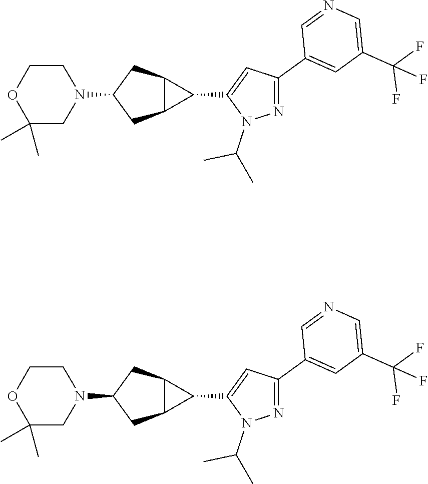 [SMILES: CC(C)n1nc(-c2cncc(C(F)(F)F)c2)cc1[C@H]1[C@@H]2C[C@@H](N3CCOC(C)(C)C3)C[C@@H]21 ; CC(C)n1nc(-c2cncc(C(F)(F)F)c2)cc1[C@H]1[C@@H]2C[C@H](N3CCOC(C)(C)C3)C[C@@H]21]

Title compounds were synthesized following a procedure similar to compound 29* using 3-(4,4,5,5-tetramethyl-1,3,2-dioxaborolan-2-yl)-5-(trifluoromethyl)pyridine in step 1. Purification of the crude mixture by reverse phase chromatography (column: Phenomenex Gemini C18 250*50 mm*10 um; mobile phase: [water (0.05% ammonia hydroxide); acetonitrile; 60%-80%, 10 min) to provide compound 33 (first peak on HPLC (basic), 24.4 mg, 23.5% yield) and compound 34 (second peak on HPLC (basic), 13.5 mg, 13% yield). LCMS (ESI) [M+H]⁺=449.3. The relative stereochemistry was determined by 2D-NMR.

Compound 33: ¹H NMR (400 MHz, CD₃OD) δ 9.15 (s, 1H), 8.73 (s, 1H), 8.41 (s, 1H), 6.43 (s, 1H), 4.82-4.75 (m, 1H), 3.75-3.65 (m, 2H), 2.42-2.32 (m, 3H), 2.29-2.18 (m, 4H), 1.83-1.73 (m, 5H), 1.53 (d, J=6.0 Hz, 6H), 1.24 (s, 6H).

Compound 34: ¹H NMR (400 MHz, CD₃OD) δ 9.15 (s, 1H), 8.73 (s, 1H), 8.41 (s, 1H), 6.38 (s, 1H), 4.82-4.75 (m, 1H), 3.71 (t, J=4.8 Hz, 2H), 2.74-2.72 (m, 1H), 2.42-2.32 (m, 2H), 2.25-2.19 (m, 2H), 2.16-2.09 (m, 3H), 1.93-1.89 (m, 2H), 1.69-1.64 (m, 2H), 1.54 (d, J=6.8 Hz, 6H), 1.24 (s, 6H).

### Example R: (1R,4R)-5-((1R,3s,5S,6R)-6-(1-Isopropyl-3-(5-(trifluoromethyl)pyridin-3-yl)-1H-pyrazol-5-yl)bicyclo[3.1.0]hexan-3-yl)-2-oxa-5-azabicyclo[2.2.1]heptane,(1S,4R)-5-((1R,3s,5S,6R)-6-(1-Isopropyl-3-(5-(trifluoromethyl) pyridin-3-yl)-1H-pyrazol-5-yl)bicyclo[3.1.0]hexan-3-yl)-2-oxa-5-azabicyclo[2.2.1]heptane, (1R,4R)-5-((1R,3r,5S,6R)-6-(1-Isopropyl-3-(5-(trifluoromethyl)pyridin-3-yl)-1H-pyrazol-5-yl)bicyclo[3.1.0]hexan-3-yl)-2-oxa-5-azabicyclo[2.2.1]heptane & (1S,4R)-5-((1R,3r,5S,6R)-6-(1-Isopropyl-3-(5-(trifluoromethyl)pyridin-3-yl)-1H-pyrazol-5-yl)bicyclo[3.1.0]hexan-3-yl)-2-oxa-5-azabicyclo[2.2.1]heptane (Compounds 35*, 36*, 37*, & 38*)

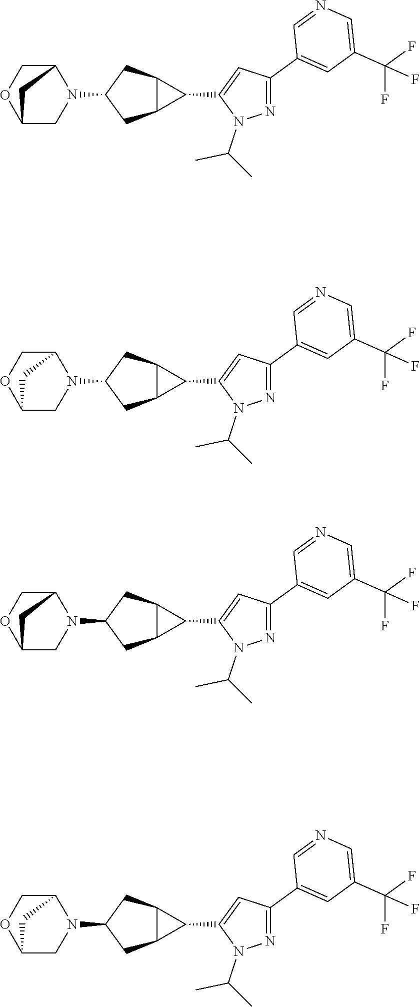 [SMILES: CC(C)n1nc(-c2cncc(C(F)(F)F)c2)cc1[C@H]1[C@@H]2C[C@@H](N3C[C@H]4C[C@@H]3CO4)C[C@@H]21 ; CC(C)n1nc(-c2cncc(C(F)(F)F)c2)cc1[C@H]1[C@@H]2C[C@@H](N3C[C@@H]4C[C@H]3CO4)C[C@@H]21 ; CC(C)n1nc(-c2cncc(C(F)(F)F)c2)cc1[C@H]1[C@@H]2C[C@H](N3C[C@H]4C[C@@H]3CO4)C[C@@H]21 ; CC(C)n1nc(-c2cncc(C(F)(F)F)c2)cc1[C@H]1[C@@H]2C[C@H](N3C[C@@H]4C[C@H]3CO4)C[C@@H]21]

Title compounds were synthesized following a procedure similar to compound 29* using 3-(4,4,5,5-tetramethyl-1,3,2-dioxaborolan-2-yl)-5-(trifluoromethyl) pyridine in step 1. Purification of the crude mixture by reverse phase chromatography (acetonitrile 70-100%/(0.05% NH₃H₂O+10 mM NH₄HCO₃ in water) to provide the cis-diastereomeric mixture (first peak on HPLC (basic), 70 mg, 32.2% yield) and the trans-diastereomeric mixture (second peak on HPLC (basic), 100 mg, 49% yield). LCMS (ESI) [M+H]⁺=433.4.

The cis mixture (peak 1) was confirmed by 2D NMR (first peak on HPLC (basic), 70 mg) was separated by chiral SFC (Daicel Chiralcel OJ-H (250 mm*30 mm, 5 μm); Supercritical CO₂/EtOH+NH₃·H₂O=85/15; 60 mL/min) to provide the cis title compound 35* (first peak on SFC, 13.9 mg, 18.9% yield) and the cis title compound 36* (second peak on SFC, 12.1 mg, 16.4% yield). LCMS (ESI) [M+H]⁺=433.4. The absolute stereochemistry was arbitrarily assigned.

Compound 35*: ¹H NMR (400 MHz, CDCl₃) δ 9.12 (d, J=1.6 Hz, 1H), 8.76 (s, 1H), 8.29 (s, 1H), 6.20 (s, 1H), 4.73-4.60 (m, 1H), 4.54-4.48 (m, 1H), 4.16-4.09 (m, 1H), 3.76-3.68 (m, 1H), 3.19-2.97 (m, 1H), 2.69-2.65 (m, 1H), 2.32-2.15 (m, 4H), 2.01-1.91 (m, 1H), 1.84-1.80 (m, 4H), 1.56 (d, J=6.8 Hz, 6H), 1.26 (s, 2H).

Compound 36*: ¹H NMR (400 MHz, CDCl₃) δ 9.12 (s, 1H), 8.76 (s, 1H), 8.29 (s, 1H), 6.21 (s, 1H), 4.66-4.58 (m, 2H), 4.25-4.18 (m, 1H), 3.79-3.72 (m, 1H), 3.19-3.16 (m, 1H), 2.75-2.69 (m, 1H), 2.35-2.15 (m, 4H), 2.04-1.95 (m, 1H), 1.86-1.82 (m, 4H), 1.58-1.55 (d, J=6.8 Hz, 6H), 1.26 (s, 2H).

The trans stereochemistry was confirmed by 2D NMR (second peak on HPLC (basic), 100 mg), and the mixture was separated by chiral SFC (Daicel Chiralcel OJ-H (250 mm*30 mm, 5 μm); supercritical CO₂/EtOH+NH₃·H₂O=75/25; 60 mL/min) to provide the trans title compound 37* (first peak on SFC, 33.9% yield) and the trans title compound 38* (second peak on SFC, 49.4 mg, 48.4% yield). The absolute stereochemistry was arbitrarily assigned. LCMS (ESI) [M+H]⁺=433.4.

Compound 37*: ¹H NMR (400 MHz, CDCl₃) δ 9.11 (s, 1H), 8.74 (s, 1H), 8.29 (s, 1H), 6.11 (s, 1H), 4.75-4.69 (m, 1H), 4.39-4.35 (m, 1H), 4.06-3.98 (m, 1H), 3.65-3.56 (m, 2H), 3.35-3.31 (m, 1H), 2.93-2.88 (m, 1H), 2.52-2.40 (m, 2H), 2.16-1.93 (m, 3H), 1.69-1.63 (m, 4H), 1.56 (d, J=6.8 Hz, 6H), 1.26 (s, 1H).

Compound 38*: ¹H NMR (400 MHz, CDCl₃) δ 9.11 (d, J=1.6 Hz, 1H), 8.74 (s, 1H), 8.29 (s, 1H), 6.12 (s, 1H), 4.72-4.68 (m, 1H), 4.44-4.37 (m, 1H), 4.03-3.98 (m, 1H), 3.74-3.62 (m, 2H), 3.34-3.31 (m, 1H), 2.93-2.89 (m, 1H), 2.54-2.51 (m, 1H), 2.41-2.38 (m, 1H), 2.20-2.17 (m, 1H), 1.93-1.89 (m, 2H), 1.82-1.70 (m, 2H), 1.69-1.65 (m, 2H), 1.56 (d, J=6.8 Hz, 6H), 1.26 (s, 1H).

### Example S: (S)-4-((1R,3s,5S,6S)-6-(1-Isopropyl-3-(5-(trifluoromethyl)pyridin-3-yl)-1H-pyrazol-5-yl)bicyclo[3.1.0]hexan-3-yl)-2-methylmorpholine, (R)-4-((1R,3s,5S,6R)-6-(1-isopropyl-3-(5-(trifluoromethyl)pyridin-3-yl)-1H-pyrazol-5-yl)bicyclo[3.1.0]hexan-3-yl)-2-methylmorpholine, (S)-4-((1R,3r,5S,6S)-6-(1-isopropyl-3-(5-(trifluoromethyl)pyridin-3-yl)-1H-pyrazol-5-yl)bicyclo[3.1.0]hexan-3-yl)-2-methylmorpholine & (R)-4-((1R,3r,5S,6R)-6-(1-isopropyl-3-(5-(trifluoromethyl)pyridin-3-yl)-1H-pyrazol-5-yl)bicycle[3.1.0]hexan-3-yl)-2-methylmorpholine (Compounds 39*, 40*, 41*, & 42*)

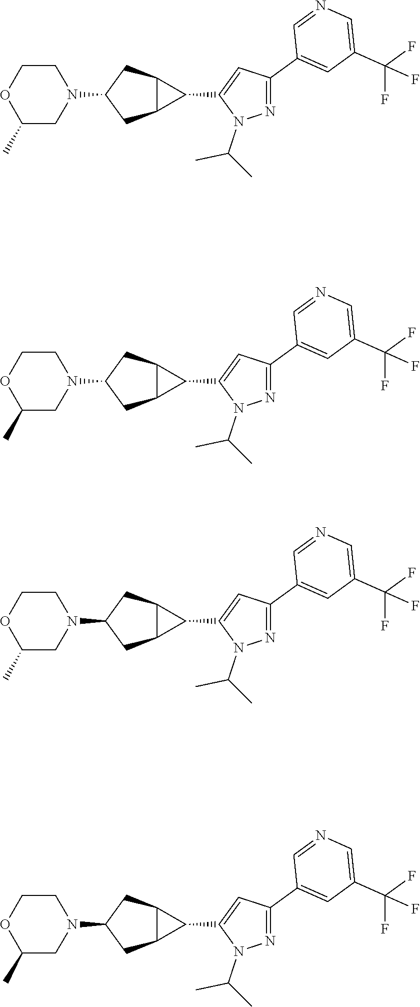 [SMILES: CC(C)n1nc(-c2cncc(C(F)(F)F)c2)cc1[C@H]1[C@@H]2C[C@@H](N3CCO[C@@H](C)C3)C[C@@H]21 ; CC(C)n1nc(-c2cncc(C(F)(F)F)c2)cc1[C@H]1[C@@H]2C[C@@H](N3CCO[C@H](C)C3)C[C@@H]21 ; CC(C)n1nc(-c2cncc(C(F)(F)F)c2)cc1[C@H]1[C@@H]2C[C@H](N3CCO[C@@H](C)C3)C[C@@H]21 ; CC(C)n1nc(-c2cncc(C(F)(F)F)c2)cc1[C@H]1[C@@H]2C[C@H](N3CCO[C@H](C)C3)C[C@@H]21]

Title compounds were synthesized following a procedure similar to compound 29* using 3-(4,4,5,5-tetramethyl-1,3,2-dioxaborolan-2-yl)-5-(trifluoromethyl) pyridine in step 1. The crude residue was purified by reverse phase chromatography (acetonitrile 70-100%/(0.05% NH₃H₂O+10 mM NH₄HCO₃ in water) to provide the cis-diastereomeric mixture (first peak on HPLC (basic), 80 mg, 39.6% yield) and the trans-diastereomeric mixture (second peak on HPLC (basic), 73 mg, 36.1% yield). LCMS (ESI) [M+H]⁺=435.4.

The cis-diastereomeric stereochemistry was confirmed by 2D NMR (first peak on HPLC (basic), 80 mg) was separated by chiral SFC (SFC-17; DAICEL CHIRALCEL OJ-H (250 mm*30 mm, 5 um); Supercritical CO₂/EtOH+NH₃·H₂O=90/10; 60 mL/min) to provide the title compound 39* (first peak on SFC, 21.9 mg, 27.4% yield) and the title compound 40* (second peak on SFC, 14.7 mg, 18.4% yield). LCMS (ESI) [M+H]⁺=435.4. The absolute stereochemistry was arbitrarily assigned.

Compound 39*: ¹H NMR (400 MHz, CDCl₃) δ 9.11 (s, 1H), 8.75 (s, 1H), 8.29 (s, 1H), 6.18 (s, 1H), 4.73-4.66 (m, 1H), 3.90-3.87 (m, 1H), 3.78-3.63 (m, 2H), 2.86-2.77 (m, 2H), 2.46-2.42 (m, 1H), 2.31-2.23 (m, 2H), 2.20-1.82 (m, 4H), 1.76-1.72 (m, 2H), 1.55 (d, J=6.8 Hz, 6H), 1.26 (s, 1H), 1.18 (d, J=6.4 Hz, 3H).

Compound 40*: ¹H NMR (400 MHz, CDCl₃) δ 9.11 (s, 1H), 8.74 (s, 1H), 8.29 (s, 1H), 6.12 (s, 1H), 4.73-4.66 (m, 1H), 3.91-3.88 (m, 1H), 3.73-3.55 (m, 2H), 2.91-2.72 (m, 4H), 2.34-2.24 (m, 2H), 2.14-1.97 (m, 1H), 1.76-1.72 (m, 4H), 1.55 (d, J=6.8 Hz, 6H), 1.26 (s, 1H), 1.18 (d, J=6.4 Hz, 3H).

The trans stereochemistry was confirmed by 2D NMR analysis and the mixture (second peak on HPLC (basic), 73 mg) was separated by chiral SFC (Daicel Chiralcel OJ-H (250 mm*30 mm, 5 μm); supercritical CO₂/EtOH+NH₃·H₂O=90/10; 60 mL/min) to provide the title compound 41* (first peak on SFC, 21.6 mg, 29.6% yield) and the title compound 42* (second peak on SFC, 18.5 mg, 25.3% yield). LCMS (ESI) [M+H]⁺=435.4. The absolute stereochemistry was arbitrarily assigned.

Compound 41*: ¹H NMR (400 MHz, CDCl₃) δ 9.11 (s, 1H), 8.74 (s, 1H), 8.29 (s, 1H), 6.18 (s, 1H), 4.74-4.62 (m, 1H), 3.90-3.87 (m, 1H), 3.80-3.65 (m, 2H), 2.86-2.78 (m, 2H), 2.46-2.42 (m, 1H), 2.30-2.23 (m, 2H), 2.07-1.76 (m, 4H), 1.75-1.74 (m, 2H), 1.55 (d, J=6.8 Hz, 6H), 1.26 (s, 1H), 1.17 (d, J=6.4 Hz, 3H).

Compound 42*: ¹H NMR (400 MHz, CDCl₃) δ 9.10 (s, 1H), 8.74 (s, 1H), 8.29 (s, 1H), 6.12 (s, 1H), 4.73-4.66 (m, 1H), 3.90-3.88 (m, 1H), 3.67-3.61 (m, 2H), 2.89-2.74 (m, 4H), 2.34-2.28 (m, 2H), 2.12-1.98 (m, 1H), 1.72-1.66 (m, 4H), 1.55 (d, J=6.8 Hz, 6H), 1.26 (s, 1H), 1.18 (d, J=6.4 Hz, 3H).

### Example T: (S)-4-((1R,3s,5S,6S)-6-(1-Isopropyl-3-(5-(trifluoromethyl)pyridin-3-yl)-1H-pyrazol-5-yl)bicyclo[3.1.0]hexan-3-yl)-3-methylmorpholine, (R)-4-((1R,3s,5S,6R)-6-(1-Isopropyl-3-(5-(trifluoromethyl)pyridin-3-yl)-1H-pyrazol-5-yl)bicyclo [3.1.0]hexan-3-yl)-3-methylmorpholine, (S)-4-((1R,3r,5S,6S)-6-(1-isopropyl-3-(5-(trifluoromethyl)pyridin-3-yl)-1H-pyrazol-5-yl)bicyclo[3.1.0]hexan-3-yl)-3-methylmorpholine & (R)-4-((1R,3r,5S,6R)-6-(1-isopropyl-3-(5-(trifluoromethyl)pyridin-3-yl)-1H-pyrazol-5-yl)bicyclo[3.1.0]hexan-3-yl)-3-methylmorpholine (Compounds 43*, 44*, 45*, & 46*)

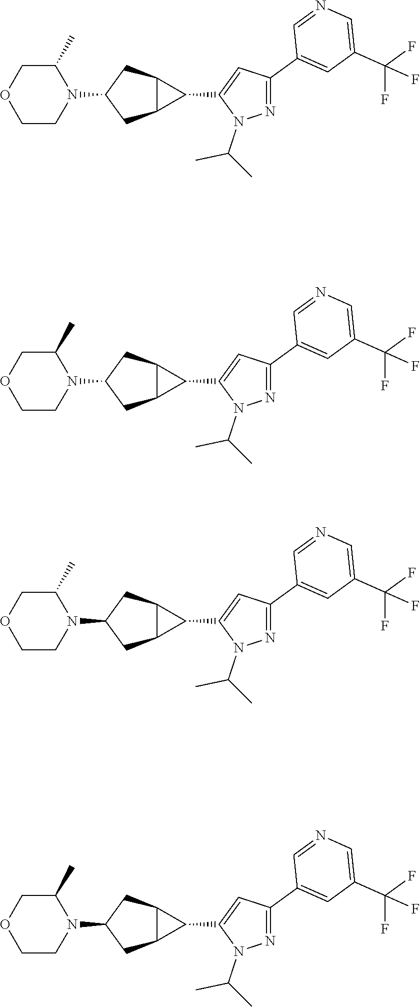 [SMILES: CC(C)n1nc(-c2cncc(C(F)(F)F)c2)cc1[C@H]1[C@@H]2C[C@@H](N3CCOC[C@@H]3C)C[C@@H]21 ; CC(C)n1nc(-c2cncc(C(F)(F)F)c2)cc1[C@H]1[C@@H]2C[C@@H](N3CCOC[C@H]3C)C[C@@H]21 ; CC(C)n1nc(-c2cncc(C(F)(F)F)c2)cc1[C@H]1[C@@H]2C[C@H](N3CCOC[C@@H]3C)C[C@@H]21 ; CC(C)n1nc(-c2cncc(C(F)(F)F)c2)cc1[C@H]1[C@@H]2C[C@H](N3CCOC[C@H]3C)C[C@@H]21]

Title compounds were synthesized following a procedure similar to compound 29* using 3-(4,4,5,5-tetramethyl-1,3,2-dioxaborolan-2-yl)-5-(trifluoromethyl) pyridine in step 1. The crude residue was purified by reverse phase chromatography (acetonitrile 70-100%/(0.05% NH₃H₂O+10 mM NH₄HCO₃ in water) to provide the cis mixture (first peak on HPLC (basic), 75 mg, 36.8% yield) and the trans mixture (second peak on HPLC (basic), 100 mg, 49.5% yield). LCMS (ESI) [M+H]⁺=435.4.

The cis stereochemistry was confirmed by 2D NMR analysis and the mixture (first peak on HPLC (basic), 75 mg) was separated by chiral SFC (Daicel Chiralcel OJ-H (250 mm*30 mm, 5 μm); supercritical CO₂/EtOH+NH₃·H₂O=90/10; 60 mL/min) to provide the title compound 43* (first peak on SFC, 12.1 mg, 16.1% yield) and the title compound 44* (second peak on SFC, 14.4 mg, 19.2% yield). LCMS (ESI) [M+H]⁺=435.4. The absolute stereochemistry was arbitrarily assigned.

Compound 43*: ¹H NMR (400 MHz, CDCl₃) δ 9.11 (d, J=1.6 Hz, 1H), 8.75 (s, 1H), 8.29 (s, 1H), 6.20 (s, 1H), 4.67-4.59 (m, 1H), 4.11-3.59 (m, 4H), 2.97-2.82 (m, 3H), 2.39-1.91 (m, 3H), 1.77-1.74 (m, 3H), 1.55 (d, J=6.4 Hz, 6H), 1.26 (s, 3H), 1.19-1.13 (m, 2H).

Compound 44*: ¹H NMR (400 MHz, CDCl₃) δ 9.11 (d, J=1.6 Hz, 1H), 8.75 (s, 1H), 8.29 (s, 1H), 6.19 (s, 1H), 4.61-4.59 (m, 1H), 4.01-3.57 (m, 4H), 3.10-2.72 (m, 3H), 2.56-2.50 (m, 1H), 2.26-2.10 (m, 2H), 2.03-1.88 (m, 1H), 1.76-1.72 (brs, 2H), 1.55 (d, J=6.4 Hz, 6H), 1.26 (s, 3H), 1.18-1.157 (m, 2H).

The trans stereochemistry was confirmed by 2DNMR and the mixture (second peak on HPLC (basic), 100 mg) was separated by chiral SFC (Daicel Chiralcel OJ-H (250 mm*30 mm, 5 μm); supercritical CO₂/EtOH+NH₃·H₂O=90/10; 60 mL/min) to provide the title compound 45* (first peak on SFC, 27.7 mg, 27.4% yield) and the title compound 46* (second peak on SFC, 26.6 mg, 26.3% yield). LCMS (ESI) [M+H]⁺=435.4. The absolute stereochemistry was arbitrarily assigned.

Compound 45*: ¹H NMR (400 MHz, CDCl₃) δ 9.11 (s, 1H), 8.74 (s, 1H), 8.29 (s, 1H), 6.13 (s, 1H), 4.80-4.58 (m, 1H), 3.90-3.61 (m, 3H), 3.55-3.40 (m, 2H), 2.85-2.81 (m, 1H), 2.64-2.61 (m, 1H), 2.49-2.42 (m, 1H), 2.35-2.09 (m, 2H), 1.88-1.72 (m, 2H), 1.68-1.65 (m, 4H), 1.56 (d, J=6.8 Hz, 6H), 1.09-1.07 (m, 2H).

Compound 46*: ¹H NMR (400 MHz, CDCl₃) δ 9.11 (d, J=1.2 Hz, 1H), 8.74 (s, 1H), 8.29 (s, 1H), 6.13 (s, 1H), 4.78-4.60 (m, 1H), 3.88-3.61 (m, 3H), 3.58-3.42 (m, 2H), 2.85-2.81 (m, 1H), 2.64-2.61 (m, 1H), 2.49-2.42 (m, 1H), 2.34-2.13 (m, 2H), 1.88-1.72 (m, 2H), 1.69-1.65 (m, 4H), 1.56 (d, J=6.8 Hz, 6H), 1.13-1.09 (m, 2H).

### Example U: 4-((1R,3S,5S,6R)-6-(1-Isopropyl-3-(6-(trifluoromethyl)pyridin-3-yl)-1H-pyrazol-5-yl)bicyclo[3.1.0]hexan-3-yl)-1,4-oxazepane (Compound 47) and 4-((1R,3S,5S,6R)-6-(1-Isopropyl-3-(6-(trifluoromethyl)pyridin-3-yl)-1H-pyrazol-5-yl)bicyclo[3.1.0]hexan-3-yl)-1,4-oxazepane (Compound 48)

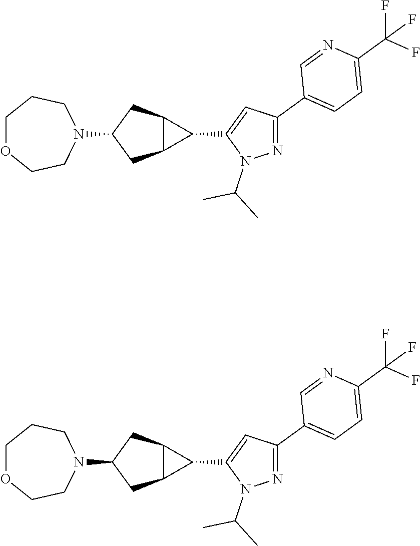 [SMILES: CC(C)n1nc(-c2ccc(C(F)(F)F)nc2)cc1[C@H]1[C@@H]2C[C@@H](N3CCCOCC3)C[C@@H]21 ; CC(C)n1nc(-c2ccc(C(F)(F)F)nc2)cc1[C@H]1[C@@H]2C[C@H](N3CCCOCC3)C[C@@H]21]

The title compounds were synthesized following a procedure similar to compound 29* using 5-(4,4,5,5-tetramethyl-1,3,2-dioxaborolan-2-yl)-2-(trifluoromethyl)pyridine in step 1. The crude residue was purified by reverse phase chromatography (water (0.05% NH₃H₂O+10 mM NH₄HCO₃)-ACN, 70%-100%) to provide the title compound 47 (second peak on SFC, 57.9 mg, 31% yield) and the title compound 48 (first peak on SFC, 46.1 mg, 25% yield). LCMS (ESI) [M+H]⁺=435.3. The relative stereochemistry was determined by 2D-NMR.

Compound 47: ¹H NMR (400 MHz, CDCl₃) δ 9.04 (s, 1H), 8.24 (d, J=8.8 Hz, 1H), 7.67 (d, J=8.0 Hz, 1H), 6.20 (s, 1H), 4.75-4.56 (m, 1H), 3.86-3.76 (m, 4H), 2.88-2.65 (m, 4H), 2.33-2.21 (dd, J=12.4, 7.2 Hz, 2H), 2.05-1.92 (m, 4H), 1.77-1.69 (s, 2H), 1.60 (d, J=3.2 Hz, 2H), 1.56 (d, J=6.8 Hz, 6H).

Compound 48: ¹H NMR (400 MHz, CDCl₃) δ 9.02 (s, 1H), 8.23 (d, J=8.0 Hz, 1H), 7.65 (d, J=8.4 Hz, 1H), 6.13 (s, 1H), 4.69-4.62 (m, 1H), 3.84-3.73 (m, 4H), 3.41-3.29 (m, 1H), 2.83-2.67 (m, 4H), 2.36 (s, 2H), 1.91 (s, 2H), 1.78 (s, 1H), 1.67 (s, 2H), 1.56 (d, J=6.8 Hz, 6H).

### Example V: 4-((1R,3s,5S,6r)-6-(1-Isopropyl-3-(6-(trifluoromethyl)pyridin-2-yl)-1H-pyrazol-5-yl)bicyclo[3.1.0]hexan-3-yl)-1,4-oxazepane (Compound 49) and 4-((1R,3r,5S,6r)-6-(1-Isopropyl-3-(6-(trifluoromethyl)pyridin-2-yl)-1H-pyrazol-5-yl)bicyclo[3.1.0]hexan-3-yl)-1,4-oxazepane (Compound 50)

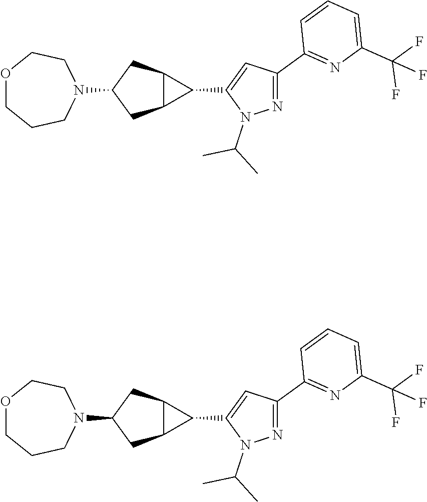 [SMILES: CC(C)n1nc(-c2cccc(C(F)(F)F)n2)cc1[C@H]1[C@@H]2C[C@@H](N3CCCOCC3)C[C@@H]21 ; CC(C)n1nc(-c2cccc(C(F)(F)F)n2)cc1[C@H]1[C@@H]2C[C@H](N3CCCOCC3)C[C@@H]21]

The title compound was synthesized following a procedure similar to compound 29* using 2-(4,4,5,5-tetramethyl-1,3,2-dioxaborolan-2-yl)-6-(trifluoromethyl)pyridine in step 1. Purification of the crude mixture by reverse phase chromatography (acetonitrile 30%-60%/0.10% NH₄OH in water) to provide compound 49 (first peak on HPLC (basic), 20.1 mg, 16% yield) and compound 50 (second peak on HPLC (basic), 16.1 mg, 12.3% yield). LCMS (ESI) [M+H]⁺=435.3. The relative stereochemistry was determined by 2D-NMR.

Compound 49: ¹H NMR (400 MHz, CD₃OD) δ 8.15 (d, J=8.0 Hz, 1H), 7.96 (t, J=8.0 Hz, 1H), 7.62 (d, J=7.6 Hz, 1H), 6.51 (s, 1H), 4.83-4.74 (m, 1H), 3.85-3.69 (m, 4H), 3.06-2.94 (m, 1H), 2.90-2.77 (m, 4H), 2.29 (dd, J=12.4, 7.2 Hz, 2H), 2.00-1.83 (m, 4H), 1.80-1.70 (m, 3H), 1.53 (d, J=6.4 Hz, 6H).

Compound 50: ¹H NMR (400 MHz, CD₃OD) δ 8.15 (d, J=8.0 Hz, 1H), 7.96 (t, J=8.0 Hz, 1H), 7.62 (d, J=7.6 Hz, 1H), 6.46 (s, 1H), 4.85-4.76 (m, 1H), 3.82-3.72 (m, 4H), 3.54-3.39 (m, 1H), 2.88-2.83 (m, 4H), 2.51-2.35 (m, 2H), 1.98-1.87 (m, 2H), 1.84-1.79 (m, 1H), 1.72-1.70 (m, 2H), 1.62-1.61 (m, 2H), 1.55 (d, J=6.4 Hz, 6H).

### Example W: 4-((1R,3r,5S,6r)-6-(1-Isopropyl-3-(5-(trifluoromethyl)pyridin-3-yl)-1H-1,2,4-triazol-5-yl)bicyclo[3.1.0]hexan-3-yl)-1,4-oxazepane (Compound 51) and 4-((1R,3s,5S,6r)-6-(1-Isopropyl-3-(5-(trifluoromethyl)pyridin-3-yl)-1H-1,2,4-triazol-5-yl)bicyclo[3.1.0]hexan-3-yl)-1,4-oxazepane (Compound 52)

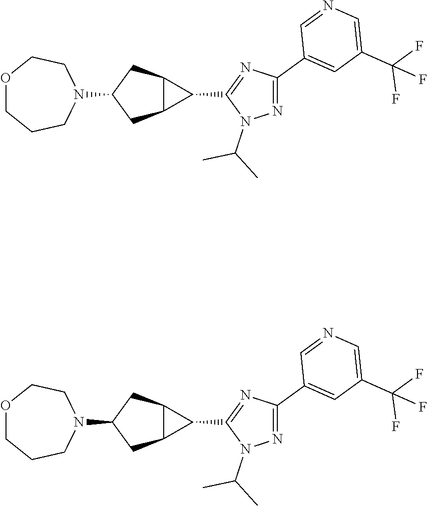 [SMILES: CC(C)n1nc(-c2cncc(C(F)(F)F)c2)nc1[C@H]1[C@@H]2C[C@@H](N3CCCOCC3)C[C@@H]21 ; CC(C)n1nc(-c2cncc(C(F)(F)F)c2)nc1[C@H]1[C@@H]2C[C@H](N3CCCOCC3)C[C@@H]21]

### Step 1: (E)-Benzyl (amino(methylthio)methylene)carbamate

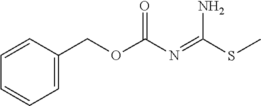

To an ice cold mixture of 2-methylisothiourea sulfurous acid (10. g, 58.06 mmol) and sodium hydroxide (34.8 mL, 69.6 mmol, 2N) in dichloromethane (100 mL) was added benzyl chloroformate (7.0 mL, 52.26 mmol). The mixture was stirred at 25° C. for 1 hour. The mixture was extracted with ethyl acetate (500 mL×2) and washed with brine (100 mL). The organic layer was dried over Na₂SO₄, filtered and concentrated. The residue was purified by silica flash chromatography (0-50% ethyl acetate in petroleum ether) to provide the title compound (10 g, 77% yield).

### Step 2: (Z)-Benzyl((3-((tert-butyldiphenylsilyl)oxy)bicyclo[3.1.0]hexane-6-carboxamido) (methylthio)methylene)carbamate

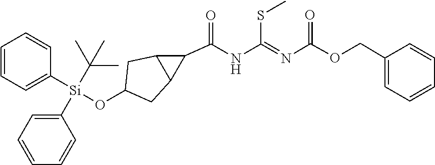 [SMILES: CS/C(=N\C(=O)OCc1ccccc1)NC(=O)C1C2CC(O[Si](c3ccccc3)(c3ccccc3)C(C)(C)C)CC21]

To a solution of 3-[tert-butyl(diphenyl)silyl]oxybicyclo[3.1.0]hexane-6-carboxylic acid (1.97 mL, 62.42 mmol), NN-diisopropylethylamine (19.4 mL, 111.4 mmol) and (E)-Benzyl (amino(methylthio) methylene)carbamate (10.0 g, 44.59 mmol) in tetrahydrofuran (80 mL) was added HATU (25.4 g, 66.88 mmol) at 20° C. Then the reaction mixture was stirred at 20° C. for 2 hours. The mixture was diluted with ethyl acetate (200 mL), washed with brine (50 mL). The organic layer was dried over Na₂SO₄, filtered and concentrated. The residue was purified by silica flash chromatography (0-20% ethyl acetate in petroleum ether) to provide the title compound (22 g, 84% yield).

### Step 3: 5-[3-[Tert-butyl(diphenyl)silyl]oxy-6-bicyclo[3.1.0]hexanyl]-1-isopropyl-1,2,4-triazol-3-amine

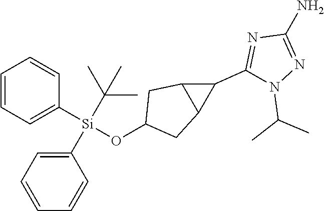 [SMILES: CC(C)n1nc(N)nc1C1C2CC(O[Si](c3ccccc3)(c3ccccc3)C(C)(C)C)CC21]

To a stirred solution of (Z)-Benzyl ((3-((tert-butyldiphenylsilyl)oxy)bicyclo[3.1.0]hexane-6-carboxamido)(methylthio)methylene)carbamate (20.0 g, 34.08 mmol) in NN-dimethylformamide (200 mL) was added isopropylhydrazinehydrochloride (18.9 g, 170.4 mmol) and trimethylamine (47.4 mL, 340.8 mmol). The reaction mixture was stirred at 160° C. for 2.5 hours. The reaction mixture was quenched by water (200 mL) and extracted with 10% methanol in ethyl acetate (100 mL×3). The combined organic layers were washed with brine (100 mL×3), dried over Na₂SO₄, filtered and concentrated under reduced pressure. The residue was purified by silica flash chromatography (0-50% ethyl acetate in petroleum ether) to provide the title compound (10 g, 64% yield). LCMS (ESI) [M+H]⁺=461.3.

### Step 4: 5-(3-((Tert-butyldiphenylsilyl)oxy)bicycle[3.1.0]hexan-6-yl)-3-iodo-1-isopropyl-1H-1, 2, 4-triazole

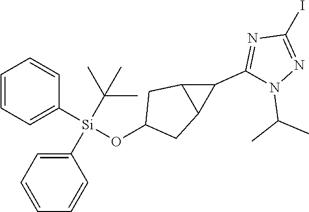 [SMILES: CC(C)n1nc(I)nc1C1C2CC(O[Si](c3ccccc3)(c3ccccc3)C(C)(C)C)CC21]

To an ice cooled solution of 5-[3-[tert-butyl(diphenyl)silyl]oxy-6-bicyclo[3.1.0]hexanyl]-1-isopropyl-1,2,4-triazol-3-amine (5000.0 mg, 10.85 mmol) in acetonitrile (50 mL) and water (10 mL) was added 4-methylbenzenesulfonic acid (9331 mg, 54.3 mmol) and sodium nitrite (1498 mg, 21.71 mmol) in water (5 mL) and stirred at 0° C. for 30 minutes. Then sodium iodide (4067.0 mg, 27.13 mmol) was added rapidly to the solution and stirred at 0° C. for 3 hours. The reaction was poured into water (50 mL) and extracted with ethyl acetate (200 mL×3). The combined organic layers were washed with brine (50 mL×3), dried over Na₂SO₄, filtered and concentrated under reduced pressure. The crude was purified by silica flash chromatography (0-20% ethyl acetate in petroleum ether) to provide the title compound (3800 mg, 61% yield). LCMS (ESI) [M+H]⁺=572.0.

### Step 5: 6-(3-Iodo-1-isopropyl-1H-1, 2, 4-triazol-5-yl)bicyclo[3.1.0]hexan-3-ol

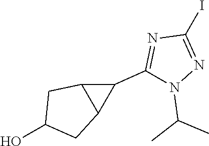 [SMILES: CC(C)n1nc(I)nc1C1C2CC(O)CC21]

To a stirred solution of tert-butyl-[[6-(5-iodo-2-isopropyl-1, 2, 4-triazol-3-yl)-3-bicyclo [3.1.0]hexanyl]oxy]-diphenyl-silane (3800.0 mg, 6.65 mmol) in tetrahydrofuran (38 mL) was added triethylamine trihydrofluoride (21.7 mL, 132.97 mmol). The reaction mixture was stirred at 70° C. for 16 hours. The reaction was quenched by saturated NaHCO₃ (100 mL) and extracted with ethyl acetate (200 mL×3). The combined organic layers were washed with brine (100 mL), dried over sodium sulfate, filtered and concentrated under reduced pressure. The crude residue was purified by silica flash chromatography (0-25% ethyl acetate in petroleum ether) to provide the title compound (2000 mg, 83% yield). LCMS (ESI) [M+H]⁺=334.0.

### Step 6: 6-(5-Iodo-2-isopropyl-1, 2, 4-triazol-3-yl)bicyclo[3.1.0]hexan-3-one

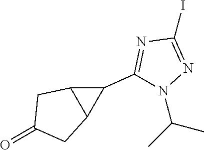 [SMILES: CC(C)n1nc(I)nc1C1C2CC(=O)CC21]

To a stirred solution of 6-(5-iodo-2-isopropyl-1,2,4-triazol-3-yl)bicyclo[3.1.0]hexan-3-ol (2000 mg, 6 mmol) in dichloromethane (20 mL) was added Dess-Martin periodinane (3055 mg, 7.2 mmol) and stirred at 25° C. for 16 hours. The reaction was quenched by saturated aqueous NaHCO₃ (80 mL) and extracted with ethyl acetate (200 mL×3). The combined organic layers were washed with brine (50 mL×3), dried over sodium sulfate, filtered and concentrated under reduced pressure. The crude residue was purified by silica column chromatography (0-50% ethyl acetate in petroleum ether) to provide the title compound (1200 mg, 59% yield). LCMS (ESI) [M+H]⁺=332.0.

### Step 7: 6-[2-Isopropyl-5-[5-(trifluoromethyl)-3-pyridyl]-1, 2, 4-triazol-3-yl]bicyclo [3.1.0]hexan-3-one

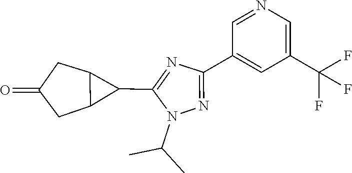 [SMILES: CC(C)n1nc(-c2cncc(C(F)(F)F)c2)nc1C1C2CC(=O)CC21]

The suspension of K₂CO₃ (626 mg, 4.53 mmol), bis(di-tert-butyl(4-dimethylaminophenyl) phosphine)dichloropalladium(II) (107 mg, 0.15 mmol), 6-(5-iodo-2-isopropyl-1,2,4-triazol-3-yl)bicyclo[3.1.0]hexan-3-one (500 mg, 1.51 mmol) and 3-(4,4,5,5-tetramethyl-1,3,2-dioxaborolan-2-yl)-5-(trifluoromethyl)pyridine (495 mg, 1.81 mmol) in 1,4-dioxane (10 mL) and water (2 mL) was stirred at 80° C. under N₂ atmosphere for 2 hours. The mixture was diluted with water (25 mL) and extracted with ethyl acetate (40 mL×3). The combined organic layers were washed with brine (20 mL×3), dried over Na₂SO₄, filtered and concentrated under reduced pressure. The residue was purified by silica flash chromatography (0-50% ethyl acetate in petroleum ether) to provide the title compound (200 mg, 27% yield). LCMS (ESI) [M+H]⁺=351.1.

### Step 8: 4-[(1R, 5S)-6-[2-Isopropyl-5-[5-(trifluoromethyl)-3-pyridyl]-1, 2, 4-triazol-3-yl]-3-bicyclo[3.1.0]hexanyl]-1,4-oxazepane

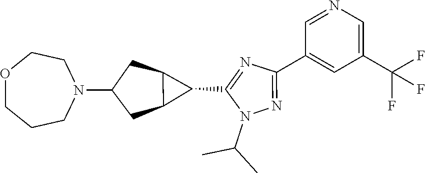

To a solution of 6-[2-isopropyl-5-[5-(trifluoromethyl)-3-pyridyl]-1,2,4-triazol-3-yl]bicyclo[3.1.0]hexan-3-one (80 mg, 0.23 mmol) and homomorpholinehydrochloride (94 mg, 0.69 mmol) in methanol (3 mL) at 25° C. was added NaBH₃CN (72 mg, 1.14 mmol). Then the reaction mixture was stirred at 80° C. for 16 hours. The reaction mixture was concentrated and the crude was purified by prep-TLC to provide the title compound (60 mg, 60% yield) as a mixture of diastereomers.

### Step 9: 4-((1R,3R,5S,6R)-6-(1-Isopropyl-3-(5-(trifluoromethyl)pyridin-3-yl)-1H-1,2,4-triazol-5-yl)bicyclo[3.1.0]hexan-3-yl)-1,4-oxazepane (Compound 51) and 4-((1R,3S,5S,6R)-6-(1-Isopropyl-3-(5-(trifluoromethyl)pyridin-3-yl)-1H-1,2,4-triazol-5-yl)bicyclo[3.1.0]hexan-3-yl)-1,4-oxazepane (Compound 52)

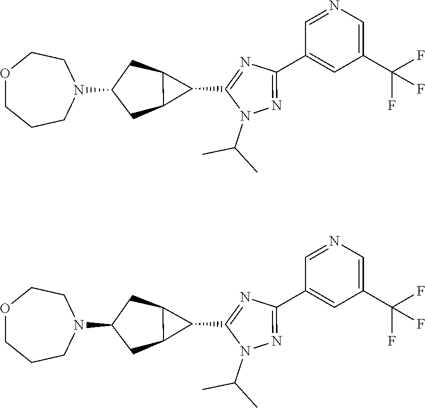 [SMILES: CC(C)n1nc(-c2cncc(C(F)(F)F)c2)nc1[C@H]1[C@@H]2C[C@@H](N3CCCOCC3)C[C@@H]21 ; CC(C)n1nc(-c2cncc(C(F)(F)F)c2)nc1[C@H]1[C@@H]2C[C@H](N3CCCOCC3)C[C@@H]21]

The mixture of diastereomers were purified by reverse phase chromatography (water (0.05% NH₃H₂O+10 mM NH₄HCO₃)-ACN, 65%-95%) to provide compound 51 (first peak on HPLC, 22.2 mg, 19% yield) and compound 52 (second peak on HPLC, 32.6 mg, 27% yield). LCMS (ESI) [M+H]⁺ 423.3. The relative stereochemistry was determined by 2D-NMR.

Compound 51: 1H NMR (400 MHz, CD₃OD) δ 9.37 (d, J=1.6 Hz, 1H), 8.87 (d, J=1.2 Hz, 1H), 8.59 (s, 1H), 4.94-4.90 (m, 1H), 3.80-3.73 (m, 4H), 3.50-3.31 (m, 1H), 2.79-2.75 (m, 4H), 2.43-2.36 (m, 2H), 2.12 (t, J=3.2 Hz, 1H), 2.05-2.01 (m, 2H), 1.90-1.87 (m, 2H), 1.75-1.65 (m, 2H), 1.58 (d, J=6.8 Hz, 6H).

Compound 52: ¹H NMR (400 MHz, CD₃OD) δ 9.37 (d, J=1.6 Hz, 1H), 8.87 (d, J=1.2 Hz, 1H), 8.59 (s, 1H), 4.94-4.90 (m, 1H), 3.80-3.74 (m, 4H), 3.43-3.31 (m, 1H), 2.79-2.73 (m, 4H), 2.45-2.35 (m, 2H), 2.04-2.02 (m, 1H), 1.96-1.88 (m, 4H), 1.75-1.65 (m, 2H), 1.55 (d, J=6.8 Hz, 6H).

### Example X: 4-((1R,3s,5S,6r)-6-(1-Ethyl-3-(5-(trifluoromethyl)pyridin-3-yl)-1H-pyrazol-5-yl)bicyclo[3.1.0]hexan-3-yl)-1,4-oxazepane (Compound 53) and 4-((1R,3r,5S,6r)-6-(1-Ethyl-3-(5-(trifluoromethyl)pyridin-3-yl)-1H-pyrazol-5-yl)bicyclo [3.1.0]hexan-3-yl)-1,4-oxazepane (Compound 54)

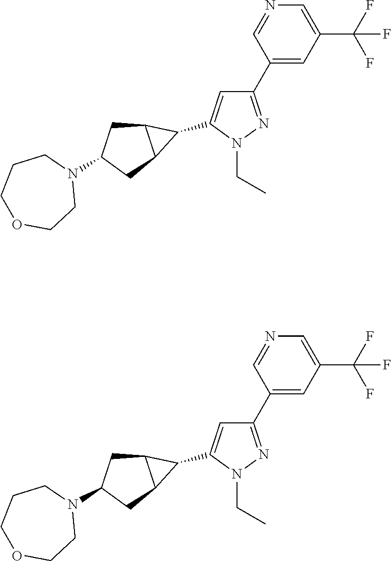 [SMILES: CCn1nc(-c2cncc(C(F)(F)F)c2)cc1[C@H]1[C@@H]2C[C@@H](N3CCCOCC3)C[C@@H]21 ; CCn1nc(-c2cncc(C(F)(F)F)c2)cc1[C@H]1[C@@H]2C[C@H](N3CCCOCC3)C[C@@H]21]

### Step 1: 3-(5-((1R,5S,6r)-3-((tert-Butyldiphenylsilyl)oxy)bicyclo[3.1.0]hexan-6-yl)-1H-pyrazol-3-yl)-5-(trifluoromethyl)pyridine

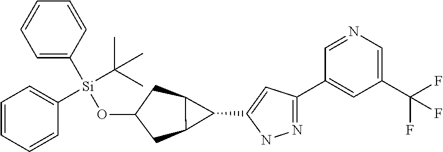

To a mixture of 5-((1R,5S,6r)-3-((tert-butyldiphenylsilyl)oxy)bicyclo[3.1.0]hexan-6-yl)-3-iodo-1H-pyrazole (2.0 g, 3.78 mmol) and 3-(4,4,5,5-tetramethyl-1,3,2-dioxaborolan-2-yl)-5-(trifluoromethyl)pyridine (1.6 g, 5.86 mmol) in 1,4-dioxane (16 mL) and water (4 mL) were added K₂CO₃ (1.6 g, 11.58 mmol) and bis(di-tert-butyl(4-dimethylaminophenyl)phosphine)dichloropalladium(II) (0.28 g, 0.40 mmol). Then the reaction mixture was placed under nitrogen atmosphere and stirred at 75° C. for 5 hours. The reaction was quenched by water (50 mL) and extracted with ethyl acetate (150 mL×3). The combined organic layers were washed with brine (50 mL), dried over Na₂SO₄, filtered and concentrated. The residue was purified by silica flash chromatography (0-30% ethyl acetate in petroleum ether) to provide the title compound (2.0 g, 88% yield). LCMS (ESI) [M+H]⁺=548.1.

### Step 2: 3-(5-((1R,5S,6r)-3-((tert-Butyldiphenylsilyl)oxy)bicyclo[3.1.0]hexan-6-yl)-1-ethyl-1H-pyrazol-3-yl)-5-(trifluoromethyl)pyridine

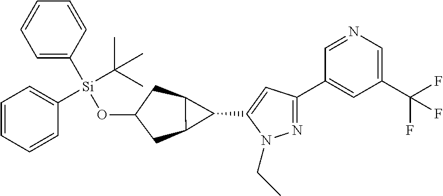

To a stirred mixture of 3-(5-((1R,5S,6r)-3-((tert-butyldiphenylsilyl)oxy)bicyclo[3.1.0]hexan-6-yl)-1H-pyrazol-3-yl)-5-(trifluoromethyl)pyridine (1.2 g, 2.19 mmol) and Cs₂CO₃ (1.4 g, 4.3 mmol) in N,N-dimethylformamide (8 mL) was added iodoethane (0.9 mL, 11.25 mmol) dropwise, and then stirred at 25° C. for 3 hours. The reaction was quenched by water (30 mL) and extracted with ethyl acetate (100 mL×3). The combined organic layers were washed with brine (30 mL), dried over Na₂SO₄, filtered and concentrated. The residue was purified by silica flash chromatography (0-15% ethyl acetate in petroleum ether) to provide the title compound (890 mg, 71% yield). LCMS (ESI) [M+H]⁺=576.2.

### Step 3: (1R,5S,6r)-6-(1-Ethyl-3-(5-(trifluoromethyl)pyridin-3-yl)-1H-pyrazol-5-yl)bicyclo[3.1.0]hexan-3-ol

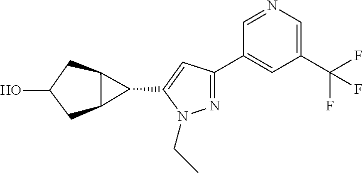

To 3-(5-((1R,5S,6r)-3-((tert-butyldiphenylsilyl)oxy)bicyclo[3.1.0]hexan-6-yl)-1-ethyl-1H-pyrazol-3-yl)-5-(trifluoromethyl)pyridine (870 mg, 1.51 mmol) in the flask was added tetrabutyl ammonium fluoride in tetrahydrofuran (12.0 mL, 12 mmol, 1 M), the mixture was stirred at 25° C. for 5 hours. Then the reaction was quenched by saturated NH₄Cl solution (15 mL) and extracted with ethyl acetate (100 mL×3). The combined organic layers were washed with brine (30 mL), dried over Na₂SO₄, filtered and concentrated. The residue was purified by silica flash chromatography (0-50% ethyl acetate in petroleum ether) to provide the title compound (500 mg, 98% yield). LCMS (ESI) [M+H]⁺=338.1.

### Step 4: (1R,5S,6r)-6-(1-Ethyl-3-(5-(trifluoromethyl)pyridin-3-yl)-1H-pyrazol-5-yl)bicyclo[3.1.0]hexan-3-one

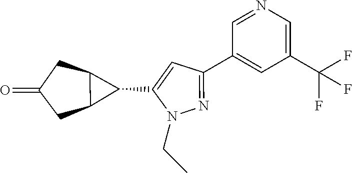 [SMILES: CCn1nc(-c2cncc(C(F)(F)F)c2)cc1[C@H]1[C@@H]2CC(=O)C[C@@H]21]

To a solution of (1R,5S,6r)-6-(1-Ethyl-3-(5-(trifluoromethyl)pyridin-3-yl)-1H-pyrazol-5-yl)bicyclo[3.1.0]hexan-3-ol (500 mg, 1.48 mmol) in anhydrous dichloromethane (12 mL) was added Dess-Martin periodinane (1000 mg, 2.36 mmol), the reaction mixture was stirred at 25° C. for 4 hours. Then the mixture was quenched by Na₂SO₃ solution (20 mL), followed by saturated NaHCO₃ solution (10 mL) and extracted with dichloromethane (100 mL×3). The combined organic layers were washed with brine (30 mL), dried over Na₂SO₄, filtered and concentrated. The residue was purified by silica flash chromatography (0-40% ethyl acetate in petroleum ether) to provide the title compound (410 mg, 83% yield). LCMS (ESI) [M+H]⁺=336.3.

### Step 5: 4-((1R,3s,5S,6r)-6-(1-Ethyl-3-(5-(trifluoromethyl)pyridin-3-yl)-1H-pyrazol-5-yl)bicyclo [3.1.0]hexan-3-yl)-1,4-oxazepane (Compound 53) and 4-((1R,3r,5S,6r)-6-(1-ethyl-3-(5-(trifluoromethyl)pyridin-3-yl)-1H-pyrazol-5-yl)bicyclo[3.1.0]hexan-3-yl)-1,4-oxazepane (Compound 54)

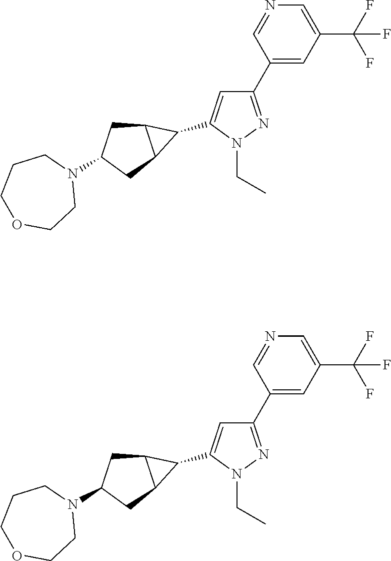 [SMILES: CCn1nc(-c2cncc(C(F)(F)F)c2)cc1[C@H]1[C@@H]2C[C@@H](N3CCCOCC3)C[C@@H]21 ; CCn1nc(-c2cncc(C(F)(F)F)c2)cc1[C@H]1[C@@H]2C[C@H](N3CCCOCC3)C[C@@H]21]

To a solution of (1R,5S,6r)-6-(1-ethyl-3-(5-(trifluoromethyl)pyridin-3-yl)-1H-pyrazol-5-yl)bicyclo [3.1.0]hexan-3-one (130 mg, 0.38 mmol) and 1,4-oxazepane hydrochloride (135 mg, 0.97 mmol) in anhydrous methanol (6 mL) was added NaBH₃CN (120 mg, 1.92 mmol) and acetic acid (0.02 mL, 0.35 mmol) dropwise at 20° C. Then the reaction mixture was heated to 70° C. and stirred for 4 hours. The reaction was quenched by saturated NaHCO₃ solution (5 mL), and extracted with dichloromethane (30 mL×2). The combined organics were washed with brine, dried over Na₂SO₄, filtered and concentrated under reduced pressure. The resulting residue was purified by reverse phase chromatography (acetonitrile 70-100%/(0.05% NH₃H₂O+10 mM NH₄HCO₃ in water) to provide the title compound 53 (second peak on SFC, 32.4 mg, 24% yield) and compound 54 (first peak on SFC, 40.3 mg, 30% yield). LCMS (ESI) [M+H]⁺=421.4. The relative stereochemistry was determined by 2D-NMR.

Compound 53: ¹H NMR (400 MHz, CDCl₃) δ 9.10 (d, J=1.6 Hz, 1H), 8.76 (d, J=1.2 Hz, 1H), 8.28 (s, 1H), 6.18 (s, 1H), 4.28-4.20 (m, 2H), 3.79-3.72 (m, 4H), 2.55-2.50 (m, 5H), 2.32-2.20 (m, 2H), 2.04-1.81 (m, 2H), 1.76-1.70 (brs, 2H), 1.57 (t, J=3.2 Hz, 1H), 1.51 (t, J=7.2 Hz, 3H).

Compound 54: ¹H NMR (400 MHz, CDCl₃) δ 9.11 (s, 1H), 8.77 (s, 1H), 8.29 (s, 1H), 6.13 (s, 1H), 4.32-4.20 (m, 2H), 3.82-3.78 (m, 4H), 3.35-3.30 (m, 1H), 2.79-2.70 (m, 3H), 2.39-2.35 (m, 2H), 1.95-1.91 (m, 2H), 1.85-1.81 (m, 2H), 1.71-1.67 (m, 4H), 1.53 (t, J=7.2 Hz, 3H).

### Example Y: 4-((1S,3S)-3-(1-Isopropyl-3-(5-(trifluoromethyl)pyridin-3-yl)-1H-pyrazol-5-yl)cyclopentyl)morpholine (Compound 58*) and 4-((1R,3R)-3-(1-Isopropyl-3-(5-(trifluoromethyl)pyridin-3-yl)-1H-pyrazol-5-yl)cyclopentyl)morpholine (Compound 59*), 4-((1R,3S)-3-(1-Isopropyl-3-(5-(trifluoromethyl)pyridin-3-yl)-1H-pyrazol-5-yl)cyclopentyl) morpholine (Compound 60*) and 4-((1S,3R)-3-(1-Isopropyl-3-(5-(trifluoromethyl)pyridin-3-yl)-1H-pyrazol-5-yl)cyclopentyl)morpholine (Compound 61*)

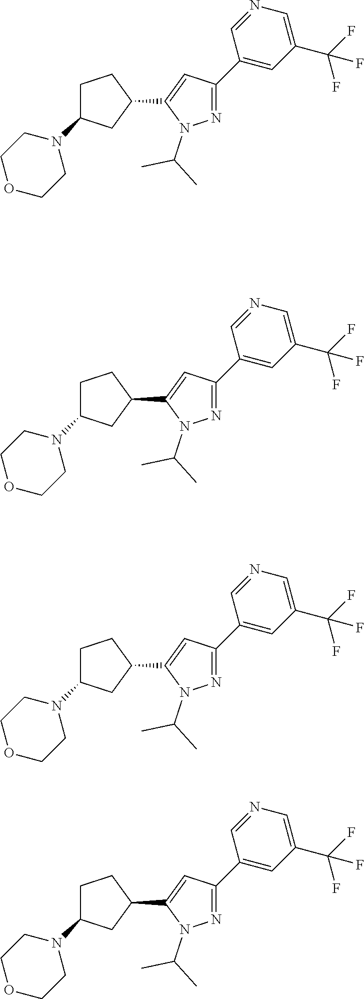 [SMILES: CC(C)n1nc(-c2cncc(C(F)(F)F)c2)cc1[C@H]1CC[C@H](N2CCOCC2)C1 ; CC(C)n1nc(-c2cncc(C(F)(F)F)c2)cc1[C@@H]1CC[C@@H](N2CCOCC2)C1 ; CC(C)n1nc(-c2cncc(C(F)(F)F)c2)cc1[C@H]1CC[C@@H](N2CCOCC2)C1 ; CC(C)n1nc(-c2cncc(C(F)(F)F)c2)cc1[C@@H]1CC[C@H](N2CCOCC2)C1]

### Step 1: 3-Bromocyclopent-2-enone

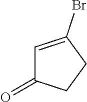

To a suspension of Ph₃PBr₂ (4.73 g, 11.2 mmol) in dichloromethane (10 mL) was added cyclopent-4-ene-1,3-dione (1.0 g, 10.2 mmol) and triethylamine (1.56 mL, 11.2 mmol) at 25° C. Then the reaction mixture was stirred at 25° C. for 18 hours. A yellow suspension was formed. The reaction mixture was concentrated under vacuum, then 2-methoxy-2-methylpropane (50 mL) was added to the above mixture, stirred and filtered. The organic layer was concentrated under reduced pressure and purified by silica flash chromatography (10% 20% ethyl acetate in petroleum ether) to provide title compound (1.10 g, 67% yield). ¹H NMR (400 MHz, DMSO-d₆) δ 6.66-6.62 (m, 1H), 3.01-2.94 (m, 2H), 2.50-2.47 (m, 2H).

### Step 2: 3-(4,4,5,5-Tetramethyl-1,3,2-dioxaborolan-2-yl)cyclopent-2-enone

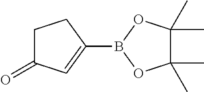

A mixture of 4-bromocyclopent-2-enone (1.10 g, 6.83 mmol), 4,4,4′,4′,5,5,5′,5′-octamethyl-2,2′-bi(1,3,2-dioxaborolane) (1.91 g, 7.52 mmol), bis(di-tert-butyl(4-dimethylaminophenyl)phosphine) dichloropalladium(II) (500 mg, 0.68 mmol) and KOAc (2.01 g, 20.5 mmol) in 1,4-dioxane (10 mL) was degassed and purged with N₂ for three times. Then the reaction mixture was stirred at 100° C. under N₂ for 12 hours. A brown suspension was formed. The reaction mixture was quenched by water (30 mL) and extracted with ethyl acetate (50 mL×3). The combined organic layers were washed with brine (50 mL), dried over anhydrous sodium sulfate, filtered and concentrated. The residue was purified by silica flash chromatography (2%-3% methanol in dichloromethane) to provide title compound (1.30 g, 91% yield). H NMR (400 MHz, DMSO-d₆) δ 6.45-6.40 (m, 1H), 2.68-2.63 (m, 2H), 2.28-2.23 (m, 2H), 1.27 (s, 12H).

### Step 3: 3-(3-Bromo-1-isopropyl-1H-pyrazol-5-yl)cyclopent-2-enone

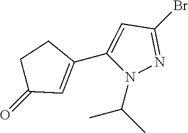

A suspension of 3-(4,4,5,5-tetramethyl-1,3,2-dioxaborolan-2-yl)cyclopent-2-enone (1.30 g, 6.25 mmol), 3,5-dibromo-1-isopropyl-1H-pyrazole (1.67 g, 6.25 mmol), Cs₂CO₃ (6.11 g, 18.7 mmol) and bis(di-tert-butyl(4-dimethylaminophenyl)phosphine)dichloropalladium(II) (457 mg, 0.62 mmol) in 1,4-dioxane (30 mL) and water (6 mL) was degassed and purged with N₂ for three times, then the reaction mixture was stirred at 100° C. under N₂ for 1.5 hours. The reaction mixture was filtered and the filtrate was concentrated. The residue was purified by silica flash chromatography (40%-50% ethyl acetate in petroleum ether) to provide title compound (1.0 g, 59% yield). ¹H NMR (400 MHz, CDCl₃) δ 6.85 (s, 1H), 6.64 (d, J=1.6 Hz, 1H), 5.04-4.94 (m, 1H), 3.31-3.28 (m, 2H), 2.90-2.84 (m, 2H), 1.85 (d, J=6.4 Hz, 6H). LCMS (ESI) [M+H]⁺=269.2.

### Step 4: 3-(1-Isopropyl-3-(5-(trifluoromethyl)pyridin-3-yl)-1H-pyrazol-5-yl)cyclopent-2-enone

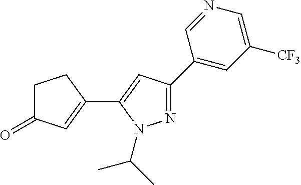 [SMILES: CC(C)n1nc(-c2cncc(C(F)(F)F)c2)cc1C1=CC(=O)CC1]

A suspension of 3-(3-bromo-1-isopropyl-1H-pyrazol-5-yl)cyclopent-2-enone (1.0 g, 3.7 mmol), 3-(4,4,5,5-tetramethyl-1,3,2-dioxaborolan-2-yl)-5-(trifluoromethyl)pyridine (1.01 g, 3.7 mmol), bis(di-tert-butyl(4-dimethylaminophenyl)phosphine)dichloropalladium(II) (270 mg, 0.4 mmol) and Cs₂CO₃ (3630 mg, 11.1 mmol) in 1,4-dioxane (20 mL) and water (4 mL) was degassed and purged with N₂ for three times, then the reaction mixture was stirred at 100° C. under N₂ for 2 hours. The reaction mixture was filtered and the filtrate was concentrated. The residue was purified by silica flash chromatography (30%-50% ethyl acetate in petroleum ether) to provide title compound (1000 mg, 80% yield). LCMS (ESI) [M+H]⁺=336.1.

### Step 5: 3-(1-Isopropyl-3-(5-(trifluoromethyl)pyridin-3-yl)-1H-pyrazol-5-yl)cyclopentanone

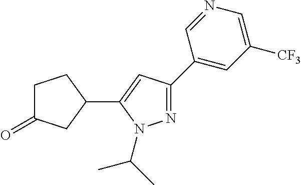 [SMILES: CC(C)n1nc(-c2cncc(C(F)(F)F)c2)cc1C1CCC(=O)C1]

To a solution of 3-(1-isopropyl-3-(5-(trifluoromethyl)pyridin-3-yl)-1H-pyrazol-5-yl)cyclopent-2-enone (1.0 g, 2.98 mmol) in methanol (20 mL) was added 10% palladium on carbon (0.32 g, 0.30 mmol) at 25° C., then the reaction mixture was stirred at 25° C. under H₂ (15 psi) for 20 hours. The reaction mixture was filtered and the filtrate cake was washed with methanol (10 mL×2). The combined organic layers were concentrated under vacuum. The residue was purified by silica flash chromatography (30%-50% ethyl acetate in petroleum ether) to provide title compound (700 mg, 70% yield). ¹H NMR (400 MHz, CDCl₃) δ 9.47 (s, 1H), 9.09 (d, J=1.2 Hz, 1H), 8.64 (s, 1H), 6.72 (s, 1H), 3.10-3.00 (m, 1H), 2.88-2.78 (m, 2H), 2.73-2.61 (m, 2H), 2.47-2.39 (m, 1H), 1.93-1.86 (m, 6H). LCMS (ESI) [M+H]+=338.1.

### Step 6: 4-(3-(1-Isopropyl-3-(5-(trifluoromethyl)pyridin-3-yl)-1H-pyrazol-5-yl)cyclopentyl) morpholine

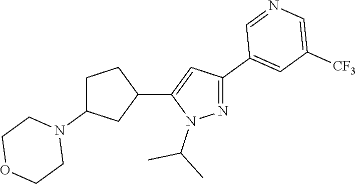 [SMILES: CC(C)n1nc(-c2cncc(C(F)(F)F)c2)cc1C1CCC(N2CCOCC2)C1]

To a solution of 3-(1-isopropyl-3-(5-(trifluoromethyl)pyridin-3-yl)-1H-pyrazol-5-yl)cyclopentanone (350 mg, 1.04 mmol), morpholine (225 mg, 2.59 mmol) and NaBH₃CN (130 mg, 2.08 mmol) in methanol (5 mL) was added acetic acid (0.03 mL), then the reaction mixture was stirred at 50° C. for 2 hours. The reaction mixture was concentrated. Then the residue was purified by silica flash chromatography (5%-10% methanol in dichloromethane) to provide title compound (320 mg, 76% yield) as a mixture of diasteromers. LCMS (ESI) [M+H]⁺=409.2.

### Step 7: 4-((1S,3S)-3-(1-Isopropyl-3-(5-(trifluoromethyl)pyridin-3-yl)-1H-pyrazol-5-yl)cyclopentyl)morpholine (Compound 58*) and 4-((1R,3R)-3-(1-Isopropyl-3-(5-(trifluoromethyl)pyridin-3-yl)-1H-pyrazol-5-yl)cyclopentyl)morpholine (Compound 59*), 4-((1R,3S)-3-(1-Isopropyl-3-(5-(trifluoromethyl)pyridin-3-yl)-1H-pyrazol-5-yl)cyclopentyl)morpholine (Compound 60*) and 4-((1S,3R)-3-(1-Isopropyl-3-(5-(trifluoromethyl)pyridin-3-yl)-1H-pyrazol-5-yl)cyclopentyl)morpholine (Compound 61*)

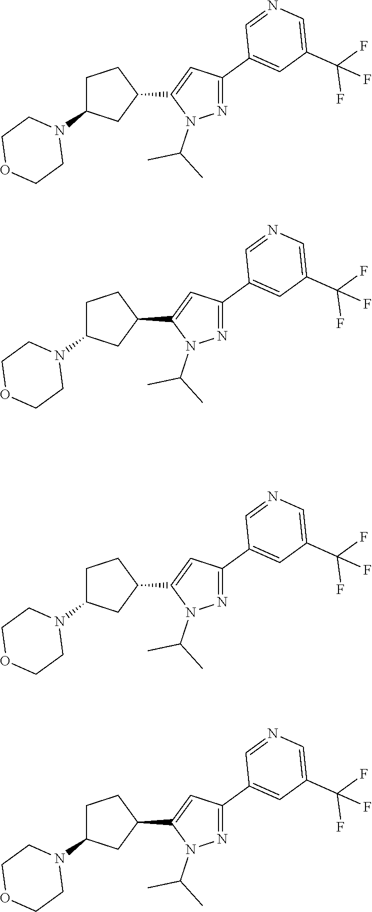 [SMILES: CC(C)n1nc(-c2cncc(C(F)(F)F)c2)cc1[C@H]1CC[C@H](N2CCOCC2)C1 ; CC(C)n1nc(-c2cncc(C(F)(F)F)c2)cc1[C@@H]1CC[C@@H](N2CCOCC2)C1 ; CC(C)n1nc(-c2cncc(C(F)(F)F)c2)cc1[C@H]1CC[C@@H](N2CCOCC2)C1 ; CC(C)n1nc(-c2cncc(C(F)(F)F)c2)cc1[C@@H]1CC[C@H](N2CCOCC2)C1]

The diasteromers of 4-(3-(1-isopropyl-3-(5-(trifluoromethyl)pyridin-3-yl)-1H-pyrazol-5-yl)cyclopentyl)morpholine (320 mg, 0.78 mmol) was purified by SFC (Column: ChiralPak AD-3 150×4.6 mm I.D., 3 um, Mobile phase: A: CO₂ B:IPA (0.05% DEA) Gradient: from 5% to 40% of B in 5.5 min, then 5% of B for 1.5 min, Flow rate: 2.5 mL/min, Column temp.: 40° C., Back pressure: 100 bar) to provide the title compound 58* (first peak on SFC, 20.1 mg, 6.2% yield), compound 59* (second peak on SFC, 27.3 mg, 8.4% yield), compound 60* (third peak on SFC, 41.7 mg, 12.2% yield) and compound 61* (fourth peak on SFC, 56.3 mg, 17.4% yield). LCMS (ESI) [M+H]⁺=409.2. The relative stereochemistry was arbitrarily assigned.

Compound 58*: ¹H NMR (400 MHz, CDCl₃) δ 9.09 (d, J=1.6 Hz, 1H), 8.69 (s, 1H), 8.25 (s, 1H), 6.30 (1H, s), 4.46-4.34 (m, 1H), 3.74-3.60 (m, 4H), 3.34-3.14 (m, 1H), 2.78-2.66 (m, 1H), 2.53-2.33 (m, 4H), 2.20-2.11 (m, 1H), 2.07-1.98 (m, 2H), 1.88-1.80 (m, 1H), 1.74-1.62 (m, 1H), 1.58-1.56 (m, 1H), 1.46 (d, J=5.6 Hz, 6H).

Compound 59*: ¹H NMR (400 MHz, CDCl₃) δ 9.09 (d, J=1.6 Hz, 1H), 8.69 (s, 1H), 8.25 (s, 1H), 6.30 (1H, s), 4.52-4.35 (m, 1H), 3.75-3.60 (m, 4H), 3.29-3.14 (m, 1H), 2.78-2.69 (m, 1H), 2.55-2.35 (m, 4H), 2.20-2.07 (m, 1H), 2.08-2.02 (m, 2H), 1.88-1.80 (m, 1H), 1.74-1.62 (m, 1H), 1.61-1.56 (m, 1H), 1.46 (d, J=5.6 Hz, 6H).

Compound 60*: ¹H NMR (400 MHz, CDCl₃) δ 9.14 (s, 1H), 8.75 (s, 1H), 8.31 (s, 1H), 6.42 (s, 1H), 4.53-4.40 (m, 1H), 3.78-3.70 (m, 4H), 3.22-3.09 (m, 1H), 2.79-2.70 (m, 1H), 2.65-2.44 (m, 4H), 2.37-2.22 (m, 1H), 2.19-2.10 (m, 1H), 2.04-1.96 (m, 1H), 1.82-1.69 (m, 3H), 1.53 (d, J=6.4 Hz, 6H).

Compound 61*: ¹H NMR (400 MHz, CDCl₃) δ 9.14 (d, J=1.2 Hz, 1H), 0.8.75 (s, 1H), 8.31 (s, 1H), 6.42 (s, 1H), 4.53-4.42 (m, 1H), 3.78-3.70 (m, 4H), 3.18-3.12 (m, 1H), 2.79-2.70 (m, 1H), 2.65-2.44 (m, 4H), 2.37-2.22 (m, 1H), 2.19-2.10 (m, 1H), 2.04-1.96 (m, 1H), 1.82-1.69 (m, 3H), 1.53 (d, J=6.4 Hz, 6H).

### Example Z: 4-((1S,3S)-3-(1-Isopropyl-3-(5-(trifluoromethyl)pyridin-3-yl)-1H-pyrazol-5-yl)cyclopentyl)-1,4-oxazepane, 4-((1R,3R)-3-(1-Isopropyl-3-(5-(trifluoromethyl)pyridin-3-yl)-1H-pyrazol-5-yl)cyclopentyl)-1,4-oxazepane, 4-((1R,3S)-3-(1-Isopropyl-3-(5-(trifluoromethyl) pyridin-3-yl)-1H-pyrazol-5-yl)cyclopentyl)-1,4-oxazepane & 4-((1S,3R)-3-(1-Isopropyl-3-(5-(trifluoromethyl)pyridin-3-yl)-1H-pyrazol-5-yl) cyclopentyl)-1,4-oxazepane (Compounds 62*, 63*, 64* & 65*)

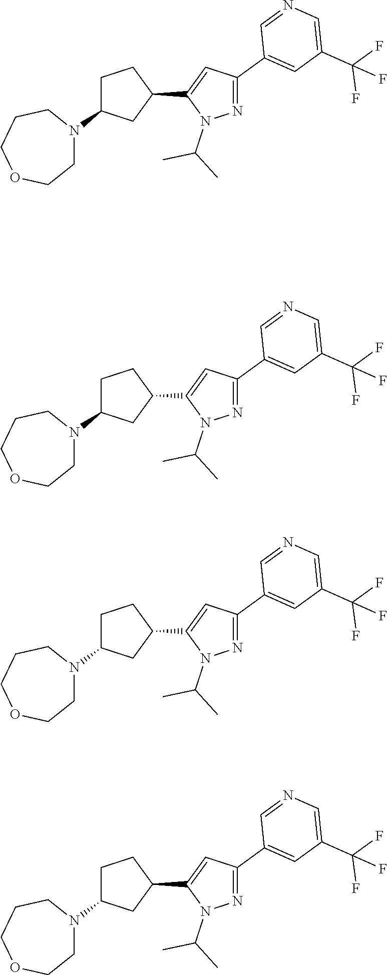 [SMILES: CC(C)n1nc(-c2cncc(C(F)(F)F)c2)cc1[C@@H]1CC[C@H](N2CCCOCC2)C1 ; CC(C)n1nc(-c2cncc(C(F)(F)F)c2)cc1[C@H]1CC[C@H](N2CCCOCC2)C1 ; CC(C)n1nc(-c2cncc(C(F)(F)F)c2)cc1[C@H]1CC[C@@H](N2CCCOCC2)C1 ; CC(C)n1nc(-c2cncc(C(F)(F)F)c2)cc1[C@@H]1CC[C@@H](N2CCCOCC2)C1]

### Step 1: (R)-3-(1-Isopropyl-3-(5-(trifluoromethyl)pyridin-3-yl)-1H-pyrazol-5-yl)cyclopentanone & (S)-3-(1-Isopropyl-3-(5-(trifluoromethyl)pyridin-3-yl)-1H-pyrazol-5-yl)cyclopentanone

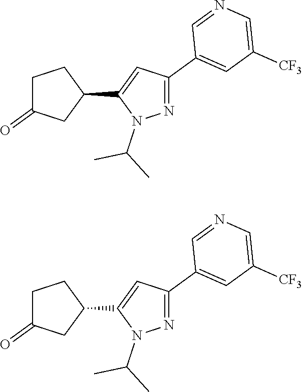 [SMILES: CC(C)n1nc(-c2cncc(C(F)(F)F)c2)cc1[C@@H]1CCC(=O)C1 ; CC(C)n1nc(-c2cncc(C(F)(F)F)c2)cc1[C@H]1CCC(=O)C1]

Following the procedures of compound 58*, 800 mg of 3-(1-isopropyl-3-(5-(trifluoromethyl) pyridin-3-yl)-1H-pyrazol-5-yl)cyclopentanone was prepared, which was purified by SFC (Column: Chiral Pak AD-3 150×4.6 mm I.D., 3 um, Mobile phase: A: CO₂ B: Ethanol (0.05% DEA), Gradient: from 5% to 40% of B in 5.5 min, then 5% of B for 1.5 min, Flow rate: 2.5 mL/min, Column temp.: 40° C., Back pressure: 100 bar) to provide (R)-3-(1-isopropyl-3-(5-(trifluoromethyl)pyridin-3-yl)-1H-pyrazol-5-yl) cyclopentanone (first peak on SFC, 350 mg, 43.7% yield) and (S)-3-(1-isopropyl-3-(5-(trifluoromethyl) pyridin-3-yl)-1H-pyrazol-5-yl)cyclopentanone (second peak on SFC, 370 mg, 46.2% yield) Both were obtained as a single unknown stereoisomer. The relative stereochemistry was arbitrarily assigned.

### Step 2: 4-((1S,3S)-3-(1-Isopropyl-3-(5-(trifluoromethyl)pyridin-3-yl)-1H-pyrazol-5-yl)cyclopentyl)-1,4-oxazepane (Compound 63*) & 4-((1R,3S)-3-(1-Isopropyl-3-(5-(trifluoromethyl)pyridin-3-yl)-1H-pyrazol-5-yl)cyclopentyl)-1,4-oxazepane (Compound 64*)

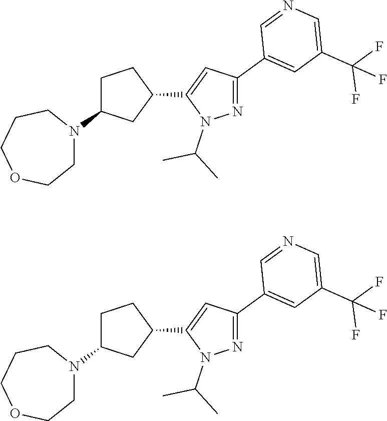 [SMILES: CC(C)n1nc(-c2cncc(C(F)(F)F)c2)cc1[C@H]1CC[C@H](N2CCCOCC2)C1 ; CC(C)n1nc(-c2cncc(C(F)(F)F)c2)cc1[C@H]1CC[C@@H](N2CCCOCC2)C1]

To a solution of (S)-3-(1-isopropyl-3-(5-(trifluoromethyl)pyridin-3-yl)-1H-pyrazol-5-yl)cyclopentanone (second peak on SFC, 100 mg, 0.3 mmol), 1,4-oxazepane (60 mg, 0.59 mmol) and NaBH₃CN (37 mg, 0.59 mmol) in methanol (2 mL) was added acetic acid (0.01 mL). Then the reaction mixture was stirred at 50° C. for 1 hour. A brown solution was formed. The reaction mixture was concentrated and the residue was purified by silica flash chromatography (5%-10% methanol in dichloromethane) to provide the diastereomeric mixture compound (120 mg, 90% yield). It was purified by chiral SFC (Column: Chiral Pak AD-3 150×4.6 mm I.D., 3 um, Mobile phase: A: CO₂ B: Ethanol (0.05% DEA), Gradient: from 5% to 40% of B in 5.5 min, then 5% of B for 1.5 min, Flow rate: 2.5 mL/min, Column temp.: 40° C., Back pressure: 100 bar) to provide compound 63* (first peak on SFC, 22.2 mg, 19% yield) and compound 64* (second peak on SFC, 32.6 mg, 27% yield). LCMS (ESI) [M+H]⁺=423.3. The relative stereochemistry was arbitrarily assigned.

Compound 63*: ¹H NMR (400 MHz, CDCl₃) δ 9.14 (d, J=1.6 Hz, 1H), 8.75 (s, 1H), 8.31 (s, 1H), 6.35 (s, 1H), 4.57-4.57 (m, 1H), 3.87-3.82 (m, 4H), 3.48-3.43 (m, 2H), 3.11-3.00 (m, 4H), 2.41-2.35 (m, 1H), 2.31-2.24 (m, 1H), 2.20-2.05 (m, 3H), 2.03-1.87 (m, 2H), 1.84-1.74 (m, 1H), 1.52 (d, J=6.8 Hz, 6H).

Compound 64*: ¹H NMR (400 MHz, CDCl₃) δ 9.15 (d, J=2.0 Hz, 1H), 8.75 (d, J=1.2 Hz, 1H), 8.31 (s, 1H), 6.42 (s, 1H), 4.54-4.43 (m, 1H), 3.84-3.77 (m, 4H), 3.21-3.09 (m, 2H), 2.89-2.76 (m, 4H), 2.38-2.29 (m, 1H), 2.19-2.10 (m, 1H), 2.04-1.93 (m, 3H), 1.84-1.76 (m, 2H), 1.75-1.65 (m, 1H), 1.52 (d, J=6.8 Hz, 6H).

### Step 3: 4-((1R,3R)-3-(1-Isopropyl-3-(5-(trifluoromethyl)pyridin-3-yl)-1H-pyrazol-5-yl)cyclopentyl)-1,4-oxazepane (Compound 65*) & 4-((1S,3R)-3-(1-Isopropyl-3-(5-(trifluoromethyl) pyridin-3-yl)-1H-pyrazol-5-yl)cyclopentyl)-1,4-oxazepane (Compound 62*)

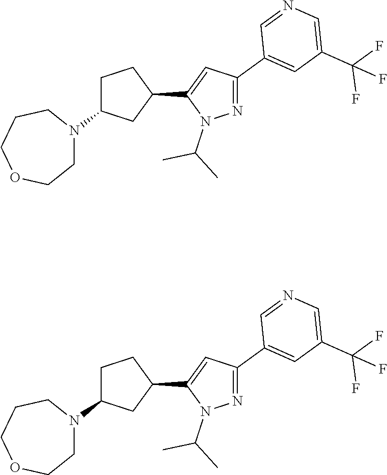 [SMILES: CC(C)n1nc(-c2cncc(C(F)(F)F)c2)cc1[C@@H]1CC[C@@H](N2CCCOCC2)C1 ; CC(C)n1nc(-c2cncc(C(F)(F)F)c2)cc1[C@@H]1CC[C@H](N2CCCOCC2)C1]

To a solution of (R)-3-(1-isopropyl-3-(5-(trifluoromethyl)pyridin-3-yl)-1H-pyrazol-5-yl)cyclopentanone (first peak on SFC, 100 mg, 0.3 mmol), 1,4-oxazepane (60 mg, 0.59 mmol) and NaBH₃CN (37 mg, 0.59 mmol) in methanol (2 mL) was added acetic acid (0.01 mL). Then the reaction mixture was stirred at 50° C. for 1 hour. A brown solution was formed. The reaction mixture was concentrated, and the residue was purified by silica flash chromatography (5%-10% methanol in dichloromethane) to provide the racemic mixture compound (120 mg, 90% yield). The racemic mixture was purified by chiral SFC (Column: Chiral Pak AD-3 150×4.6 mm I.D., 3 um, Mobile phase: A: CO₂ B: Ethanol (0.05% DEA), Gradient: from 5% to 40% of B in 5.5 min, then 5% of B for 1.5 min, Flow rate: 2.5 mL/min, Column temp.: 40° C., Back pressure: 100 bar) to provide compound 65* (first peak on SFC, 16.7 mg, 13.1% yield) and compound 62* (second peak on SFC, 37.8 mg, 31.2% yield). LCMS (ESI) [M+H]⁺=423.3. The relative stereochemistry was arbitrarily assigned.

Compound 65*: ¹H NMR (400 MHz, CDCl₃) δ 9.15 (d, J=1.6 Hz, 1H), 8.75 (s, 1H), 8.31 (s, 1H), 6.36 (s, 1H), 4.52-4.44 (m, 1H), 3.85-3.70 (m, 4H), 3.40-3.10 (m, 2H), 2.90-2.65 (m, 4H), 2.26-2.18 (m, 1H), 2.13-2.10 (m, 1H), 1.97-1.91 (m, 3H), 1.90-1.83 (m, 2H), 1.78-1.71 (m, 1H), 1.56-1.47 (m, 6H).

Compound 62*: ¹H NMR (400 MHz, CDCl₃) δ 9.16 (d, J=1.6 Hz, 1H), 8.75 (s, 1H), 8.32 (s, 1H), 6.46 (s, 1H), 4.49-4.44 (m, 1H), 3.85-3.80 (m, 4H), 3.34-3.23 (m, 1H), 3.19-3.10 (m, 1H), 2.98-2.90 (m, 4H), 2.41-2.33 (m, 1H), 2.19-2.01 (m, 1H), 2.11-1.98 (m, 3H), 1.90-1.83 (m, 2H), 1.81-1.75 (m, 1H), 1.56-1.47 (m, 6H).

### Example AA: (S)-4-((1R,3r,5S,6S)-6-(1-Isopropyl-3-(3-(trifluoromethyl)phenyl)-1H-1,2,4-triazol-5-yl)bicyclo[3.1.0]hexan-3-yl)-3-methylmorpholin and (S)-4-((1R,3s,5S,6S)-6-(1-Isopropyl-3-(3-(trifluoromethyl)phenyl)-1H-1,2,4-triazol-5-yl)bicyclo[3.1.0]hexan-3-yl)-3-methylmorpholine (Compounds 66 & 67)

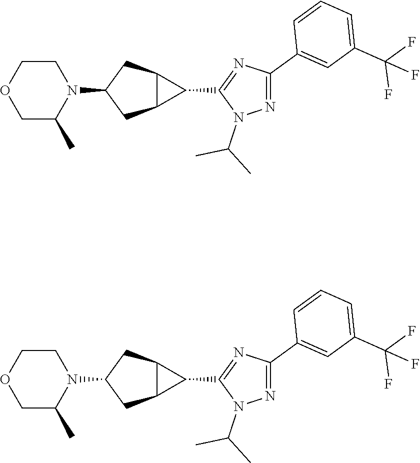 [SMILES: CC(C)n1nc(-c2cccc(C(F)(F)F)c2)nc1[C@H]1[C@@H]2C[C@H](N3CCOC[C@@H]3C)C[C@@H]21 ; CC(C)n1nc(-c2cccc(C(F)(F)F)c2)nc1[C@H]1[C@@H]2C[C@@H](N3CCOC[C@@H]3C)C[C@@H]21]

The title compound was synthesized following a procedure similar to compound 68 using 3-(trifluoromethyl)benzimidamide hydrochloride and (1R,5S)-3-((tert-butyldiphenylsilyl)oxy) bicyclo[3.1.0]hexane-6-carboxylic acid in step 1. Purification of the crude mixture by prep-TLC (10% methanol in dichloromethane) to provide compound 66 (first peak on SFC, 36.41 mg, 28.7% yield) and compound 67 (second peak on SFC, 30.31 mg, 24% yield). LCMS (ESI) [M+H]⁺=435.2. The relative stereochemistry was determined by 2D-NMR.

Compound 66: ¹H NMR (400 MHz, CD₃OD) δ 8.30 (s, 1H), 8.21 (d, J=7.6 Hz, 1H), 7.58 (d, J=6.8 Hz, 1H), 7.52-7.48 (m, 1H), 4.70-4.65 (m, 1H), 3.79-3.58 (m, 5H), 3.40-3.35 (m, 1H), 2.90-2.82 (s, 1H), 2.61-2.48 (m, 3H), 2.45-2.00 (m, 4H), 1.58 (d, J=2.0 Hz, 6H), 1.30-1.22 (m, 1H), 1.18-1.09 (m, 3H).

Compound 67: ¹H NMR (400 MHz, CD₃OD) δ 8.31 (s, 1H), 8.22 (d, J=7.6 Hz, 1H), 7.59 (d, J=6.8 Hz, 1H), 7.52-7.49 (m, 1H), 4.67-4.64 (m, 1H), 3.80-3.70 (m, 3H), 3.60-3.46 (m, 1H), 2.92-2.82 (m, 2H), 2.69 (s, 1H), 2.48 (s, 1H), 2.20-2.08 (m, 4H), 1.96 (s, 2H), 1.58 (d, J=2.0 Hz, 6H), 1.26 (s, 1H), 1.10 (d, J=5.6 Hz, 3H).

### Example AB: 4-((1R,3s,5S,6r)-6-(3-(5-(Difluoromethoxy)pyridin-3-yl)-1-isopropyl-1H-1,2,4-triazol-5-yl)bicyclo[3.1.0]hexan-3-yl)-1,4-oxazepane and 4-((1R,3r,5S,6r)-6-(3-(5-(Difluoromethoxy) pyridin-3-yl)-1-isopropyl-1H-1,2,4-triazol-5-yl)bicyclo[3.1.0]hexan-3-yl)-1,4-oxazepane (Compounds 68 & 69)

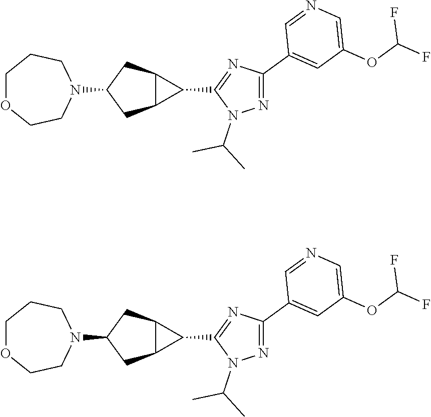 [SMILES: CC(C)n1nc(-c2cncc(OC(F)F)c2)nc1[C@H]1[C@@H]2C[C@@H](N3CCCOCC3)C[C@@H]21 ; CC(C)n1nc(-c2cncc(OC(F)F)c2)nc1[C@H]1[C@@H]2C[C@H](N3CCCOCC3)C[C@@H]21]

### Step 1: 3-Bromo-5-(5-((1R,5S,6r)-3-((tert-butyldiphenylsilyl)oxy)bicyclo[3.1.0]hexan-6-yl)-1-isopropyl-1H-1,2,4-triazol-3-yl)pyridine

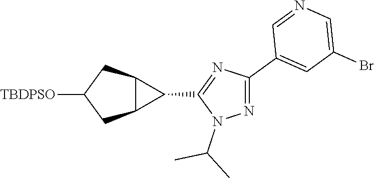

To a solution of (1R,5S,6r)-3-((tert-butyldiphenylsilyl)oxy)bicyclo[3.1.0]hexane-6-carboxylic acid (2 g, 5.26 mmol) and 5-bromonicotinimidamide hydrochloride (1857 mg, 7.88 mmol) in N,N-dimethylformamide (20 mL) was added N,N-diisopropylethylamine (3 mL, 17.22 mmol) and HATU (2198 mg, 5.78 mmol). The reaction mixture was stirred at 20° C. for 1 hour. To above mixture was added isopropylhydrazine hydrochloride (872 mg, 7.88 mmol) and acetic acid (3 mL, 52.56 mmol) and stirred at 80° C. for another 1.5 hours. The reaction mixture was diluted with water (20 mL) and extracted with ethyl acetate (100 mL×2). The combined organic layer was washed with brine (30 mL×3) and concentrated. The residue was purified by silica flash chromatography (0-10% ethyl acetate in petroleum ether) to afford the title compound (2.5 g, 79% yield). LCMS (ESI), [M+H]⁺=602.2.

### Step 2: 3-(5-((1R,5S,6r)-3-((tert-Butyldiphenylsilyl)oxy)bicyclo[3.1.0]hexan-6-yl)-1-isopropyl-1H-1,2,4-triazol-3-yl)-5-(difluoromethoxy)pyridine, (1R,5S)-6-[3-[5-(Difluoromethoxy)-3-pyridyl]-1H-1,2,4-triazol-5-yl]bicyclo[3.1.0]hexan-3-ol

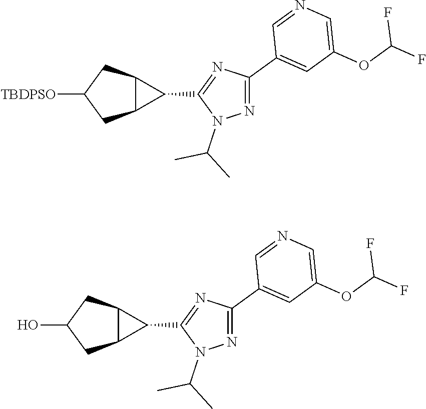

A solution of [(1S,5R)-6-[5-(5-bromo-3-pyridyl)-2-isopropyl-1,2,4-triazol-3-yl]-3-bicyclo[3.1.0]hexanyl]oxy-tert-butyl-diphenyl-silane (900 mg, 1.5 mmol), 2-di-tert-butylphosphino-2′,4′,6′-triisopropyl biphenyl (51 mg, 0.12 mmol), Pd₂(dba)₃ (27 mg, 0.03 mmol) and KOH (168 mg, 2.99 mmol) in dioxane (4 mL) and water (2 mL) was stirred at 100° C. for 16 hours under N₂. The reaction mixture was concentrated under reduced pressure and the residue was dissolved in acetonitrile (6 mL). Then KOH (2693 mg, 48 mmol) and difluoromethyl trifluoromethanesulfonate (891 mg, 4.45 mmol) were added at 25° C. and stirred for 30 min. The reaction mixture was diluted with water (15 mL) and the resulting solution was extracted with ethyl acetate (50 mL×3). The combined organic layers were washed with water (20 mL×2), dried over anhydrous sodium sulfate, filtered and concentrated under vacuum. The residue was purified by silica flash chromatography (0-100% ethyl acetate in petroleum ether) to provide title protected compound (600 mg, 68% yield), LCMS (ESI), [M+H]⁺=589.3 and title deprotected compound (200 mg, 38% yield. LCMS (ESI), [M+H]⁺=351.1.

### Step 3: (1R,5S,6r)-6-(3-(5-(Difluoromethoxy)pyridin-3-yl)-1-isopropyl-1H-1,2,4-triazol-5-yl)bicyclo[3.1.0]hexan-3-ol

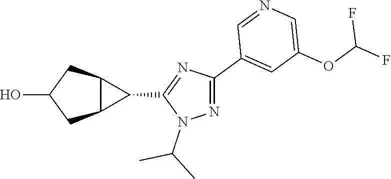

To a stirred solution of tert-butyl-[[(1R,5S)-6-[5-[5-(difluoromethoxy)-3-pyridyl]-2-isopropyl-1,2,4-triazol-3-yl]-3-bicyclo[3.1.0]hexanyl]oxy]-diphenyl-silane (600 mg, 1.02 mmol) in THF (10 mL) was added triethylamine trihydrofluoride (8 mL, 49.08 mmol). The reaction mixture was stirred at 70° C. for 16 hours. The reaction mixture was quenched by sat. NaHCO₃. The aqueous was extracted with ethyl acetate (50 mL×3). The combined organic layers were washed with brine (20 mL×2), dried over sodium sulfate, filtered and concentrated under reduced pressure. The residue was purified by silica flash chromatography (0-80% ethyl acetate in petroleum ether) to provide title compound (300 mg, 84% yield). LCMS (ESI), [M+H]⁺=351.1.

### Step 4: (1R,5S,6r)-6-(3-(5-(Difluoromethoxy)pyridin-3-yl)-1-isopropyl-1H-1,2,4-triazol-5-yl)bicyclo[3.1.0]hexan-3-one

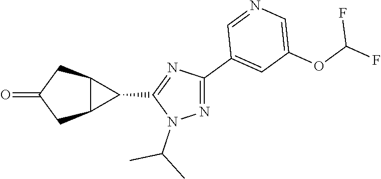 [SMILES: CC(C)n1nc(-c2cncc(OC(F)F)c2)nc1[C@H]1[C@@H]2CC(=O)C[C@@H]21]

A solution of (1R,5S,6r)-6-(3-(5-(difluoromethoxy)pyridin-3-yl)-1-isopropyl-1H-1,2,4-triazol-5-yl)bicyclo[3.1.0]hexan-3-ol (500 mg, 1.43 mmol) in DCM (10 mL) was added Dess-martin periodinane (908 mg, 2.14 mmol). The resulting solution was stirred at 25° C. for 16 hours. The reaction mixture was diluted with water (15 mL) and the resulting solution was extracted with ethyl acetate (50 mL×2). The combined organic layer was washed with Na₂SO₃ (10 mL×3), dried over anhydrous sodium sulfate, filtered and concentrated under vacuum. The residue was purified by silica flash chromatography (0-80% ethyl acetate in petroleum ether) to provide title compound (270 mg, 54% yield).

### Step 5: 4-((1R,3s,5S,6r)-6-(3-(5-(Difluoromethoxy)pyridin-3-yl)-1-isopropyl-1H-1,2,4-triazol-5-yl)bicyclo[3.1.0]hexan-3-yl)-1,4-oxazepane and 4-((1R,3r,5S,6r)-6-(3-(5-(Difluoromethoxy)pyridin-3-yl)-1-isopropyl-1H-1,2,4-triazol-5-yl)bicyclo[3.1.0]hexan-3-yl)-1,4-oxazepane (Compounds 68 and 69)

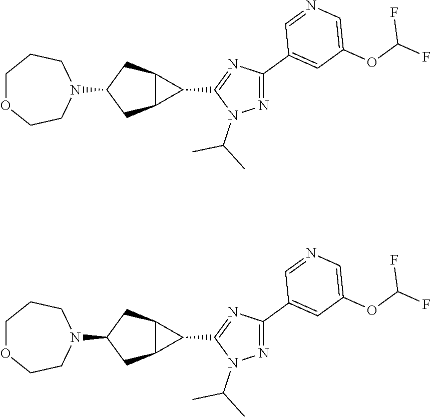 [SMILES: CC(C)n1nc(-c2cncc(OC(F)F)c2)nc1[C@H]1[C@@H]2C[C@@H](N3CCCOCC3)C[C@@H]21 ; CC(C)n1nc(-c2cncc(OC(F)F)c2)nc1[C@H]1[C@@H]2C[C@H](N3CCCOCC3)C[C@@H]21]

To a solution of (1R,5S,6r)-6-(3-(5-(difluoromethoxy)pyridin-3-yl)-1-isopropyl-1H-1,2,4-triazol-5-yl)bicyclo[3.1.0]hexan-3-one (130 mg, 0.37 mmol), homomorpholinehydrochloride (62 mg, 0.45 mmol) and acetic acid (22 mg, 0.37 mmol) in methyl alcohol (5 mL) was added sodiumcyanoborohydride (117 mg, 1.87 mmol) and stirred at 50° C. for 16 hours. The reaction mixture was diluted with water (10 mL) and adjusted pH to 9 with NaHCO₃. The resulting solution was extracted with ethyl acetate (50 mL×2). The combined organic layers were washed with water (20 mL×2), dried over anhydrous sodium sulfate, filtered and concentrated under vacuum. The residue was purified by reverse phase chromatography (water (0.05% NH₃H₂O+10 mM NH₄HCO₃)-ACN, 50%-80%, 25 mL/min) to provide compound 68 (second peak on SFC, 44.55 mg, 26% yield) and compound 69 (first peak on SFC, 35.38 mg, 22% yield). LCMS (ESI), [M+H]⁺=434.3. The relative stereochemistry was determined by 2D-NMR.

Compound 68: ¹H NMR (400 MHz, CD₃OD) δ 9.01 (d, J=1.6 Hz, 1H), 8.43 (d, J=2.8 Hz, 1H), 8.14 (s, 1H), 7.22-6.82 (m, 1H), 4.92-4.90 (m, 1H), 3.79 (t, J=6.0 Hz, 2H), 3.77-3.74 (m, 2H), 3.01-2.92 (m, 1H), 2.82-2.76 (m, 4H), 2.29 (dd, J=7.2, 12.4 Hz, 2H), 2.07-2.05 (m, 2H), 2.03-2.00 (m, 1H), 1.95-1.88 (m, 4H), 1.54 (d, J=6.8 Hz, 6H).

Compound 69: ¹H NMR (400 MHz, CD₃OD) δ 9.00 (d, J=1.6 Hz, 1H), 8.43 (d, J=2.8 Hz, 1H), 8.14 (s, 1H), 7.22-6.81 (m, 1H), 4.94-4.90 (m, 1H), 3.79 (t, J=6.0 Hz, 2H), 3.77-3.73 (m, 2H), 3.48-3.37 (m, 1H), 2.78-2.75 (m, 4H), 2.43-2.36 (m, 2H), 2.12-2.09 (m, 1H), 2.04-2.01 (m, 2H), 1.93-1.88 (m, 2H), 1.69 (dd, J=8.4, 14.0 Hz, 2H), 1.57 (d, J=6.4 Hz, 6H).

### Example AC: (R)-4-((1R,3r,5S,6R)-6-(1-isopropyl-3-(3-(trifluoromethyl)phenyl)-1H-1,2,4-triazol-5-yl)bicyclo[3.1.0]hexan-3-yl)-3-methylmorpholine and (R)-4-((1R,3s,5S,6R)-6-(1-isopropyl-3-(3-(trifluoromethyl)phenyl)-1H-1,2,4-triazol-5-yl)bicyclo [3.1.0]hexan-3-yl)-3-methylmorpholine (Compounds 70*& 71*)

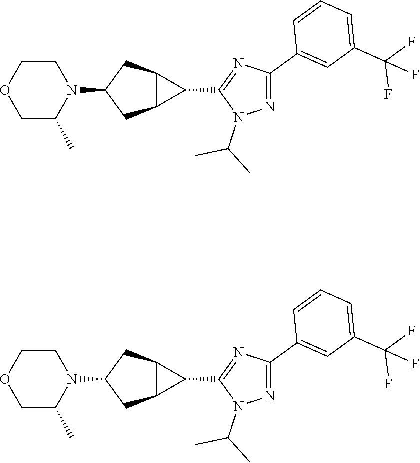 [SMILES: CC(C)n1nc(-c2cccc(C(F)(F)F)c2)nc1[C@H]1[C@@H]2C[C@H](N3CCOC[C@H]3C)C[C@@H]21 ; CC(C)n1nc(-c2cccc(C(F)(F)F)c2)nc1[C@H]1[C@@H]2C[C@@H](N3CCOC[C@H]3C)C[C@@H]21]

The title compound was synthesized following a procedure similar to compound 68 using 3-(trifluoromethyl)benzimidamide hydrochloride and (1R,5S)-3-((tert-butyldiphenylsilyl)oxy)bicyclo [3.1.0]hexane-6-carboxylic acid in step 1. Purification of the crude mixture by prep-TLC (10% methanol in dichloromethane) to provide the compound 70* (first peak on SFC, 41.35 mg, 33% yield) and compound 71* (second peak on SFC, 43.68 mg, 35% yield). LCMS (ESI) [M+H]⁺=435.3. The relative stereochemistry was arbitrarily assigned.

Compound 70*: ¹H NMR (400 MHz, CDCl₃) δ 8.30 (s, 1H), 8.21 (d, J=8.0 Hz, 1H), 7.60-7.56 (m, 1H), 7.52-7.47 (m, 1H), 4.70-4.67 (m, 1H), 3.82-3.56 (m, 4H), 3.38-3.35 (m, 1H), 2.88-2.84 (m, 1H), 2.72-2.44 (m, 2H), 2.33-2.16 (m, 2H), 2.05-2.00 (m, 3H), 1.84-1.69 (m, 2H), 1.58 (d, J=6.4 Hz, 6H), 1.09 (s, 3H).

Compound 71*: ¹H NMR (400 MHz, CDCl₃) δ 8.31 (s, 1H), 8.22 (d, J=8.0 Hz, 1H), 7.61-7.57 (m, 1H), 7.53-7.48 (m, 1H), 4.68-4.63 (m, 1H), 4.04-3.41 (m, 4H), 3.39-2.32 (m, 4H), 2.21-1.90 (m, 6H), 1.68 (s, 1H), 1.57 (d, J=6.4 Hz, 6H), 1.16 (s, 3H).

### Example AD: (R)-4-((1R,3s,5S,6R)-6-(1-Isopropyl-3-(6-(trifluoromethyl)pyridin-2-yl)-1H-1,2,4-triazol-5-yl)bicyclo[3.1.0]hexan-3-yl)-3-methylmorpholine and (R)-4-((1R,3r,5S,6R)-6-(1-isopropyl-3-(6-(trifluoromethyl)pyridin-2-yl)-1H-1,2,4-triazol-5-yl)bicyclo[3.1.0]hexan-3-yl)-3-methylmorpholine (Compounds 72 & 73)

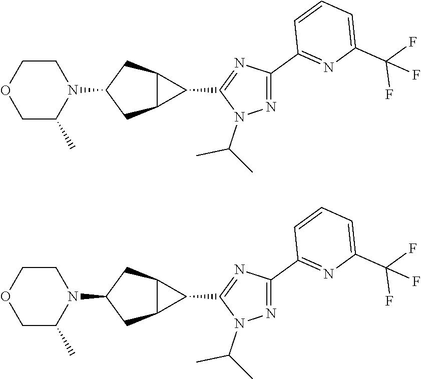 [SMILES: CC(C)n1nc(-c2cccc(C(F)(F)F)n2)nc1[C@H]1[C@@H]2C[C@@H](N3CCOC[C@H]3C)C[C@@H]21 ; CC(C)n1nc(-c2cccc(C(F)(F)F)n2)nc1[C@H]1[C@@H]2C[C@H](N3CCOC[C@H]3C)C[C@@H]21]

The title compound was synthesized following a procedure similar to compound 51 using (R)-3-methylmorpholine in last step. Purification of the crude mixture by reverse phase chromatography (water (0.05% NH₃H₂O+10 mM NH₄HCO₃)-ACN, 55%-85%) to provide compound 72 (first peak on HPLC, 32.76 mg, 17.4% yield) and compound 73 (second peak on HPLC, 69.52 mg, 36.9% yield). LCMS (ESI), [M+H]⁺=436.2. The relative stereochemistry was determined by 2D-NMR.

Compound 72: ¹H NMR (400 MHz, CD₃OD) δ 8.25 (d, J=7.6 Hz, 1H), 7.91 (t, J=8.0 Hz, 1H), 7.64 (d, J=7.6 Hz, 1H), 4.75-4.85 (m, 1H), 3.77 (brs, 3H), 3.53 (dd, J=3.6, 11.2 Hz, 1H), 2.96-2.78 (m, 2H), 2.70 (brs, 1H), 2.49 (d, J=9.6 Hz, 1H), 2.20-2.07 (m, 4H), 1.98 (brs, 2H), 1.70 (t, J=3.2 Hz, 1H), 1.59 (dd, J=2.8, 6.8 Hz, 6H), 1.11 (d, J=6.4 Hz, 3H).

Compound 73: ¹H NMR (400 MHz, CD₃OD) δ 8.25 (d, J=8.0 Hz, 1H), 7.91 (t, J=8.0 Hz, 1H), 7.64 (d, J=7.6 Hz, 1H), 4.76-4.70 (m, 1H), 3.79 (d, J=11.2 Hz, 1H), 3.70-3.62 (m, 2H), 3.60-3.55 (m, 1H), 3.35-3.32 (m, 1H), 2.86 (brs, 1H), 2.62-2.57 (m, 1H), 2.46 (d, J=12.0 Hz, 1H), 2.28-2.22 (m, 1H), 2.21-2.14 (m, 1H), 2.12-2.00 (m, 3H), 1.84 (d, J=6.4 Hz, 1H), 1.72 (dd, J=6.4, 13.2 Hz, 1H), 1.60 (d, J=6.8 Hz, 6H), 1.08 (d, J=6.4 Hz, 3H).

### Example AE: 4-((1R,3s,5S,6r)-6-(3-(3-(Difluoromethoxy)phenyl)-1-isopropyl-1H-1,2,4-triazol-5-yl)bicyclo[3.1.0]hexan-3-yl)-1,4-oxazepane and 4-((1R,3r,5S,6r)-6-(3-(3-(Difluoromethoxy)phenyl)-1-isopropyl-1H-1,2,4-triazol-5-yl)bicyclo[3.1.0]hexan-3-yl)-1,4-oxazepane (Compounds 74 & 75)

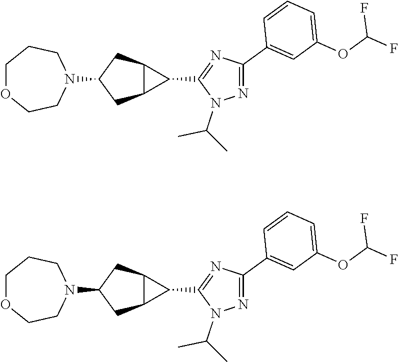 [SMILES: CC(C)n1nc(-c2cccc(OC(F)F)c2)nc1[C@H]1[C@@H]2C[C@@H](N3CCCOCC3)C[C@@H]21 ; CC(C)n1nc(-c2cccc(OC(F)F)c2)nc1[C@H]1[C@@H]2C[C@H](N3CCCOCC3)C[C@@H]21]

The title compound was synthesized following a procedure similar to compound 68 using 3-bromobenzimidamide hydrochloride in step 1. Purification of the crude mixture by reverse phase chromatography (water (0.05% NH₃H₂O+10 mM NH₄HCO₃)-ACN, 60%-90%) to provide compound 74 (first peak on SFC, 35.84 mg, 28.2% yield) and compound 75 (second peak on SFC, 35.65 mg, 27% yield). LCMS (ESI), [M+H]⁺=433.2. The relative stereochemistry was determined by 2D-NMR.

Compound 74: ¹H NMR (400 MHz, CD₃OD) δ 7.83 (d, J=7.6 Hz, 1H), 7.72 (s, 1H), 7.44 (t, J=8.0 Hz, 1H), 7.15 (dd, J=2.4, 8.0 Hz, 1H), 7.06-6.66 (m, 1H), 4.86-4.81 (m, 1H), 3.79 (t, J=6.0 Hz, 2H), 3.77-3.73 (m, 2H), 3.00-2.90 (m, 1H), 2.81-2.74 (m, 4H), 2.28 (dd, J=7.2, 12.4 Hz, 2H), 2.06-2.02 (m, 2H), 1.99-1.96 (m, 1H), 1.95-1.85 (m, 4H), 1.54 (d, J=6.8 Hz, 6H).

Compound 75: ¹H NMR (400 MHz, CD₃OD) δ 7.82 (d, J=8.0 Hz, 1H), 7.71 (s, 1H), 7.44 (t, J=8.0 Hz, 1H), 7.15 (dd, J=2.0, 8.4 Hz, 1H), 7.06-6.66 (m, 1H), 4.86-4.83 (m, 1H), 3.82-3.74 (m, 4H), 3.51-3.39 (m, 1H), 2.86-2.74 (m, 4H), 2.46-2.38 (m, 2H), 2.09-2.07 (m, 1H), 2.05-2.01 (m, 2H), 1.97-1.88 (m, 2H), 1.69 (dd, J=8.4, 13.6 Hz, 2H), 1.56 (d, J=6.4 Hz, 6H).

### Example AF: (R)-4-((1R,3r,5S,6R)-6-(3-(3-(Difluoromethoxy)phenyl)-1-isopropyl-1H-1,2,4-triazol-5-yl)bicyclo[3.1.0]hexan-3-yl)-3-methylmorpholine and (R)-4-((1R,3s,5S,6R)-6-(3-(3-(Difluoromethoxy)phenyl)-1-isopropyl-1H-1,2,4-triazol-5-yl)bicyclo[3.1.0]hexan-3-yl)-3-methylmorpholine (Compounds 76 & 77)

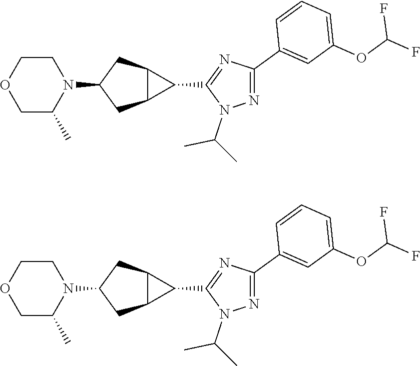 [SMILES: CC(C)n1nc(-c2cccc(OC(F)F)c2)nc1[C@H]1[C@@H]2C[C@H](N3CCOC[C@H]3C)C[C@@H]21 ; CC(C)n1nc(-c2cccc(OC(F)F)c2)nc1[C@H]1[C@@H]2C[C@@H](N3CCOC[C@H]3C)C[C@@H]21]

The title compound was synthesized following a procedure similar to compound 74 using (3R)-3-methylmorpholine in last step. Purification of the crude mixture by prep-TLC (ethyl acetate/methanol=10/1) to provide compound 76 (41 mg, 32% yield) and compound 77 (35.76 mg, 28% yield). LCMS (ESI), [M+H]⁺=433.3. The relative stereochemistry was determined by 2D-NMR.

Compound 76: ¹H NMR (400 MHz, CD₃OD) δ 7.83 (d, J=8.0 Hz, 1H), 7.72 (s, 1H), 7.44 (t, J=8.0 Hz, 1H), 7.15 (dd, J=2.4, 8.0 Hz, 1H), 7.06-6.67 (m, 1H), 4.86-4.80 (m, 1H), 3.78-3.71 (m, 1H), 3.71-3.62 (m, 2H), 3.54-3.43 (m, 2H), 2.89-2.81 (m, 1H), 2.69-2.61 (m, 1H), 2.54-2.46 (m, 1H), 2.34-2.19 (m, 2H), 2.12-2.08 (m, 1H), 2.05-1.98 (m, 2H), 1.84-1.70 (m, 2H), 1.56 (d, J=6.4 Hz, 6H), 1.09 (d, J=6.4 Hz, 3H).

Compound 77: ¹H NMR (400 MHz, CD₃OD) δ 7.83 (d, J=7.2 Hz, 1H), 7.72 (s, 1H), 7.44 (t, J=8.0 Hz, 1H), 7.15 (dd, J=2.4, 8.0 Hz, 1H), 7.06-6.67 (m, 1H), 4.86-4.83 (m, 1H), 3.78-3.66 (m, 3H), 3.55-3.45 (m, 1H), 3.12 (brs, 1H), 2.89-2.70 (m, 2H), 2.55-2.45 (m, 1H), 2.24-2.12 (m, 2H), 2.09-2.01 (m, 3H), 1.97-1.88 (m, 2H), 1.54 (dd, J=2.4, 6.4 Hz, 6H), 1.13 (d, J=6.4 Hz, 3H).

### Example AG: (S)-4-((1R,4S)-4-(1-Isopropyl-3-(6-(trifluoromethyl) pyridin-2-yl)-1H-1,2,4-triazol-5-yl)cyclohexyl)-3-methylmorpholine and (S)-4-((1S,4R)-4-(1-Isopropyl-3-(6-(trifluoromethyl)pyridin-2-yl)-1H-1,2,4-triazol-5-yl)cyclohexyl)-3-methyl morpholine (Compounds 78* & 79*), (R)-4-((1R,4R)-4-(1-Isopropyl-3-(6-(trifluoromethyl)pyridin-2-yl)-1H-1,2,4-triazol-5-yl)cyclohexyl)-3-methylmorpholine and (R)-4-((1S,4S)-4-(1-Isopropyl-3-(6-(trifluoromethyl)pyridin-2-yl)-1H-1,2,4-triazol-5-yl) cyclohexyl)-3-methylmorpholine (Compounds 80* & 81*)

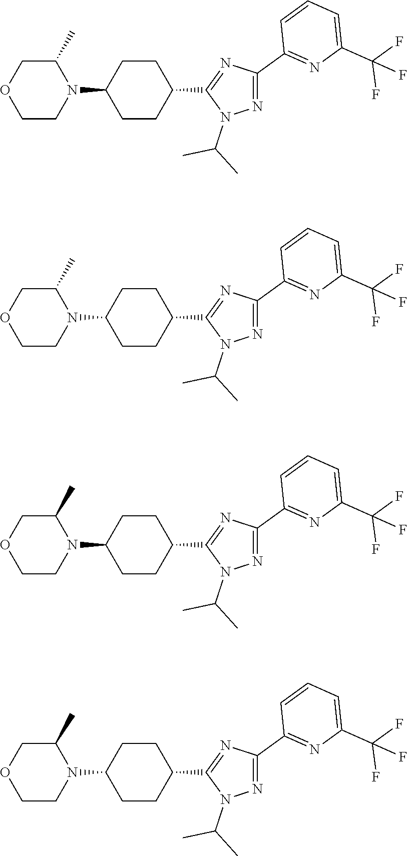 [SMILES: CC(C)n1nc(-c2cccc(C(F)(F)F)n2)nc1[C@H]1CC[C@H](N2CCOC[C@@H]2C)CC1 ; CC(C)n1nc(-c2cccc(C(F)(F)F)n2)nc1[C@H]1CC[C@@H](N2CCOC[C@@H]2C)CC1 ; CC(C)n1nc(-c2cccc(C(F)(F)F)n2)nc1[C@H]1CC[C@H](N2CCOC[C@H]2C)CC1 ; CC(C)n1nc(-c2cccc(C(F)(F)F)n2)nc1[C@H]1CC[C@@H](N2CCOC[C@H]2C)CC1]

### Step 1: 2-(1-Isopropyl-5-(1,4-dioxaspiro[4.5]decan-8-yl)-1H-1,2,4-triazol-3-yl)-6-(trifluoromethyl)pyridine

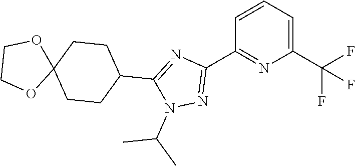 [SMILES: CC(C)n1nc(-c2cccc(C(F)(F)F)n2)nc1C1CCC2(CC1)OCCO2]

To a solution of 6-(trifluoromethyl)pyridine-2-carboxamidine;hydrochloride (300.0 mg, 1.33 mmol) and 1,4-dioxaspiro[4.5]decane-8-carboxylic acid (371.43 mg, 1.99 mmol) in N,N-dimethylformamide (12 mL)) was added HATU (556 mg, 1.46 mmol)) and NN-diisopropylethylamine (0.68 mL, 3.99 mmol) and stirred at 20 C for 1 hour. Then isopropylhydrazine hydrochloride (221 mg, 1.99 mmol) and acetic acid (0.76 mL, 13.3 mmol) was added and stirred at 80° C. for 1.5 hours. The mixture was diluted with ethyl acetate (500 mL) and washed with water and brine (50 mL×3). The organic layer was dried over sodium sulfate, filtered and concentrated to provide title compound (520 mg, 98.6% yield). LCMS (ESI) [M+H]⁺=397.2.

### Step 2: 4-(1-Isopropyl-3-(6-(trifluoromethyl)pyridin-2-yl)-1H-1,2,4-triazol-5-yl)cyclohexanone

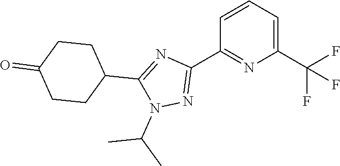 [SMILES: CC(C)n1nc(-c2cccc(C(F)(F)F)n2)nc1C1CCC(=O)CC1]

To a solution of 2-[5-(1,4-dioxaspiro[4.5]decan-8-yl)-1-isopropyl-1,2,4-triazol-3-yl]-6-(trifluoromethyl)pyridine (520.0 mg, 1.31 mmol) in Tetrahydrofuran (2 mL) was added hydrochloric acid (7.44 mL, 7.44 mmol, 1.0 M) at 25° C. and stirred at 25° C. for 16 hours. The mixture was adjusted to pH=9 with Na₂CO₃ and diluted with water (10 mL). The resulting mixture was extracted with ethyl acetate (30 mL×2). The combined organic layers were concentrated under reduced pressure. The residue was purified by silica flash chromatography (0-30% ethyl acetate in petroleum ether) to provide title compound (270 mg, 58.4% yield). LCMS (ESI) [M+H]⁺=353.2

### Step 3: (S)-4-((1R,4S)-4-(1-Isopropyl-3-(6-(trifluoromethyl)pyridin-2-yl)-1H-1,2,4-triazol-5-yl)cyclohexyl)-3-methylmorpholine and (S)-4-((1s,4R)-4-(1-Isopropyl-3-(6-(trifluoromethyl)pyridin-2-yl)-1H-1,2,4-triazol-5-yl)cyclohexyl)-3-methylmorpholine (Compounds 78* & 79*)

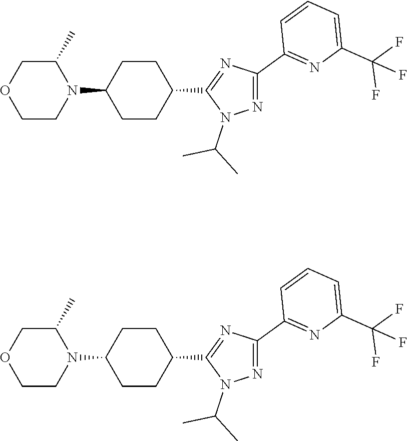 [SMILES: CC(C)n1nc(-c2cccc(C(F)(F)F)n2)nc1[C@H]1CC[C@H](N2CCOC[C@@H]2C)CC1 ; CC(C)n1nc(-c2cccc(C(F)(F)F)n2)nc1[C@H]1CC[C@@H](N2CCOC[C@@H]2C)CC1]

To a solution of 4-[2-isopropyl-5-[6-(trifluoromethyl)-2-pyridyl]-1,2,4-triazol-3-yl]cyclohexanone (100 mg, 0.28 mmol) in methyl alcohol (4 mL) was added (S)-3-methylmorpholine (287 mg, 2.84 mmol), acetic acid (17 mg, 0.28 mmol) and sodium cyanoborohydride (54 mg, 0.85 mmol) and stirred at 70° C. for 30 hours. The reaction mixture was diluted with water (20 mL) and adjust to pH=9 with saturated Na₂CO3. The resulting solution was extracted with dichloromethane (30 mL×3), and the combined organic layers were concentrated under vacuum. The residue was purified by reverse phase chromatography (30%-60% acetonitrile/0.05% ammonia hydroxide in water) to provide compound 78* (second peak on SFC, 37.54 mg, 29% yield) and compound 79* (first peak on SFC, 14.25 mg, 11% yield). The relative stereochemistry was arbitrarily assigned.

Compound 78*: ¹H NMR (400 MHz, CD₃OD) δ 8.33 (d, J=7.6 Hz, 1H), 8.12 (t, J=8.0 Hz, 1H), 7.82 (d, J=7.6 Hz, 1H), 4.86-4.76 (m, 1H), 3.87-3.77 (m, 1H), 3.70 (dd, J=2.8, 11.2 Hz, 1H), 3.67-3.56 (m, 1H), 3.04-2.93 (m, 2H), 2.89-2.81 (m, 1H), 2.80-2.78 (m, 1H), 2.65-2.52 (m, 1H), 2.10-1.99 (m, 3H), 1.99-1.79 (m, 3H), 1.78-1.66 (m, 1H), 1.56 (d, J=6.8 Hz, 6H), 1.53-1.38 (m, 1H), 1.05 (d, J=6.0 Hz, 3H).

Compound 79*: ¹H NMR (400 MHz, CD₃OD) δ 8.36 (d, J=8.0 Hz, 1H), 8.12 (t, J=8.0 Hz, 1H), 7.81 (d, J=7.6 Hz, 1H), 4.82-4.71 (m, 1H), 3.79-3.65 (m, 3H), 3.42 (dd, J=6.4, 10.8 Hz, 1H), 3.29-3.23 (m, 1H), 2.95-2.91 (m, 1H), 2.85-2.79 (m, 1H), 2.75-2.67 (m, 1H), 2.66-2.58 (m, 1H), 2.25-2.04 (m, 4H), 1.89-1.74 (m, 2H), 1.74-1.65 (m, 1H), 1.64-1.58 (m, 1H), 1.55 (d, J=6.4 Hz, 6H), 1.05 (d, J=6.4 Hz, 3H).

### Step 4: (R)-4-((1R,4R)-4-(1-isopropyl-3-(6-(trifluoromethyl)pyridin-2-yl)-1H-1,2,4-triazol-5-yl)cyclohexyl)-3-methylmorpholine and (R)-4-((1s,4S)-4-(1-isopropyl-3-(6-(trifluoromethyl)pyridin-2-yl)-1H-1,2,4-triazol-5-yl)cyclohexyl)-3-methylmorpholine (Compounds 80* & 81*)

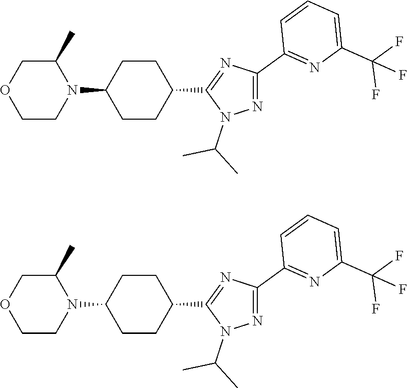 [SMILES: CC(C)n1nc(-c2cccc(C(F)(F)F)n2)nc1[C@H]1CC[C@H](N2CCOC[C@H]2C)CC1 ; CC(C)n1nc(-c2cccc(C(F)(F)F)n2)nc1[C@H]1CC[C@@H](N2CCOC[C@H]2C)CC1]

To a solution of 4-[2-isopropyl-5-[6-(trifluoromethyl)-2-pyridyl]-1,2,4-triazol-3-yl]cyclohexanone (130 mg, 0.37 mmol) in methanol (6 mL) was added (R)-3-methylmorpholine (373 mg, 3.69 mmol), acetic acid (22 mg, 0.37 mmol) and sodium cyanoborohydride (70 mg, 1.11 mmol) and stirred at 70° C. for 30 hours. The reaction mixture was diluted with water (20 mL) and adjust to pH=9 with saturated Na₂CO₃. The resulting solution was extracted with dichloromethane (30 mL×3), and the combined organic layers were concentrated under vacuum. The resulting residue was purified by reverse phase chromatography (30%-60% acetonitrile/0.05% ammonia hydroxide in water) to provide compound 80* (second peak on SFC, 7.79 mg, 5% yield) and compound 81* (first peak on SFC, 32.82 mg, 20% yield). LCMS (ESI) [M+H]⁺=438.1. The relative stereochemistry was arbitrarily assigned.

Compound 80*: ¹H NMR (400 MHz, CD₃OD) δ 8.33 (d, J=8.0 Hz, 1H), 8.12 (t, J=8.0 Hz, 1H), 7.83 (d, J=7.6 Hz, 1H), 4.84-4.82 (m, 1H), 3.86 (d, J=11.6 Hz, 1H), 3.76 (d, J=11.2 Hz, 1H), 3.71-3.60 (m, 1H), 3.40-3.32 (m, 1H), 3.25-2.56 (m, 5H), 2.12-1.67 (m, 7H), 1.56 (d, J=6.8 Hz, 6H), 1.52-1.49 (m, 1H), 1.11 (d, J=5.6 Hz, 3H).

Compound 81*: ¹H NMR (400 MHz, CD₃OD) δ 8.36 (d, J=8.0 Hz, 1H), 8.12 (t, J=7.6 Hz, 1H), 7.82 (d, J=7.6 Hz, 1H), 4.77-4.71 (m, 1H), 3.75-3.68 (m, 3H), 3.42 (dd, J=6.4, 11.2 Hz, 1H), 3.31-3.28 (m, 1H), 2.95-2.91 (m, 1H), 2.85-2.80 (m, 1H), 2.75-2.67 (m, 1H), 2.67-2.59 (m, 1H), 2.19-2.08 (m, 4H), 1.90-1.78 (m, 2H), 1.74-1.66 (m, 1H), 1.65-1.58 (m, 1H), 1.55 (d, J=6.4 Hz, 6H), 1.05 (d, J=6.0 Hz, 3H).

### Example AH: 1-((1R,3R)-3-(1-Isopropyl-3-(6-(trifluoromethyl)pyridin-3-yl)-1H-1,2,4-triazol-5-yl)cyclopentyl)-4-(2-methoxyethyl)piperazine and 1-((1S,3R)-3-(1-Isopropyl-3-(6-(trifluoromethyl) pyridin-3-yl)-1H-1,2,4-triazol-5-yl)cyclopentyl)-4-(2-methoxyethyl) piperazine (Compounds 82 & 83)

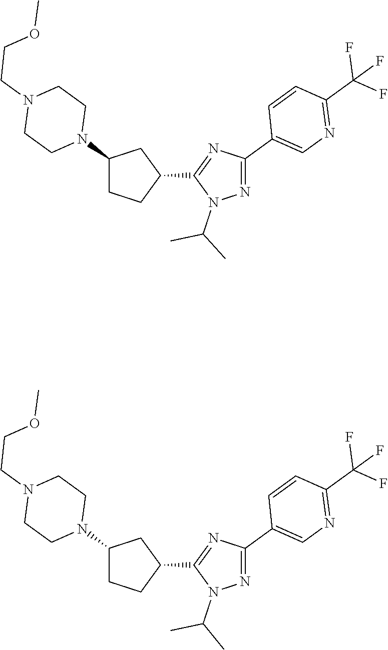 [SMILES: COCCN1CCN([C@@H]2CC[C@@H](c3nc(-c4ccc(C(F)(F)F)nc4)nn3C(C)C)C2)CC1 ; COCCN1CCN([C@H]2CC[C@@H](c3nc(-c4ccc(C(F)(F)F)nc4)nn3C(C)C)C2)CC1]

The title compound was synthesized following a procedure similar to compound 68 using 6-(trifluoromethyl)nicotinimidamide hydrochloride and (R)-3-oxocyclopentanecarboxylic acid in step 1. Purification of the crude mixture by silica flash chromatography (0-10% methanol in dichloromethane) to provide the title compound (50 mg, 68.2% yield) as a mixture of diastereomers. The mixture of diastereomers was separated using chiral SFC (Daicel Chiralpak SFC-21; DAICEL CHIRALCEL OD-H (250 mm*30 mm, 5 um); 0.1% NH₃ in ETOH; 15/15; 60 mL/min) to provide compound 82 (first peak on SFC, 3.65 mg, 6.9% yield) and compound 83 (second peak on SFC, 30.03 mg, 59.5% yield). LCMS (ESI), [M+H]⁺=467.3. The relative stereochemistry was determined by 2D-NMR.

Compound 82: ¹H NMR (400 MHz, CD₃OD) δ 9.32 (s, 1H), 8.61 (dd, J=1.6, 8.4 Hz, 1H), 7.90 (d, J=8.0 Hz, 1H), 4.80-4.73 (m, 1H), 3.65-3.56 (m, 3H), 3.35 (s, 3H), 3.18-3.06 (m, 1H), 2.73 (brs, 9H), 2.37-2.19 (m, 4H), 2.13-2.08 (m, 1H), 2.02-1.93 (m, 1H), 1.74-1.67 (m, 1H), 1.54 (dd, J=2.4, 6.4 Hz, 6H).

Compound 83: ¹H NMR (400 MHz, CD₃OD) δ 9.32 (s, 1H), 8.60 (dd, J=1.6, 8.4 Hz, 1H), 7.90 (d, J=8.4 Hz, 1H), 4.79-4.72 (m, 1H), 3.55 (t, J=5.2 Hz, 2H), 3.51-3.44 (m, 1H), 3.34 (s, 3H), 2.89-2.83 (m, 1H), 2.63-2.60 (m, 10H), 2.41-2.35 (m, 1H), 2.20-2.13 (m, 1H), 2.10-2.03 (m, 2H), 2.01-1.92 (m, 1H), 1.87-1.80 (m, 1H), 1.53 (dd, J=2.8, 6.8 Hz, 6H).

### Example AI: 1-Cyclobutyl-4-((1R,3s,5S,6r)-6-(1-isopropyl-3-(6-(trifluoromethyl) pyridin-2-yl)-1H-pyrazol-5-yl)bicyclo[3.1.0]hexan-3-yl)piperazine and 1-Cyclobutyl-4-((1R,3r,5S,6r)-6-(1-isopropyl-3-(6-(trifluoromethyl)pyridin-2-yl)-1H-pyrazol-5-yl)bicyclo[3.1.0]hexan-3-yl)piperazine (Compounds 84 & 85)

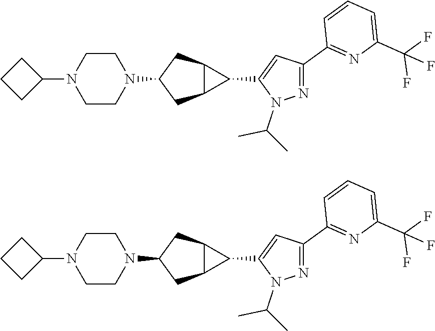 [SMILES: CC(C)n1nc(-c2cccc(C(F)(F)F)n2)cc1[C@H]1[C@@H]2C[C@@H](N3CCN(C4CCC4)CC3)C[C@@H]21 ; CC(C)n1nc(-c2cccc(C(F)(F)F)n2)cc1[C@H]1[C@@H]2C[C@H](N3CCN(C4CCC4)CC3)C[C@@H]21]

The title compound was synthesized following a procedure similar to compound 29* using (1R,5S,6r)-6-(3-iodo-1-isopropyl-1H-pyrazol-5-yl)bicyclo[3.1.0]hexan-3-one and 2-(4,4,5,5-tetramethyl-1,3,2-dioxaborolan-2-yl)-6-(trifluoromethyl)pyridine in step 1. Purification of the crude mixture by reverse phase chromatography (Phenomenex Gemini-NX C18 75*30 mm*3 um water (0.05% NH₃H₂O+10 mM NH₄HCO₃)-CAN; 57%-87%) to provide compound 84 (second peak on SFC, 33.71 mg, 25% yield) and compound 85 (first peak on SFC, 35.5 mg, 26% yield). LCMS (ESI) [M+H]⁺=474.3. The relative stereochemistry was determined by 2D-NMR.

Compound 84: ¹H NMR (400 MHz, CD₃OD) δ 8.15 (d, J=8.0 Hz, 1H), 7.97 (t, J=8.0 Hz, 1H), 7.62 (d, J=7.6 Hz, 1H), 6.51 (s, 1H), 4.84-4.74 (m, 1H), 2.80-2.76 (m, 1H), 2.73-2.33 (m, 8H), 2.29 (dd, J=7.2, 12.4 Hz, 3H), 2.08-2.04 (m, 2H), 1.96-1.68 (m, 9H), 1.53 (d, J=6.8 Hz, 6H).

Compound 85: ¹H NMR (400 MHz, CD₃OD) δ 8.15 (d, J=8.0 Hz, 1H), 7.97 (t, J=8.0 Hz, 1H), 7.62 (d, J=7.6 Hz, 1H), 6.46 (s, 1H), 4.85-4.78 (m, 1H), 3.00 (q, J=8.4 Hz, 1H), 2.81-2.76 (m, 1H), 2.73-2.10 (m, 9H), 2.08-2.01 (m, 2H), 1.96-1.67 (m, 8H), 1.61 (dd, J=8.4, 13.6 Hz, 2H), 1.55 (d, J=6.4 Hz, 6H).

### Example AJ: 2-(4-((1R,3R)-3-(1-Isopropyl-3-(6-(trifluoromethyl) pyridin-3-yl)-1H-pyrazol-5-yl)cyclopentyl)piperazin-1-yl)ethanol and 2-(4-((1S,3R)-3-(1-Isopropyl-3-(6-(trifluoromethyl)pyridin-3-yl)-1H-pyrazol-5-yl)cyclopentyl)piperazin-1-yl)ethanol (Compounds 86* & 87*)

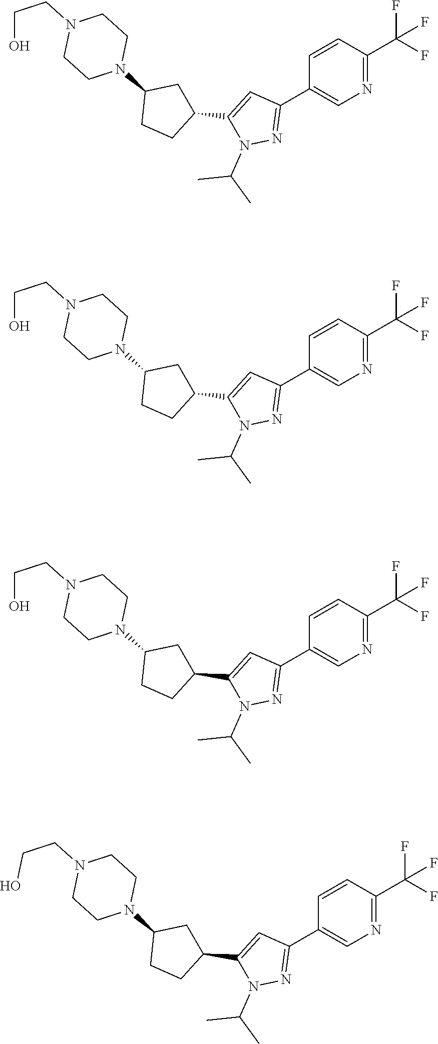 [SMILES: CC(C)n1nc(-c2ccc(C(F)(F)F)nc2)cc1[C@@H]1CC[C@@H](N2CCN(CCO)CC2)C1 ; CC(C)n1nc(-c2ccc(C(F)(F)F)nc2)cc1[C@@H]1CC[C@H](N2CCN(CCO)CC2)C1 ; CC(C)n1nc(-c2ccc(C(F)(F)F)nc2)cc1[C@H]1CC[C@H](N2CCN(CCO)CC2)C1 ; CC(C)n1nc(-c2ccc(C(F)(F)F)nc2)cc1[C@H]1CC[C@@H](N2CCN(CCO)CC2)C1]

### Step 1: (R)-3-(1-Isopropyl-3-(6-(trifluoromethyl)pyridin-3-yl)-1H-pyrazol-5-yl)cyclopentanone and (S)-3-(1-Isopropyl-3-(6-(trifluoromethyl)pyridin-3-yl)-1H-pyrazol-5-yl)cyclopentanone

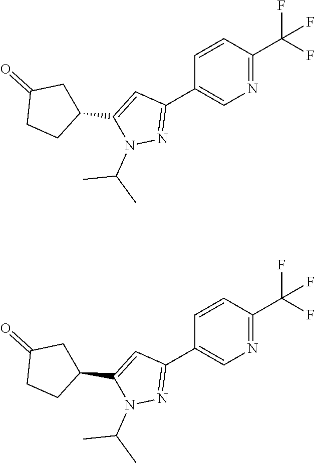 [SMILES: CC(C)n1nc(-c2ccc(C(F)(F)F)nc2)cc1[C@@H]1CCC(=O)C1 ; CC(C)n1nc(-c2ccc(C(F)(F)F)nc2)cc1[C@H]1CCC(=O)C1]

13741 Following the procedures of compound 58*, 2400 mg of 3-(1-Isopropyl-3-(6-(trifluoromethyl)pyridin-3-yl)-1H-pyrazol-5-yl)cyclopentanone was prepared, which was purified by SFC (SFC-11, Phenomenex-Cellulose-2 (250 mm*50 mm, 10 μm, 0.1% NH₃H₂O ETOH, 25/25, 200 ml/min) to provide title compound-1 (first peak on SFC, 1 g, 42% yield) and title compound-2 (second peak on SFC, 1 g, 42% yield). LCMS (ESI), [M+H]⁺=338.1. The relative stereochemistry was arbitrarily assigned.

### Step 2: 2-(4-((3R)-3-(1-Isopropyl-3-(6-(trifluoromethyl)pyridin-3-yl)-1H-pyrazol-5-yl)cyclopentyl) piperazin-1-yl)ethanol

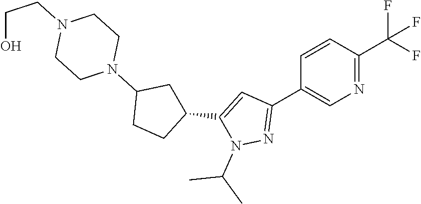 [SMILES: CC(C)n1nc(-c2ccc(C(F)(F)F)nc2)cc1[C@@H]1CCC(N2CCN(CCO)CC2)C1]

To a solution of (R)-3-(1-Isopropyl-3-(6-(trifluoromethyl)pyridin-3-yl)-1H-pyrazol-5-yl)cyclopentanone (100 mg, 0.30 mmol), 2-(piperazin-1-yl)ethanol (116 mg, 0.89 mmol), acetic acid (36 mg, 0.59 mmol) and 4AMS in anhydrous 1,2-dichloroethane (3 mL) were stirred at 25° C. for 2 hours. Then NaBH(OAc)₃ (314 mg, 1.48 mmol) was added and stirred at 25° C. for 16 hours. The mixture was diluted with water (15 mL) and adjusted pH to 9 with NaHCO₃ aq. The resulting mixture was extracted with ethyl acetate (50 mL×3). The combined organic layer was dried over anhydrous sodium sulfate and concentrated under vacuum. The residue was purified by silica flash chromatography (0-5% methanol in dichloromethane) to afford title compound (100 mg, 75% yield) as a mixture of diastereomers. LCMS (ESI), [M+H]⁺=452.2.

### Step 3: 2-(4-((1R,3R)-3-(1-Isopropyl-3-(6-(trifluoromethyl)pyridin-3-yl)-1H-pyrazol-5-yl)cyclopentyl)piperazin-1-yl)ethanol and 2-(4-((1S,3R)-3-(1-Isopropyl-3-(6-(trifluoromethyl)pyridin-3-yl)-1H-pyrazol-5-yl)cyclopentyl)piperazin-1-yl)ethanol (Compounds 86* & 87*)

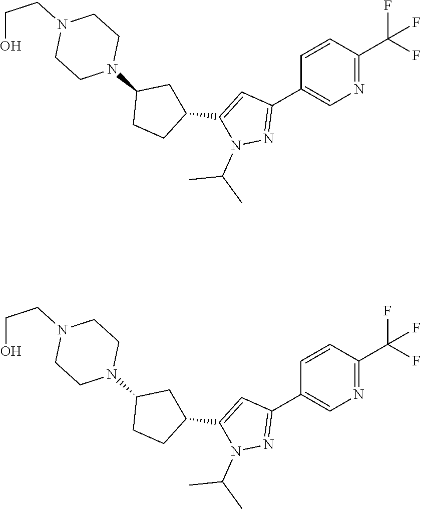 [SMILES: CC(C)n1nc(-c2ccc(C(F)(F)F)nc2)cc1[C@@H]1CC[C@@H](N2CCN(CCO)CC2)C1 ; CC(C)n1nc(-c2ccc(C(F)(F)F)nc2)cc1[C@@H]1CC[C@H](N2CCN(CCO)CC2)C1]

The mixture of diastereomers (120 mg, 0.27 mmol) was separated using chiral SFC (SFC-17, DAICEL CHIRALPAK IG (250 mm*30 mm, 10 um), 0.1% NH₃H₂O ETOH, 40/40, 70 ml/min) to provide compound 86* (anti isomer, first peak on SFC, 17.76 mg, 15% yield) and compound 87* (syn isomer, second peak on SFC, 75.74 mg, 63% yield). The relative stereochemistry was arbitrarily assigned.

Compound 86*: ¹H NMR (400 MHz, CD₃OD) δ 9.10 (d, J=1.2 Hz, 1H), 8.37 (dd, J=1.6, 8.0 Hz, 1H), 7.82 (d, J=8.4 Hz, 1H), 6.69 (s, 1H), 4.69-4.60 (m, 1H), 3.70 (t, J=6.0 Hz, 2H), 3.30-3.24 (m, 1H), 2.88-2.47 (m, 1H), 3.05-2.44 (m, 11H), 2.44-2.33 (m, 1H), 2.24-2.15 (m, 1H), 2.11-1.99 (m, 1H), 1.86-1.73 (m, 2H), 1.68-1.55 (m, 1H), 1.53-1.45 (m, 6H).

Compound 87*: ¹H NMR (400 MHz, CD₃OD) δ 9.10 (d, J=1.6 Hz, 1H), 8.37 (dd, J=1.6, 8.4 Hz, 1H), 7.82 (d, J=8.4 Hz, 1H), 6.69 (s, 1H), 4.69-4.59 (m, 1H), 3.35 (s, 3H), 3.29-3.23 (m, 1H), 2.96-2.77 (m, 3H), 2.44-2.26 (m, 3H), 2.23-2.15 (m, 1H), 2.10-2.02 (m, 1H), 2.00-1.92 (m, 2H), 1.90-1.54 (m, 6H), 1.53-1.45 (m, 6H).

### Example AK: 2-(4-((1S,3S)-3-(1-Isopropyl-3-(6-(trifluoromethyl)pyridin-3-yl)-1H-pyrazol-5-yl)cyclopentyl)piperazin-1-yl)ethanol & 2-(4-((1R,3S)-3-(1-isopropyl-3-(6-(trifluoromethyl)pyridin-3-yl)-1H-pyrazol-5-yl)cyclopentyl) piperazin-1-yl)ethanol (Compounds 88* & 89*)

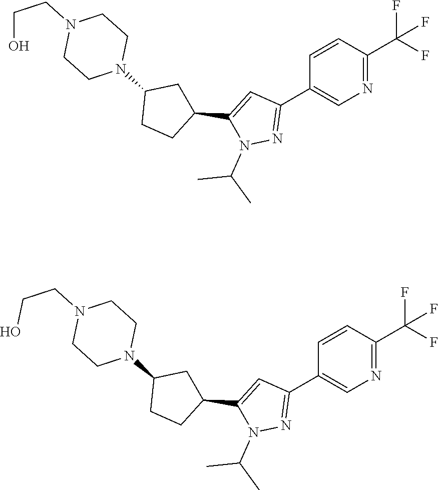 [SMILES: CC(C)n1nc(-c2ccc(C(F)(F)F)nc2)cc1[C@H]1CC[C@H](N2CCN(CCO)CC2)C1 ; CC(C)n1nc(-c2ccc(C(F)(F)F)nc2)cc1[C@H]1CC[C@@H](N2CCN(CCO)CC2)C1]

### Step 1: 2-(4-((3S)-3-(1-Isopropyl-3-(6-(trifluoromethyl)pyridin-3-yl)-1H-pyrazol-5-yl)cyclopentyl)piperazin-1-yl)ethanol

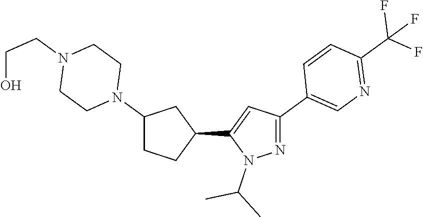 [SMILES: CC(C)n1nc(-c2ccc(C(F)(F)F)nc2)cc1[C@H]1CCC(N2CCN(CCO)CC2)C1]

To a solution of (S)-3-(1-Isopropyl-3-(6-(trifluoromethyl)pyridin-3-yl)-1H-pyrazol-5-yl) cyclopentanone (120.0 mg, 0.36 mmol), 2-(piperazin-1-yl)ethanol (139 mg, 1.07 mmol), acetic acid (43 mg, 0.71 mmol) and 4 A MS in anhydrous 1,2-dichloroethane (3 mL) were stirred at 25° C. for 2 hours. Then NaBH(OAc)₃ (377 mg, 1.78 mmol) was added and stirred at 25° C. for 16 hours. The mixture was diluted with water (25 mL) and adjusted pH to 9 with NaHCO₃ aq. The resulting mixture was extracted with ethyl acetate (50 mL×3). The combined organic layer was dried over anhydrous sodium sulfate, filtered and concentrated under vacuum. The residue was purified by silica flash chromatography (0-5% methanol in dichloromethane) to afford title compound (120 mg, 74% yield) as a mixture of diastereomers. LCMS (ESI), [M+H]⁺=452.2.

### Step 2: 2-(4-((1S,3S)-3-(1-Isopropyl-3-(6-(trifluoromethyl)pyridin-3-yl)-1H-pyrazol-5-yl)cyclopentyl)piperazin-1-yl)ethanol and 2-(4-((1R,3S)-3-(1-Isopropyl-3-(6-(trifluoromethyl)pyridin-3-yl)-1H-pyrazol-5-yl)cyclopentyl)piperazin-1-yl)ethanol

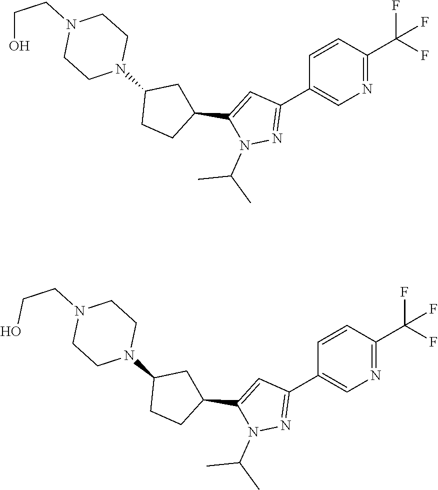 [SMILES: CC(C)n1nc(-c2ccc(C(F)(F)F)nc2)cc1[C@H]1CC[C@H](N2CCN(CCO)CC2)C1 ; CC(C)n1nc(-c2ccc(C(F)(F)F)nc2)cc1[C@H]1CC[C@@H](N2CCN(CCO)CC2)C1]

The mixture of diastereomers (120 mg, 0.27 mmol) was separated using chiral SFC (SFC-13, Phenomenex-Cellulose-2 (250 mm*50 mm, 10 um), 0.1% NH₃H₂O ETOH, 35/35, 60 ml/min) to afford compound 88* (anti isomer, first peak on SFC, 13.53 mg, 11% yield) and compound 89* (syn isomer, second peak on SFC, 75.77 mg, 63% yield). The relative stereochemistry was arbitrarily assigned.

Compound 88*: ¹H NMR (400 MHz, CD₃OD) δ 9.10 (s, 1H), 8.39-8.31 (m, 1H), 7.82 (d, J=8.4 Hz, 1H), 6.65 (s, 1H), 4.77-4.55 (m, 1H), 3.71 (t, J=6.0 Hz, 2H), 3.46-3.36 (m, 1H), 3.02-2.93 (m, 1H), 2.90-2.59 (m, 10H), 2.30-2.23 (m, 1H), 2.20-2.10 (m, 2H), 2.02-1.96 (m, 1H), 1.84-1.73 (m, 1H), 1.71-1.60 (m, 1H), 1.55-1.45 (m, 6H).

Compound 89*: ¹H NMR (400 MHz, CD₃OD) δ 9.10 (d, J=1.6 Hz, 1H), 8.37 (dd, J=1.2, 8.4 Hz, 1H), 7.82 (d, J=8.4 Hz, 1H), 6.69 (s, 1H), 4.69-4.56 (m, 1H), 3.70 (t, J=6.0 Hz, 2H), 3.30-3.24 (m, 1H), 2.88-2.45 (m, 11H), 2.44-2.36 (m, 1H), 2.22-2.03 (m, 2H), 1.85-1.72 (m, 2H), 1.69-1.61 (m, 1H), 1.55-1.43 (m, 6H).

### Example AL: 1-((1R,3R)-3-(1-isopropyl-3-(6-(trifluoromethyl)pyridin-3-yl)-1H-pyrazol-5-yl)cyclopentyl)-4-(2-methoxyethyl)piperazine and 1-((1S,3R)-3-(1-isopropyl-3-(6-(trifluoromethyl) pyridin-3-yl)-1H-pyrazol-5-yl)cyclopentyl)-4-(2-methoxyethyl)piperazine (Compounds 90* & 91*)

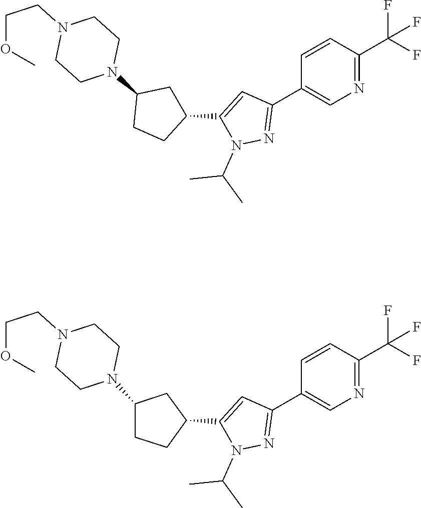 [SMILES: COCCN1CCN([C@@H]2CC[C@@H](c3cc(-c4ccc(C(F)(F)F)nc4)nn3C(C)C)C2)CC1 ; COCCN1CCN([C@H]2CC[C@@H](c3cc(-c4ccc(C(F)(F)F)nc4)nn3C(C)C)C2)CC1]

The title compound was synthesized following a procedure similar to compound 86* using 1-(2-methoxyethyl)piperazine and 3-[2-isopropyl-5-[6-(trifluoromethyl)-3-pyridyl]pyrazol-3-yl]cyclopentanone (first peak on SFC) in reductive amination step. Purification of the crude mixture by Prep-TLC (10% methanol in dichloromethane) to provide title compound (100 mg, 95% yield) as a mixture of diastereomers. The mixture of diastereomers (100 mg, 0.21 mmol) was separated using chiral SFC (SFC-11; Daicel Chiralpak AD (250 mm*30 mm, 10 um)); 0.1% NH₃H₂O; ETOH; 20/20; 60 mL/min) to provide compound 90* (first peak on SFC, 12.48 mg, 12% yield) and compound 91* (second peak on SFC, 53.25 mg, 52% yield). LCMS (ESI) [M+H]⁺=466.3. The relative stereochemistry was arbitrarily assigned.

Compound 90*: ¹H NMR (400 MHz, CD₃OD) δ 9.06 (s, 1H), 8.26 (dd, J=1.6, 8.0 Hz, 1H), 7.66 (d, J=8.0 Hz, 1H), 6.36 (s, 1H), 4.53-4.45 (m, 1H), 3.54 (t, J=5.6 Hz, 2H), 3.36 (s, 3H), 2.95 (s, 1H), 2.88-2.60 (m, 10H), 2.38-2.16 (m, 4H), 1.94-1.90 (m, 1H), 1.82-1.68 (m, 2H), 1.52 (t, J=6.8 Hz, 6H).

Compound 91*: ¹H NMR (400 MHz, CD₃OD) δ 9.07 (d, J=1.6 Hz, 1H), 8.25 (dd, J=1.6, 8.0 Hz, 1H), 7.67 (d, J=8.0 Hz, 1H), 6.43 (s, 1H), 4.52-4.45 (m, 1H), 3.55 (t, J=5.2 Hz, 2H), 3.36 (s, 3H), 3.16-3.14 (m, 1H), 2.83-2.50 (m, 10H), 2.36-2.33 (m, 1H), 2.17-2.14 (m, 1H), 2.03-2.01 (m, 1H), 1.87-1.64 (m, 4H), 1.54 (d, J=6.8 Hz, 6H).

### Example AM: 1-((1S,3S)-3-(1-Isopropyl-3-(6-(trifluoromethyl)pyridin-3-yl)-1H-pyrazol-5-yl)cyclopentyl)-4-(2-methoxyethyl)piperazine and 1-((1R,3S)-3-(1-Isopropyl-3-(6-(trifluoromethyl) pyridin-3-yl)-1H-pyrazol-5-yl)cyclopentyl)-4-(2-methoxyethyl)piperazine (Compound 92* & Compound 93*)

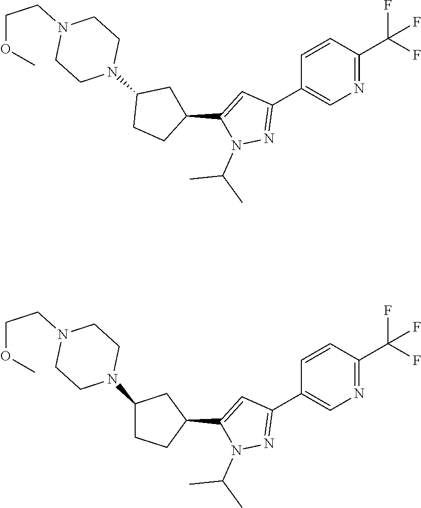 [SMILES: COCCN1CCN([C@H]2CC[C@H](c3cc(-c4ccc(C(F)(F)F)nc4)nn3C(C)C)C2)CC1 ; COCCN1CCN([C@@H]2CC[C@H](c3cc(-c4ccc(C(F)(F)F)nc4)nn3C(C)C)C2)CC1]

The title compound was synthesized following a procedure similar to compound 86* using 1-(2-methoxyethyl)piperazine and 3-[2-isopropyl-5-[6-(trifluoromethyl)-3-pyridyl]pyrazol-3-yl]cyclopentanone (second peak on SFC) in final reductive amination step. Purification of the crude mixture by Prep-TLC (10% methanol in dichloromethane) to provide title compound (90 mg, 90% yield) as a mixture of diastereomers. The mixture of diastereomers (90 mg, 0.19 mmol) was separated using chiral SFC (Daicel Chiralpak AD (250 mm*30 mm, 10 um); 0.1% NH₃H₂O; IPA; 15/15; 60 mL/min) to provide compound 92* (first peak on SFC, 7.54 mg, 7% yield) and compound 93* (second peak on SFC, 53.51 mg, 52% yield). LCMS (ESI) [M+H]⁺=466.3. The relative stereochemistry was arbitrarily assigned.

Compound 92*: ¹H NMR (400 MHz, CD₃OD) δ 9.06 (s, 1H), 8.26 (d, J=8.8 Hz, 1H), 7.67 (d, J=8.0 Hz, 1H), 6.37 (s, 1H), 4.54-4.47 (m, 1H), 3.54 (t, J=5.2 Hz, 2H), 3.36 (s, 4H), 2.98-2.59 (m, 10H), 2.25-2.15 (m, 4H), 1.96-1.91 (m, 1H), 1.89-1.77 (m, 2H), 1.53 (d, J=6.8 Hz, 6H).

Compound 93*: ¹H NMR (400 MHz, CD₃OD) δ 9.06 (s, 1H), 8.24 (dd, J=1.6, 8.4 Hz, 1H), 7.66 (d, J=8.0 Hz, 1H), 6.42 (s, 1H), 4.53-4.47 (m, 1H), 3.52 (t, J=5.6 Hz, 2H), 3.35 (s, 3H), 3.19-3.07 (m, 1H), 2.72-2.61 (m, 2H), 2.60-2.50 (m, 8H), 2.33-2.30 (m, 1H), 2.21-1.96 (m, 2H), 1.88-1.60 (m, 4H), 1.52 (d, J=6.8 Hz, 6H).

### Example AN: 4-((1S,3S)-3-(1-Isopropyl-3-(2-(trifluoromethyl)pyrimidin-5-yl)-1H-1,2,4-triazol-5-yl)cyclopentyl)morpholine and 4-((1R,3S)-3-(1-Isopropyl-3-(2-(trifluoromethyl)pyrimidin-5-yl)-1H-1,2,4-triazol-5-yl)cyclopentyl)morpholine (Compounds 94 & 95)

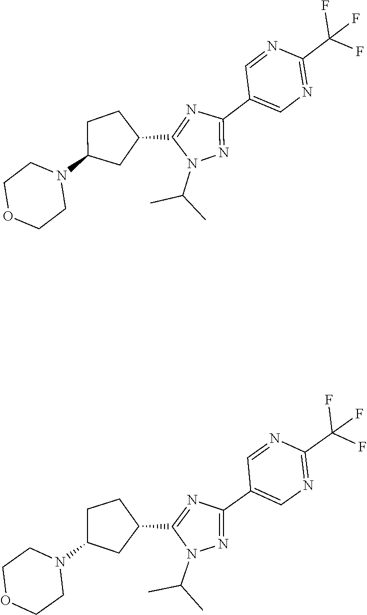 [SMILES: CC(C)n1nc(-c2cnc(C(F)(F)F)nc2)nc1[C@H]1CC[C@H](N2CCOCC2)C1 ; CC(C)n1nc(-c2cnc(C(F)(F)F)nc2)nc1[C@H]1CC[C@@H](N2CCOCC2)C1]

The title compound was synthesized following a procedure similar to compound 68 using 2-(trifluoromethyl)pyrimidine-5-carboximidamide hydrochloride and (S)-3-oxocyclopentanecarboxylic acid in step 1. Purification of the crude mixture by silica flash chromatography (0-10% methanol in dichloromethane) to provide the title compound (150 mg, 73% yield) as a mixture of diastereomers. The mixture of diastereomers (150 mg, 0.37 mmol) was separated using chiral SFC (DAICEL CHIRALPAK AD (250 mm*30 mm, 10 um); 0.1% NH₃H₂O+MEOH, 20/20; 60 mL/min) to provide compound 94 (first peak on SFC, 33.31 mg, 20.4% yield) and compound 95 (second peak on SFC, 73.33 mg, 46% yield) as a colorless oil. LCMS (ESI) [M+H]⁺=411.2. The relative stereochemistry was determined by 2D-NMR.

Compound 94: ¹H NMR (400 MHz, CD₃OD) δ 9.51 (s, 2H), 4.84-4.74 (m, 1H), 3.74 (t, J=4.8 Hz, 4H), 3.65-3.57 (m, 1H), 3.02-2.94 (m, 1H), 2.64-2.53 (m, 4H), 2.29-2.23 (m, 2H), 2.21-2.16 (m, 1H), 2.11-2.04 (m, 1H), 2.01-1.91 (m, 1H), 1.71-1.61 (m, 1H), 1.54 (dd, J=2.0, 6.8 Hz, 6H).

Compound 95: ¹H NMR (400 MHz, CD₃OD) δ 9.51 (s, 2H), 4.83-4.73 (m, 1H), 3.73 (t, J=4.8 Hz, 4H), 3.54-3.46 (m, 1H), 2.88-2.79 (m, 1H), 2.64-2.52 (m, 4H), 2.42-2.36 (m, 1H), 2.23-2.14 (m, 1H), 2.12-2.01 (m, 2H), 1.98-1.93 (m, 1H), 1.91-1.90 (m, 1H), 1.88-1.78 (m, 1H), 1.54 (dd, J=2.4, 6.8 Hz, 6H).

### Example AO: (R)-4-((1R,3s,5S,6R)-6-(1-Isopropyl-3-(2-(trifluoromethyl)pyrimidin-5-yl)-1H-pyrazol-5-yl)bicyclo[3.1.0]hexan-3-yl)-3-methylmorpholine and (R)-4-((1R,3r,5S,6R)-6-(1-Isopropyl-3-(2-(trifluoromethyl)pyrimidin-5-yl)-1H-pyrazol-5-yl) bicyclo[3.1.0]hexan-3-yl)-3-methylmorpholine (Compounds 96 & 97)

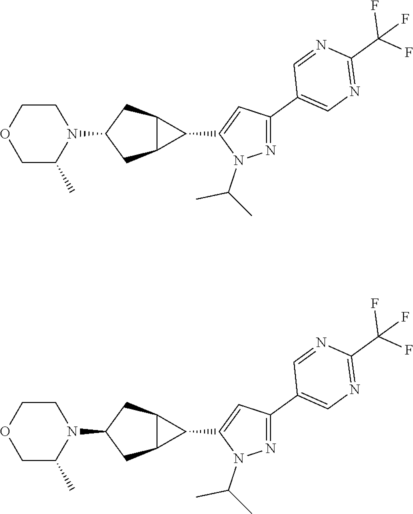 [SMILES: CC(C)n1nc(-c2cnc(C(F)(F)F)nc2)cc1[C@H]1[C@@H]2C[C@@H](N3CCOC[C@H]3C)C[C@@H]21 ; CC(C)n1nc(-c2cnc(C(F)(F)F)nc2)cc1[C@H]1[C@@H]2C[C@H](N3CCOC[C@H]3C)C[C@@H]21]

The title compounds were synthesized following a procedure similar to compound 29* using (1R,5S,6r)-6-(3-iodo-1-isopropyl-1H-pyrazol-5-yl)bicyclo[3.1.0]hexan-3-one and 5-(4,4,5,5-tetramethyl-1,3,2-dioxaborolan-2-yl)-2-(trifluoromethyl)pyrimidine in step 1. Purification of the crude mixture by reverse phase chromatography (Boston Prime C18 150*30 mm*5 um; water (0.05% NH₃H₂O+10 mM NH₄HCO₃)-ACN; 65%-95%) to provide compound 96 (first peak on SFC, 28.44 mg, 22% yield) and compound 97 (second peak on SFC, 56.2 mg, 45% yield). LCMS (ESI), [M+H]⁺=436.2. The relative stereochemistry was determined by 2D-NMR.

Compound 96: ¹H NMR (400 MHz, CD₃OD) δ 9.27 (s, 2H), 6.50 (s, 1H), 4.83-4.79 (m, 1H), 3.73-3.69 (m, 3H), 3.48 (dd, J=4.4, 11.2 Hz, 1H), 3.10-3.01 (m, 1H), 2.88-2.68 (m, 2H), 2.50 (d, J=11.6 Hz, 1H), 2.32-2.18 (m, 2H), 1.93-1.75 (m, 5H), 1.53 (d, J=6.8 Hz, 6H), 1.12 (t, J=6.0 Hz 3H).

Compound 97: ¹H NMR (400 MHz, CD₃OD) δ 9.26 (s, 2H), 6.46 (s, 1H), 4.85-4.82 (m, 1H), 3.72-3.66 (m, 3H), 3.51-3.47 (m, 2H), 2.82 (s, 1H), 2.70-2.64 (m, 1H), 2.51-2.47 (m, 1H), 2.35-2.18 (m, 2H), 1.86 (t, J=2.8 Hz, 1H), 1.76-1.67 (m, 4H), 1.55 (d, J=6.4 Hz, 6H), 1.09 (d, J=6.4 Hz, 3H).

### Example AP: 4-((1R,3s,5S,6r)-6-(1-Isopropyl-3-(2-(trifluoromethyl)pyrimidin-4-yl)-1H-pyrazol-5-yl)bicyclo[3.1.0]hexan-3-yl)morpholine and 4-((1R,3r,5S,6r)-6-(1-Isopropyl-3-(2-(trifluoromethyl) pyrimidin-4-yl)-1H-pyrazol-5-yl)bicyclo[3.1.0]hexan-3-yl)morpholine (Compounds 98 & 99)

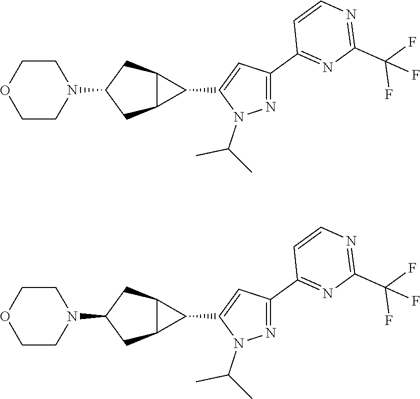 [SMILES: CC(C)n1nc(-c2ccnc(C(F)(F)F)n2)cc1[C@H]1[C@@H]2C[C@@H](N3CCOCC3)C[C@@H]21 ; CC(C)n1nc(-c2ccnc(C(F)(F)F)n2)cc1[C@H]1[C@@H]2C[C@H](N3CCOCC3)C[C@@H]21]

### Step 1: 1-(3-((tert-Butyldiphenylsilyl)oxy)bicyclo[3.1.0]hexan-6-yl)-3-(2-(trifluoromethyl) pyrimidin-4-yl)propane-1,3-dione

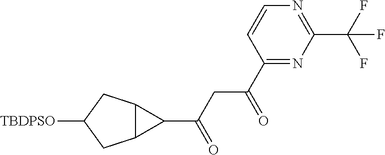

To a solution of 1-[3-[tert-butyl(diphenyl)silyl]oxy-6-bicyclo[3.1.0]hexanyl]ethanone (1.6 g, 4.23 mmol) in tetrahydrofuran (30 mL) was added NaH (254 mg, 6.34 mmol, 60% in mineral oil) in portions at 0° C. under N₂ and stirred for 0.5 hour. A solution of methyl 2-(trifluoromethyl) pyrimidine-4-carboxylate (1.05 g, 5.07 mmol) in THF (5 mL) was added at 0° C. under N₂. Then the reaction mixture was stirred at 20° C. for 2 hours. The reaction mixture was quenched with saturated aqueous NH₄Cl (10 mL), diluted with water (30 mL). The resulting mixture was extracted with ethyl acetate (50 mL×3). The combined organic layers were dried over sodium sulfate, filtered and concentrated under reduced pressure. The residue was purified by silica flash chromatography (0-20% ethyl acetate in petroleum ether) to provide title compound (2 g, 74% yield). LCMS (ESI) [M+H]⁺=553.0.

### Step 2: 4-(5-((1R,5S,6r)-3-((tert-Butyldiphenylsilyl)oxy)bicyclo[3.1.0]hexan-6-yl)-1-isopropyl-1H-pyrazol-3-yl)-2-(trifluoromethyl)pyrimidine

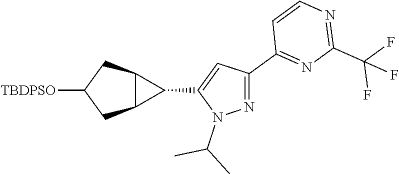

To a solution of 1-[3-[tert-butyl(diphenyl)silyl]oxy-6-bicyclo[3.1.0]hexanyl]-3-[2-(trifluoromethyl) pyrimidin-4-yl]propane-1,3-dione (2 g, 3.62 mmol) in ethanol (20 mL) was added isopropylhydrazine hydrochloride (424 mg, 3.83 mmol) and triethylamine (0.55 mL, 3.83 mmol) and stirred at 25° C. for 15 hours. The mixture was concentrated in vacuo, and the residue was purified by silica flash chromatography (0-5% ethyl acetate in petroleum ether) to provide title compound (1.5 g, 70% yield). LCMS (ESI) [M+H]⁺=591.6.

### Step 3: (1R,5S,6r)-6-(1-Isopropyl-3-(2-(trifluoromethyl)pyrimidin-4-yl)-1H-pyrazol-5-yl)bicyclo [3.1.0]hexan-3-ol

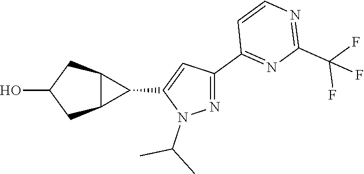 [SMILES: CC(C)n1nc(-c2ccnc(C(F)(F)F)n2)cc1[C@H]1[C@@H]2CC(O)C[C@@H]21]

To a solution of tert-butyl-[[(1S, 5R)-6-[2-isopropyl-5-[2-(trifluoromethyl)pyrimidin-4-yl]pyrazol-3-yl]-3-bicyclo[3.1.0]hexanyl]oxy]-diphenyl-silane (1.5 g, 2.54 mmol) in tetrahydrofuran (10 mL) was added TABF (5 mL, 30.4 mmol, 1 M in THF) and stirred at 70° C. for 15 hours. The reaction mixture was concentrated under reduced pressure. The crude residue was diluted with water (10 mL), then adjusted pH=7 with NaOH (2M). The resulting mixture was extracted with ethyl acetate (50 mL×3). The combined organic layers were dried over sodium sulfate, filtered, and concentrated under reduced pressure. The residue was purified by silica flash chromatography (0-50% ethyl acetate in petroleum ether) to provide title compound (680 mg, 74% yield). LCMS (ESI) [M+H]⁺=352.9.

### Step 4: (1R,5S,6r)-6-(1-Isopropyl-3-(2-(trifluoromethyl)pyrimidin-4-yl)-1H-pyrazol-5-yl)bicyclo [3.1.0]hexan-3-one

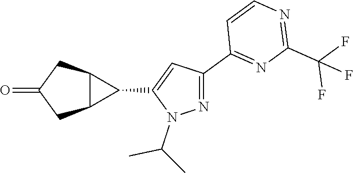 [SMILES: CC(C)n1nc(-c2ccnc(C(F)(F)F)n2)cc1[C@H]1[C@@H]2CC(=O)C[C@@H]21]

To a solution of (1R,5S)-6-[2-isopropyl-5-[2-(trifluoromethyl)pyrimidin-4-yl]pyrazol-3-yl]bicyclo [3.1.0]hexan-3-ol (680 mg, 1.93 mmol) in anhydrous dichloromethane (5 mL) was added Dess-Martin periodinane (1.22 g, 2.89 mmol) and stirred at 25° C. for 16 hours. Then the mixture was diluted with H₂O (5 mL), followed by aq. Na₂SO₃ (10 mL), aq. NaHCO₃ (10 mL). The resulting mixture was extracted with ethyl acetate (30 mL×3). The combined organic layers were dried over Na₂SO₄, filtered, and concentrated under residue pressure. The residue was purified by silica flash chromatography (0-40% ethyl acetate in petroleum ether) to provide title compound (430 mg, 62% yield). LCMS (ESI) [M+H]⁺=351.2.

### Step 5: 4-((1R,3s,5S,6r)-6-(1-Isopropyl-3-(2-(trifluoromethyl)pyrimidin-4-yl)-1H-pyrazol-5-yl)bicyclo [3.1.0]hexan-3-yl)morpholine and 4-((1R,3r,5S,6r)-6-(1-Isopropyl-3-(2-(trifluoromethyl) pyrimidin-4-yl)-1H-pyrazol-5-yl)bicyclo[3.1.0]hexan-3-yl)morpholine (Compounds 98 & 99)

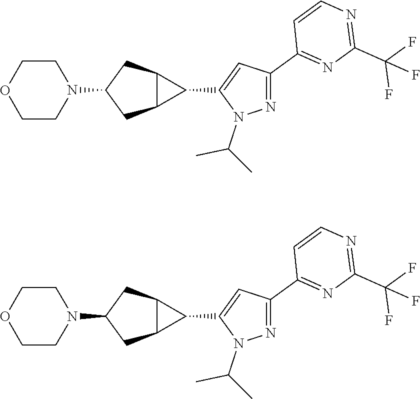 [SMILES: CC(C)n1nc(-c2ccnc(C(F)(F)F)n2)cc1[C@H]1[C@@H]2C[C@@H](N3CCOCC3)C[C@@H]21 ; CC(C)n1nc(-c2ccnc(C(F)(F)F)n2)cc1[C@H]1[C@@H]2C[C@H](N3CCOCC3)C[C@@H]21]

To a mixture of (1R,5S)-6-[2-isopropyl-5-[2-(trifluoromethyl)pyrimidin-4-yl]pyrazol-3-yl]bicyclo [3.1.0]hexan-3-one (80 mg, 0.23 mmol) in methyl alcohol (8 mL) was added morpholine (0.1 mL, 1.14 mmol) and acetic acid (0.06 mL, 0.69 mmol), NaBH₃CN (43 mg, 0.69 mmol) and stirred at 60° C. for 16 hours. The reaction was diluted with water (20 mL) and extracted with ethyl acetate (50 mL×3). The combined organics were washed with brine (50 mL×2), dried over sodium sulfate, filtered and concentrated. The residue was purified by reverse phase chromatography (Boston Prime C18 150*30 mm*5 um; water (0.05% NH₃H₂O+10 mM NH₄HCO₃)-ACN; 60%-90%) to provide compound 98 (first peak on SFC, 43.49 mg, 45% yield) and compound 99 (second peak on SFC, 26.71 mg, 27% yield). LCMS (ESI) [M+H]⁺=422.1. The relative stereochemistry was determined by 2D-NMR.

Compound 98: ¹H NMR (400 MHz, CD₃OD) δ 8.83 (d, J=5.6 Hz, 1H), 8.12 (d, J=5.2 Hz, 1H), 6.64 (s, 1H), 4.84-4.77 (m, 1H), 3.70 (t, J=4.8 Hz, 4H), 2.51-2.49 (m, 5H), 2.29 (dd, J=7.2, 12.4 Hz, 2H), 1.78-1.76 (m, 5H), 1.53 (d, J=6.8 Hz, 6H).

Compound 99: ¹H NMR (400 MHz, CD₃OD) δ 8.84 (d, J=5.2 Hz, 1H), 8.13 (d, J=5.2 Hz, 1H), 6.59 (s, 1H), 4.86-4.77 (m, 1H), 3.69 (t, J=4.8 Hz, 4H), 2.97-2.94 (m, 1H), 2.48 (brs, 4H), 2.36-2.34 (m, 2H), 1.90 (t, J=2.8 Hz, 1H), 1.74-1.67 (m, 4H), 1.56 (d, J=6.8 Hz, 6H).

### Example AO: (S)-4-((1R,3s,5S,6S)-6-(1-Isopropyl-3-(2-(trifluoromethyl)pyrimidin-4-yl)-1H-pyrazol-5-yl)bicyclo[3.1.0]hexan-3-yl)-3-methylmorpholine and (S)-4-((1R,3r,5S,6S)-6-(1-Isopropyl-3-(2-(trifluoromethyl)pyrimidin-4-yl)-1H-pyrazol-5-yl)bicyclo[3.1.0]hexan-3-yl)-3-methylmorpholine (Compounds 100 & 101)

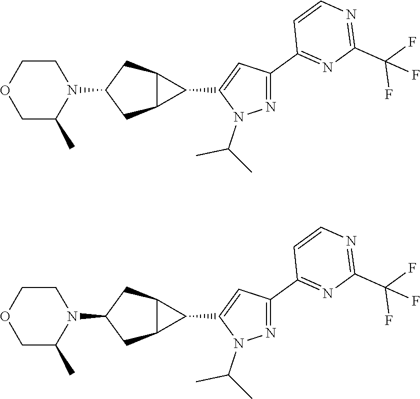 [SMILES: CC(C)n1nc(-c2ccnc(C(F)(F)F)n2)cc1[C@H]1[C@@H]2C[C@@H](N3CCOC[C@@H]3C)C[C@@H]21 ; CC(C)n1nc(-c2ccnc(C(F)(F)F)n2)cc1[C@H]1[C@@H]2C[C@H](N3CCOC[C@@H]3C)C[C@@H]21]

The title compounds were synthesized following a procedure similar to compound 98 using (1R,5S,6r)-6-(1-Isopropyl-3-(2-(trifluoromethyl)pyrimidin-4-yl)-1H-pyrazol-5-yl)bicyclo[3.1.0]hexan-3-one and (S)-3-methylmorpholine in final reductive amination step. Purification of the crude mixture by reverse phase chromatography (Boston Prime C18 150*30 mm*5 um; water (0.05% NH₃H₂O+10 mM NH₄HCO₃)-ACN; 65%-95%) to provide compound 100 (second peak on SFC, 33.93 mg, 27% yield) and compound 101 (first peak on SFC, 34.36 mg, 28% yield). LCMS (ESI) [M+H]⁺=436.3. The relative stereochemistry was determined by 2D-NMR.

Compound 100: ¹H NMR (400 MHz, CD₃OD) δ 8.84 (d, J=5.2 Hz, 1H), 8.12 (d, J=5.6 Hz, 1H), 6.64 (s, 1H), 4.86-4.83 (m, 1H), 3.72-3.68 (m, 3H), 3.47 (dd, J=4.4, 11.2 Hz, 1H), 3.08-3.00 (m, 1H), 2.79 (t, J=6.8 Hz, 1H), 2.75-2.67 (m, 1H), 2.51-2.45 (m, 1H), 2.24-2.16 (m, 2H), 1.95-1.88 (m, 2H), 1.83-1.80 (m, 1H), 1.77-1.63 (m, 2H), 1.54 (d, J=6.0 Hz, 6H), 1.10 (d, J=6.4 Hz, 3H).

Compound 101: ¹H NMR (400 MHz, CD₃OD) δ 8.84 (d, J=5.6 Hz, 1H), 8.13 (d, J=5.2 Hz, 1H), 6.60 (s, 1H), 4.87-4.83 (m, 1H), 3.81-3.66 (m, 3H), 3.50-3.46 (m, 2H), 2.92-2.88 (m, 1H), 2.68-2.64 (m, 1H), 2.50-2.48 (m, 1H), 2.35-2.20 (m, 2H), 1.86 (t, J=3.2 Hz, 1H), 1.74-1.69 (m, 4H), 1.56 (d, J=6.8 Hz, 6H), 1.09 (d, J=6.4 Hz, 3H).

### Example AR: (R)-4-((1R,3s,5S,6S)-6-(1-Isopropyl-3-(2-(trifluoromethyl)pyrimidin-4-yl)-1H-pyrazol-5-yl)bicyclo[3.1.0]hexan-3-yl)-3-methylmorpholine and (R)-4-((1R,3r,5S,6S)-6-(1-Isopropyl-3-(2-(trifluoromethyl)pyrimidin-4-yl)-1H-pyrazol-5-yl)bicyclo[3.1.0]hexan-3-yl)-3-methylmorpholine (Compounds 102 & 103)

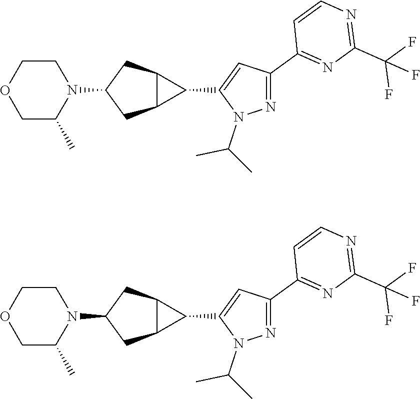 [SMILES: CC(C)n1nc(-c2ccnc(C(F)(F)F)n2)cc1[C@H]1[C@@H]2C[C@@H](N3CCOC[C@H]3C)C[C@@H]21 ; CC(C)n1nc(-c2ccnc(C(F)(F)F)n2)cc1[C@H]1[C@@H]2C[C@H](N3CCOC[C@H]3C)C[C@@H]21]

The title compounds were synthesized following a procedure similar to compound 98 using (1R,5S,6r)-6-(1-Isopropyl-3-(2-(trifluoromethyl)pyrimidin-4-yl)-1H-pyrazol-5-yl)bicyclo [3.1.0]hexan-3-one and (R)-3-methylmorpholine in final reductive amination step. Purification of the crude mixture by reverse phase chromatography (Boston Prime C18 150*30 mm*5 um; water (0.05% NH₃H₂O+10 mM NH₄HCO₃)-ACN; 65%-95%) to provide compound 102 (second peak on SFC, 27.8 mg, 18% yield) and compound 103 (first peak on SFC, 34.53 mg, 22% yield). LCMS (ESI)[M+H]⁺=436.3 The relative stereochemistry was determined by 2D-NMR.

Compound 102: ¹H NMR (400 MHz, CD₃OD) δ8.83 (d, J=5.2 Hz, 1H), 8.12 (d, J=5.6 Hz, 1H), 6.64 (s, 1H), 4.84-4.81 (m, 1H), 3.82-3.68 (m, 2H), 3.47 (dd, J=4.4, 11.2 Hz, 1H), 3.10-3.00 (m, 1H), 2.88-2.75 (m, 1H), 2.74-2.68 (m, 1H), 2.50-2.41 (m, 1H), 2.33-2.16 (m, 2H), 1.94-1.76 (m, 5H), 1.54 (d, J=6.8 Hz, 6H), 1.13-1.09 (m, 3H).

Compound 103: ¹H NMR (400 MHz, CD₃OD) δ 8.84 (d, J=5.6 Hz, 1H), 8.12 (d, J=5.2 Hz, 1H), 6.59 (s, 1H), 4.85-4.83 (m, 1H), 3.72-3.66 (m, 3H), 3.49 (dd, J=4.4, 11.2 Hz, 2H), 2.85-2.80 (m, 1H), 2.68-2.64 (m, 1H), 2.50-2.46 (m, 1H), 2.36-2.20 (m, 2H), 1.86 (t, J=3.2 Hz, 1H), 1.77-1.69 (m, 4H), 1.56 (d, J=6.8 Hz, 6H), 1.09 (d, J=6.4 Hz, 3H).

### Example AS: 4-((1R,3R)-3-(1-Isopropyl-3-(6-(trifluoromethyl)pyridin-3-yl)-1H-1,2,4-triazol-5-yl)cyclopentyl)morpholine and 4-((1S,3R)-3-(1-Isopropyl-3-(6-(trifluoromethyl)pyridin-3-yl)-1H-1,2,4-triazol-5-yl)cyclopentyl)morpholine (Compounds 104 & 105)

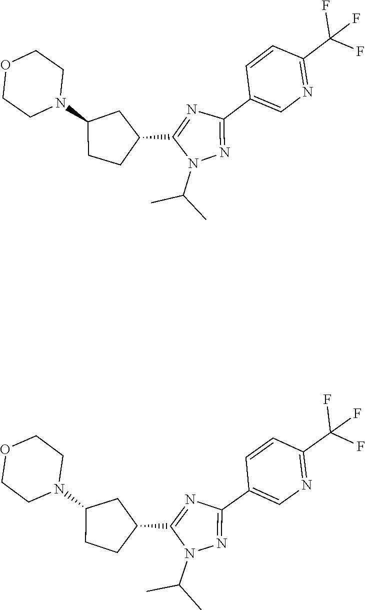 [SMILES: CC(C)n1nc(-c2ccc(C(F)(F)F)nc2)nc1[C@@H]1CC[C@@H](N2CCOCC2)C1 ; CC(C)n1nc(-c2ccc(C(F)(F)F)nc2)nc1[C@@H]1CC[C@H](N2CCOCC2)C1]

The title compound was synthesized following a procedure similar to compound 68 using 6-(trifluoromethyl)nicotinimidamide hydrochloride and (5)-3-oxocyclopentanecarboxylic acid in step 1. Purification of the crude mixture by silica flash chromatography (0-65% ethyl acetate in petroleum ether) to provide the title compounds (90 mg, 73.6% yield) as a mixture of diastereomers. The mixture of diastereomers (90.0 mg, 0.22 mmol) was separated using chiral SFC (DAICEL CHIRALPAK OD (250 mm*30 mm, 10 um); 0.1% NH₃H₂O ETOH 15/15; 60 mL/min) to provide compound 104 (first peak on SFC, 14.39 mg, 15.3% yield) and compound 105 (second peak on SFC, 41.4 mg, 45.5% yield). LCMS (ESI) [M+H]⁺=410.2. The relative stereochemistry was determined by 2D-NMR.

Compound 104: ¹H NMR (400 MHz, CD₃OD) δ 9.32 (s, 1H), 8.60 (d, J=8.4 Hz, 1H), 7.90 (d, J=8.4 Hz, 1H), 4.80-4.73 (m, 1H), 3.73 (t, J=4.8 Hz, 4H), 3.63-3.55 (m, 1H), 3.01-2.93 (m, 1H), 2.59 (brs, 4H), 2.26-2.15 (m, 3H), 2.11-2.03 (m, 1H), 2.00-1.91 (m, 1H), 1.70-1.60 (m, 1H), 1.54 (dd, J=2.0, 6.4 Hz, 6H).

Compound 105: ¹H NMR (400 MHz, CD₃OD) δ 9.32 (s, 1H), 8.60 (d, J=8.4 Hz, 1H), 7.90 (d, J=8.4 Hz, 1H), 4.79-4.73 (m, 1H), 3.73 (t, J=4.8 Hz, 4H), 3.53-3.44 (m, 1H), 2.88-2.80 (m, 1H), 2.61 (d, J=4.0 Hz, 4H), 2.42-2.35 (m, 1H), 2.20-2.16 (m, 1H), 2.11-2.03 (m, 2H), 2.00-1.93 (m, 1H), 1.87-1.82 (m, 1H), 1.54 (dd, J=2.0, 6.8 Hz, 6H).

### Example AT: (R)-4-((1R,3s,5S,6R)-6-(1-Isopropyl-3-(6-(trifluoromethyl)pyrimidin-4-yl)-1H-pyrazol-5-yl)bicyclo[3.1.0]hexan-3-yl)-3-methylmorpholine and (R)-4-((1R,3r,5S,6R)-6-(1-Isopropyl-3-(6-(trifluoromethyl)pyrimidin-4-yl)-1H-pyrazol-5-yl)bicyclo[3.1.0]hexan-3-yl)-3-methylmorpholine (Compounds 202 & 106)

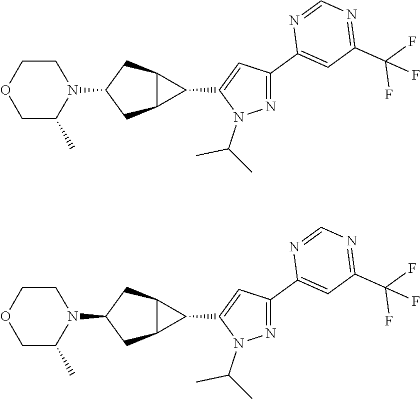 [SMILES: CC(C)n1nc(-c2cc(C(F)(F)F)ncn2)cc1[C@H]1[C@@H]2C[C@@H](N3CCOC[C@H]3C)C[C@@H]21 ; CC(C)n1nc(-c2cc(C(F)(F)F)ncn2)cc1[C@H]1[C@@H]2C[C@H](N3CCOC[C@H]3C)C[C@@H]21]

### Step 1: 1-(3-((tert-Butyldiphenylsilyl)oxy)bicyclo[3.1.0]hexan-6-yl)-3-(6-(trifluoromethyl) pyrimidin-4-yl)propane-1,3-dione

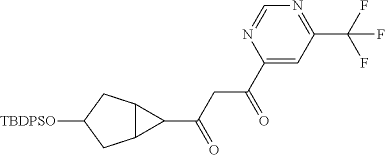

To a solution of 1-[3-[tert-butyl(diphenyl)silyl]oxy-6-bicyclo[3.1.0]hexanyl]ethanone (1.0 g, 2.64 mmol) in tetrahydrofuran (10 mL) was added NaH (0.16 g, 3.96 mmol, 60% in mineral oil) at 0° C. under N₂ and stirred at 0° C. for 1 hour. Then methyl 6-(trifluoromethyl)pyrimidine-4-carboxylate (0.82 g, 3.96 mmol) was added to above reaction mixture and stirred at 20° C. for 2 hours. The reaction was quenched with NH₄Cl a.q. (20 mL) and extracted with dichloromethane (50 mL×3). The combined organic layer was dried over anhydrous Na₂SO₄, filtered and concentrated in vacuo. The residue was purified by silica flash chromatography (0-5% ethyl acetate in petroleum ether) to provide the title compound (1200 mg, 95.7% yield). LCMS (ESI), [M+H]⁺=553.2.

### Step 2: 4-(5-((1R,5S,6r)-3-((tert-Butyldiphenylsilyl)oxy)bicyclo[3.1.0]hexan-6-yl)-1-isopropyl-1H-pyrazol-3-yl)-6-(trifluoromethyl)pyrimidine

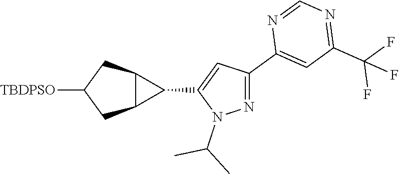

To a solution of 1-[3-[tert-butyl(diphenyl)silyl]oxy-6-bicyclo[3.1.0]hexanyl]-3-[6-(trifluoromethyl) pyrimidin-4-yl]propane-1,3-dione (1200.0 mg, 2.17 mmol) and isopropylhydrazine hydrochloride (480.0 mg, 4.34 mmol) in ethanol (20 mL) was added triethylamine (0.6 mL, 4.34 mmol) and stirred at 25° C. for 15 hours. The reaction mixture was concentrated under reduced pressure and the residue was purified by silica flash chromatography (0-25% ethyl acetate in petroleum ether) to provide the title compound (1000 mg, 78% yield). LCMS (ESI), [M+H]⁺=591.2.

### Step 3: 4-(5-((1R,5S,6r)-3-((tert-Butyldiphenylsilyl)oxy)bicyclo[3.1.0]hexan-6-yl)-1-isopropyl-1H-pyrazol-3-yl)-6-(trifluoromethyl)pyrimidine

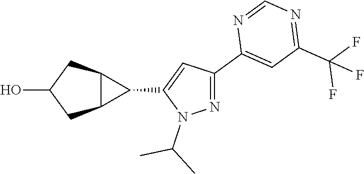 [SMILES: CC(C)n1nc(-c2cc(C(F)(F)F)ncn2)cc1[C@H]1[C@@H]2CC(O)C[C@@H]21]

To a stirred solution of tert-butyl-diphenyl-[[rac-(1R,5S)-6-[2-isopropyl-5-[6-(trifluoromethyl) pyrimidin-4-yl]pyrazol-3-yl]-3-bicyclo[3.1.0]hexanyl]oxy]silane (1000.0 mg, 1.69 mmol) in tetrahydrofuran (5 mL) was added triethylamine trihydrofluoride (10.0 mL, 61.45 mmol) and stirred at 70° C. for 8 hours. The reaction mixture was adjusted to pH=9 with NaOH.aq. (4 M). The resulting solution was extracted with dichloromethane (50 mL×3). The combined organic layer was dried over anhydrous Na₂SO₄, filtered and concentrated in vacuo. The residue was purified by silica flash chromatography (0-50% ethyl acetate in petroleum ether) to provide the title compound (550 mg, 91.3% yield). LCMS (ESI), [M+H]⁺=353.2.

### Step 4: (1R,5S,6r)-6-(1-Isopropyl-3-(6-(trifluoromethyl)pyrimidin-4-yl)-1H-pyrazol-5-yl)bicycle [3.1.0]hexan-3-one

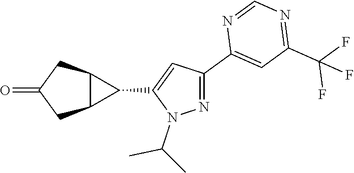 [SMILES: CC(C)n1nc(-c2cc(C(F)(F)F)ncn2)cc1[C@H]1[C@@H]2CC(=O)C[C@@H]21]

To a solution of (1R,5S)-6-[2-isopropyl-5-[6-(trifluoromethyl)pyrimidin-4-yl]pyrazol-3-yl]bicyclo[3.1.0]hexan-3-ol (550.0 mg, 1.56 mmol) in anhydrous dichloromethane (10 mL) was added Dess-martin periodinane (993.0 mg, 2.34 mmol) at 0° C. under N₂. The reaction mixture was stirred at 25° C. for 2 hours. The reaction mixture was quenched with Na₂S₂O₃ aq. (10 mL) and extracted with dichloromethane (50 mL×3). The combined organic layer was washed with NaHCO₃. aq. (20 mL), dried over anhydrous Na₂SO₄, filtered and concentrated in vacuo. The residue was purified by silica flash chromatography (0-25% ethyl acetate in petroleum ether) to provide the title compound (350 mg, 63.4% yield). LCMS (ESI), [M+H]⁺=351.3. H NMR (400 MHz, CD₃OD) δ 9.24 (s, 1H), 8.27 (d, J=1.2 Hz, 1H), 6.77 (s, 1H), 4.85-4.80 (m, 1H), 2.81-2.74 (m, 2H), 2.43-2.38 (m, 2H), 2.04-2.00 (m, 2H), 1.65 (t, J=3.6 Hz, 1H), 1.55 (d, J=6.4 Hz, 6H).

### Step 5: (R)-7-(1-(4-(Trifluoromethyl)phenyl)piperidin-3-yl)-2-thia-7-azaspiro[3.5]nonane 2,2-dioxide and (S)-7-(1-(4-(trifluoromethyl)phenyl)piperidin-3-yl)-2-thia-7-azaspiro[3.5]nonane 2,2-dioxide (Compounds 202 & 106)

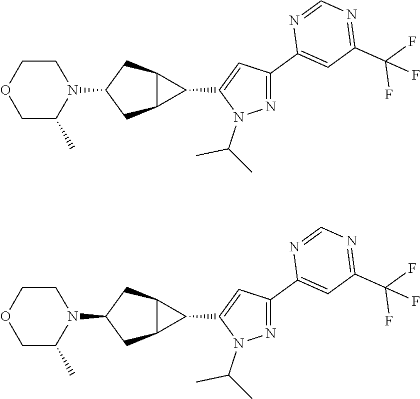 [SMILES: CC(C)n1nc(-c2cc(C(F)(F)F)ncn2)cc1[C@H]1[C@@H]2C[C@@H](N3CCOC[C@H]3C)C[C@@H]21 ; CC(C)n1nc(-c2cc(C(F)(F)F)ncn2)cc1[C@H]1[C@@H]2C[C@H](N3CCOC[C@H]3C)C[C@@H]21]

To a solution of rac-(1R,5S)-6-[2-isopropyl-5-[6-(trifluoromethyl)pyrimidin-4-yl]pyrazol-3-yl]bicyclo[3.1.0]hexan-3-one (100 mg, 0.29 mmol) in methyl alcohol (4 mL) was added (3R)-3-methylmorpholine (0.09 mL, 0.86 mmol), acetic acid (0.08 mL, 1.43 mmol) and sodium cyanoborohydride (54 mg, 0.86 mmol) and stirred at 60° C. for 16 hours. The reaction mixture was adjusted to pH=8 with saturated NaHCO₃ (10 mL) at 0° C. and extracted with ethyl acetate (40 mL×2). The combined extracts were washed with brine (10 mL) and dried over Na₂SO₄, filtered and concentrated under reduced pressure. The residue was purified by reverse phase chromatography (water (0.05% NH₃H₂O+10 mM NH₄HCO₃)-ACN, 65%-95%) to provide compound 202 (first peak on HPLC, 21.28 mg, 15.7% yield) and compound 106 (second peak on HPLC, 27.83 mg, 28.3% yield). LCMS (ESI) [M+H]⁺=436.2. The relative stereochemistry was determined by 2D-NMR.

Compound 202: ¹H NMR (400 MHz, CD₃OD) δ 9.22 (s, 1H), 8.25 (d, J=1.2 Hz, 1H), 6.67 (s, 1H), 4.85-4.81 (m, 1H), 3.75-3.65 (m, 3H), 3.50-3.46 (m, 1H), 3.06-3.04 (m, 1H), 2.81 (brs, 1H), 2.73-2.67 (m, 1H), 2.49 (d, J=12.0 Hz, 1H), 2.23-2.12 (m, 2H), 1.93-1.86 (m, 2H), 1.83 (t, J=3.2 Hz, 1H), 1.79-1.70 (m, 2H), 1.55 (d, J=6.8 Hz, 6H), 1.11 (d, J=6.8 Hz, 3H).

Compound 106: ¹H NMR (400 MHz, CD₃OD) δ 9.22 (s, 1H), 8.25 (d, J=1.2 Hz, 1H), 6.63 (s, 1H), 4.85-4.81 (m, 1H), 3.75-3.64 (m, 3H), 3.51-3.47 (m, 2H), 2.85 (brs, 1H), 2.69-2.63 (m, 1H), 2.52-2.47 (m, 1H), 2.34-2.21 (m, 2H), 1.88 (t, J=2.8 Hz, 1H), 1.76-1.65 (m, 4H), 1.57 (d, J=6.8 Hz, 6H), 1.11 (d, J=6.4 Hz, 3H).

### Example AU: 4-((1R,3s,5S,6r)-6-(1-Isopropyl-3-(6-(trifluoromethyl)pyrazin-2-yl)-1H-pyrazol-5-yl)bicyclo[3.1.0]hexan-3-yl)morpholine and 4-((1R,3r,5S,6r)-6-(1-Isopropyl-3-(6-(trifluoromethyl) pyrazin-2-yl)-1H-pyrazol-5-yl)bicyclo[3.1.0]hexan-3-yl)morpholine (Compounds 107 & 108)

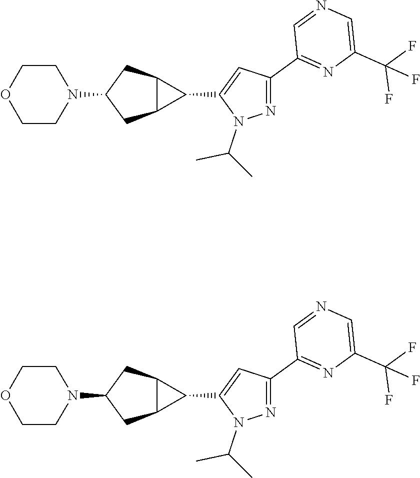 [SMILES: CC(C)n1nc(-c2cncc(C(F)(F)F)n2)cc1[C@H]1[C@@H]2C[C@@H](N3CCOCC3)C[C@@H]21 ; CC(C)n1nc(-c2cncc(C(F)(F)F)n2)cc1[C@H]1[C@@H]2C[C@H](N3CCOCC3)C[C@@H]21]

The title compound was synthesized following a procedure similar to compound 202 using methyl 6-(trifluoromethyl)pyrazine-2-carboxylate and 1-[3-[tert-butyl(diphenyl)silyl]oxy-6-bicyclo [3.1.0]hexanyl]ethanone in step 1. Purification of the crude mixture by reverse phase chromatography (water (0.05% NH₃H₂O+10 mM NH₄HCO₃)-ACN, 55%-80%) to provide title compound 107 (first peak on HPLC, 8.8 mg, 6.9% yield) and title compound 108 (second peak on HPLC, 11 mg, 8.8% yield). LCMS (ESI) [M+H]⁺=422.3. The relative stereochemistry was determined by 2D-NMR.

Compound 107: ¹H NMR (400 MHz, CD₃OD) δ 9.38 (s, 1H), 8.83 (s, 1H), 6.57 (s, 1H), 4.84-4.78 (m, 1H), 3.71 (t, J=4.4 Hz, 3H), 2.55-2.45 (m, 5H), 2.33-2.29 (m, 2H), 1.89-1.83 (m, 2H), 1.81-1.75 (m, 3H), 1.55 (d, J=6.8 Hz, 6H).

Compound 108: ¹H NMR (400 MHz, CD₃OD) δ9.38 (s, 1H), 8.83 (s, 1H), 6.58 (s, 1H), 4.84-4.78 (m, 1H), 3.69 (t, J=4.4 Hz, 4H), 3.04-2.90 (m, 1H), 2.48 (brs, 4H), 2.38-2.29 (m, 2H), 1.85 (t, J=3.2 Hz, 1H), 1.76-1.65 (m, 4H), 1.57 (d, J=6.8 Hz, 6H).

### Example AV: (R)-2-ethyl-4-((1R,3S)-3-(1-Isopropyl-3-(2-(trifluoromethyl) pyrimidin-5-yl)-1H-1,2,4-triazol-5-yl)cyclopentyl)morpholine and (S)-2-ethyl-4-((1R,3S)-3-(1-Isopropyl-3-(2-(trifluoromethyl)pyrimidin-5-yl)-1H-1,2,4-triazol-5-yl)cyclopentyl)morpholine (Compounds 109* & 110*)

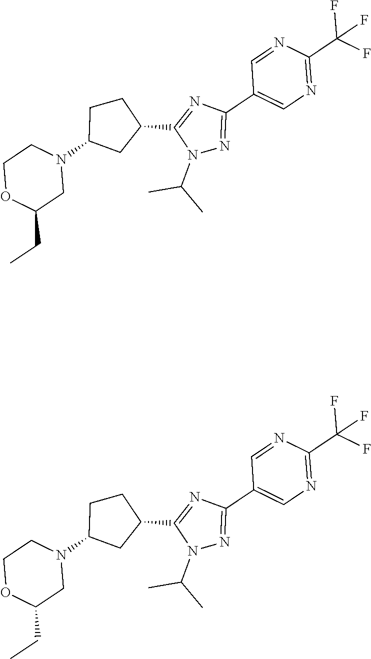 [SMILES: CC[C@@H]1CN([C@@H]2CC[C@H](c3nc(-c4cnc(C(F)(F)F)nc4)nn3C(C)C)C2)CCO1 ; CC[C@H]1CN([C@@H]2CC[C@H](c3nc(-c4cnc(C(F)(F)F)nc4)nn3C(C)C)C2)CCO1]

The title compound was synthesized following a procedure similar to compound 68 using 2-(trifluoromethyl)pyrimidine-5-carboximidamide hydrochloride and (S)-3-oxocyclopentanecarboxylic acid in step 1. Purification of the crude mixture by silica flash chromatography (0-10% methanol in dichloromethane) to provide the title compounds (300 mg, 56.3% yield) as a mixture of diastereomers.

The mixture of diastereomers (300 mg, 0.68 mmol) was separated using chiral SFC (DAICEL CHIRALPAK IE (250 mm*30 mm, 10 um); Hexane-IPA (0.1% NH₄OH), 10/10; 25 mL/min) to provide compound 109* (first peak on SFC, 50.08 mg, 16% yield) and compound 110* (second peak on SFC, 45.7 mg, 14% yield). LCMS (ESI), [M+H]⁺=439.3. The relative stereochemistry was arbitrarily assigned.

Compound 109*: ¹H NMR (400 MHz, CD₃OD) δ9.51 (s, 2H), 4.81-4.74 (m, 1H), 3.88 (dd, J=2.0, 11.6 Hz, 1H), 3.69-3.63 (m, 1H), 3.54-3.40 (m, 2H), 2.99-2.81 (m, 3H), 2.42-2.36 (m, 1H), 2.28-2.14 (m, 2H), 2.12-2.04 (m, 2H), 2.02-1.88 (m, 2H), 1.86-1.81 (m, 1H), 1.54 (dd, J=2.4, 6.4 Hz, 6H), 1.51-1.42 (m, 2H), 0.96 (t, J=7.6 Hz, 3H).

Compound 110*: ¹H NMR (400 MHz, CD₃OD) δ9.51 (s, 2H), 4.81-4.75 (m, 1H), 3.88 (dd, J=2.0, 11.6 Hz, 1H), 3.69-3.64 (m, 1H), 3.55-3.40 (m, 2H), 2.93 (t, J=11.2 Hz, 2H), 2.87-2.79 (m, 1H), 2.42-2.36 (m, 1H), 2.25-2.14 (m, 2H), 2.11-2.00 (m, 2H), 1.99-1.91 (m, 2H), 1.88-1.79 (m, 1H), 1.54 (dd, J=3.6, 6.4 Hz, 6H), 1.50-1.42 (m, 2H), 0.95 (t, J=7.6 Hz, 3H).

### Example AW: 4-((1R,3S)-3-(3-(3-Fluoro-4-(trifluoromethyl)phenyl)-1-isopropyl-1H-1,2,4-triazol-5-yl)cyclopentyl)morpholine and 4-((1S,3S)-3-(3-(3-fluoro-4-(trifluoromethyl)phenyl)-1-isopropyl-1H-1,2,4-triazol-5-yl)cyclopentyl)morpholine (Compounds 111* & 112*)

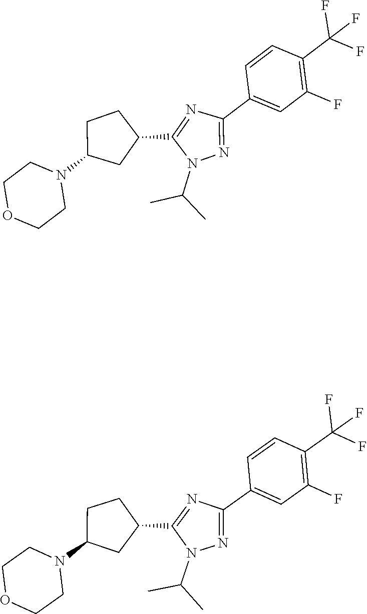 [SMILES: CC(C)n1nc(-c2ccc(C(F)(F)F)c(F)c2)nc1[C@H]1CC[C@@H](N2CCOCC2)C1 ; CC(C)n1nc(-c2ccc(C(F)(F)F)c(F)c2)nc1[C@H]1CC[C@H](N2CCOCC2)C1]

The title compound was synthesized following a procedure similar to compound 68 using 3-fluoro-4-(trifluoromethyl)benzimidamide and (S)-3-oxocyclopentanecarboxylic acid in step 1. Purification of the crude mixture by reverse phase chromatography (Boston Prime C18 150*30 mm*5 um/water (NH₃H₂O+NH₄HCO₃)-ACN), 35%-65%) to provide compound 111* (second peak on SFC, 18.1 mg, 12.4% yield) and compound 112* (first peak on SFC, 11.91 mg, 8.2% yield). LCMS (ESI), [M+H]⁺=427.1. The relative stereochemistry was arbitrarily assigned.

Compound 111*: ¹H NMR (400 MHz, CDCl₃) δ 7.97-7.91 (m, 2H), 7.62 (t, J=8.0 Hz, 1H), 4.56-4.49 (m, 1H), 3.85-3.75 (m, 4H), 3.29-3.24 (m, 1H), 2.82-2.42 (m, 4H), 2.37-2.31 (m, 1H), 2.14-2.04 (m, 4H), 1.54 (d, J=6.8 Hz, 8H).

Compound 112*: ¹H NMR (400 MHz, CDCl₃) 7.97-7.91 (m, 2H), 7.62 (t, J=8.0 Hz, 1H), 4.56-4.53 (m, 1H), 3.93-3.79 (m, 4H), 3.45-3.38 (m, 1H), 2.97-2.92 (m, 1H), 2.62-2.54 (m, 3H), 2.21-1.99 (m, 5H), 1.54 (d, J=6.4 Hz, 8H).

### Example AX: 4-((1S,3R)-3-(3-(3-Fluoro-4-(trifluoromethyl)phenyl)-1-isopropyl-1H-1,2,4-triazol-5-yl)cyclopentyl)morpholine and 4-((1R,3R)-3-(3-(3-fluoro-4-(trifluoromethyl)phenyl)-1-isopropyl-1H-1,2,4-triazol-5-yl)cyclopentyl)morpholine (Compounds 113* & 114*)

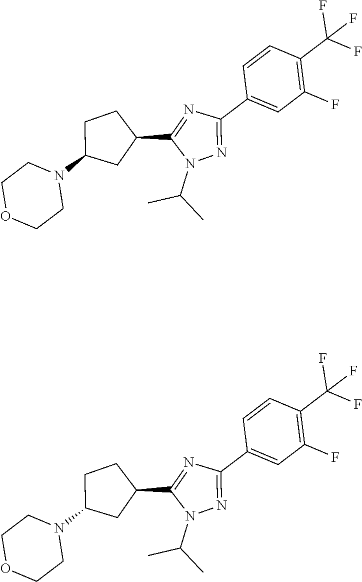 [SMILES: CC(C)n1nc(-c2ccc(C(F)(F)F)c(F)c2)nc1[C@@H]1CC[C@H](N2CCOCC2)C1 ; CC(C)n1nc(-c2ccc(C(F)(F)F)c(F)c2)nc1[C@@H]1CC[C@@H](N2CCOCC2)C1]

The title compound was synthesized following a procedure similar to compound 68 using 3-fluoro-4-(trifluoromethyl)benzimidamide and (R)-3-oxocyclopentanecarboxylic acid in step 1. Purification of the crude mixture by reverse phase chromatography (Boston Prime C18 150*30 mm*5 um/water (0.5% NH₃H₂O+NH₄HCO₃)-CAN, 35%-65%) to provide title compound 113* (second peak on SFC, 11.65 mg, 16% yield) and compound 114* (first peak on SFC, 6.71 mg, 8.9% yield). LCMS (ESI), [M+H]⁺=427.2. The relative stereochemistry was arbitrarily assigned.

Compound 113*: ¹H NMR (400 MHz, CDCl₃) δ 7.97-7.91 (m, 2H), 7.62 (t, J=7.6 Hz, 1H), 4.58-4.49 (m, 1H), 3.80 (s, 4H), 3.30-3.21 (m, 1H), 2.61 (s, 5H), 2.34-2.31 (m, 1H), 2.14-1.90 (m, 5H), 1.54 (d, J=6.8 Hz, 6H).

Compound 114*: ¹H NMR (400 MHz, CDCl₃) δ 7.97-7.91 (m, 2H), 7.62 (t, J=7.8 Hz, 1H), 4.57-4.50 (m, 1H), 3.80 (s, 4H), 3.42-3.39 (m, 1H), 2.99-2.98 (m 1H), 2.61 (s, 4H), 2.33-1.96 (m, 5H), 1.53 (d, J=6.8 Hz, 6H), 1.26 (s, 1H).

### Example AY: 4-((1R,3R)-3-(1-Isopropyl-3-(5-(trifluoromethyl)pyridin-3-yl)-1H-1,2,4-triazol-5-yl)cyclopentyl)morpholine and 4-((1S,3S)-3-(1-Isopropyl-3-(5-(trifluoromethyl) pyridin-3-yl)-1H-1,2,4-triazol-5-yl)cyclopentyl)morpholine and 4-((1S,3R)-3-(1-Isopropyl-3-(5-(trifluoromethyl)pyridin-3-yl)-1H-1,2,4-triazol-5-yl) cyclopentyl) morpholine and 4-((1R,3S)-3-(1-Isopropyl-3-(5-(trifluoromethyl)pyridin-3-yl)-1H-1,2,4-triazol-5-yl)cyclopentyl)morpholine (Compounds 115* & 116* & 117* & 118*)

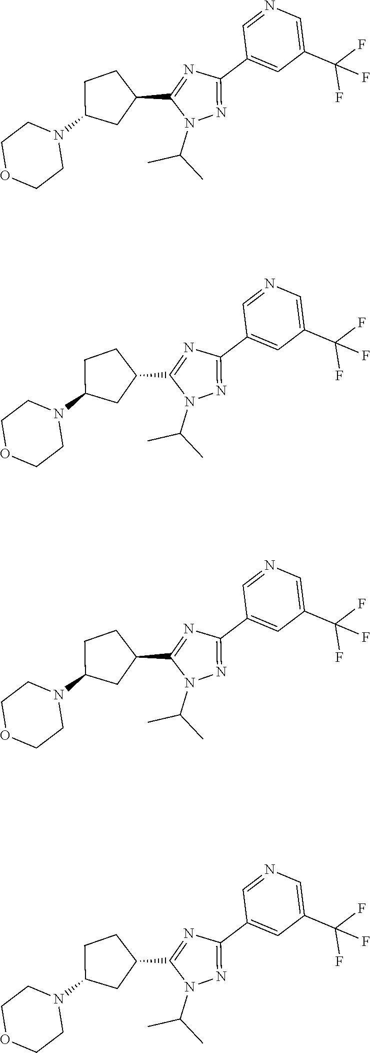 [SMILES: CC(C)n1nc(-c2cncc(C(F)(F)F)c2)nc1[C@@H]1CC[C@@H](N2CCOCC2)C1 ; CC(C)n1nc(-c2cncc(C(F)(F)F)c2)nc1[C@H]1CC[C@H](N2CCOCC2)C1 ; CC(C)n1nc(-c2cncc(C(F)(F)F)c2)nc1[C@@H]1CC[C@H](N2CCOCC2)C1 ; CC(C)n1nc(-c2cncc(C(F)(F)F)c2)nc1[C@H]1CC[C@@H](N2CCOCC2)C1]

### Step 1: 3-(5-Bromo-1-isopropyl-1H-1,2,4-triazol-3-yl)-5-(trifluoromethyl)pyridine

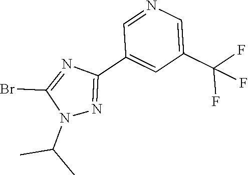 [SMILES: CC(C)n1nc(-c2cncc(C(F)(F)F)c2)nc1Br]

To a solution of 3,5-dibromo-1-isopropyl-1H-1,2,4-triazole (2.0 g, 7.44 mmol) and 3-(4,4,5,5-tetramethyl-1,3,2-dioxaborolan-2-yl)-5-(trifluoromethyl)pyridine (1.83 g, 6.69 mmol) in 1,4-dioxane (40 mL) and water (8 mL) was added Pd(dppf)Cl₂ (544.0 mg, 0.74 mmol) and K₂CO₃ (3.08 g, 22.31 mmol). The reaction mixture was degassed and purged with N₂ for three times, and stirred at 90° C. under N₂ for 3 hours. The reaction mixture was filtered and the filtrate was concentrated under reduced pressure. The residue was purified by flash chromatography (0-10% ethyl acetate in petroleum ether) to provide title compound (600 mg, 24.1% yield). LCMS (ESI), [M+H]⁺=335.0.

### Step 2: 3-(1-Isopropyl-3-(5-(trifluoromethyl)pyridin-3-yl)-1H-1,2,4-triazol-5-yl)cyclopent-2-enone

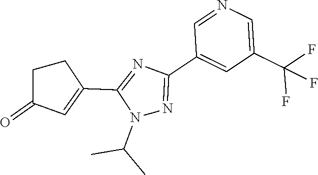 [SMILES: CC(C)n1nc(-c2cncc(C(F)(F)F)c2)nc1C1=CC(=O)CC1]

To a solution of 3-(5-bromo-1-isopropyl-1H-1,2,4-triazol-3-yl)-5-(trifluoromethyl)pyridine (600.0 mg, 1.79 mmol) and 3-(4,4,5,5-tetramethyl-1,3,2-dioxaborolan-2-yl)cyclopent-2-enone (559 mg, 2.69 mmol) in 1,4-dioxane (10 mL) and water (2 mL) was added Pd(dppf)Cl₂ (131.0 mg, 0.18 mmol) and K₂CO₃ (742 mg, 5.37 mmol). The reaction mixture was degassed and purged with N₂ for three times, and stirred at 100° C. under N₂ for 3 hours. The reaction mixture was filtered and the filtrate was concentrated under reduced pressure. The residue was purified by flash chromatography (0-20% ethyl acetate in petroleum ether) to provide title compound (350 mg, 58.1% yield). LCMS (ESI), [M+H]⁺=337.2.

### Step 3: 3-(1-Isopropyl-3-(5-(trifluoromethyl)pyridin-3-yl)-1H-1,2,4-triazol-5-yl)cyclopentanone

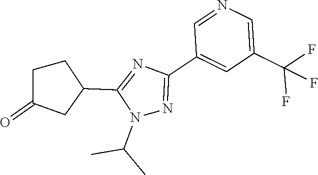 [SMILES: CC(C)n1nc(-c2cncc(C(F)(F)F)c2)nc1C1CCC(=O)C1]

To a solution of 3-(1-isopropyl-3-(5-(trifluoromethyl)pyridin-3-yl)-1H-1,2,4-triazol-5-yl)cyclopent-2-enone (300.0 mg, 0.89 mmol) in ethyl acetate (10 mL) was added R^{h}/C (367 mg). The reaction mixture was degassed and purged with H₂ for three times and stirred at 25° C. for 1 hour under H₂ (15 psi). The mixture was filtered, and the filtrate was concentrated to give title compound (280 mg, 92.8% yield). LCMS (ESI), [M+H]⁺=339.1.

### Step 4: 4-(3-(1-Isopropyl-3-(5-(trifluoromethyl)pyridin-3-yl)-1H-1,2,4-triazol-5-yl)cyclopentyl)morpholine

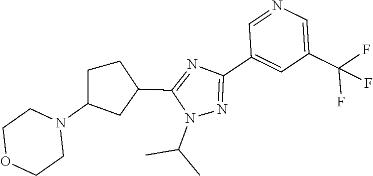 [SMILES: CC(C)n1nc(-c2cncc(C(F)(F)F)c2)nc1C1CCC(N2CCOCC2)C1]

To a solution of 3-(1-isopropyl-3-(5-(trifluoromethyl)pyridin-3-yl)-1H-1,2,4-triazol-5-yl)cyclopentanone (260.0 mg, 0.77 mmol) and morpholine (0.2 mL, 2.31 mmol) in methyl alcohol (4 mL) was added acetic acid (0.18 mL, 3.07 mmol) and sodium cyanoborohydride (242 mg, 3.84 mmol). The mixture was stirred at 60° C. for 1 hour. Then the mixture was adjusted to pH 7-8 with NaHCO₃ (aq.) and extracted with dichloromethane (20 mL×3). The combined organics were washed with brine (20 mL×2), dried over anhydrous Na₂SO₄, filtered and concentrated. The residue was purified by flash chromatography (0-10% methanol in dichloromethane) to provide title compound (200 mg, 64% yield). LCMS (ESI), [M+H]⁺=410.1.

### Step 5: 4-((1R,3R)-3-(1-isopropyl-3-(5-(trifluoromethyl)pyridin-3-yl)-1H-1,2,4-triazol-5-yl)cyclopentyl)morpholine and 4-((1S,3S)-3-(1-isopropyl-3-(5-(trifluoromethyl)pyridin-3-yl)-1H-1,2,4-triazol-5-yl)cyclopentyl)morpholine and 4-((1S,3R)-3-(1-isopropyl-3-(5-(trifluoromethyl)pyridin-3-yl)-1H-1,2,4-triazol-5-yl)cyclopentyl)morpholine and 4-((1R,3S)-3-(1-isopropyl-3-(5-(trifluoromethyl) pyridin-3-yl)-1H-1,2,4-triazol-5-yl)cyclopentyl)morpholine (Compounds 115*& 116* & 117* & 118*)

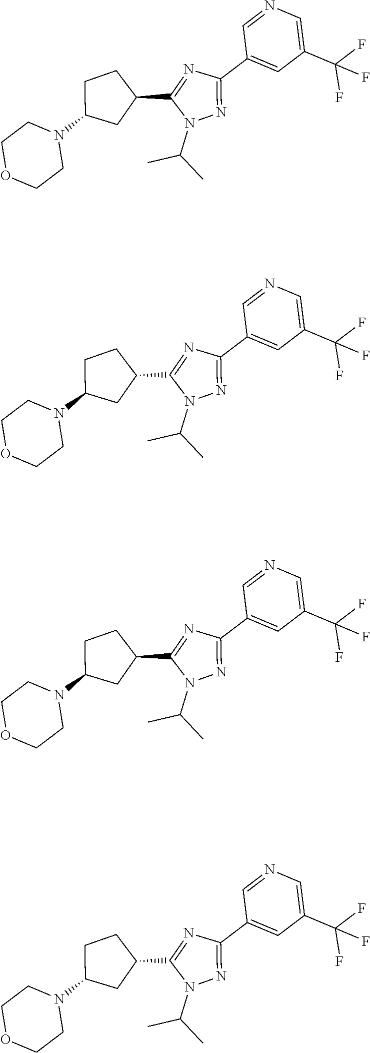 [SMILES: CC(C)n1nc(-c2cncc(C(F)(F)F)c2)nc1[C@@H]1CC[C@@H](N2CCOCC2)C1 ; CC(C)n1nc(-c2cncc(C(F)(F)F)c2)nc1[C@H]1CC[C@H](N2CCOCC2)C1 ; CC(C)n1nc(-c2cncc(C(F)(F)F)c2)nc1[C@@H]1CC[C@H](N2CCOCC2)C1 ; CC(C)n1nc(-c2cncc(C(F)(F)F)c2)nc1[C@H]1CC[C@@H](N2CCOCC2)C1]

The mixture of diastereomers (200.0 mg, 0.49 mmol) was separated using chiral SFC (Daicel Chiralcel OD-H (250 mm*30 mm, 5 um)/0.1% NH₃H₂O IPA, 15/15) to provide compound 115* (first peak on SFC, 10.22 mg, 5.1% yield), compound 116* (second peak on SFC, 9.39 mg, 4.7% yield), compound 117* (third peak on SFC, 19.62 mg, 9.8% yield) and compound 118* (fourth peak on SFC, 31.73 mg, 15.9% yield). LCMS (ESI), [M+H]⁺=410.1. The relative stereochemistry was arbitrarily assigned.

Compound 115*: ¹H NMR (400 MHz, CD₃OD) δ9.43 (d, J=1.6 Hz, 1H), 8.90 (s, 1H), 8.65 (s, 1H), 4.81-4.74 (m, 1H), 3.73 (t, J=4.8 Hz, 4H), 3.64-3.55 (m, 1H), 3.01-2.93 (m, 1H), 2.59 (s, 4H), 2.28-2.16 (m, 3H), 2.11-1.93 (m, 2H), 1.70-1.60 (m, 1H), 1.54 (dd, J=2.0, 6.4 Hz, 6H).

Compound 116*: ¹H NMR (400 MHz, CD₃OD) δ9.43 (s, 1H), 8.90 (s, 1H), 8.65 (s, 1H), 4.81-4.74 (m, 1H), 3.73 (t, J=4.4 Hz, 4H), 3.64-3.55 (m, 1H), 3.01-2.93 (m, 1H), 2.59 (s, 4H), 2.28-2.16 (m, 3H), 2.11-2.04 (m, 1H), 2.01-1.92 (m, 1H), 1.70-1.60 (m, 1H), 1.54 (dd, J=2.0, 6.8 Hz, 6H).

Compound 117*: ¹H NMR (400 MHz, CD₃OD) δ9.43 (d, J=1.6 Hz, 1H), 8.90 (s, 1H), 8.65 (s, 1H), 4.80-4.73 (m, 1H), 3.73 (t, J=4.8 Hz, 4H), 3.53-3.44 (m, 1H), 2.87-2.79 (m, 1H), 2.61-2.60 (m, 4H), 2.42-2.36 (m, 1H), 2.23-2.14 (m, 1H), 2.11-2.03 (m, 2H), 1.99-1.93 (m, 1H), 1.88-1.79 (m, 1H), 1.54 (dd, J=2.0, 6.4 Hz, 6H).

Compound 118*: ¹H NMR (400 MHz, CD₃OD) δ9.43 (d, J=1.6 Hz, 1H), 8.89 (s, 1H), 8.65 (s, 1H), 4.80-4.73 (m, 1H), 3.73 (t, J=4.8 Hz, 4H), 3.53-3.44 (m, 1H), 2.87-2.79 (m, 1H), 2.61-2.60 (m, 4H), 2.42-2.36 (m, 1H), 2.21-2.14 (m, 1H), 2.11-2.05 (m, 2H), 1.99-1.93 (m, 1H), 1.88-1.79 (m, 1H), 1.54 (dd, J=2.0, 6.4 Hz, 6H).

### Example AZ: (R)-4-((1R,3s,5S,6R)-6-(1-Isopropyl-3-(5-(trifluoromethyl)pyridin-3-yl)-1H-1,2,4-triazol-5-yl)bicyclo[3.1.0]hexan-3-yl)-3-methylmorpholine and (R)-4-((1R,3r,5S,6R)-6-(1-Isopropyl-3-(5-(trifluoromethyl)pyridin-3-yl)-1H-1,2,4-triazol-5-yl)bicyclo[3.1.0]hexan-3-yl)-3-methylmorpholine (Compounds 119 & 120)

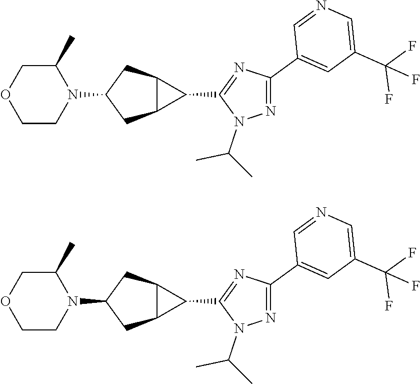 [SMILES: CC(C)n1nc(-c2cncc(C(F)(F)F)c2)nc1[C@H]1[C@@H]2C[C@@H](N3CCOC[C@H]3C)C[C@@H]21 ; CC(C)n1nc(-c2cncc(C(F)(F)F)c2)nc1[C@H]1[C@@H]2C[C@H](N3CCOC[C@H]3C)C[C@@H]21]

The title compound was synthesized following a procedure similar to compound 68 using 5-(Trifluoromethyl)nicotinimidamide hydrochloride and (1R,5S)-3-[tert-butyl(diphenyl)silyl]oxybicyclo [3.1.0]hexane-6-carboxylic acid in step 1. Purification of the crude mixture by reverse phase chromatography (water (NH₃H₂O+NH₄HCO₃)-ACN, 55%-85%, 25 ml/min) to provide compound 119 (first peak on HPLC, 15.3 mg, 20.3% yield) and compound 120 (second peak on HPLC, 23.5 mg, 31.2% yield). LCMS (ESI), [M+H]⁺=436.2. The relative stereochemistry was determined by 2D-NMR.

Compound 119: ¹H NMR (400 MHz, CD₃OD) δ 9.38 (s, 1H), 8.88 (s, 1H), 8.60 (s, 1H), 4.93 (s, 1H), 3.74-3.71 (m, 3H), 3.50-3.46 (m, 1H), 3.18-3.04 (m, 1H), 2.87-2.73 (m, 2H), 2.52-2.46 (m, 1H), 2.32-2.14 (m, 2H), 2.09-2.04 (m, 3H), 1.96-1.87 (m, 2H), 1.56 (dd, J=2.8, 6.4 Hz, 6H), 1.12 (d, J=6.4 Hz, 3H).

Compound 120: ¹H NMR (400 MHz, CD₃OD) δ 9.38 (s, 1H), 8.88 (s, 1H), 8.60 (s, 1H), 4.92 (s, 1H), 3.89-3.59 (m, 3H), 3.48-3.43 (m, 1H), 3.18-3.04 (m, 1H), 2.90-2.63 (m, 2H), 2.58-2.46 (m, 1H), 2.36-2.05 (m, 5H), 1.99-1.86 (m, 2H), 1.56 (dd, J=2.8, 6.8 Hz, 6H), 1.12 (d, J=6.4 Hz, 3H).

### Example BA: (S)-4-((1R,3s,5S,6R)-6-(1-Isopropyl-3-(5-(trifluoromethyl)pyridin-3-yl)-1H-1,2,4-triazol-5-yl)bicyclo[3.1.0]hexan-3-yl)-3-methylmorpholine and (S)-4-((1R,3r,5S,6R)-6-(1-Isopropyl-3-(5-(trifluoromethyl)pyridin-3-yl)-1H-1,2,4-triazol-5-yl)bicyclo[3.1.0]hexan-3-yl)-3-methylmorpholine (Compounds 121 & 122)

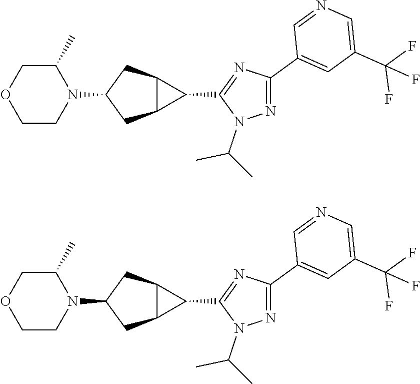 [SMILES: CC(C)n1nc(-c2cncc(C(F)(F)F)c2)nc1[C@H]1[C@@H]2C[C@@H](N3CCOC[C@@H]3C)C[C@@H]21 ; CC(C)n1nc(-c2cncc(C(F)(F)F)c2)nc1[C@H]1[C@@H]2C[C@H](N3CCOC[C@@H]3C)C[C@@H]21]

The title compound was synthesized following a procedure similar to compound 68 using 5-(Trifluoromethyl)nicotinimidamide hydrochloride and (1R,5S)-3-[tert-butyl(diphenyl)silyl]oxybicyclo [3.1.0]hexane-6-carboxylic acid in step 1. Purification of the crude mixture by reverse phase chromatography (water (NH₃H₂O+NH₄HCO₃)-ACN, 50%-80%, 25 ml/min) to provide compound 121 (first peak on HPLC, 22.04 mg, 27.8% yield) and compound 122 (second peak on HPLC, 26 mg, 34.5% yield). LCMS (ESI), [M+H]⁺=436.2. The relative stereochemistry was determined by 2D-NMR.

Compound 121: ¹H NMR (400 MHz, CD₃OD) δ 9.38 (s, 1H), 8.88 (s, 1H), 8.60 (s, 1H), 4.92 (s, 1H), 3.85-3.62 (m, 3H), 3.49-3.45 (m, 1H), 3.13-3.09 (m, 1H), 2.87-2.71 (m, 2H), 2.55-2.48 (m, 1H), 2.35-2.05 (m, 5H), 1.97-1.87 (m, 2H), 1.56 (dd, J=2.8, 6.8 Hz, 6H), 1.12 (d, J=6.8 Hz, 3H).

Compound 122: ¹H NMR (400 MHz, CD₃OD) δ 9.37 (s, 1H), 8.87 (s, 1H), 8.59 (s, 1H), 4.93 (s, 1H), 3.77-3.63 (m, 3H), 3.54-3.45 (m, 2H), 2.88-2.85 (m, 1H), 2.69-2.63 (m, 1H), 2.53-2.49 (m, 1H), 2.33-2.20 (m, 2H), 2.16-2.15 (m, 1H), 2.04 (brs, 2H), 1.85-1.72 (m, 2H), 1.58 (d, J=6.4 Hz, 6H), 1.09 (d, J=6.4 Hz, 3H).

### Example BB: (S)-2-Ethyl-4-((1R,3S)-3-(3-(5-fluoropyridin-3-yl)-1-isopropyl-1H-1,2,4-triazol-5-yl)cyclopentyl)morpholine (Compound 123)

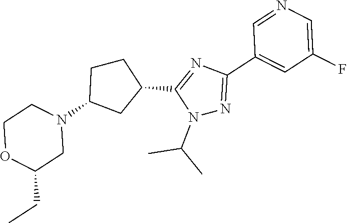 [SMILES: CC[C@H]1CN([C@@H]2CC[C@H](c3nc(-c4cncc(F)c4)nn3C(C)C)C2)CCO1]

The title compound was synthesized following a procedure similar to compound 68 using 5-Fluoronicotinimidamide and (S)-3-oxocyclopentanecarboxylic acid in step 1. Purification of the crude mixture by silica flash chromatography (0-10% methanol in dichloromethane) to provide the title compound as a mixture of diastereomers (80 mg), which was purified using chiral SFC (SFC-16; DAICEL CHIRALPAK IG (250 mm*30 mm, 10 um); 0.1% NH₃H₂O MEOH, 35/35; 70 mL/min) to provide compound 123 (59.61 mg, 75% yield). LCMS (ESI) [M+H]⁺=388.3. The relative stereochemistry was determined by 2D-NMR.

Compound 123: ¹H NMR (400 MHz, CDCl₃) δ 9.11 (s, 1H), 8.43 (d, J=2.8 Hz, 1H), 8.05 (dd, J=1.6, 10.8 Hz, 1H), 4.68-4.52 (m, 1H), 3.94 (dd, J=1.6, 11.2 Hz, 1H), 3.75-3.65 (m, 1H), 3.52-3.47 (m, 1H), 3.30-3.20 (m, 1H), 2.92-2.89 (m, 2H), 2.88-2.85 (m, 1H), 2.36-2.22 (m, 2H), 2.15-2.10 (m, 2H), 2.08-2.05 (m, 2H), 1.95-1.90 (m, 2H), 1.54 (d, J=6.4 Hz, 6H), 1.53-1.46 (m, 2H), 0.91 (t, J=7.6 Hz, 3H).

### Example BC: (R)-2-Ethyl-4-((1R,3S)-3-(3-(5-fluoropyridin-3-yl)-1-isopropyl-1H-1,2,4-triazol-5-yl)cyclopentyl)morpholine (Compound 124)

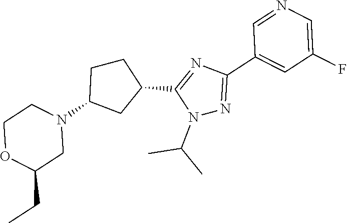 [SMILES: CC[C@@H]1CN([C@@H]2CC[C@H](c3nc(-c4cncc(F)c4)nn3C(C)C)C2)CCO1]

The title compound was synthesized following a procedure similar to compound 68 using 5-Fluoronicotinimidamide and (S)-3-oxocyclopentanecarboxylic acid in step 1. Purification of the crude mixture by silica flash chromatography (0-10% methanol in dichloromethane) to provide the title compound as a mixture of diastereomers (80 mg), which was purified using chiral SFC (SFC-16; DAICEL CHIRALPAK IG (250 mm*30 mm, 10 um); 0.1% NH₃H₂O MEOH, 35/35; 70 mL/min) to provide compound 124 (48.82 mg, 61% yield). LCMS (ESI) [M+H]⁺=388.3. The relative stereochemistry was determined by 2D-NMR.

Compound 124: H NMR (400 MHz, CDCl₃) δ 9.11 (s, 1H), 8.47 (s, 1H), 8.06 (d, J=8.8 Hz, 1H), 4.55-4.52 (m, 1H), 3.92-3.90 (m, 1H), 3.77-3.74 (m, 1H), 3.55-3.51 (m, 1H), 3.31-3.24 (m, 1H), 2.95 (d, J=11.6 Hz, 1H), 2.88-2.85 (m, 2H), 2.33-2.29 (m, 2H), 2.12-2.08 (m, 3H), 2.03-1.96 (m, 1H), 1.93-1.90 (m, 2H), 1.55 (d, J=6.4 Hz, 6H), 1.51-1.50 (m, 2H), 1.01 (t, J=7.6 Hz, 3H).

### Example BD: (S)-4-((1S,3S)-3-(3-(5-Chloropyridin-3-yl)-1-isopropyl-1H-1,2,4-triazol-5-yl) cyclopentyl)-3-methylmorpholine, (S)-4-((1R,3S)-3-(3-(5-Chloropyridin-3-yl)-1-isopropyl-1H-1,2,4-triazol-5-yl)cyclopentyl)-3-methylmorpholine (Compounds 125 & 126)

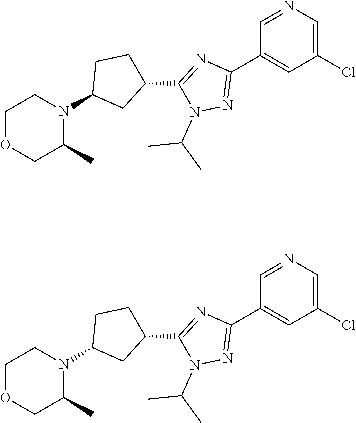 [SMILES: CC(C)n1nc(-c2cncc(Cl)c2)nc1[C@H]1CC[C@H](N2CCOC[C@@H]2C)C1 ; CC(C)n1nc(-c2cncc(Cl)c2)nc1[C@H]1CC[C@@H](N2CCOC[C@@H]2C)C1]

The title compound was synthesized following a procedure similar to compound 68 using 5-chloronicotinimidamide and (S)-3-oxocyclopentanecarboxylic acid in step 1. Purification of the crude mixture by silica flash chromatography (0-10% methanol in dichloromethane) to provide the title compound (110 mg, 83% yield) as a mixture of diastereomers. The diastereomers was separated using chiral SFC (SFC-16; DAICEL CHIRALPAK IG (250 mm*30 mm, 10 um); 0.1% NH₃H₂O MEOH, 35/35; 70 mL/min) to provide compound 125 (first peak on SFC, 6.7 mg, 6% yield) and compound 126 (second peak on SFC, 62.8 mg, 57% yield). LCMS (ESI) [M+H]⁺=390.2. The relative stereochemistry was determined by 2D-NMR.

Compound 125: ¹H NMR (400 MHz, CD₃OD) δ 9.09 (d, J=1.6 Hz, 1H), 8.57 (s, 1H), 8.42 (t, J=2.4 Hz, 1H), 4.80-4.72 (1H), 3.75-3.72 (m, 3H), 3.57-3.45 (m, 3H), 2.94-2.76 (m, 2H), 2.62-2.59 (m, 1H), 2.24-2.20 (m, 2H), 2.10-2.04 (m, 2H), 2.00-1.89 (m, 1H), 1.80-1.67 (m, 1H), 1.53 (d, J=6.8 Hz, 6H), 1.15 (d, J=6.4 Hz, 3H).

Compound 126: ¹H NMR (400 MHz, CD₃OD) δ 9.10 (d, J=1.6 Hz, 1H), 8.57 (d, J=2.4 Hz, 1H), 8.43 (t, J=2.0 Hz, 1H), 4.78-4.76 (m, 1H), 3.82-3.72 (m, 3H), 3.48-3.42 (m, 3H), 2.83-2.81 (m, 2H), 2.67-2.55 (m, 1H), 2.27-2.10 (m, 2H), 2.08-1.98 (m, 3H), 1.94-1.85 (m, 1H), 1.54 (dd, J=3.2, 6.4 Hz, 6H), 1.13 (d, J=6.4 Hz, 3H).

### Example BE: (R)-2-Ethyl-4-((1R,3S)-3-(1-isopropyl-3-(6-(trifluoromethyl)pyridin-3-yl)-1H-1,2,4-triazol-5-yl)cyclopentyl)morpholine (Compound 127)

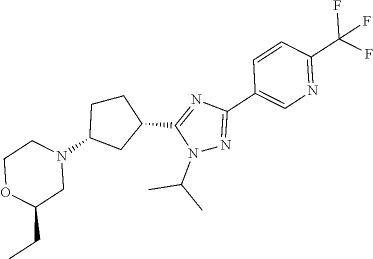 [SMILES: CC[C@@H]1CN([C@@H]2CC[C@H](c3nc(-c4ccc(C(F)(F)F)nc4)nn3C(C)C)C2)CCO1]

The title compound was synthesized following a procedure similar to compound 68 using 6-(trifluoromethyl)nicotinimidamide hydrochloride and (5)-3-oxocyclopentanecarboxylic acid in step 1. Purification of the crude mixture by silica flash chromatography (0-10% methanol in dichloromethane) to provide the title compound (100 mg) as a mixture of diastereomers. The diastereomers was separated using chiral SFC (DAICEL CHIRALPAK AD (250 mm*30 mm, 10 um); 0.1% NH₄OH; IPA, 15/15, 70 mL/min) to provide compound 127 (76.74 mg, 77% yield). LCMS (ESI), [M+H]⁺=438.2. The relative stereochemistry was determined by 2D-NMR.

Compound 127: H NMR (400 MHz, CD₃OD) δ 9.32 (s, 1H), 8.61 (dd, J=1.6, 8.0 Hz, 1H), 7.90 (d, J=8.4 Hz, 1H), 4.77-4.76 (m, 1H), 3.88 (dd, J=2.0, 12.0 Hz, 1H), 3.70-3.63 (m, 1H), 3.52-3.40 (m, 2H), 2.95-2.89 (m, 2H), 2.87-2.79 (m, 1H), 2.41-2.35 (m, 1H), 2.25-2.14 (m, 2H), 2.11-2.01 (m, 2H), 2.00-1.90 (m, 2H), 1.88-1.79 (m, 1H), 1.54 (dd, J=3.2, 6.4 Hz, 6H), 1.50-1.44 (m, 2H), 0.95 (t, J=7.6 Hz, 3H).

### Example BF: (S)-2-Ethyl-4-((1R,3S)-3-(1-isopropyl-3-(6-(trifluoromethyl)pyridin-3-yl)-1H-1,2,4-triazol-5-yl)cyclopentyl)morpholine (Compound 128)

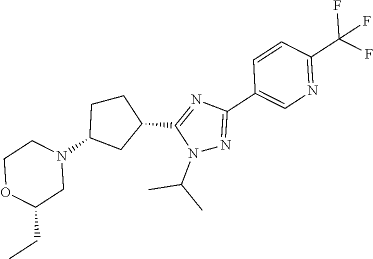 [SMILES: CC[C@H]1CN([C@@H]2CC[C@H](c3nc(-c4ccc(C(F)(F)F)nc4)nn3C(C)C)C2)CCO1]

The title compound was synthesized following a procedure similar to compound 68 using 6-(trifluoromethyl)nicotinimidamide hydrochloride and (S)-3-oxocyclopentanecarboxylic acid in step 1. Purification of the crude mixture by silica flash chromatography (0-10% methanol in dichloromethane) to provide the title compound (100 mg) as a mixture of diastereomers. The mixture of diastereomers was separated using chiral SFC (DAICEL CHIRALPAK AD (250 mm*30 mm, 10 um); 0.1% NH₄OH; EtOH, 10/10, 70 mL/min) to provide compound 128 (76.8 mg, 77% yield). LCMS (ESI), [M+H]⁺=438.2. The relative stereochemistry was determined by 2D-NMR.

Compound 128: ¹H NMR (400 MHz, CD₃OD) δ 9.32 (d, J=1.2 Hz, 1H), 8.61 (dd, J=1.6, 8.0 Hz, 1H), 7.90 (d, J=8.0 Hz, 1H), 4.78-4.74 (m, 1H), 3.88 (dd, J=2.0, 12.0 Hz, 1H), 3.70-3.63 (m, 1H), 3.52-3.41 (m, 2H), 2.98-2.95 (m, 2H), 2.89-2.79 (m, 1H), 2.41-2.35 (m, 1H), 2.28-2.14 (m, 2H), 2.11-2.02 (m, 2H), 1.98-1.88 (m, 2H), 1.86-1.78 (m, 1H), 1.54 (dd, J=2.4, 6.4 Hz, 6H), 1.50-1.44 (m, 2H), 0.96 (t, J=7.6 Hz, 3H).

### Example BG: (R)-4-((1R, 3S)-3-(3-(5-Chloropyridin-3-yl)-1-isopropyl-1H-1,2,4-triazol-5-yl)cyclopentyl)-2-ethylmorpholine (Compound 129)

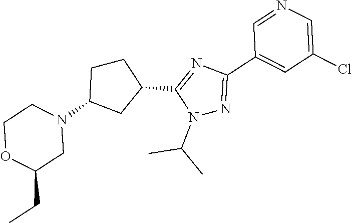 [SMILES: CC[C@@H]1CN([C@@H]2CC[C@H](c3nc(-c4cncc(Cl)c4)nn3C(C)C)C2)CCO1]

The title compound was synthesized following a procedure similar to compound 68 using 5-chloronicotinimidamide and (S)-3-oxocyclopentanecarboxylic acid in step 1. Purification of the crude mixture by silica flash chromatography (0-10% methanol in dichloromethane) to provide the title compound (120 mg, 91% yield) as a mixture of diastereomers. The diastereomers (100 mg) was separated using chiral SFC (SFC-16; DAICEL CHIRALPAK AS (250 mm*30 mm, 10 um); 0.1% NH₃H₂O ETOH, 40/40, 80 mL/min) to provide compound 129 (62.9 mg, 63% yield). LCMS (ESI), [M+H]⁺=404.2. The relative stereochemistry was determined by 2D-NMR.

Compound 129: ¹H NMR (400 MHz, CD₃OD) δ 9.10 (d, J=1.6 Hz, 1H), 8.57 (d, J=2.4 Hz, 1H), 8.42 (t, J=2.0 Hz, 1H), 4.76-4.73 (m, 1H), 3.88 (dd, J=2.4, 11.6 Hz, 1H), 3.67-3.64 (m, 1H), 3.57-3.36 (m, 2H), 3.01-2.80 (m, 3H), 2.44-2.36 (m, 1H), 2.24-2.12 (m, 2H), 2.12-1.95 (m, 2H), 1.89-1.78 (m, 1H), 1.58-1.48 (m, 8H), 0.96 (t, J=7.6 Hz, 3H).

### Example BH: (R)-2-(Fluoromethyl)-4-((1S,3S)-3-(1-isopropyl-3-(2-(trifluoromethyl)pyrimidin-5-yl)-1H-pyrazol-5-yl)cyclopentyl)morpholine and (R)-2-(Fluoromethyl)-4-((1R,3S)-3-(1-isopropyl-3-(2-(trifluoromethyl)pyrimidin-5-yl)-1H-pyrazol-5-yl)cyclopentyl)morpholine (Compounds 130*& 131*)

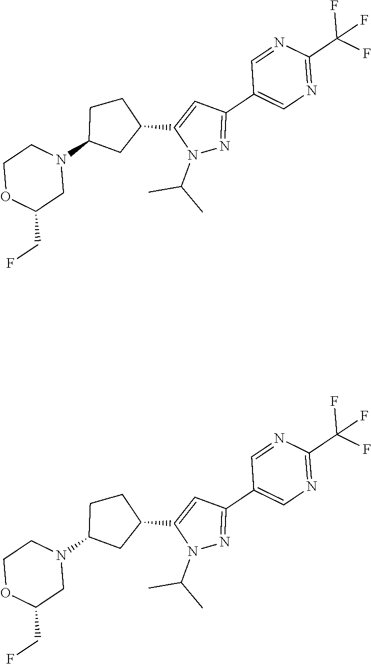 [SMILES: CC(C)n1nc(-c2cnc(C(F)(F)F)nc2)cc1[C@H]1CC[C@H](N2CCO[C@@H](CF)C2)C1 ; CC(C)n1nc(-c2cnc(C(F)(F)F)nc2)cc1[C@H]1CC[C@@H](N2CCO[C@@H](CF)C2)C1]

### Step 1: (S)-3-(1-Isopropyl-3-(2-(trifluoromethyl)pyrimidin-5-yl)-1H-pyrazol-5-yl)cyclopentanone

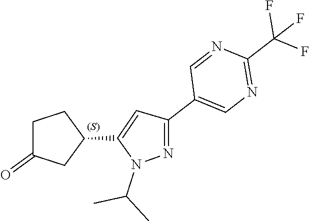

To a mixture of (S)-3-(3-bromo-1-isopropyl-1H-pyrazol-5-yl)cyclopentanone (500 mg, 1.84 mmol, second peak on SFC), (2-(trifluoromethyl)pyrimidin-5-yl)boronic acid (708 mg, 3.69 mmol), K₂CO₃ (765 mg, 5.53 mmol) and bis(di-tert-butyl(4-dimethylaminophenyl)phosphine) dichloropalladium(II) (131 mg, 0.18 mmol) in 1,4-dioxane (8 mL) and water (2 mL) were placed under nitrogen atmosphere and stirred at 100° C. for 4 hours. The reaction mixture was filtered and concentrated. The residue was purified by silica flash chromatography (0-20% ethyl acetate in petroleum ether) to provide title compound (600 mg, 93% yield). LCMS (ESI), [M+H]⁺=338.9.

### Step 2: ((2R)-4-((3S)-3-(1-Isopropyl-3-(2-(trifluoromethyl)pyrimidin-5-yl)-1H-pyrazol-5-yl)cyclopentyl)morpholin-2-yl)methanol

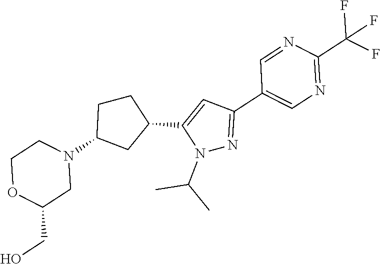 [SMILES: CC(C)n1nc(-c2cnc(C(F)(F)F)nc2)cc1[C@H]1CC[C@@H](N2CCO[C@@H](CO)C2)C1]

To a mixture of (S)-3-(1-isopropyl-3-(2-(trifluoromethyl)pyrimidin-5-yl)-1H-pyrazol-5-yl)cyclopentanone (300 mg, 0.89 mmol) in dichloromethane (3 mL) was added (R)-morpholin-2-ylmethanol hydrochloride (272 mg, 1.77 mmol) and N,N-diisopropylethylamine (344 mg, 2.66 mmol) and stirred at 25° C. for 16 hours. Then NaBH(OAc)₃ (564 mg, 2.66 mmol) was added and stirred at 25° C. for 16 hours. The mixture was diluted with water (10 mL) and adjusted pH to 9 with NaHCO₃ aq. The resulting mixture was extracted with ethyl acetate (50 mL×3). The combined organic layer was dried over anhydrous sodium sulfate, filtered and concentrated under vacuum. The residue was purified by silica flash chromatography (0-2% methanol in dichloromethane) to provide title compound (300 mg, 77% yield) as a mixture of diastereomers. LCMS (ESI), [M+H]⁺=440.2.

### Step 3: (2R)-2-(Fluoromethyl)-4-((3S)-3-(1-isopropyl-3-(2-(trifluoromethyl)pyrimidin-5-yl)-1H-pyrazol-5-yl)cyclopentyl)morpholine

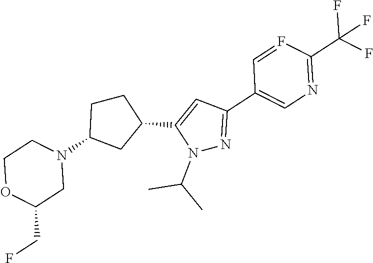

To a solution of ((2R)-4-((3S)-3-(1-isopropyl-3-(2-(trifluoromethyl)pyrimidin-5-yl)-1H-pyrazol-5-yl)cyclopentyl)morpholin-2-yl)methanol (150 mg, 0.34 mmol) in dichloromethane (5 mL) was added (bis-(2-methoxyethyl)amino)sulfur trifluoride (240 mg, 1.08 mmol) at 0° C. and stirred at 25° C. for 16 hours. The mixture was diluted with water (25 mL) and adjusted pH to 9 with NaHCO₃ aq. The resulting mixture was extracted with ethyl acetate (50 mL×3). The combined organic layer was dried over anhydrous sodium sulfate, filtered and concentrated in vacuum. The residue was purified by prep-TLC (petroleum ether/ethyl acetate/ethanol=4/3/1) to provide title compound (100 mg, 66% yield) as a mixture of diastereomers. LCMS (ESI), [M+H]⁺=442.3.

### Step 4: (R)-2-(Fluoromethyl)-4-((1S,3S)-3-(1-isopropyl-3-(2-(trifluoromethyl)pyrimidin-5-yl)-1H-pyrazol-5-yl)cyclopentyl)morpholine and (R)-2-(Fluoromethyl)-4-((1R,3S)-3-(1-isopropyl-3-(2-(trifluoromethyl)pyrimidin-5-yl)-1H-pyrazol-5-yl)cyclopentyl)morpholine (Compounds 130*and 131*)

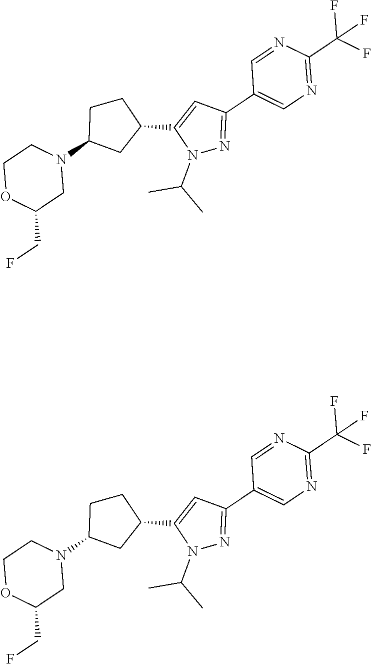 [SMILES: CC(C)n1nc(-c2cnc(C(F)(F)F)nc2)cc1[C@H]1CC[C@H](N2CCO[C@@H](CF)C2)C1 ; CC(C)n1nc(-c2cnc(C(F)(F)F)nc2)cc1[C@H]1CC[C@@H](N2CCO[C@@H](CF)C2)C1]

The diastereomers (100 mg, 0.23 mmol) was separated using chiral SFC (SFC-22, DAICEL CHIRALPAK AD(250 mm*30 mm, 10 um), 0.1% NH₃H₂O ETOH, 15/15, 80 ml/min) to provide compound 130* (anti isomer, first peak on SFC, 8.34 mg, 8% yield) and compound 131* (syn isomer, second peak on SFC, 28.78 mg, 28% yield). LCMS (ESI), [M+H]⁺=442.3. The relative stereochemistry was arbitrarily assigned.

Compound 130*: ¹H NMR (400 MHz, CD₃OD) δ9.31 (s, 2H), 6.74 (s, 1H), 4.70-4.64 (m, 1H), 4.46 (d, J=4.0 Hz, 1H), 4.34 (d, J=4.0 Hz, 1H), 3.97-3.91 (m, 1H), 3.84-3.64 (m, 2H), 3.52-3.36 (m, 1H), 3.03-2.85 (m, 3H), 2.29-2.11 (m, 4H), 2.10-1.95 (m, 2H), 1.84-1.62 (m, 2H), 1.52 (t, J=6.4 Hz, 6H).

Compound 131*: ¹H NMR (400 MHz, CD₃OD) δ9.31 (s, 2H), 6.78 (s, 1H), 4.70-4.63 (m, 1H), 4.47 (d, J=4.0 Hz, 1H), 4.35 (d, J=4.4 Hz, 1H), 3.96-3.88 (m, 1H), 3.84-3.74 (m, 1H), 3.73-3.66 (m, 1H), 3.38-3.32 (m, 1H), 3.01-2.96 (m, 1H), 2.92-2.77 (m, 2H), 2.44-2.37 (m, 1H), 2.31-2.16 (m, 2H), 2.11-2.01 (m, 2H), 1.86-1.74 (m, 2H), 1.72-1.63 (m, 1H), 1.52 (d, J=6.4 Hz, 6H).

### Example BI: (S)-2-(Fluoromethyl)-4-((1S,3S)-3-(1-isopropyl-3-(2-(trifluoromethyl) pyrimidin-5-yl)-1H-pyrazol-5-yl)cyclopentyl)morpholine and (S)-2-(Fluoromethyl)-4-((1R,3S)-3-(1-isopropyl-3-(2-(trifluoromethyl)pyrimidin-5-yl)-1H-pyrazol-5-yl)cyclopentyl)morpholine (Compounds 132* & 133*)

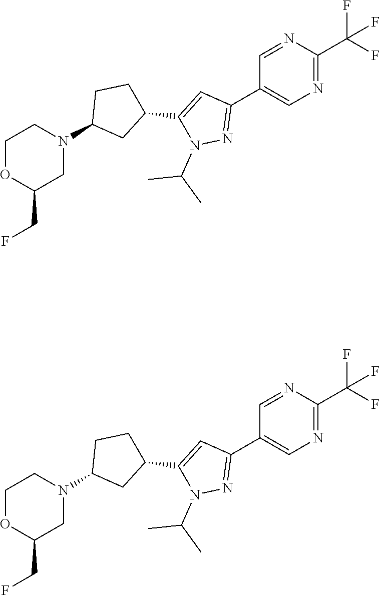 [SMILES: CC(C)n1nc(-c2cnc(C(F)(F)F)nc2)cc1[C@H]1CC[C@H](N2CCO[C@H](CF)C2)C1 ; CC(C)n1nc(-c2cnc(C(F)(F)F)nc2)cc1[C@H]1CC[C@@H](N2CCO[C@H](CF)C2)C1]

### Step 1: ((2S)-4-((3S)-3-(1-Isopropyl-3-(2-(trifluoromethyl)pyrimidin-5-yl)-1H-pyrazol-5-yl)cyclopentyl)morpholin-2-yl)methanol

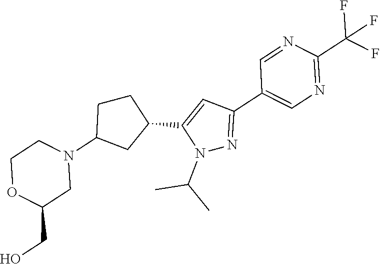 [SMILES: CC(C)n1nc(-c2cnc(C(F)(F)F)nc2)cc1[C@H]1CCC(N2CCO[C@H](CO)C2)C1]

To a mixture of (S)-3-(1-isopropyl-3-(2-(trifluoromethyl)pyrimidin-5-yl)-1H-pyrazol-5-yl)cyclopentanone (300 mg, 0.89 mmol) in dichloromethane (3 mL) was added (S)-morpholin-2-ylmethanol hydrochloride (272 mg, 1.77 mmol) and N,N-diisopropylethylamine (344 mg, 2.66 mmol) and stirred at 25° C. for 16 hours. Then NaBH(OAc)₃ (564 mg, 2.66 mmol) was added and stirred at 25° C. for 16 hours. The mixture was diluted with water (25 mL) and adjusted pH to 9 with NaHCO₃ aq. The resulting mixture was extracted with ethyl acetate (50 mL×3). The combined organic layer was dried over anhydrous sodium sulfate, filtered, and concentrated under vacuum. The residue was purified by silica flash chromatography (0-2% methanol in dichloromethane) to provide title compound (350 mg, 90% yield) as a mixture of diastereomers. LCMS (ESI), [M+H]⁺=440.3

### Step 2: (2S)-2-(Fluoromethyl)-4-((3S)-3-(1-isopropyl-3-(2-(trifluoromethyl)pyrimidin-5-yl)-1H-pyrazol-5-yl)cyclopentyl)morpholine

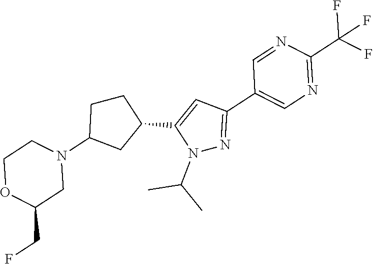 [SMILES: CC(C)n1nc(-c2cnc(C(F)(F)F)nc2)cc1[C@H]1CCC(N2CCO[C@H](CF)C2)C1]

((2S)-4-((3S)-3-(1-Isopropyl-3-(2-(trifluoromethyl)pyrimidin-5-yl)-1H-pyrazol-5-yl)cyclopentyl) morpholin-2-yl)methanol (150 mg, 0.34 mmol) in dichloromethane (5 mL) was added (bis-(2-methoxyethyl)amino)sulfur trifluoride (240 mg, 1.08 mmol) at 0° C. and stirred at 25° C. for 16 hours. The mixture was diluted with water (25 mL) and adjusted pH to 9 with NaHCO₃ aq. The resulting mixture was extracted with ethyl acetate (50 mL×3). The combined organic layer was dried over anhydrous sodium sulfate, filtered and concentrated under vacuum. The residue was purified by prep-TLC (petroleum ether/ethyl acetate/ethanol=4/3/1) to provide title compound (100 mg, 66% yield) as a mixture of diastereomers. LCMS (ESI), [M+H]⁺=442.3

### Step 3: (S)-2-(Fluoromethyl)-4-((1S,3S)-3-(1-isopropyl-3-(2-(trifluoromethyl)pyrimidin-5-yl)-1H-pyrazol-5-yl)cyclopentyl)morpholine and (S)-2-(Fluoromethyl)-4-((1R,3S)-3-(1-isopropyl-3-(2-(trifluoromethyl)pyrimidin-5-yl)-1H-pyrazol-5-yl)cyclopentyl)morpholine (Compounds 132* & 133*)

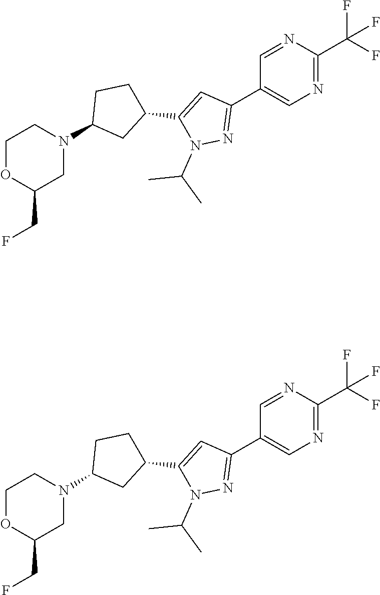 [SMILES: CC(C)n1nc(-c2cnc(C(F)(F)F)nc2)cc1[C@H]1CC[C@H](N2CCO[C@H](CF)C2)C1 ; CC(C)n1nc(-c2cnc(C(F)(F)F)nc2)cc1[C@H]1CC[C@@H](N2CCO[C@H](CF)C2)C1]

The diastereomers (100 mg, 0.23 mmol) was separated using chiral SFC (SFC-11, DAICEL CHIRALPAK AD (250 mm*30 mm, 10 um), 0.1% NH₃H₂O MEOH, 20/20, 70 ml/min) to provide compound 132* (anti isomer, first peak on SFC, 11.93 mg, 12% yield) and compound 133* (syn isomer, second peak on SFC, 37.02 mg, 37% yield). LCMS (ESI), [M+H]⁺=442.3.

Compound 132*: ¹H NMR (400 MHz, CD₃OD) δ9.31 (s, 2H), 6.74 (s, 1H), 4.71-4.64 (m, 1H), 4.47 (d, J=4.0 Hz, 1H), 4.35 (d, J=4.0 Hz, 1H), 3.96-3.92 (m, 1H), 3.81-3.66 (m, 2H), 3.47-3.38 (m, 1H), 2.99-2.97 (m, 1H), 2.93-2.85 (m, 2H), 2.29-2.11 (m, 4H), 2.10-1.94 (m, 2H), 1.84-1.73 (m, 1H), 1.71-1.62 (m, 1H), 1.54-1.51 (m, 6H).

Compound 133*: ¹H NMR (400 MHz, CD₃OD) δ9.31 (s, 2H), 6.78 (s, 1H), 4.70-4.64 (m, 1H), 4.46 (d, J=4.4 Hz, 1H), 4.35 (d, J=4.4 Hz, 1H), 3.95-3.90 (m, 1H), 3.83-3.66 (m, 2H), 3.36-3.32 (m, 1H), 3.00-2.76 (m, 3H), 2.43-2.37 (m, 1H), 2.27-2.15 (m, 2H), 2.12-2.01 (m, 2H), 1.86-1.62 (m, 3H), 1.54-1.51 (m, 6H).

### Example BJ: (R)-4-((1R,3S)-3-(1-Isopropyl-3-(2-(trifluoromethyl)pyrimidin-5-yl)-1H-1,2,4-triazol-5-yl)cyclopentyl)-2-methylmorpholine and (R)-4-((1S,3S)-3-(1-Isopropyl-3-(2-(trifluoromethyl)pyrimidin-5-yl)-1H-1,2,4-triazol-5-yl)cyclopentyl)-2-methylmorpholine (Compounds 134 & 135)

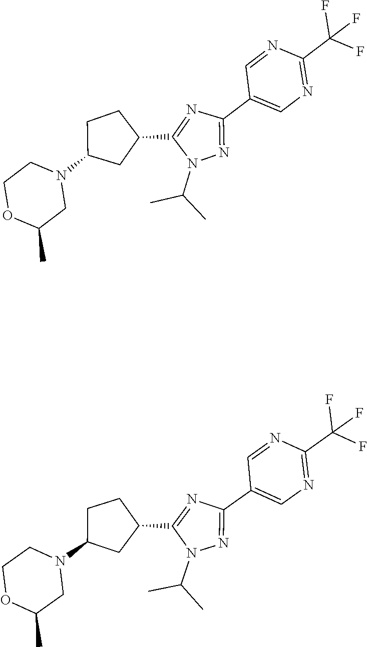 [SMILES: CC(C)n1nc(-c2cnc(C(F)(F)F)nc2)nc1[C@H]1CC[C@@H](N2CCO[C@H](C)C2)C1 ; CC(C)n1nc(-c2cnc(C(F)(F)F)nc2)nc1[C@H]1CC[C@H](N2CCO[C@H](C)C2)C1]

The title compound was synthesized following a procedure similar to compound 68 using 2-(trifluoromethyl)pyrimidine-5-carboximidamide and (S)-3-oxocyclopentanecarboxylic acid in step 1. Purification of the crude mixture by silica flash chromatography (0-5% methanol in dichloromethane) to provide the title compound (100.0 mg, 80% yield) as a mixture of diastereomers. The diastereomers was separated using chiral SFC (Daicel Chiralpak AS (250 mm*30 mm, 10 μm); 0.1% NH₄OH; EtOH, 25/25, 70 ml/min) to provide compound 134 (second peak on SFC, 43.96 mg, 44% yield) and compound 135 (first peak on SFC, 4.53 mg, 4.5% yield). LCMS (ESI), [M+H]⁺=425.2. The relative stereochemistry was determined by 2D-NMR.

Compound 134: ¹H NMR (400 MHz, CD₃OD) δ9.51 (s, 2H), 4.81-4.76 (m, 1H), 3.87-3.85 (m, 1H), 3.71-3.68 (m, 1H), 3.65-3.63 (m, 1H), 3.54-3.45 (m, 1H), 2.96-2.90 (m, 2H), 2.88-2.79 (m, 1H), 2.42-2.36 (m, 1H), 2.25-2.22 (m, 1H), 2.19-2.14 (m, 1H), 2.10-2.04 (m, 2H), 1.95-1.92 (m, 2H), 1.88-1.81 (m, 1H), 1.54 (dd, J=3.2, 6.8 Hz, 6H), 1.14 (d, J=6.4 Hz, 3H).

Compound 135: ¹H NMR (400 MHz, CD₃OD) δ9.51 (s, 2H), 4.80-4.79 (m, 1H), 3.87-3.85 (m, 1H), 3.70-3.67 (m, 1H), 3.66-3.56 (m, 2H), 2.98-2.95 (m, 2H), 2.89-2.87 (m, 1H), 2.26-2.17 (m, 4H), 2.12-2.04 (m, 1H), 1.99-1.93 (m, 1H), 1.91-1.85 (m, 1H), 1.71-1.60 (m, 1H), 1.54 (dd, J=2.4, 6.8 Hz, 6H), 1.16 (d, J=6.4 Hz, 3H).

### Example BK: (R)-2-Ethyl-4-((1R,3S)-3-(3-(5-fluoro-6-(trifluoromethyl)pyridin-3-yl)-1-isopropyl-1H-1,2,4-triazol-5-yl)cyclopentyl)morpholine (Compound 146)

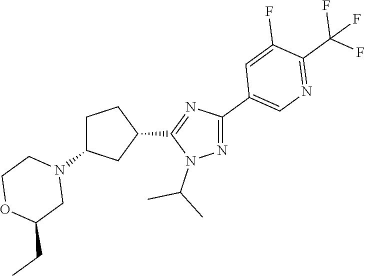 [SMILES: CC[C@@H]1CN([C@@H]2CC[C@H](c3nc(-c4cnc(C(F)(F)F)c(F)c4)nn3C(C)C)C2)CCO1]

The title compound was synthesized following a procedure similar to compound 68 using 5-fluoro-6-(trifluoromethyl)nicotinimidamide and (S)-3-oxocyclopentane carboxylic acid in step 1. Purification of the crude mixture by silica flash chromatography (0-50% ethyl acetate in petroleum ether) to provide title compound 146 (25.0 mg, 45% yield). LCMS (ESI), [M+H]⁺=456.2. The relative stereochemistry was determined by 2D-NMR.

Compound 146: ¹H NMR (400 MHz, CD₃OD) δ 9.14 (s, 1H), 8.36 (d, J=11.2 Hz, 1H), 4.78-4.73 (m, 1H), 3.89-3.86 (m, 1H), 3.69-3.64 (m, 1H), 3.50-3.40 (m, 2H), 2.92 (t, J=11.6 Hz, 2H), 2.86-2.78 (m, 1H), 2.41-2.35 (m, 1H), 2.25-2.13 (m, 2H), 2.11-2.04 (m, 2H), 2.01-1.89 (m, 2H), 1.88-1.82 (m, 1H), 1.53 (dd, J=3.6, 6.4 Hz, 6H), 1.50-1.44 (m, 2H), 0.95 (t, J=7.6 Hz, 3H).

### Example BL: (S)-2-Ethyl-4-((1R,3S)-3-(3-(5-fluoro-6-(trifluoromethyl)pyridin-3-yl)-1-isopropyl-1H-1,2,4-triazol-5-yl)cyclopentyl)morpholine (Compound 147)

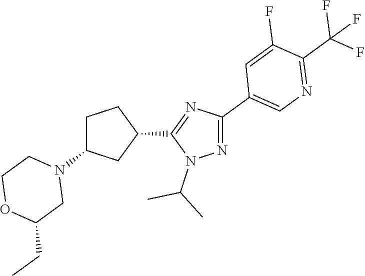 [SMILES: CC[C@H]1CN([C@@H]2CC[C@H](c3nc(-c4cnc(C(F)(F)F)c(F)c4)nn3C(C)C)C2)CCO1]

The title compound was synthesized following a procedure similar to compound 68 using 5-fluoro-6-(trifluoromethyl)nicotinimidamide and (S)-3-oxocyclopentane carboxylic acid in step 1. Purification of the crude mixture by silica flash chromatography (0-50% ethyl acetate in petroleum ether) to provide title compound 147 (23.9 mg, 13.5% yield). LCMS (ESI), [M+H]⁺=456.2. The relative stereochemistry was determined by 2D-NMR.

Compound 147: ¹H NMR (400 MHz, CD₃OD) δ 9.14 (s, 1H), 8.36 (d, J=11.2 Hz, 1H), 4.80-4.73 (m, 1H), 3.90-3.86 (m, 1H), 3.69-3.63 (m, 1H), 3.53-3.41 (m, 2H), 3.00-2.87 (m, 2H), 2.84-2.79 (m, 1H), 2.44-2.35 (m, 1H), 2.29-2.22 (m, 1H), 2.21-2.12 (m, 1H), 2.11-2.01 (m, 2H), 1.99-1.92 (m, 2H), 1.89-1.82 (m, 1H), 1.53 (dd, J=2.4, 6.4 Hz, 6H), 1.51-1.45 (m, 2H), 0.96 (t, J=7.2 Hz, 3H).

### Example BM: (R)-4-((1S,3R)-3-(1-Isopropyl-3-(2-(trifluoromethyl)pyrimidin-5-yl)-1H-1,2,4-triazol-5-yl)cyclopentyl)-2-methylmorpholine and (R)-4-((1R,3R)-3-(1-Isopropyl-3-(2-(trifluoromethyl)pyrimidin-5-yl)-1H-1,2,4-triazol-5-yl)cyclopentyl)-2-methylmorpholine (Compounds 148 & 149)

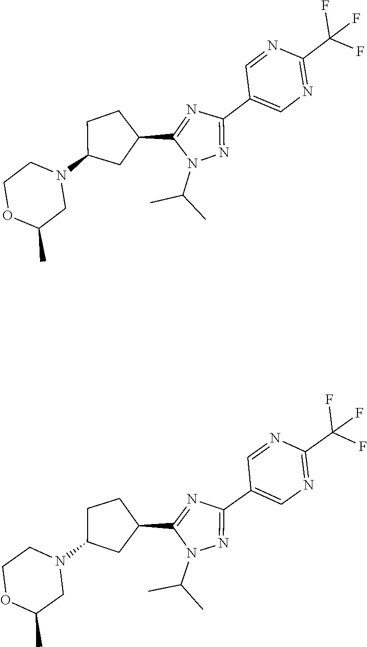 [SMILES: CC(C)n1nc(-c2cnc(C(F)(F)F)nc2)nc1[C@@H]1CC[C@H](N2CCO[C@H](C)C2)C1 ; CC(C)n1nc(-c2cnc(C(F)(F)F)nc2)nc1[C@@H]1CC[C@@H](N2CCO[C@H](C)C2)C1]

The title compound was synthesized following a procedure similar to compound 68 using 2-(trifluoromethyl)pyrimidine-5-carboximidamide and (R)-3-oxocyclopentanecarboxylic acid in step 1. Purification of the crude mixture by reverse phase chromatography (Boston Prime C18 150*30 mm*5 um/water (NH₃H₂O+NH₄HCO₃)-CAN, 35%-65%) to provide compound 148 (second peak on SFC, 54.48 mg, 43.1% yield) and compound 149 (first peak on SFC, 7.18 mg, 4.7% yield). LCMS (ESI), [M+H]⁺=425.2. The relative stereochemistry was determined by 2D-NMR.

Compound 148: ¹H NMR (400 MHz, CD₃OD) δ9.51 (s, 2H), 4.81-4.73 (m, 1H), 3.87-3.84 (m, 1H), 3.71-3.62 (m, 2H), 3.54-3.45 (m, 1H), 2.99-2.96 (m, 1H), 2.90-2.79 (m, 2H), 2.42-2.36 (m, 1H), 2.27-2.14 (m, 2H), 2.10-2.02 (m, 2H), 1.99-1.81 (m, 3H), 1.54 (dd, J=2.4, 6.4 Hz, 6H), 1.15 (d, J=6.4 Hz, 3H).

Compound 149: ¹H NMR (400 MHz, CD₃OD) δ 9.51 (s, 2H), 4.80-4.77 (m, 1H), 3.90-3.85 (m, 1H), 3.71-3.59 (m, 3H), 3.03-2.91 (m, 1H), 2.28-2.18 (m, 4H), 2.12-2.05 (m, 1H), 1.99-1.95 (m, 1H), 1.92-1.88 (m, 2H), 1.72-1.62 (m, 1H), 1.54 (dd, J=3.2, 6.8 Hz, 6H), 1.14 (d, J=6.4 Hz, 3H)

### Example BN: (S)-4-((1S,3R)-3-(1-Isopropyl-3-(2-(trifluoromethyl)pyrimidin-5-yl)-1H-1,2,4-triazol-5-yl)cyclopentyl)-2-methylmorpholine and (S)-4-((1R,3R)-3-(1-Isopropyl-3-(2-(trifluoromethyl)pyrimidin-5-yl)-1H-1,2,4-triazol-5-yl)cyclopentyl)-2-methylmorpholine (Compounds 150 & 151)

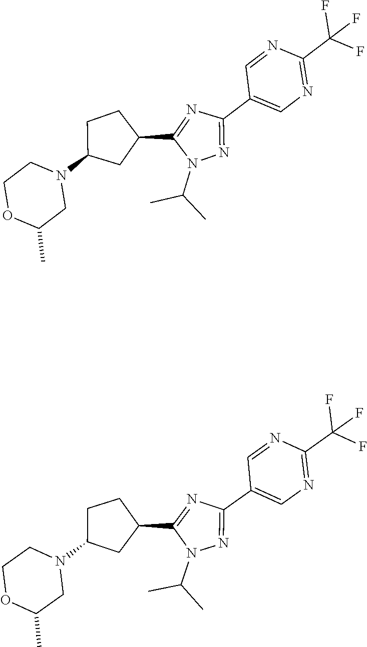 [SMILES: CC(C)n1nc(-c2cnc(C(F)(F)F)nc2)nc1[C@@H]1CC[C@H](N2CCO[C@@H](C)C2)C1 ; CC(C)n1nc(-c2cnc(C(F)(F)F)nc2)nc1[C@@H]1CC[C@@H](N2CCO[C@@H](C)C2)C1]

The title compound was synthesized following a procedure similar to compound 68 using 2-(trifluoromethyl)pyrimidine-5-carboximidamide and (R)-3-oxocyclopentanecarboxylic acid in step 1. Purification of the crude mixture by reverse phase chromatography (Boston Prime C18 150*30 mm*5 um/water (NH₃H₂O+NH₄HCO₃)-CAN, 35%-65%) to provide compound 150 (second peak on SFC, 68.53 mg, 44.7% yield) and compound 151 (first peak on SFC, 16.03 mg, 10.4% yield). LCMS (ESI), [M+H]⁺=425.1. The relative stereochemistry was determined by 2D-NMR.

Compound 150: ¹H NMR (400 MHz, CD₃OD) δ 9.51 (s, 2H), 4.90-4.73 (m, 1H), 3.87-3.84 (m, 1H), 3.71-3.62 (m, 2H), 3.54-3.45 (m, 1H), 2.97-2.91 (m, 2H), 2.84-2.82 (m, 1H), 2.43-2.36 (m, 1H), 2.25-2.14 (m, 2H), 2.10-2.02 (m, 2H), 1.99-1.90 (m, 2H), 1.88-1.82 (m, 1H), 1.54 (dd, J=3.2, 6.8 Hz, 6H), 1.14 (d, J=6.4 Hz, 3H).

Compound 151: ¹H NMR (400 MHz, CD₃OD) δ 9.51 (s, 2H), 4.86-4.77 (m, 1H), 3.88-3.84 (m, 1H), 3.71-3.58 (m, 3H), 2.98-2.95 (m, 2H), 2.88 (dd, J=1.6, 11.6 Hz, 1H), 2.28-2.16 (m, 4H), 2.12-2.04 (m, 1H), 1.99-1.93 (m, 1H), 1.89 (dd, J=10.8, 11.2 Hz, 1H), 1.71-1.63 (m, 1H), 1.54 (dd, J=2.4, 6.8 Hz, 6H), 1.16 (d, J=6.4 Hz, 3H).

### Example BO: (R)-2-Ethyl-4-((1S,3R)-3-(1-isopropyl-3-(2-(trifluoromethyl) pyrimidin-5-yl)-1H-1,2,4-triazol-5-yl)cyclopentyl)morpholine and (R)-2-Ethyl-4-((1R,3R)-3-(1-isopropyl-3-(2-(trifluoromethyl)pyrimidin-5-yl)-1H-1,2,4-triazol-5-yl)cyclopentyl) morpholine (Compounds 152 & 153)

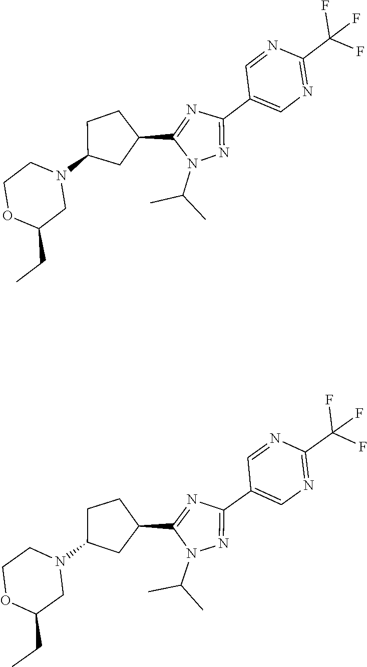 [SMILES: CC[C@@H]1CN([C@H]2CC[C@@H](c3nc(-c4cnc(C(F)(F)F)nc4)nn3C(C)C)C2)CCO1 ; CC[C@@H]1CN([C@@H]2CC[C@@H](c3nc(-c4cnc(C(F)(F)F)nc4)nn3C(C)C)C2)CCO1]

The title compound was synthesized following a procedure similar to compound 68 using 2-(trifluoromethyl)pyrimidine-5-carboximidamide and (R)-3-oxocyclopentanecarboxylic acid in step 1. Purification of the crude mixture by reverse phase chromatography (Boston Prime C18 150*30 mm*5 um/water (NH₃H₂O+NH₄HCO₃)-CAN, 30%-70%) to provide compound 152 (second peak on SFC, 48.92 mg, 37.5% yield) and 153 (first peak on SFC, 5.59 mg, 3.2% yield). LCMS (ESI), [M+H]⁺=439.2. The relative stereochemistry was determined by 2D-NMR.

Compound 152: H NMR (400 MHz, CD₃OD) δ 9.51 (s, 2H), 4.81-4.75 (m, 1H), 3.88-3.84 (m, 1H), 3.69-3.62 (m, 1H), 3.54-3.47 (m, 1H), 3.45-3.40 (m, 1H), 2.98-2.95 (m, 1H), 2.89-2.86 (m, 1H), 2.84-2.78 (m, 1H), 2.42-2.36 (m, 1H), 2.27-2.20 (m, 1H), 2.19-2.14 (m, 1H), 2.11-2.03 (m, 2H), 2.01-1.96 (m, 1H), 1.93-1.80 (m, 2H), 1.54 (dd, J=2.4, 6.4 Hz, 6H), 1.50-1.42 (m, 2H), 0.96 (t, J=7.6 Hz, 3H).

Compound 153: H NMR (400 MHz, CD₃OD) δ 9.51 (s, 2H), 4.80-4.77 (m, 1H), 3.92-3.89 (m, 1H), 3.69-3.57 (m, 2H), 3.43-3.42 (m, 1H), 2.99-2.90 (m, 3H), 2.30-2.16 (m, 4H), 2.09-2.04 (m, 1H), 1.97-1.88 (m, 2H), 1.71-1.61 (m, 1H), 1.54 (dd, J=3.6, 6.4 Hz, 6H), 1.50-1.46 (m, 2H), 0.97-0.94 (m, 3H).

### Example BP: (S)-2-Ethyl-4-((1S,3R)-3-(1-isopropyl-3-(2-(trifluoromethyl) pyrimidin-5-yl)-1H-1,2,4-triazol-5-yl)cyclopentyl)morpholine and (S)-2-Ethyl-4-((1R,3R)-3-(1-isopropyl-3-(2-(trifluoromethyl)pyrimidin-5-yl)-1H-1,2,4-triazol-5-yl)cyclopentyl) morpholine (Compounds 154 & 155)

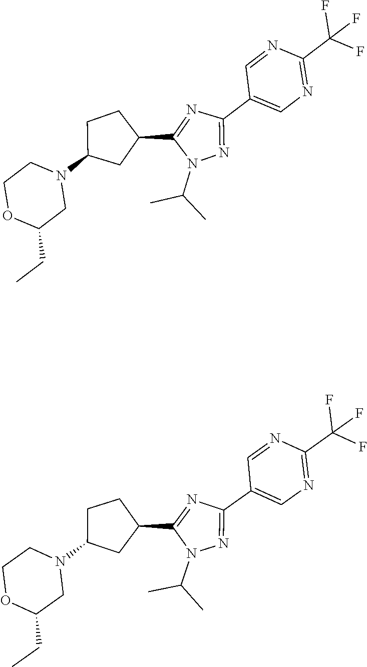 [SMILES: CC[C@H]1CN([C@H]2CC[C@@H](c3nc(-c4cnc(C(F)(F)F)nc4)nn3C(C)C)C2)CCO1 ; CC[C@H]1CN([C@@H]2CC[C@@H](c3nc(-c4cnc(C(F)(F)F)nc4)nn3C(C)C)C2)CCO1]

The title compound was synthesized following a procedure similar to compound 68 using 2-(trifluoromethyl)pyrimidine-5-carboximidamide and (R)-3-oxocyclopentane carboxylic acid in step 1. Purification of the crude mixture by reverse phase chromatography (Boston Prime C18 150*30 mm*5 um/water (NH₃H₂O+NH₄HCO₃)-CAN, 30%-70%) to provide compound 154 (second peak on SFC, 43.92 mg, 33.6% yield) and 155 (first peak on SFC, 4.71 mg, 2.9% yield). LCMS (ESI), [M+H]⁺=439.2. The relative stereochemistry was determined by 2D-NMR.

Compound 154: H NMR (400 MHz, CD₃OD) δ9.51 (s, 2H), 4.81-4.75 (m, 1H), 3.88-3.84 (m, 1H), 3.69-3.63 (m, 1H), 3.54-3.39 (m, 2H), 2.95-2.90 (m, 2H), 2.87-2.78 (m, 1H), 2.42-2.36 (m, 1H), 2.25-2.14 (m, 2H), 2.10-2.02 (m, 2H), 1.99-1.90 (m, 2H), 1.88-1.81 (m, 1H), 1.54 (dd, J=3.2, 6.4 Hz, 6H), 1.50-1.44 (m, 2H), 0.95 (t, J=7.6 Hz, 3H).

Compound 155: ¹H NMR (400 MHz, CD₃OD) δ 9.51 (s, 2H), 4.80-4.75 (m, 1H), 3.89-3.86 (m, 1H), 3.69-3.59 (m, 2H), 3.41-3.40 (m, 1H), 2.98-2.95 (m, 2H), 2.90-2.87 (m, 1H), 2.26-2.17 (m, 4H), 2.09-2.04 (m, 1H), 1.96-1.87 (m, 2H), 1.71-1.64 (m, 1H), 1.54 (dd, J=2.4, 6.8 Hz, 6H), 1.52-1.47 (m, 2H), 0.97 (t, J=7.6 Hz, 3H).

### Example BQ: (R)-4-((1R,3S)-3-(3-(6-(Difluoromethyl)pyridin-3-yl)-1-isopropyl-1H-1,2,4-triazol-5-yl)cyclopentyl)-2-ethylmorpholine (Compound 156)

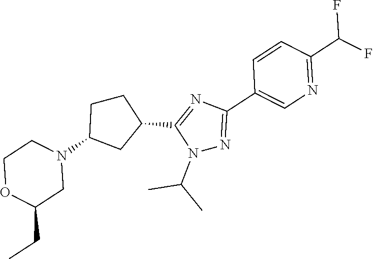 [SMILES: CC[C@@H]1CN([C@@H]2CC[C@H](c3nc(-c4ccc(C(F)F)nc4)nn3C(C)C)C2)CCO1]

The title compound was synthesized following a procedure similar to compound 68 using 6-(difluoromethyl)nicotinimidamide and (S)-3-oxocyclopentanecarboxylic acid in step 1. Purification of the crude mixture by silica flash chromatography (0-10% methanol in dichloromethane) to provide the title compound (60 mg, 0.13 mmol, 70% yield) as a mixture of diasteromers. The mixture of diasteromers (60 mg, 0.13 mmol) was separated using chiral SFC (Daicel Chiralpak AD (250 mm*30 mm, 10 um); 0.1% NH₃H₂O; IPA; 60/60, 60 mL/min) to provide compound 156 (first peak on SFC, 25.39 mg, 41% yield). LCMS (ESI) [M+H]⁺=420.3. The relative stereochemistry was determined by 2D-NMR.

Compound 156: ¹H NMR (400 MHz, CD₃OD) δ 9.25 (s, 1H), 8.56 (d, J=8.4 Hz, 1H), 7.78 (d, J=8.0 Hz, 1H), 6.76 (t, J=15.2 Hz, 1H), 4.76-4.73 (m, 1H), 3.88 (dd, J=2.4, 12.0 Hz, 1H), 3.67-3.65 (m, 1H), 3.55-3.43 (m, 2H), 2.93 (t, J=12.4 Hz, 2H), 2.88-2.80 (m, 1H), 2.43-2.35 (m, 1H), 2.28-2.15 (m, 2H), 2.11-2.00 (m, 2H), 1.99-1.90 (m, 2H), 1.89-1.80 (m, 1H), 1.53 (dd, J=3.6, 6.4 Hz, 6H), 1.48-1.46 (m, 1H), 0.95 (t, J=7.6 Hz, 3H).

### Example BR*: 4-((1S,3R)-3-(3-(6-fluoro-5-(trifluoromethyl)pyridin-3-yl)-1-isopropyl-1H-1,2,4-triazol-5-yl)cyclopentyl)morpholine, 4-((1R,3S)-3-(3-(6-fluoro-5-(trifluoromethyl)pyridin-3-yl)-1-isopropyl-1H-1,2,4-triazol-5-yl)cyclopentyl)morpholine and 4-((1S,3S)-3-(3-(6-fluoro-5-(trifluoromethyl)pyridin-3-yl)-1-isopropyl-1H-1,2,4-triazol-5-yl)cyclopentyl)morpholine (Compounds 157*, 158* & 159*)

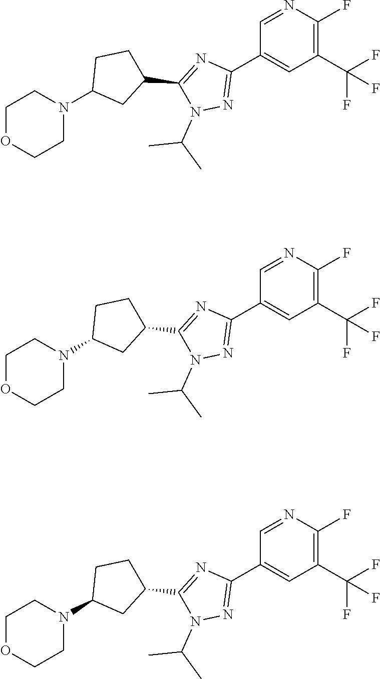

### Step 1: 5-(5-Bromo-1-isopropyl-1H-1,2,4-triazol-3-yl)-3-(trifluoromethyl)pyridin-2-amine

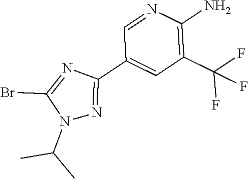 [SMILES: CC(C)n1nc(-c2cnc(N)c(C(F)(F)F)c2)nc1Br]

A suspension of 3,5-dibromo-1-isopropyl-1,2,4-triazole (2 g, 7.44 mmol), 5-(4,4,5,5-tetramethyl-1,3,2-dioxaborolan-2-yl)-3-(trifluoromethyl)pyridin-2-amine (1.9 g, 6.69 mmol), potassium carbonate (3 g, 22.31 mmol) and 1,1′-bis(diphenylphosphino)ferrocene palladium dichloride (540 mg, 0.74 mmol) in 1,4-dioxane (20 mL)) and water (4 mL) was degassed and purged with N₂ for three times, then the reaction mixture was stirred at 90° C. under N₂ for 1 hour. A black suspension was formed. The reaction mixture was concentrated under vacuum. The residue was purified by silica flash chromatography (0-30% ethyl acetate in petroleum ether) to provide the title compound (850 mg, 32.6% yield). LCMS (ESI) [M+H]⁺=350.2.

### Step 2: 3-(3-(6-Amino-5-(trifluoromethyl)pyridin-3-yl)-1-isopropyl-1H-1,2,4-triazol-5-yl)cyclopent-2-enone

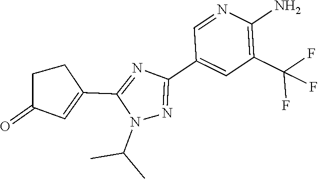 [SMILES: CC(C)n1nc(-c2cnc(N)c(C(F)(F)F)c2)nc1C1=CC(=O)CC1]

A suspension of 1,1′-bis(diphenylphosphino)ferrocene palladium dichloride (177 mg, 0.24 mmol), potassium carbonate (1 g, 7.27 mmol), 5-(5-bromo-1-isopropyl-1,2,4-triazol-3-yl)-3-(trifluoromethyl) pyridin-2-amine (850 mg, 2.43 mmol) and 3-(4,4,5,5-tetramethyl-1,3,2-dioxaborolan-2-yl)cyclopent-2-en-1-one (757 mg, 3.64 mmol) in 1,4-dioxane (10 mL)) and water (2 mL) was degassed and purged with N₂ for three times, then the reaction mixture was stirred at 90° C. under N₂ for 4 hours. The reaction mixture was concentrated under vacuum. The residue was purified by silica flash chromatography (0-30% ethyl acetate in petroleum ether) to provide the title compound (500 mg, 58.6% yield). LCMS (ESI) [M+H]⁺=352.1.

### Step 3: 3-(3-(6-Amino-5-(trifluoromethyl)pyridin-3-yl)-1-isopropyl-1H-1,2,4-triazol-5-yl)cyclopentanone

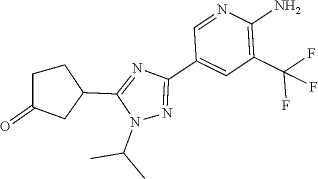 [SMILES: CC(C)n1nc(-c2cnc(N)c(C(F)(F)F)c2)nc1C1CCC(=O)C1]

To a solution of 3-[5-[6-amino-5-(trifluoromethyl)-3-pyridyl]-2-isopropyl-1,2,4-triazol-3-yl]cyclopent-2-en-1-one (200 mg, 0.57 mmol) in ethanol (3 mL) was added 10% palladium in carbon (242 mg, 0.11 mmol) at 25° C. and stirred at 20° C. under H₂ (15 psi) for 12 hours. The reaction mixture was filtered, and the filtered cake was washed with methyl alcohol (10 mL×2). The combined organic layers were concentrated under vacuum provide the title compounds (200 mg, 99.4% yield). LCMS (ESI) [M+H]⁺=354.1.

### Step 4: 5-(1-Isopropyl-5-(3-morpholinocyclopentyl)-1H-1,2,4-triazol-3-yl)-3-(trifluoromethyl) pyridin-2-amine

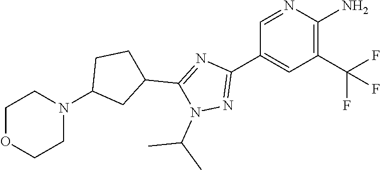 [SMILES: CC(C)n1nc(-c2cnc(N)c(C(F)(F)F)c2)nc1C1CCC(N2CCOCC2)C1]

To a solution of 3-[5-[6-amino-5-(trifluoromethyl)-3-pyridyl]-2-isopropyl-1,2,4-triazol-3-yl]cyclopentanone (670 mg, 1.9 mmol) in acetonitrile (10 mL) was added morpholine (1.6 mL, 18.96 mmol), acetic acid (0.6 mL, 11.3 mmol) and NaBH(OAc)₃ (1607 mg, 7.58 mmol). The mixture was stirred at 60° C. for 2 hours. The reaction mixture was diluted with ethyl acetate (60 mL) and the resulting mixture was washed with brine (30 mL×3). The organic layer was dried over Na₂SO₄, filtered and concentrated in vacuo. The residue was purified by silica flash chromatography (0-10% methanol in dichloromethane) to provide the title compound (570 mg, 70.8% yield). LCMS (ESI) [M+H]⁺=425.2.

### Step 5: 5-(1-Isopropyl-5-((1R,3S)-3-morpholinocyclopentyl)-1H-1,2,4-triazol-3-yl)-3-(trifluoromethyl)pyridin-2-amine, 5-(1-isopropyl-5-((1S,3S)-3-morpholinocyclopentyl)-1H-1,2,4-triazol-3-yl)-3-(trifluoromethyl)pyridin-2-amine

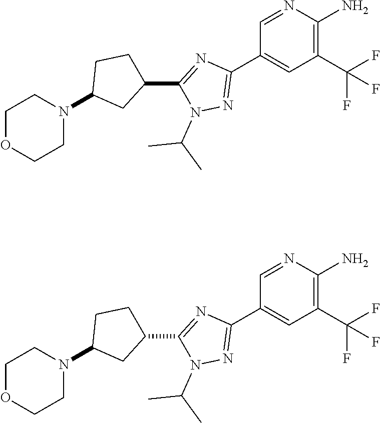

5-[1-isopropyl-5-(3-morpholinocyclopentyl)-1,2,4-triazol-3-yl]-3-(trifluoromethyl)pyridin-2-amine (570 mg, 1.34 mmol) was separated using chiral SFC (SFC-14; DAICEL CHIRALPAK AD-H (250 mm*30 mm, 5 um)); 0.1% NH₃H₂O+ETOH, 30/30; 60 mL/min) to provide the title compound-1 (first peak on SFC (mixture of diastereomers, 175 mg, 30.7% yield) and title compound-2 (second peak on SFC, 170 mg, 29.8% yield).

### Step 4: 4-((1S,3R)-3-(3-(6-fluoro-5-(trifluoromethyl)pyridin-3-yl)-1-isopropyl-1H-1,2,4-triazol-5-yl)cyclopentyl)morpholine and 5-(1-isopropyl-5-((1S,3R)-3-morpholinocyclopentyl)-1H-1,2,4-triazol-3-yl)-3-(trifluoromethyl)pyridin-2-amine (Compounds 157* & 158*)

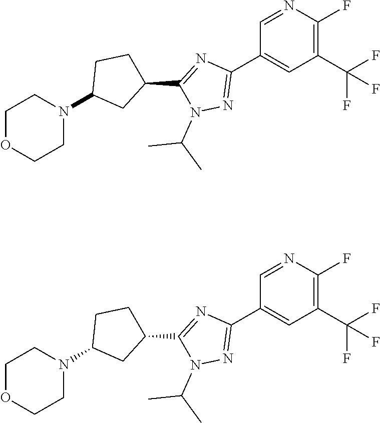 [SMILES: CC(C)n1nc(-c2cnc(F)c(C(F)(F)F)c2)nc1[C@@H]1CC[C@H](N2CCOCC2)C1 ; CC(C)n1nc(-c2cnc(F)c(C(F)(F)F)c2)nc1[C@H]1CC[C@@H](N2CCOCC2)C1]

To a mixture of 5-[1-isopropyl-5-[rac-(1R,5S)-3-morpholino-6-bicyclo[3.1.0]hexanyl]pyrazol-3-yl]-3-(trifluoromethyl)pyridin-2-amine (140 mg, 0.33 mmol) in pyridine (1 mL) was added hydrogen fluoride-pyridine (2 ml, 0.52 mmol) at 0° C., followed by sodium nitrite (14 mg, 0.21 mmol). The mixture was stirred at 0° C. for 60 mins, then stirred at 25° C. for 1 hour. The reaction mixture was quenched by sodium bicarbonate (10 mL) and extracted with dichloromethane (20 mL×3). The combined organic layer was dried over sodium sulfate, filter and concentrated. The residue was purified by reverse phase chromatography (water (NH₃H₂O+NH₄HCO₃)-CAN, 25%-55%). The mixture of diastereomers (70 mg) was separated using chiral SFC (Daicel Chiralpak AD (250 mm*30 mm, 10 um)); 0.1% NH₄OH+MeOH; 40/40; 60 mL/min) to provide compound 157* (first peak on SFC, 11.5 mg, 16% yield) and compound 158* (second peak on SFC, 49 mg, 70% yield). LCMS (ESI) [M+H]⁺=428.3. The relative stereochemistry was arbitrarily assigned.

Compound 157*: ¹H NMR (400 MHz, CD₃OD) δ 8.79 (d, J=1.6 Hz, 1H), 8.34 (d, J=1.6 Hz, 1H), 4.73-4.66 (m, 1H), 3.72 (t, J=4.8 Hz, 4H), 3.50-3.38 (m, 1H), 2.89-2.75 (m, 1H), 2.59-2.49 (m, 4H), 2.39-2.31 (m, 1H), 2.21-1.80 (m, 5H), 1.52-1.50 (m, 6H).

Compound 158*: H NMR (400 MHz, CD₃OD) δ 9.06 (s, 1H), 8.84-8.65 (m, 1H), 4.78-4.72 (m, 1H), 3.74 (t, J=4.4 Hz, 4H), 3.54-3.44 (m, 1H), 2.89 (s, 1H), 2.66 (s, 4H), 2.48-2.35 (m, 1H), 2.22-1.86 (m, 5H), 1.59-1.50 (m, 6H).

### Step 6: 4-((1S,3S)-3-(3-(6-Fluoro-5-(trifluoromethyl)pyridin-3-yl)-1-isopropyl-1H-1,2,4-triazol-5-yl)cyclopentyl)morpholine (Compound 159*)

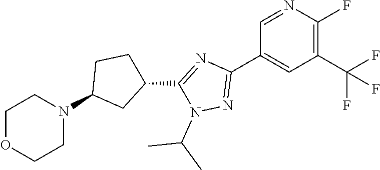 [SMILES: CC(C)n1nc(-c2cnc(F)c(C(F)(F)F)c2)nc1[C@H]1CC[C@H](N2CCOCC2)C1]

To a mixture of 5-[1-isopropyl-5-(3-morpholinocyclopentyl)-1,2,4-triazol-3-yl]-3-(trifluoromethyl) pyridin-2-amine (50 mg, 0.12 mmol) in pyridine (1 mL) was added hydrogen fluoride-pyridine (2 mL, 0.12 mmol) at 0° C., followed by sodium nitrite (10 mg, 0.15 mmol). The mixture was stirred at 0° C. for 60 mins, then stirred at 25° C. for 1 hour. The reaction mixture was quenched by sodium bicarbonate (10 mL), and the resulting solution was extracted with dichloromethane (20 mL×3). The combined organics were dried over sodium sulfate, filtered and concentrated. The residue was purified by reverse phase chromatography (water (NH₃H₂O+NH₄HCO₃)-CAN, 25%-55%) to provide compound 159* (20 mg, 38.1% yield). LCMS (ESI) [M+H]⁺=428.2. The relative stereochemistry was arbitrarily assigned.

Compound 159*: ¹H NMR (400 MHz, CD₃OD) δ 9.06 (s, 1H), 8.75 (d, J=7.2 Hz, 1H), 4.79-4.72 (m, 1H), 3.78 (t, J=4.4 Hz, 4H), 3.58-3.48 (m, 1H), 3.12-3.01 (m, 1H), 2.82-2.78 (m, 4H), 2.51-2.38 (m, 1H), 2.19-1.89 (m, 5H), 1.55-1.50 (m, 6H).

### Example BS: (S)-4-((1R,3S)-3-(3-(3-Fluoro-4-(trifluoromethyl)phenyl)-1-isopropyl-1H-1,2,4-triazol-5-yl)cyclopentyl)-2-(fluoromethyl)morpholine (Compound 160)

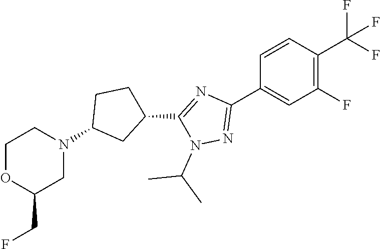 [SMILES: CC(C)n1nc(-c2ccc(C(F)(F)F)c(F)c2)nc1[C@H]1CC[C@@H](N2CCO[C@H](CF)C2)C1]

The title compound was synthesized following a procedure similar to compound 130* using 3-fluoro-4-(trifluoromethyl)benzimidamide and (S)-3-oxocyclopentanecarboxylic acid in step 1. Purification of the crude mixture by prep-TLC (10% methanol in dichloromethane) to provide compound 160 (23.67 mg, 25.9% yield). LCMS (ESI), [M+H]⁺=459.1. The relative stereochemistry was determined by 2D-NMR.

Compound 160: H NMR (400 MHz, CD₃OD) δ8.01 (d, J=8.4 Hz, 1H), 7.93 (d, J=11.6 Hz, 1H), 7.75 (t, J=8.0 Hz, 1H), 4.77-4.71 (m, 1H), 4.46 (d, J=4.0 Hz, 1H), 4.35 (d, J=4.0 Hz, 1H), 3.92-3.86 (m, 1H), 3.78-3.67 (m, 2H), 3.51-3.43 (m, 1H), 2.99-2.90 (m, 2H), 2.88-2.81 (m, 1H), 2.41-2.34 (m, 1H), 2.28-2.22 (m, 1H), 2.20-2.12 (m, 2H), 2.09-2.04 (m, 2H), 2.01-1.93 (m, 1H), 1.89-1.82 (m, 1H), 1.53 (dd, J=2.8, 6.8 Hz, 6H).

### Example BT: (S)-2-(Fluoromethyl)-4-((1R,3S)-3-(1-isopropyl-3-(2-(trifluoromethyl) pyrimidin-5-yl)-1H-1,2,4-triazol-5-yl)cyclopentyl)morpholine (Compound 161)

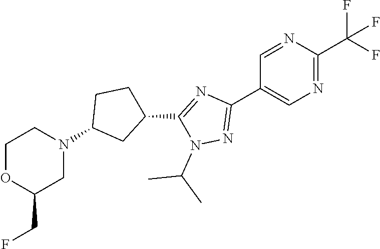 [SMILES: CC(C)n1nc(-c2cnc(C(F)(F)F)nc2)nc1[C@H]1CC[C@@H](N2CCO[C@H](CF)C2)C1]

The title compound was synthesized following a procedure similar to compound 130* using 2-(trifluoromethyl)pyrimidine-5-carboximidamide and (S)-3-oxocyclopentanecarboxylic acid in step 1. Purification of the crude mixture by prep-TLC [(petroleum ether/(ethyl acetate/ethanol=3/1)]=1/1) to provide the title compound 161 (25.23 mg, 58.4% yield). LCMS (ESI), [M+H]⁺=443.2. The relative stereochemistry was determined by 2D-NMR.

Compound 161: ¹H NMR (400 MHz, CD₃OD) δ9.51 (s, 2H), 4.81-4.75 (m, 1H), 4.46 (d, J=4.4 Hz, 1H), 4.35 (d, J=4.4 Hz, 1H), 3.96-3.93 (m, 1H), 3.73-3.64 (m, 1H), 3.55-3.42 (m, 2H), 2.99-2.89 (m, 2H), 2.87-2.81 (m, 1H), 2.43-2.36 (m, 1H), 2.28-2.21 (m, 1H), 2.19-1.96 (m, 5H), 1.89-1.82 (m, 1H), 1.54 (dd, J=3.2, 6.4 Hz, 6H).

### Example BU: (R)-4-((1R,3s,5S,6R)-6-(1-Isopropyl-3-(5-(trifluoromethyl)pyridin-3-yl)-1H-1,2,4-triazol-5-yl)bicyclo[3.1.0]hexan-3-yl)-2-methylmorpholine and (R)-4-((1R,3r,5S,6R)-6-(1-Isopropyl-3-(5-(trifluoromethyl)pyridin-3-yl)-1H-1,2,4-triazol-5-yl)bicyclo[3.1.0]hexan-3-yl)-2-methylmorpholine (Compounds 162 & 163)

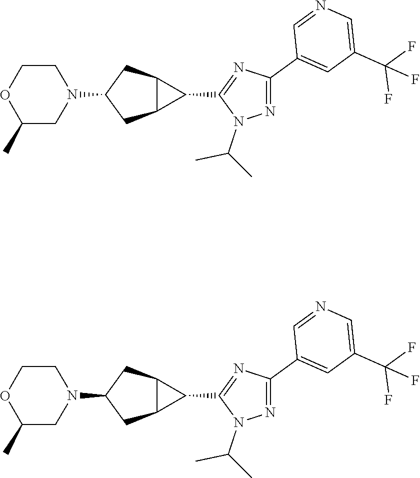 [SMILES: CC(C)n1nc(-c2cncc(C(F)(F)F)c2)nc1[C@H]1[C@@H]2C[C@@H](N3CCO[C@H](C)C3)C[C@@H]21 ; CC(C)n1nc(-c2cncc(C(F)(F)F)c2)nc1[C@H]1[C@@H]2C[C@H](N3CCO[C@H](C)C3)C[C@@H]21]

The title compound was synthesized following a procedure similar to compound 51 using (1R,5S,6r)-6-(1-isopropyl-3-(5-(trifluoromethyl)pyridin-3-yl)-1H-1,2,4-triazol-5-yl)bicyclo[3.1.0]hexan-3-one and (2R)-2-methylmorpholine in final step. Purification of the crude mixture by reverse phase chromatography (water (NH₃H₂O+NH₄HCO₃)-CAN, 20%-50%) to provide title compound 162 (first peak on HPLC (basic), 20.1 mg, 22.9% yield) and the title compound 163 (second peak on HPLC (basic), 27.1 mg, 30.8% yield). LCMS (ESI) [M+H]⁺=436.1. The relative stereochemistry was determined by 2D-NMR.

Compound 162: ¹H NMR (400 MHz, CD₃OD) δ 9.37 (d, J=1.6 Hz, 1H), 8.87 (d, J=1.2 Hz, 1H), 8.59 (s, 1H), 4.87-4.85 (m, 1H), 3.92-3.80 (m, 1H), 3.71-3.56 (m, 2H), 2.91-2.76 (m, 2H), 2.61-2.46 (m, 1H), 2.37-2.25 (m, 2H), 2.17-2.06 (m, 3H), 2.03-1.99 (m, 1H), 1.90-1.78 (m, 3H), 1.55 (d, J=6.4 Hz, 6H), 1.13 (d, J=6.4 Hz, 3H)

Compound 163: ¹H NMR (400 MHz, CD₃OD) δ 9.37 (d, J=1.6 Hz, 1H), 8.88 (d, J=1.2 Hz, 1H), 8.59 (s, 1H), 4.87-4.80 (m, 1H), 3.93-3.81 (m, 1H), 3.68-3.56 (m, 2H), 3.02-2.81 (m, 3H), 2.41-2.30 (m, 2H), 2.17 (t, J=3.2 Hz, 1H), 2.10-1.99 (m, 3H), 1.83-1.70 (m, 3H), 1.58 (d, J=6.4 Hz, 6H), 1.14 (d, J=6.4 Hz, 3H).

### Example BV: (R)-4-((1R,3R,5S,6R)-6-(1-Isopropyl-3-((1R,4R)-4-(trifluoromethyl) cyclohexyl)-1H-1,2,4-triazol-5-yl)bicyclo[3.1.0]hexan-3-yl)-3-methylmorpholine and (R)-4-((1R,3S,5S,6R)-6-(1-Isopropyl-3-((1R,4R)-4-(trifluoromethyl) cyclohexyl)-1H-1,2,4-triazol-5-yl)bicyclo[3.1.0]hexan-3-yl)-3-methylmorpholine and (R)-4-((1R,3R,5S,6R)-6-(1-Isopropyl-3-((1s,4S)-4-(trifluoromethyl)cyclohexyl)-1H-1,2,4-triazol-5-yl)bicyclo[3.1.0]hexan-3-yl)-3-methylmorpholine and (R)-4-((1R,3S,5S,6R)-6-(1-Isopropyl-3-((1s,4S)-4-(trifluoromethyl) cyclohexyl)-1H-1,2,4-triazol-5-yl)bicyclo[3.1.0]hexan-3-yl)-3-methylmorpholine (Compounds 164* & 165* & 166* & 167*)

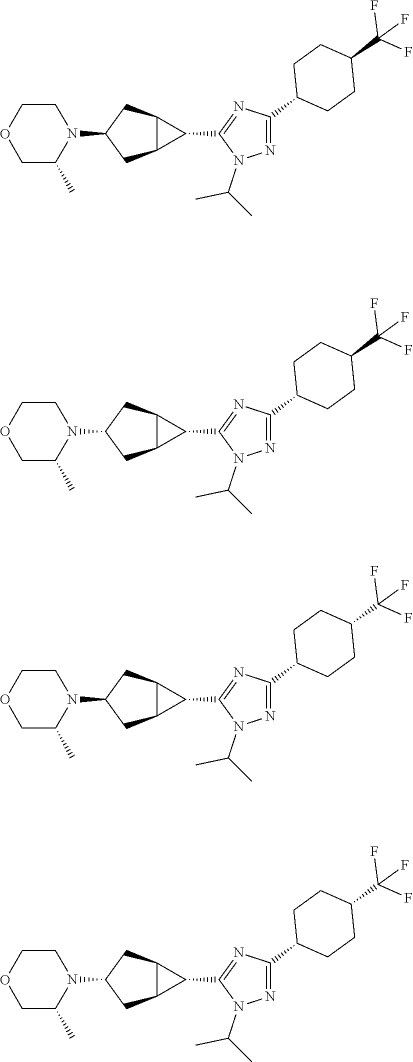 [SMILES: CC(C)n1nc([C@H]2CC[C@H](C(F)(F)F)CC2)nc1[C@H]1[C@@H]2C[C@H](N3CCOC[C@H]3C)C[C@@H]21 ; CC(C)n1nc([C@H]2CC[C@H](C(F)(F)F)CC2)nc1[C@H]1[C@@H]2C[C@@H](N3CCOC[C@H]3C)C[C@@H]21 ; CC(C)n1nc([C@H]2CC[C@@H](C(F)(F)F)CC2)nc1[C@H]1[C@@H]2C[C@H](N3CCOC[C@H]3C)C[C@@H]21 ; CC(C)n1nc([C@H]2CC[C@@H](C(F)(F)F)CC2)nc1[C@H]1[C@@H]2C[C@@H](N3CCOC[C@H]3C)C[C@@H]21]

### Step 1: 5-((1R,5S,6r)-3-((tert-Butyldiphenylsilyl)oxy)bicyclo[3.1.0]hexan-6-yl)-1-isopropyl-3-(4-(trifluoromethyl)cyclohex-1-en-1-yl)-1H-1,2,4-triazole

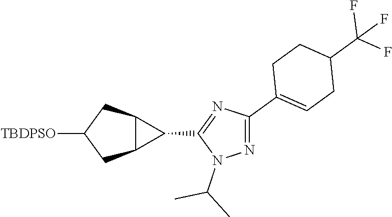

A suspension of tert-butyl-diphenyl-[[rac-(1R, 5S)-6-(5-iodo-2-isopropyl-1,2,4-triazol-3-yl)-3-bicyclo[3.1.0]hexanyl]oxy]silane (synthesized in example BY, compound 51) (720 mg, 1.26 mmol), potassium carbonate (522 mg, 3.78 mmol), 4,4,5,5-tetramethyl-2-[4-(trifluoromethyl)-1-cyclohexen-1-yl]-1,3,2-dioxaborolane (540 mg, 1.96 mmol) and Pd(dppf)Cl₂ (92 mg, 0.13 mmol) in 1,4-dioxane (10 mL)) and water (2 mL) was degassed and purged with N₂ for three times and stirred at 90° C. under N₂ for 4 hours. The reaction mixture was concentrated under vacuum. The residue was purified by silica flash chromatography (0-10% ethyl acetate in petroleum ether) to provide title compound (700 mg, 93.6% yield). LCMS (ESI) [M+H]⁺=594.4.

### Step 2: (Z)-Benzyl (((1R,5S)-3-((tert-butyldiphenylsilyl)oxy)bicyclo[3.1.0]hexane-6-carboxamido)(methylthio)methylene)carbamate

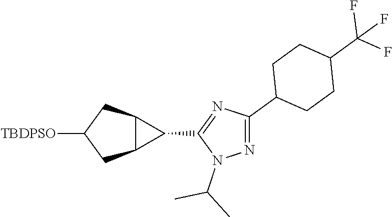

To a solution of tert-butyl-[[(1S,5R)-6-[2-isopropyl-5-[4-(trifluoromethyl)cyclohexen-1-yl]-1,2,4-triazol-3-yl]-3-bicyclo[3.1.0]hexanyl]oxy]-diphenyl-silane (700 mg, 1.18 mmol) in ethanol (10 mL) was added 10% palladium in carbon (250 mg, 0.24 mmol) at 25° C., then the reaction mixture was stirred at 25° C. under H₂ (15 psi) for 12 hours. The reaction mixture was filtered, and the filtered cake was washed with methyl alcohol (10 mL×2). The combined organic layers were concentrated under vacuum to provide the title compound (700 mg, 99.4% yield).

### Step 3: (1R,5S,6r)-6-(1-Isopropyl-3-(4-(trifluoromethyl)cyclohexyl)-1H-1,2,4-triazol-5-yl)bicyclo[3.1.0]hexan-3-ol

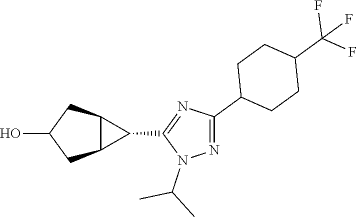

To a stirred solution of tert-butyl-diphenyl-[[rac-(1R,5S)-6-[2-isopropyl-5-[4-(trifluoromethyl) cyclohexyl]-1,2,4-triazol-3-yl]-3-bicyclo[3.1.0]hexanyl]oxy]silane (700 mg, 1.17 mmol) in THF (5 mL) was added triethylamine trihydrofluoride (4 mL, 23.6 mmol). The reaction mixture was stirred at 70° C. for 8 hours. The reaction mixture was adjusted to pH=9 with aq. NaOH (4M). The aqueous was extracted with ethyl acetate (30 mL×3). The combined organic layers were washed with brine (20 mL×2), dried over sodium sulfate, filtered, and concentrated. The residue was purified by silica flash chromatography (0-50% ethyl acetate in petroleum ether) to provide the title compound (280 mg, 66.7% yield). LCMS (ESI) [M+H]⁺=358.3.

### Step 4: (1R,5S,6r)-6-(1-Isopropyl-3-(4-(trifluoromethyl)cyclohexyl)-1H-1,2,4-triazol-5-yl)bicyclo[3.1.0]hexan-3-one

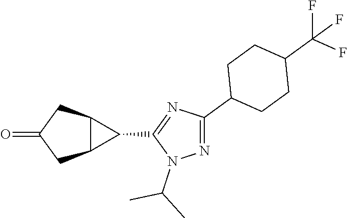 [SMILES: CC(C)n1nc(C2CCC(C(F)(F)F)CC2)nc1[C@H]1[C@@H]2CC(=O)C[C@@H]21]

To a solution of rac-(1R,5S)-6-[2-isopropyl-5-[4-(trifluoromethyl)cyclohexyl]-1,2,4-triazol-3-yl]bicyclo[3.1.0]hexan-3-ol (280 mg, 0.78 mmol) in dichloromethane (5 mL) was added Dess-martin periodinane (665 mg, 1.58 mmol) at 0° C. and stirred at 25° C. for 2 hours. Then the mixture was diluted with dichloromethane (30 mL), washed with Na₂SO₃ solution (20 mL) and NaHCO₃ solution (10 mL). The combined organic layers were dried over Na₂SO₄, filtered, and concentrated. The residue was purified by silica flash chromatography (0-50% ethyl acetate in petroleum ether) to provide the title compound (220 mg, 76% yield). LCMS (ESI) [M+H]⁺=356.2.

### Step 5: (1R,5S,6r)-6-(1-Isopropyl-3-((1R,4R)-4-(trifluoromethyl)cyclohexyl)-1H-1,2,4-triazol-5-yl)bicyclo[3.1.0]hexan-3-one and (1R,5S,6r)-6-(1-isopropyl-3-((1s,4S)-4-(trifluoromethyl)cyclohexyl)-1H-1,2,4-triazol-5-yl)bicyclo[3.1.0]hexan-3-one

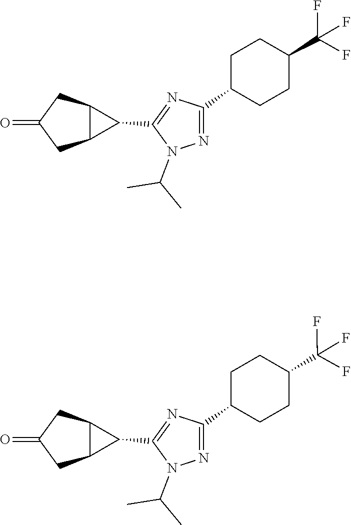 [SMILES: CC(C)n1nc([C@H]2CC[C@H](C(F)(F)F)CC2)nc1[C@H]1[C@@H]2CC(=O)C[C@@H]21 ; CC(C)n1nc([C@H]2CC[C@@H](C(F)(F)F)CC2)nc1[C@H]1[C@@H]2CC(=O)C[C@@H]21]

5-[1-Isopropyl-5-(3-morpholinocyclopentyl)-1,2,4-triazol-3-yl]-3-(trifluoromethyl)pyridin-2-amine (220 mg, 0.52 mmol) was separated using chiral SFC (SFC-13; Phenomenex-Cellulose-2 (250 mm*30 mm, 10 um)); 0.1% NH₃H₂O/IPA=30/30; 60 mL/min) to provide the title compound-1 (first peak on SFC, 50 mg, 22.7% yield) and title compound-2 (second peak on SFC, 95 mg, 43% yield). LCMS (ESI) [M+H]⁺=356.2. The relative stereochemistry was arbitrarily assigned.

### Step 6: (R)-4-((1R,3R,5S,6R)-6-(1-Isopropyl-3-((1R,4R)-4-(trifluoromethyl)cyclohexyl)-1H-1,2,4-triazol-5-yl)bicyclo[3.1.0]hexan-3-yl)-3-methylmorpholine and (R)-4-((1R, 3S,5S,6R)-6-(1-isopropyl-3-((1R,4R)-4-(trifluoromethyl)cyclohexyl)-1H-1,2,4-triazol-5-yl)bicyclo[3.1.0]hexan-3-yl)-3-methylmorpholine (Compounds 164* & 165*)

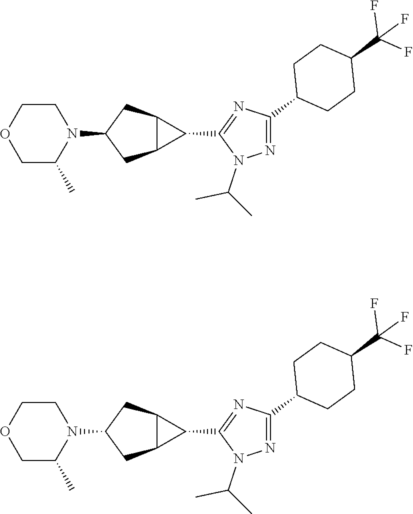 [SMILES: CC(C)n1nc([C@H]2CC[C@H](C(F)(F)F)CC2)nc1[C@H]1[C@@H]2C[C@H](N3CCOC[C@H]3C)C[C@@H]21 ; CC(C)n1nc([C@H]2CC[C@H](C(F)(F)F)CC2)nc1[C@H]1[C@@H]2C[C@@H](N3CCOC[C@H]3C)C[C@@H]21]

To a solution of (3R)-3-methylmorpholine;hydrochloride (155 mg, 1.13 mmol) and (1R,5S,6r)-6-(1-isopropyl-3-((1R,4R)-4-(trifluoromethyl)cyclohexyl)-1H-1,2,4-triazol-5-yl)bicyclo[3.1.0]hexan-3-one (50 mg, 0.14 mmol) in anhydrous 1,2-dichloroethane (2 mL) was added molecular sieve and N,N-diisopropylethylamine (0.12 mL, 0.7 mmol) at 25° C. and stirred at 25° C. for 16 hours. Then NaBH(OAc)₃ (149 mg, 0.7 mmol) was added and stirred at 65° C. for 16 hours. Ethyl acetate (40 mL) was added and the resulting mixture was washed with brine (30 mL×3). The organic was concentrated in vacuo. The residue was purified by reverse phase chromatography (water (NH₃H₂O+NH₄HCO₃)-CAN, 20%-50%) to provide compound 164* (first peak on HPLC (basic), 5.98 mg, 9.6% yield) and compound 165* (second peak on HPLC (basic), 4.9 mg, 7.9% yield). LCMS (ESI) [M+H]⁺=441.2. The relative stereochemistry was arbitrarily assigned.

Compound 164*: ¹H NMR (400 MHz, CD₃OD) δ4.79-4.72 (m, 1H), 3.80-3.64 (m, 3H), 3.54-3.42 (m, 1H), 3.15-3.02 (m, 1H), 2.86-2.68 (m, 2H), 2.65-2.56 (m, 1H), 2.50-2.48 (m, 1H), 2.26-2.08 (m, 3H), 2.05-2.03 (m, 4H), 1.98-1.71 (m, 5H), 1.65-1.52 (m, 2H), 1.42-1.29 (m, 8H), 1.11 (d, J=6.4 Hz, 3H)

Compound 165*: ¹H NMR (400 MHz, CD₃OD) δ4.77-4.70 (m, 1H), 3.77-3.70 (m, 1H), 3.69-3.58 (m, 2H), 3.54-3.40 (m, 2H), 2.87-2.78 (m, 1H), 2.70-2.55 (m, 2H), 2.54-2.44 (m, 1H), 2.32-2.10 (m, 3H), 2.08-1.96 (m, 5H), 1.89-1.88 (m, 2H), 1.82-1.64 (m, 2H), 1.63-1.51 (m, 2H), 1.43-1.35 (m, 8H), 1.08 (d, J=6.4 Hz, 3H).

### Step 7: (R)-4-((1R,3R,5S,6R)-6-(1-Isopropyl-3-((1s,4S)-4-(trifluoromethyl)cyclohexyl)-1H-1,2,4-triazol-5-yl)bicyclo[3.1.0]hexan-3-yl)-3-methylmorpholine and (R)-4-((1R, 3S,5S,6R)-6-(1-Isopropyl-3-((1s, 4S)-4-(trifluoromethyl)cyclohexyl)-1H-1,2,4-triazol-5-yl)bicyclo[3.1.0]hexan-3-yl)-3-methylmorpholine (Compounds 166* & 167*)

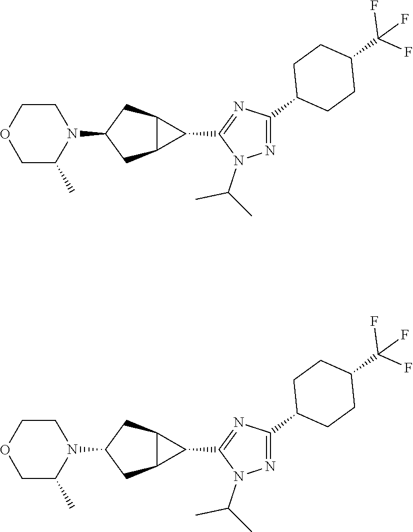 [SMILES: CC(C)n1nc([C@H]2CC[C@@H](C(F)(F)F)CC2)nc1[C@H]1[C@@H]2C[C@H](N3CCOC[C@H]3C)C[C@@H]21 ; CC(C)n1nc([C@H]2CC[C@@H](C(F)(F)F)CC2)nc1[C@H]1[C@@H]2C[C@@H](N3CCOC[C@H]3C)C[C@@H]21]

To a solution of (3R)-3-methylmorpholine;hydrochloride (310 mg, 2.25 mmol) and (1R,5S,6r)-6-(1-isopropyl-3-((Is, 4S)-4-(trifluoromethyl)cyclohexyl)-1H-1,2,4-triazol-5-yl)bicyclo[3.1.0]hexan-3-one (95 mg, 0.28 mmol) in anhydrous 1,2-dichloroethane (4 mL) was added molecular sieve and N,N-diisopropylethylamine (0.25 mL, 1.41 mmol) at 25° C. and stirred at 25° C. for 16 hours. Then NaBH(OAc)₃ (149 mg, 0.7 mmol) was added and stirred at 65° C. for 16 hours. Ethyl acetate (40 mL) was added and the resulting mixture was washed with brine (30 mL×3). The organic was concentrated in vacuo. The residue was purified by reverse phase chromatography (water (NH₃H₂O+NH₄HCO₃)-CAN, 20%-50%) to provide compound 166* (first peak on HPLC (basic), 14.1 mg, 11.4% yield) and compound 167* (second peak on HPLC (basic), 10.9 mg, 8.8% yield). LCMS (ESI) [M+H]⁺=441.2. The relative stereochemistry was arbitrarily assigned.

Compound 166*: ¹H NMR (400 MHz, CD₃OD) δ4.78-4.71 (m, 1H), 3.76-3.63 (m, 3H), 3.51-3.44 (m, 1H), 3.12-3.01 (m, 1H), 3.00-2.92 (m, 1H), 2.83-2.66 (m, 2H), 2.52-2.43 (m, 1H), 2.30-2.07 (m, 5H), 1.99-1.84 (m, 5H), 1.75-1.65 (m, 6H), 1.50-1.44 (m, 6H), 1.10 (d, J=6.4 Hz, 3H).

Compound 167*: H NMR (400 MHz, CD₃OD) δ4.75-4.69 (m, 1H), 3.79-3.59 (m, 3H), 3.56-3.40 (m, 2H), 2.96-2.95 (m, 1H), 2.88-2.77 (m, 1H), 2.69-2.59 (m, 1H), 2.54-2.44 (m, 1H), 2.34-2.10 (m, 5H), 2.01-2.00 (m, 1H), 1.89 (brs, 2H), 1.76-1.63 (m, 8H), 1.48 (d, J=6.4 Hz, 6H), 1.08 (d, J=6.4 Hz, 3H).

### Example BW: (S)-2-(Fluoromethyl)-4-((1S,3S)-3-(1-isopropyl-3-(6-(trifluoromethyl)pyridin-3-yl)-1H-1,2,4-triazol-5-yl)cyclopentyl)morpholine and (S)-2-(Fluoromethyl)-4-((1R,3S)-3-(1-isopropyl-3-(6-(trifluoromethyl)pyridin-3-yl)-1H-1,2,4-triazol-5-yl)cyclopentyl)morpholine (Compounds 168* & 169*)

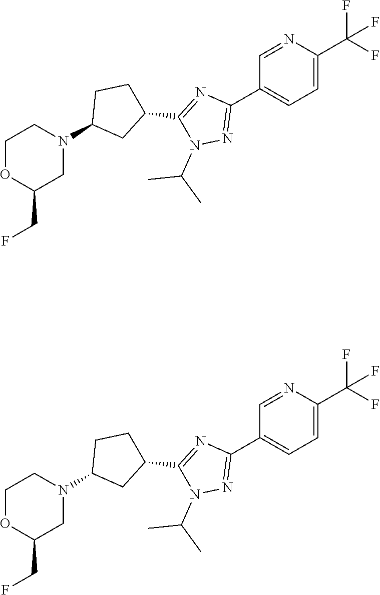 [SMILES: CC(C)n1nc(-c2ccc(C(F)(F)F)nc2)nc1[C@H]1CC[C@H](N2CCO[C@H](CF)C2)C1 ; CC(C)n1nc(-c2ccc(C(F)(F)F)nc2)nc1[C@H]1CC[C@@H](N2CCO[C@H](CF)C2)C1]

### Step 1: ((S)-4-((1S,3S)-3-(1-Isopropyl-3-(6-(trifluoromethyl)pyridin-3-yl)-1H-1,2,4-triazol-5-yl)cyclopentyl)morpholin-2-yl)methanol and ((S)-4-((1R,3S)-3-(1-isopropyl-3-(6-(trifluoromethyl)pyridin-3-yl)-1H-1,2,4-triazol-5-yl)cyclopentyl)morpholin-2-yl)methanol

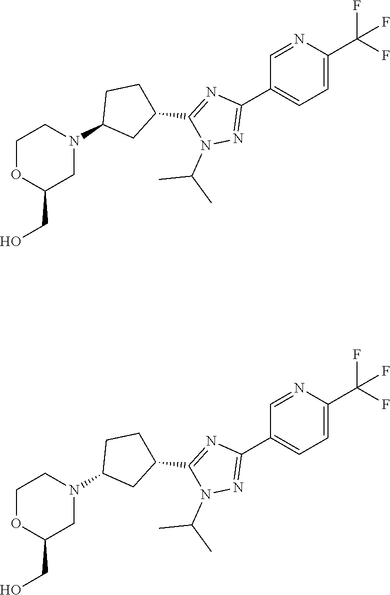 [SMILES: CC(C)n1nc(-c2ccc(C(F)(F)F)nc2)nc1[C@H]1CC[C@H](N2CCO[C@H](CO)C2)C1 ; CC(C)n1nc(-c2ccc(C(F)(F)F)nc2)nc1[C@H]1CC[C@@H](N2CCO[C@H](CO)C2)C1]

To a solution of (S)-3-(1-isopropyl-3-(6-(trifluoromethyl)pyridin-3-yl)-1H-1,2,4-triazol-5-yl)cyclopentanone (120.0 mg, 0.35 mmol) in dichloromethane (3 mL) was added [(2S)-morpholin-2-yl]methanol hydrochloride (109 mg, 0.71 mmol) and NN-diisopropylethylamine (0.25 mL, 1.42 mmol) at 25° C. The reaction mixture was stirred at 25° C. for 12 hrs. Then NaBH(OAc)₃ (376 mg, 1.77 mmol) was added to above mixture and stirred at 60° C. for another 2 hrs. The reaction mixture was quenched with water (5 mL) and extracted with dichloromethane (10 mL×3). The combined organic layer was dried over anhydrous Na₂SO₄, filtered and concentrated in vacuo. The residue was purified by silica flash chromatography (0-10% methanol in dichloromethane) to provide the title compound (150 mg, 96% yield) as the mixture of diastereomers. LCMS (ESI), [M+H]⁺=440.3.

### Step 2: (S)-2-(Fluoromethyl)-4-((1S,3S)-3-(1-isopropyl-3-(6-(trifluoromethyl)pyridin-3-yl)-1H-1,2,4-triazol-5-yl)cyclopentyl)morpholine and (S)-2-(Fluoromethyl)-4-((1R,3S)-3-(1-isopropyl-3-(6-(trifluoromethyl)pyridin-3-yl)-1H-1,2,4-triazol-5-yl)cyclopentyl)morpholine. (Compounds 168 & 169

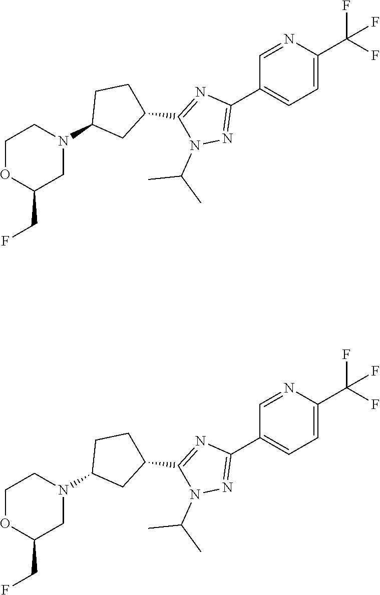 [SMILES: CC(C)n1nc(-c2ccc(C(F)(F)F)nc2)nc1[C@H]1CC[C@H](N2CCO[C@H](CF)C2)C1 ; CC(C)n1nc(-c2ccc(C(F)(F)F)nc2)nc1[C@H]1CC[C@@H](N2CCO[C@H](CF)C2)C1]

To a solution of ((2S)-4-((3S)-3-(1-isopropyl-3-(6-(trifluoromethyl)pyridin-3-yl)-1H-1,2,4-triazol-5-yl)cyclopentyl)morpholin-2-yl)methanol (150.0 mg, 0.34 mmol) in dichloromethane (3 mL) was added Deoxo-Fluor (226.54 mg, 1.02 mmol) at 25° C. and stirred at 25° C. for 6 h. The mixture was adjusted to pH 7-8 with NaHCO₃ (aq.), and extracted with dichloromethane (20 mL×3). The combined organics were washed with brine (20 mL×2), dried over sodium sulfate, filtered and concentrated. The residue was purified by silica flash chromatography (0-10% methanol in dichloromethane) to give the title compound (70 mg, 0.16 mmol, 49%), which was provided as a mixture of diastereoisomers. LCMS (ESI) [M+H]⁺=442.2. The mixture of diastereoisomers (70 mg, 0.16 mmol) was separated using chiral SFC (Daicel Chiralcel OD-H (250 mm*30 mm, 5 um); 0.1% NH₃ in H₂O; ETOH; 15/15; 60 mL/min) to provide the compound 168* (first peak on SFC, 11.07 mg, 15.3% yield) and the title compound 169* (second peak on SFC, 54.16 mg, 76.6% yield). The relative stereochemistry was arbitrarily assigned.

Compound 168*: ¹H NMR (400 MHz, CD₃OD) δ 9.32 (s, 1H), 8.61 (dd, J=1.6, 8.0 Hz, 1H), 7.90 (d, J=8.0 Hz, 1H), 4.82-4.74 (m, 1H), 4.46 (d, J=4.4 Hz, 1H), 4.34 (d, J=4.0 Hz, 1H), 3.94-3.90 (m, 1H), 3.82-3.66 (m, 2H), 3.64-3.56 (m, 1H), 3.03-2.91 (m, 3H), 2.29-2.16 (m, 4H), 2.12-2.04 (m, 2H), 2.01-1.91 (m, 1H), 1.72-1.62 (m, 1H), 1.54 (dd, J=2.8, 6.4 Hz, 6H),

Compound 169*: ¹H NMR (400 MHz, CD₃OD) δ 9.32 (s, 1H), 8.61 (dd, J=1.2, 8.0 Hz, 1H), 7.90 (d, J=8.4 Hz, 1H), 4.80-4.73 (m, 1H), 4.47 (d, J=4.4 Hz, 1H), 4.35 (d, J=4.0 Hz, 1H), 3.94-3.91 (m, 1H), 3.82-3.66 (m, 2H), 3.53-3.45 (m, 1H), 3.01-2.98 (m, 1H), 2.90-2.81 (m, 2H), 2.42-2.36 (m, 1H), 2.30-2.24 (m, 1H), 2.21-2.14 (m, 1H), 2.11-2.03 (m, 3H), 2.02-1.94 (m, 1H), 1.90-1.80 (m, 1H), 1.54 (dd, J=2.4, 6.8 Hz, 6H).

### Example BX: 4-((1R,3r,5S,6r)-6-(1-Isopropyl-3-(3-(trifluoromethyl)phenyl)-1H-pyrazol-5-yl)bicyclo[3.1.0]hexan-3-yl)morpholine (Compound 170)

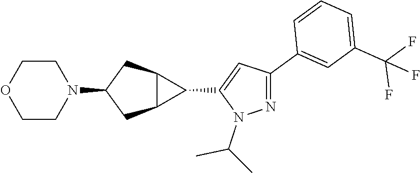 [SMILES: CC(C)n1nc(-c2cccc(C(F)(F)F)c2)cc1[C@H]1[C@@H]2C[C@H](N3CCOCC3)C[C@@H]21]

### Step 1: (1R,5S,6r)-6-(3-Iodo-1-isopropyl-1H-pyrazol-5-yl)bicyclo[3.1.0]hexan-3-one

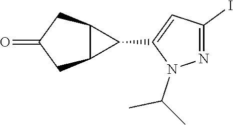 [SMILES: CC(C)n1nc(I)cc1[C@H]1[C@@H]2CC(=O)C[C@@H]21]

The title compound was synthesized following a procedure described in WO2015091889.

### Step 2: (1R,5S,6r)-6-(1-Isopropyl-3-(3-(trifluoromethyl)phenyl)-1H-pyrazol-5-yl)bicyclo[3.1.0]hexan-3-one

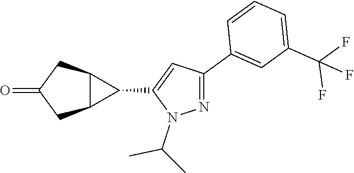 [SMILES: CC(C)n1nc(-c2cccc(C(F)(F)F)c2)cc1[C@H]1[C@@H]2CC(=O)C[C@@H]21]

A solution of (1R,5S,6r)-6-(3-iodo-1-isopropyl-1H-pyrazol-5-yl)bicyclo[3.1.0]hexan-3-one (66 mg, 0.20 mmol), potassium phosphate (85 mg, 0.40 mmol), SPhos Pd G₃ (7.8 mg, 0.010 mmol) and (3-(trifluoromethyl)phenyl)boronic acid (57 mg, 0.30 mmol) in 1,4-dioxane (1.0 mL) and water (0.25 mL) was stirred at 60° C. for 18 h. The reaction mixture was diluted with 1N aq. NH₄Cl (1 mL) and dichloromethane (5 mL). The aqueous layer was extracted with dichloromethane (2×5 mL). The combined organic layer was dried over anhydrous Na₂SO₄ and concentrated under reduced pressure to give the crude title compound (170 mg, 100% yield). LCMS (ESI) [M+H]⁺=349.2.

### Step 3: 4-((1R,3r,5S,6r)-6-(1-Isopropyl-3-(3-(trifluoromethyl)phenyl)-1H-pyrazol-5-yl)bicyclo[3.1.0]hexan-3-yl)morpholine (Compound 170)

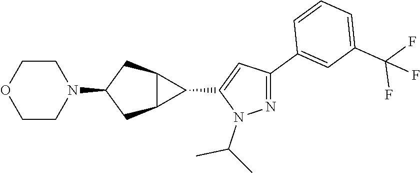 [SMILES: CC(C)n1nc(-c2cccc(C(F)(F)F)c2)cc1[C@H]1[C@@H]2C[C@H](N3CCOCC3)C[C@@H]21]

To ((1R,5S,6r)-6-(1-isopropyl-3-(3-(trifluoromethyl)phenyl)-1H-pyrazol-5-yl)bicyclo[3.1.0]hexan-3-one (69 mg, 0.20 mmol) in toluene (1.0 mL) was slowly added morpholine (52 mg, 0.60 mmol), sodium cyanoborohydride (19 mg, 0.30 mmol) and acetic acid (0.12 mL, 2.0 mmol). The reaction mixture was stirred at 80° C. for 3 h and diluted with 1N aq. NH₄Cl (1 mL) and 10% methanol in dichloromethane (5 mL). The aqueous layer was extracted with 10% methanol in dichloromethane (2×5 mL). The combined organic layer was concentrated under reduced pressure. The residue was purified by prep-HPLC (acetonitrile/water gradient with 0.10% NH₄OH) to give the title compound (29 mg) as a pure single stereoisomer. LCMS (ESI) [M+H]⁺=420.3.

Compound 170: ¹H NMR (400 MHz, DMSO-d₆) δ 8.05-7.97 (m, 2H), 7.65-7.55 (m, 2H), 6.45 (s, 1H), 4.79-4.64 (m, 1H), 3.56 (t, J=4.6 Hz, 4H), 2.85-2.73 (m, 1H), 2.39-2.31 (m, 4H), 2.17-2.06 (m, 2H), 2.01 (t, J=3.4 Hz, 1H), 1.74 (dd, J=13.7, 6.1 Hz, 2H), 1.69-1.61 (m, 2H), 1.46 (d, J=6.6 Hz, 6H). The relative stereochemistry was assigned based on ¹H NMR analysis.

### Example BY: 4-((1R,3r,5S,6r)-6-(1-Isopropyl-3-(5-(trifluoromethyl)pyridin-3-yl)-1H-pyrazol-5-yl)bicyclo[3.1.0]hexan-3-yl)morpholine (Compound 171)

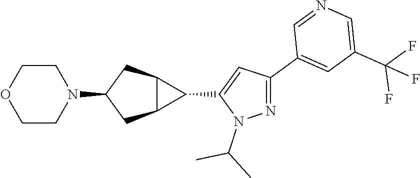 [SMILES: CC(C)n1nc(-c2cncc(C(F)(F)F)c2)cc1[C@H]1[C@@H]2C[C@H](N3CCOCC3)C[C@@H]21]

The title compound was synthesized following a procedure similar to compound 170 using (5-(trifluoromethyl)pyridin-3-yl)boronic acid in step 2. The residue was purified by prep-HPLC (acetonitrile/water gradient with 0.1% NH₄OH) afforded the title compound. LCMS (ESI) [M+H]⁺=421.3.

Compound 171: ¹H NMR (400 MHz, DMSO-d₆) δ 9.22 (d, J=2.0 Hz, 1H), 8.84 (s, 1H), 8.36 (s, 1H), 6.60 (s, 1H), 4.81-4.67 (m, 1H), 3.56 (t, J=4.6 Hz, 4H), 2.85-2.74 (m, 1H), 2.39-2.31 (m, 4H), 2.17-2.07 (m, 2H), 2.04 (t, J=3.3 Hz, 1H), 1.75 (dd, J=13.8, 6.1 Hz, 2H), 1.69-1.61 (m, 2H), 1.47 (d, J=6.6 Hz, 6H). The relative stereochemistry was assigned based on ¹H NMR analysis.

### Example BZ: 4-((1R,3r,5S,6r)-6-(1-Isopropyl-3-(2-(trifluoromethyl)pyridin-4-yl)-1H-pyrazol-5-yl)bicyclo[3.1.0]hexan-3-yl)morpholine (Compound 172)

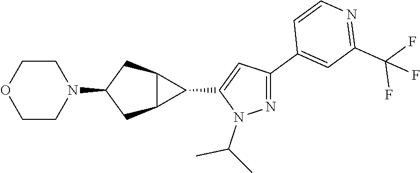 [SMILES: CC(C)n1nc(-c2ccnc(C(F)(F)F)c2)cc1[C@H]1[C@@H]2C[C@H](N3CCOCC3)C[C@@H]21]

The title compound was synthesized following a procedure similar to compound 170 using (2-(trifluoromethyl)pyridin-4-yl)boronic acid in step 2. The residue was purified by prep-HPLC (acetonitrile/water gradient with 0.1% NH₄OH) afforded the title compound. LCMS (ESI) [M+H]⁺=421.1. The relative stereochemistry was assigned based on ¹H NMR analysis.

Compound 172: ¹H NMR (400 MHz, DMSO-d₆) δ 8.71 (d, J=5.1 Hz, 1H), 8.10 (s, 1H), 7.97 (dd, J=5.1, 1.6 Hz, 1H), 6.68 (s, 1H), 4.81-4.69 (m, 1H), 3.56 (t, J=4.7 Hz, 4H), 2.85-2.74 (m, 1H), 2.40-2.30 (m, 4H), 2.16-2.07 (m, 2H), 2.05 (t, J=3.3 Hz, 1H), 1.75 (dd, J=13.7, 6.0 Hz, 2H), 1.68-1.63 (m, 2H), 1.47 (d, J=6.6 Hz, 6H).

### Example CA: 4-((1R,3r,5S,6r)-6-(1-Isopropyl-3-(4-(trifluoromethyl)phenyl)-1H-pyrazol-5-yl)bicyclo[3.1.0]hexan-3-yl)morpholine (Compound 173)

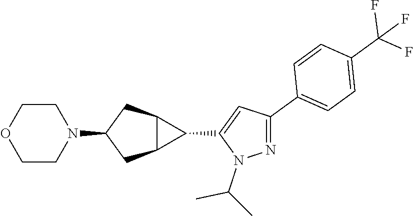 [SMILES: CC(C)n1nc(-c2ccc(C(F)(F)F)cc2)cc1[C@H]1[C@@H]2C[C@H](N3CCOCC3)C[C@@H]21]

The title compound was synthesized following a procedure similar to compound 170 using (4-(trifluoromethyl)phenyl)boronic acid in step 2. The residue was purified by prep-HPLC (acetonitrile/water gradient with 0.1% NH₄OH) afforded the title compound. LCMS (ESI) [M+H]⁺=420.1. The relative stereochemistry was assigned based on ¹H NMR analysis.

Compound 173: ¹H NMR (400 MHz, DMSO-d₆) δ 7.93 (d, J=8.1 Hz, 2H), 7.71 (d, J=8.2 Hz, 2H), 6.42 (s, 1H), 4.80-4.66 (m, 1H), 3.56 (t, J=4.6 Hz, 4H), 2.85-2.74 (m, 1H), 2.41-2.31 (m, 4H), 2.17-2.06 (m, 2H), 2.02 (t, J=3.4 Hz, 1H), 1.74 (dd, J=13.7, 6.1 Hz, 2H), 1.70-1.61 (m, 2H), 1.47 (d, J=6.6 Hz, 6H).

### Example CB: 4-((1R,3r,5S,6r)-6-(1-Isopropyl-3-(5-(trifluoromethyl)pyridin-2-yl)-1H-pyrazol-5-yl)bicyclo[3.1.0]hexan-3-yl)morpholine (Compound 174)

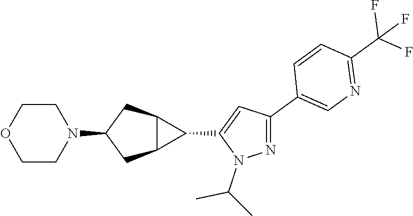 [SMILES: CC(C)n1nc(-c2ccc(C(F)(F)F)nc2)cc1[C@H]1[C@@H]2C[C@H](N3CCOCC3)C[C@@H]21]

The title compound was synthesized following a procedure similar to compound 170 using (6-(trifluoromethyl)pyridin-3-yl)boronic acid in step 2. The residue was purified by prep-HPLC (acetonitrile/water gradient with 0.1% NH₄OH) afforded the title compound. LCMS (ESI) [M+H]⁺=421.2. The relative stereochemistry was assigned based on ¹H NMR analysis.

Compound 174: ¹H NMR (400 MHz, DMSO-d₆) δ 8.89 (s, 1H), 8.16 (dd, J=8.5, 2.4 Hz, 1H), 8.06 (d, J=8.3 Hz, 1H), 6.47 (s, 1H), 4.81-4.72 (m, 1H), 3.58-3.53 (m, 4H), 2.84-2.73 (m, 1H), 2.38-2.31 (m, 4H), 2.15-2.03 (m, 3H), 1.75 (dd, J=13.8, 6.1 Hz, 2H), 1.68-1.64 (m, 2H), 1.48 (d, J=6.6 Hz, 6H).

### Example CC: 4-((1R,3r,5S,6r)-6-(1-Isopropyl-3-(3-(trifluoromethyl)phenyl)-1H-pyrazol-5-yl)bicyclo[3.1.0]hexan-3-yl)-1,4-oxazepane and 4-((1R,3s,5S,6r)-6-(1-isopropyl-3-(3-(trifluoromethyl)phenyl)-1H-pyrazol-5-yl)bicyclo[3.1.0]hexan-3-yl)-1,4-oxazepane (Compounds 175* & 176*)

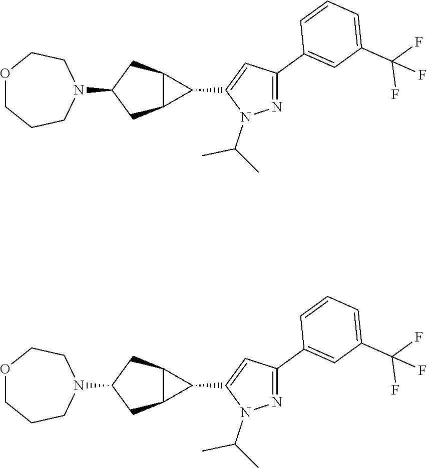 [SMILES: CC(C)n1nc(-c2cccc(C(F)(F)F)c2)cc1[C@H]1[C@@H]2C[C@H](N3CCCOCC3)C[C@@H]21 ; CC(C)n1nc(-c2cccc(C(F)(F)F)c2)cc1[C@H]1[C@@H]2C[C@@H](N3CCCOCC3)C[C@@H]21]

### Step 1: 4-((1R,5S,6r)-6-(3-Iodo-1-isopropyl-1H-pyrazol-5-yl)bicyclo[3.1.0]hexan-3-yl)-1,4-oxazepane

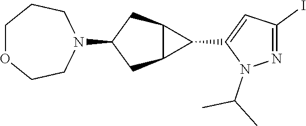 [SMILES: CC(C)n1nc(I)cc1[C@H]1[C@@H]2C[C@H](N3CCCOCC3)C[C@@H]21]

To (1R,5S,6r)-6-(3-iodo-1-isopropyl-1H-pyrazol-5-yl)bicyclo[3.1.0]hexan-3-one (330 mg, 1.00 mmol) and 1,4-oxazepane (304 mg, 3.00 mmol) in m-xylene (4.0 mL) was added sodium triacetoxyborohydride (212 mg, 2.00 mmol) and acetic acid (0.286 mL, 5.00 mmol). The reaction mixture was stirred at 65° C. for 18 h and concentrated under reduced pressure. The mixture was purified by column chromatography on silica gel eluting with THF/petroleum ether (2:1) to give the title compound (312 mg, 75% yield). LCMS (ESI) [M+H]⁺=416.1.

### Step 2: 4-((1R,3r,5S,6r)-6-(1-Isopropyl-3-(3-(trifluoromethyl)phenyl)-1H-pyrazol-5-yl)bicyclo[3.1.0]hexan-3-yl)-1,4-oxazepane and 4-((1R,3s,5S,6r)-6-(1-isopropyl-3-(3-(trifluoromethyl)phenyl)-1H-pyrazol-5-yl)bicyclo[3.1.0]hexan-3-yl)-1,4-oxazepane (Compounds 175* & 176*)

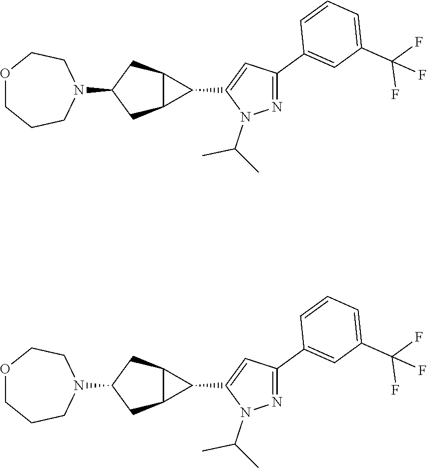 [SMILES: CC(C)n1nc(-c2cccc(C(F)(F)F)c2)cc1[C@H]1[C@@H]2C[C@H](N3CCCOCC3)C[C@@H]21 ; CC(C)n1nc(-c2cccc(C(F)(F)F)c2)cc1[C@H]1[C@@H]2C[C@@H](N3CCCOCC3)C[C@@H]21]

A solution of 4-((1R,5S,6r)-6-(3-iodo-1-isopropyl-1H-pyrazol-5-yl)bicyclo[3.1.0]hexan-3-yl)-1,4-oxazepane (83 mg, 0.20 mmol), aq. potassium phosphate (0.267 mL, 0.40 mmol, 1.5 M in water), bis(di-tert-butyl(4-dimethylaminophenyl)phosphine)dichloropalladium(II) (5.7 mg, 0.008 mmol) and (3-(trifluoromethyl)phenyl)boronic acid (57 mg, 0.30 mmol) in 1,4-dioxane (2.0 mL) was stirred at 100° C. for 16 h. The reaction mixture was concentrated under reduced pressure and purified by prep-HPLC (acetonitrile/water gradient with 0.10% NH₄OH) to provide compound 175* and compound 176*. The relative stereochemistry was arbitrarily assigned. LCMS (ESI) [M+H]⁺=434.2.

Compound 175*: ¹H NMR (400 MHz, DMSO-d₆) δ 8.07-7.95 (m, 2H), 7.66-7.54 (m, 2H), 6.48 (s, 1H), 4.73 (m, 1H), 3.69-3.54 (m, 4H), 2.94-2.83 (m, 1H), 2.64-2.56 (m, 4H), 2.14-2.05 (m, 2H), 1.81-1.59 (m, 7H), 1.43 (d, J=6.6 Hz, 6H).

Compound 176*: ¹H NMR (400 MHz, DMSO-d₆) δ 8.08-7.94 (m, 2H), 7.66-7.51 (m, 2H), 6.45 (s, 1H), 4.82-4.65 (m, 1H), 3.72-3.55 (m, 4H), 3.30-3.22 (m, 1H), 2.66-2.55 (m, 4H), 2.18 (m, 2H), 1.90 (t, J=3.0 Hz, 1H), 1.76 (m, 2H), 1.67-1.55 (m, 4H), 1.45 (d, J=6.6 Hz, 6H).

### Example CD: 4-((1R,3s,5S,6r)-6-(3-(5-Chloropyridin-3-yl)-1-isopropyl-1H-pyrazol-5-yl)bicyclo[3.1.0]hexan-3-yl)-1,4-oxazepane and 4-((1R,3r,5S,6r)-6-(3-(5-chloropyridin-3-yl)-1-isopropyl-1H-pyrazol-5-yl)bicyclo[3.1.0]hexan-3-yl)-1,4-oxazepane (Compounds 177* & 178*)

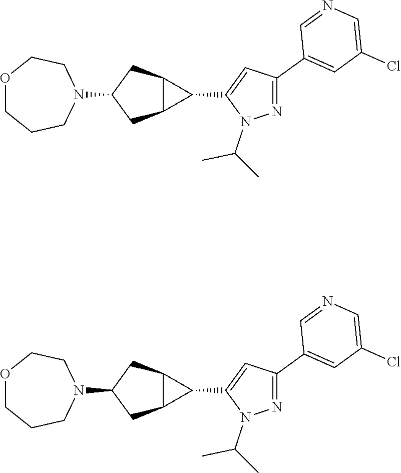 [SMILES: CC(C)n1nc(-c2cncc(Cl)c2)cc1[C@H]1[C@@H]2C[C@@H](N3CCCOCC3)C[C@@H]21 ; CC(C)n1nc(-c2cncc(Cl)c2)cc1[C@H]1[C@@H]2C[C@H](N3CCCOCC3)C[C@@H]21]

The titled compounds were synthesized following a procedure similar to synthesize compounds 175* and 176* using (5-chloropyridin-3-yl)boronic acid in step 2. The two diastereoisomers were purified by prep-HPLC (acetonitrile/water gradient with 0.1% NH₄OH) to provide compounds 177* and 178*). The relative stereochemistry was arbitrarily assigned. LCMS (ESI) [M+H]⁺=401.1.

Compound 177*: ¹H NMR (400 MHz, DMSO-d₆) δ 8.88 (d, J=1.7 Hz, 1H), 8.50 (d, J=2.3 Hz, 1H), 8.14 (t, J=2.0 Hz, 1H), 6.50 (s, 1H), 4.75 (td, J=6.6, 13.2 Hz, 1H), 3.68-3.59 (m, 4H), 3.30-3.25 (m, 1H), 2.64 (m, 4H), 2.23-2.15 (m, 2H), 1.92 (t, J=2.9 Hz, 1H), 1.79-1.74 (m, 2H), 1.64-1.59 (m, 4H), 1.45 (d, J=6.6 Hz, 6H).

Compound 178*: ¹H NMR (400 MHz, DMSO-d₆) 8.88 (d, J=1.7 Hz, 1H), 8.49 (d, J=2.3 Hz, 1H), 8.49 (d, J=2.3 Hz, 1H), 8.14 (t, J=2.1 Hz, 1H), 6.53 (s, 1H), 4.74 (dt, J=13.1, 6.6 Hz, 1H), 3.70-3.54 (m, 4H), 2.94-2.82 (m, 1H), 2.69-2.55 (m, 4H), 2.17-2.05 (m, 2H), 1.84-1.54 (m, 7H), 1.43 (d, J 6.6 Hz, 6H), 1.14 (d, J=13.2 Hz, 1H).s

### Example CE: 4-(5-((1R,3r,5S,6r)-3-(1,4-Oxazepan-4-yl)bicyclo[3.1.0]hexan-6-yl)-1-isopropyl-1H-pyrazol-3-yl)benzonitrile and 4-(5-((1R,3s,5S,6r)-3-(1,4-oxazepan-4-yl)bicyclo[3.1.0]hexan-6-yl)-1-isopropyl-1H-pyrazol-3-yl)benzonitrile (Compounds 179* & 180*)

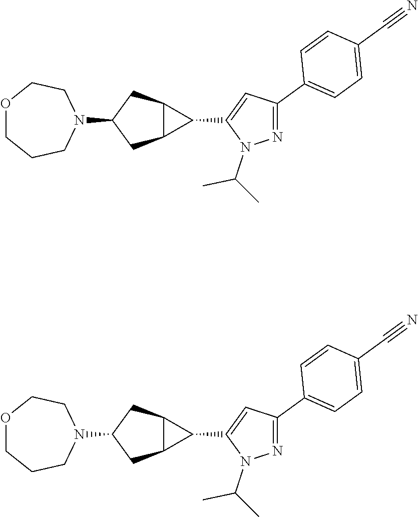 [SMILES: CC(C)n1nc(-c2ccc(C#N)cc2)cc1[C@H]1[C@@H]2C[C@H](N3CCCOCC3)C[C@@H]21 ; CC(C)n1nc(-c2ccc(C#N)cc2)cc1[C@H]1[C@@H]2C[C@@H](N3CCCOCC3)C[C@@H]21]

The titled compounds were synthesized following a procedure similar to synthesize compounds 175* and 176* using (4-cyanophenyl)boronic acid in step 2. The two diastereoisomers were purified by prep-HPLC (acetonitrile/water gradient with 0.1% NH₄OH) to provide compounds 179* and 180*. The relative stereochemistry was arbitrarily assigned. LCMS (ESI) [M+H]⁺=391.2.

Compound 179*: ¹H NMR (400 MHz, DMSO-d₆) δ 7.98-7.73 (m, 4H), 6.45 (s, 1H), 4.84-4.66 (m, 1H), 3.75-3.50 (m, 4H), 3.30-3.22 (m, 1H), 2.67-2.57 (m, 4H), 2.24-2.11 (m, 2H), 1.91 (t, J=3.1 Hz, 1H), 1.75 (m, 2H), 1.66-1.55 (m, 4H), 1.45 (d, J=6.6 Hz, 6H).

Compound 180*: ¹H NMR (400 MHz, DMSO-d₆) δ 7.93-7.75 (m, 4H), 6.48 (s, 1H), 4.74 (m, 1H), 3.69-3.54 (m, 4H), 2.94-2.81 (m, 1H), 2.63-2.57 (m, 4H), 2.15-2.05 (m, 2H), 1.81-1.60 (m, 7H), 1.43 (d, J=6.6 Hz, 6H).

### Example CF: 4-((1R,3r,5S,6r)-6-(1-Isopropyl-3-(4-(trifluoromethyl)phenyl)-1H-pyrazol-5-yl)bicyclo[3.1.0]hexan-3-yl)-1,4-oxazepane and 4-((1R,3s,5S,6r)-6-(1-isopropyl-3-(4-(trifluoromethyl)phenyl)-1H-pyrazol-5-yl)bicyclo[3.1.0]hexan-3-yl)-1,4-oxazepane (Compounds 181* and 182*)

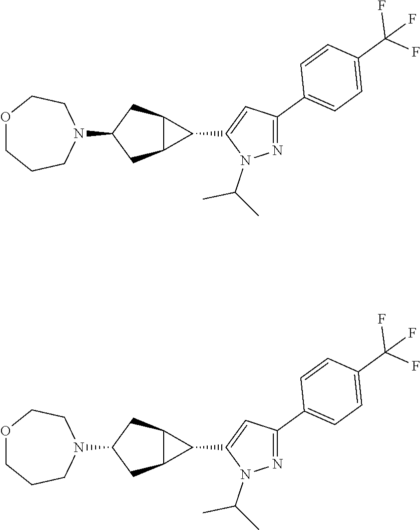 [SMILES: CC(C)n1nc(-c2ccc(C(F)(F)F)cc2)cc1[C@H]1[C@@H]2C[C@H](N3CCCOCC3)C[C@@H]21 ; CC(C)n1nc(-c2ccc(C(F)(F)F)cc2)cc1[C@H]1[C@@H]2C[C@@H](N3CCCOCC3)C[C@@H]21]

The titled compounds were synthesized following a procedure similar to synthesize compounds 175* and 176* using (4-(trifluoromethyl)phenyl)boronic acid in step 2. The two diastereoisomers were purified by prep-HPLC (acetonitrile/water gradient with 0.10% NH₄OH) to provide compounds 181* and 182*. The relative stereochemistry was arbitrarily assigned. LCMS (ESI) [M+H]⁺=434.3.

Compound 181*: ¹H NMR (400 MHz, CDCl₃) δ 7.84 (d, J=8.0 Hz, 2H), 7.58 (d, J=8.3 Hz, 2H), 6.08 (s, 1H), 4.67 (m, 1H), 3.84-3.69 (m, 4H), 3.34 (m, 1H), 2.76-2.67 (m, 4H), 2.39-2.26 (m, 2H), 1.88 (m, 2H), 1.77-1.70 (m, 1H), 1.66-1.60 (m, 4H), 1.55 (d, J=6.8 Hz, 6H).

Compound 182*: ¹H NMR (400 MHz, CDCl₃) δ 7.85 (d, J=8.0 Hz, 2H), 7.59 (d, J=8.3 Hz, 2H), 6.12 (s, 1H), 4.64 (m, 1H), 3.85-3.71 (m, 4H), 2.86-2.66 (m, 5H), 2.21 (dd, J=7.0, 12.5 Hz, 2H), 1.94-1.80 (m, 4H), 1.69-1.66 (m, 2H), 1.54 (m, 7H).

### Example CG: 4-((1R,3r,5S,6r)-6-(3-(4-Fluorophenyl)-1-isopropyl-1H-pyrazol-5-yl)bicyclo[3.1.0]hexan-3-yl)-1,4-oxazepane and 4-((1R,3s,5S,6r)-6-(3-(4-fluorophenyl)-1-isopropyl-1H-pyrazol-5-yl)bicyclo[3.1.0]hexan-3-yl)-1,4-oxazepane (Compounds 183* & 184*)

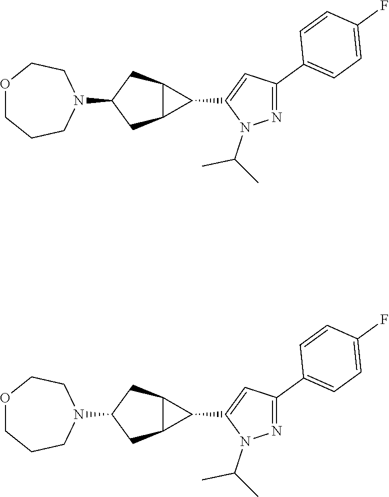 [SMILES: CC(C)n1nc(-c2ccc(F)cc2)cc1[C@H]1[C@@H]2C[C@H](N3CCCOCC3)C[C@@H]21 ; CC(C)n1nc(-c2ccc(F)cc2)cc1[C@H]1[C@@H]2C[C@@H](N3CCCOCC3)C[C@@H]21]

The titled compounds were synthesized following a procedure similar to synthesize compounds 175* and 176* using (4-fluorophenyl)boronic acid in step 2. The two diastereoisomers were purified by prep-HPLC (acetonitrile/water gradient with 0.1% NH₄OH) to provide compounds 183* and 184*. The relative stereochemistry was arbitrarily assigned. LCMS (ESI) [M+H]⁺=384.2.

Compound 183*: ¹H NMR (400 MHz, DMSO-d₆) δ 7.78-7.66 (m, 2H), 7.22-7.13 (m, 2H), 6.24 (s, 1H), 4.76-4.64 (m, 1H), 3.70-3.54 (m, 4H), 3.29-3.19 (m, 1H), 2.69-2.57 (m, 4H), 2.17 (br s, 2H), 1.87 (t, J=3.0 Hz, 1H), 1.75 (m, 2H), 1.65-1.53 (m, 4H), 1.44 (d, J=6.6 Hz, 6H).

Compound 184*: ¹H NMR (400 MHz, DMSO-d₆) δ 7.74 (dd, J=5.6, 8.7 Hz, 2H), 7.17 (t, J=8.8 Hz, 2H), 6.28 (s, 1H), 4.78-4.62 (m, 1H), 3.73-3.51 (m, 4H), 2.89 (d, J=8.0 Hz, 1H), 2.60 (d, J=4.5 Hz, 4H), 2.09 (dd, J=6.9, 12.2 Hz, 2H), 1.83-1.57 (m, 7H), 1.41 (d, J=6.5 Hz, 6H).

### Example CH: 4-((1R,3r,5S,6r)-6-(1-Isopropyl-3-(4-(trifluoromethoxy)phenyl)-1H-pyrazol-5-yl)bicyclo[3.1.0]hexan-3-yl)-1,4-oxazepane and 4-((1R,3s,5S,6r)-6-(1-isopropyl-3-(4-(trifluoromethoxy)phenyl)-1H-pyrazol-5-yl)bicyclo[3.1.0]hexan-3-yl)-1,4-oxazepane (Compounds 185* & 186*)

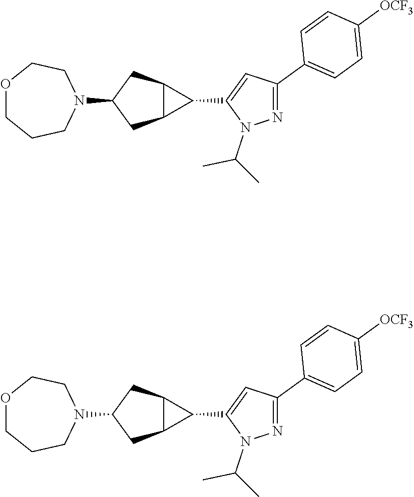 [SMILES: CC(C)n1nc(-c2ccc(OC(F)(F)F)cc2)cc1[C@H]1[C@@H]2C[C@H](N3CCCOCC3)C[C@@H]21 ; CC(C)n1nc(-c2ccc(OC(F)(F)F)cc2)cc1[C@H]1[C@@H]2C[C@@H](N3CCCOCC3)C[C@@H]21]

The titled compounds were synthesized following a procedure similar to synthesize compounds 175* and 176* using (4-fluorophenyl)boronic acid in step 2. The two diastereoisomers were purified by prep-HPLC (acetonitrile/water gradient with 0.1% NH₄OH) to provide compounds 185* and 186*. The relative stereochemistry was arbitrarily assigned. LCMS (ESI) [M+H]⁺=450.3.

Compound 185*: ¹H NMR (400 MHz, DMSO-d₆) δ 7.82 (d, J=8.8 Hz, 2H), 7.34 (d, J=8.3 Hz, 2H), 6.31 (s, 1H), 4.72 (m, 1H), 3.73-3.52 (m, 4H), 3.28 (m, 1H), 2.76-2.55 (m, 4H), 2.18 (m, 2H), 1.89 (m, 1H), 1.76 (m, 2H), 1.60 (m, 4H), 1.44 (d, J=6.8 Hz, 6H).

Compound 186*: ¹H NMR (400 MHz, DMSO-d₆) δ 7.82 (d, J=8.8 Hz, 2H), 7.34 (d, J=8.3 Hz, 2H), 6.34 (s, 1H), 4.72 (quin, J=6.6 Hz, 1H), 3.71-3.52 (m, 4H), 2.94-2.81 (m, 1H), 2.64-2.55 (m, 4H), 2.10 (dd, J=12.3, 7.0 Hz, 2H), 1.42 (d, J=6.5 Hz, 6H) 1.83-1.56 (m, 7H).

### Example CI: 4-((1R,3r,5S,6r)-6-(3-(5-Fluoropyridin-3-yl)-1-isopropyl-1H-pyrazol-5-yl)bicyclo[3.1.0]hexan-3-yl)-1,4-oxazepane and 4-((1R,3s,5S,6r)-6-(3-(5-fluoropyridin-3-yl)-1-isopropyl-1H-pyrazol-5-yl)bicyclo[3.1.0]hexan-3-yl)-1,4-oxazepane (Compounds 187* & 188*)

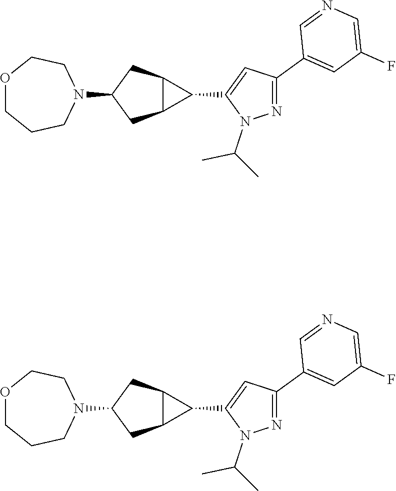 [SMILES: CC(C)n1nc(-c2cncc(F)c2)cc1[C@H]1[C@@H]2C[C@H](N3CCCOCC3)C[C@@H]21 ; CC(C)n1nc(-c2cncc(F)c2)cc1[C@H]1[C@@H]2C[C@@H](N3CCCOCC3)C[C@@H]21]

The titled compounds were synthesized following a procedure similar to synthesize compounds 175* and 176* using (4-fluorophenyl)boronic acid in step 2. The two diastereoisomers were purified by prep-HPLC (acetonitrile/water gradient with 0.1% NH₄OH) to provide compounds 187* and 188*. The relative stereochemistry was arbitrarily assigned. LCMS (ESI) [M+H]⁺=385.2.

Compound 187*: ¹H NMR (400 MHz, DMSO-d₆) δ 8.81 (t, J=1.7 Hz, 1H), 8.45 (d, J=2.7 Hz, 1H), 7.99-7.81 (m, 1H), 6.48 (s, 1H), 4.75 (m, 1H), 3.75-3.50 (m, 4H), 3.30-3.22 (m, 1H), 2.66-2.57 (m, 4H), 2.24-2.11 (m, 2H), 1.92 (t, J=3.1 Hz, 1H), 1.75 (m, 2H), 1.66-1.54 (m, 4H), 1.45 (d, J=6.6 Hz, 6H).

Compound 188*: ¹H NMR (400 MHz, DMSO-d₆) δ 8.82 (s, 1H), 8.45 (d, J=2.8 Hz, 1H), 7.96-7.88 (m, 1H), 6.64 (s, 1H), 6.52 (s, 1H), 5.77 (s, 1H), 4.83-4.66 (m, 1H), 3.70-3.55 (m, 4H), 2.95-2.84 (m, 1H), 2.64-2.58 (m, 4H), 2.35-2.31 (m, 1H), 2.15-2.07 (m, 2H), 1.84-1.59 (m, 7H), 1.44 (d, J=6.6 Hz, 6H), 1.15 (d, J=13.3 Hz, 1H).

### Example CJ: 4-((1R,3r,5S,6r)-6-(1-isopropyl-3-(3-(trifluoromethoxy)phenyl)-1H-pyrazol-5-yl)bicyclo[3.1.0]hexan-3-yl)-1,4-oxazepane and 4-((1R,3s,5S,6r)-6-(1-isopropyl-3-(3-(trifluoromethoxy)phenyl)-1H-pyrazol-5-yl)bicyclo[3.1.0]hexan-3-yl)-1,4-oxazepane (Compounds 189* & 190*)

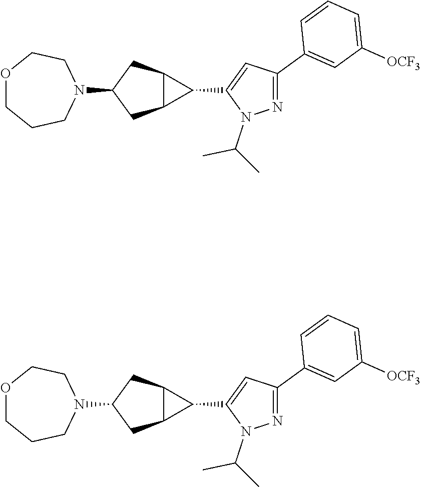 [SMILES: CC(C)n1nc(-c2cccc(OC(F)(F)F)c2)cc1[C@H]1[C@@H]2C[C@H](N3CCCOCC3)C[C@@H]21 ; CC(C)n1nc(-c2cccc(OC(F)(F)F)c2)cc1[C@H]1[C@@H]2C[C@@H](N3CCCOCC3)C[C@@H]21]

The titled compounds were synthesized following a procedure similar to synthesize compounds 175* and 176* using (3-(trifluoromethoxy)phenyl)boronic acid in step 2. The two diastereoisomers were purified by prep-HPLC (acetonitrile/water gradient with 0.1% NH₄OH) to provide compounds 189* and 190*. The relative stereochemistry was arbitrarily assigned. LCMS (ESI) [M+H]⁺=450.3.

Compound 189*: ¹H NMR (400 MHz, DMSO-d₆) δ 7.75 (d, J=8.0 Hz, 1H), 7.64 (s, 1H), 7.49 (t, J=8.0 Hz, 1H), 7.23 (d, J=8.3 Hz, 1H), 6.39 (s, 1H), 4.83-4.63 (m, 1H), 3.69-3.55 (m, 4H), 3.29-3.21 (m, 1H), 2.66-2.58 (m, 4H), 2.17 (m, 2H), 1.89 (t, J=3.0 Hz, 1H), 1.75 (m, 2H), 1.66-1.54 (m, 4H), 1.44 (d, J=6.8 Hz, 6H).

Compound 190*: ¹H NMR (400 MHz, DMSO-d₆) δ 7.75 (d, J=8.0 Hz, 1H), 7.64 (s, 1H), 7.49 (t, J=8.0 Hz, 1H), 7.23 (d, J=8.3 Hz, 1H), 6.42 (s, 1H), 4.72 (m, 1H), 3.70-3.53 (m, 4H), 2.93-2.81 (m, 1H), 2.64-2.56 (m, 4H), 2.10 (dd, J=7.0, 12.3 Hz, 2H), 1.82-1.58 (m, 7H), 1.42 (d, J=6.5 Hz, 6H).

### Example CK: 3-(5-((1R,3r,5S,6r)-3-(1,4-Oxazepan-4-yl)bicyclo[3.1.0]hexan-6-yl)-1-isopropyl-1H-pyrazol-3-yl)benzonitrile and 3-(5-((1R,3s,5S,6r)-3-(1,4-oxazepan-4-yl)bicyclo[3.1.0]hexan-6-yl)-1-isopropyl-1H-pyrazol-3-yl)benzonitrile (Compounds 191* & 192*)

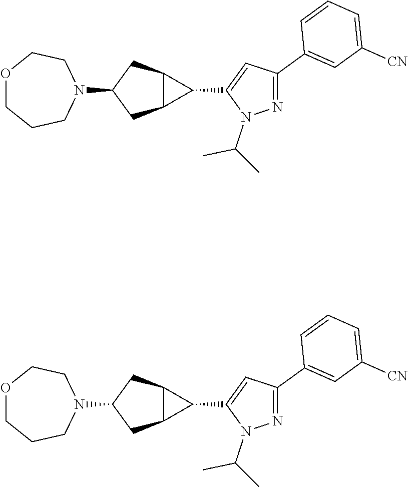 [SMILES: CC(C)n1nc(-c2cccc(C#N)c2)cc1[C@H]1[C@@H]2C[C@H](N3CCCOCC3)C[C@@H]21 ; CC(C)n1nc(-c2cccc(C#N)c2)cc1[C@H]1[C@@H]2C[C@@H](N3CCCOCC3)C[C@@H]21]

The titled compounds were synthesized following a procedure similar to synthesize compounds 175* and 176* using (3-(trifluoromethoxy)phenyl)boronic acid in step 2. The two diastereoisomers were purified by prep-HPLC (acetonitrile/water gradient with 0.1% NH₄OH) to provide compounds 191* and 192*. The relative stereochemistry was arbitrarily assigned. LCMS (ESI) [M+H]⁺=391.2.

Compound 191*: ¹H NMR (400 MHz, DMSO-d₆) δ 8.18-8.01 (m, 2H), 7.76-7.51 (m, 2H), 6.45 (s, 1H), 4.85-4.65 (m, 1H), 3.94-3.44 (m, 4H), 3.29-3.25 (m, 1H), 2.64 (m, 4H), 2.19 (m, 2H), 1.91 (t, J=2.9 Hz, 1H), 1.83-1.70 (m, 2H), 1.67-1.53 (m, 4H), 1.51-1.40 (m, 6H).

Compound 192*: ¹H NMR (400 MHz, DMSO-d₆) δ 8.11 (s, 1H), 8.05 (d, J=7.8 Hz, 1H), 7.70 (d, J=7.6 Hz, 1H), 7.60-7.53 (m, 1H), 6.47 (d, J=2.0 Hz, 1H), 4.79-4.67 (m, 1H), 3.69-3.55 (m, 4H), 2.93-2.82 (m, 1H), 2.60 (d, J=3.4 Hz, 4H), 2.13-2.06 (m, 2H), 1.82-1.75 (m, 3H), 1.69 (t, J=10.4 Hz, 2H), 1.61 (br s, 2H), 1.45-1.40 (m, 6H).

### Example CL: 4-((1R,3r,5S,6r)-6-(3-(2-Fluorophenyl)-1-isopropyl-1H-pyrazol-5-yl)bicyclo[3.1.0]hexan-3-yl)-1,4-oxazepane and 4-((1R,3s,5S,6r)-6-(3-(2-fluorophenyl)-1-isopropyl-1H-pyrazol-5-yl)bicyclo[3.1.0]hexan-3-yl)-1,4-oxazepane (Compounds 193* & 194*)

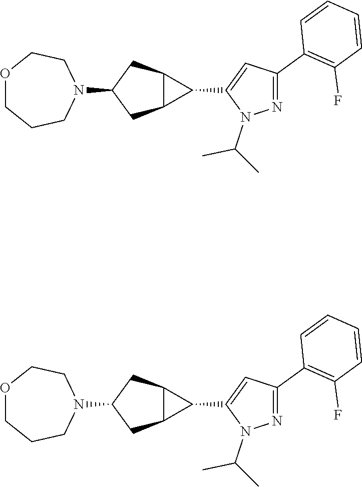 [SMILES: CC(C)n1nc(-c2ccccc2F)cc1[C@H]1[C@@H]2C[C@H](N3CCCOCC3)C[C@@H]21 ; CC(C)n1nc(-c2ccccc2F)cc1[C@H]1[C@@H]2C[C@@H](N3CCCOCC3)C[C@@H]21]

The titled compounds were synthesized following a procedure similar to synthesize compounds 175* and 176* using (2-fluorophenyl)boronic acid in step 2. The two diastereoisomers were purified by prep-HPLC (acetonitrile/water gradient with 0.1% NH₄OH) to provide compounds 193* and 194*. The relative stereochemistry was arbitrarily assigned.

Compound 193*: ¹H NMR (400 MHz, DMSO-d₆) δ 7.89 (dt, J=1.6, 7.9 Hz, 1H), 7.40-7.12 (m, 3H), 6.15 (d, J=4.2 Hz, 1H), 4.84-4.65 (m, 1H), 3.67-3.53 (m, 4H), 3.29-3.21 (m, 1H), 2.66-2.57 (m, 4H), 2.22-2.10 (m, 2H), 1.93-1.87 (m, 1H), 1.75 (m, 2H), 1.65-1.54 (m, 4H), 1.45 (d, J=6.5 Hz, 6H). LCMS (ESI) [M+H]⁺=384.2.

Compound 194*: ¹H NMR (400 MHz, DMSO-d₆) δ not determined. LCMS (ESI) [M+H]⁺=384.2.

### Example CM: 4-((1R,3r,5S,6r)-6-(3-(3-Fluorophenyl)-1-isopropyl-1H-pyrazol-5-yl)bicyclo[3.1.0]hexan-3-yl)-1,4-oxazepane and 4-((1R,3s,5S,6r)-6-(3-(3-fluorophenyl)-1-isopropyl-1H-pyrazol-5-yl)bicyclo[3.1.0]hexan-3-yl)-1,4-oxazepane (Compounds 195* & 196*)

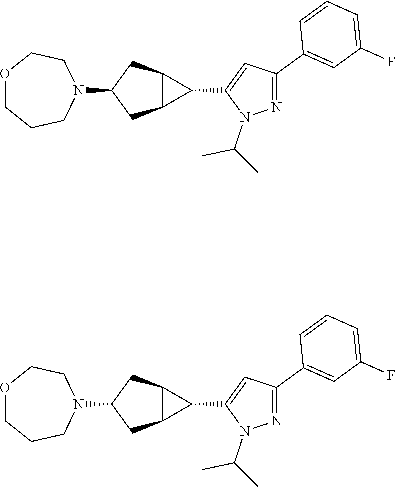 [SMILES: CC(C)n1nc(-c2cccc(F)c2)cc1[C@H]1[C@@H]2C[C@H](N3CCCOCC3)C[C@@H]21 ; CC(C)n1nc(-c2cccc(F)c2)cc1[C@H]1[C@@H]2C[C@@H](N3CCCOCC3)C[C@@H]21]

The titled compounds were synthesized following a procedure similar to synthesize compounds 175* and 176* using (3-fluorophenyl)boronic acid in step 2. The two diastereoisomers were purified by prep-HPLC (acetonitrile/water gradient with 0.1% NH₄OH) to provide compounds 195* and 196*. The relative stereochemistry was arbitrarily assigned. LCMS (ESI) [M+H]⁺=384.2.

Compound 195*: ¹H NMR (400 MHz, DMSO-d₆) δ 7.59-7.33 (m, 3H), 7.10-7.01 (m, 1H), 6.33 (s, 1H), 4.82-4.62 (m, 1H), 3.69-3.55 (m, 4H), 3.30-3.21 (m, 1H), 2.66-2.57 (m, 4H), 2.23-2.11 (m, 2H), 1.88 (t, J=3.1 Hz, 1H), 1.82-1.68 (m, 2H), 1.65-1.51 (m, 4H), 1.44 (d, J=6.5 Hz, 6H).

Compound 196*: ¹H NMR (400 MHz, DMSO-d₆) δ 7.60-7.34 (m, 3H), 7.13-6.99 (m, 1H), 6.37 (s, 1H), 4.71 (m, 1H), 3.70-3.53 (m, 4H), 2.93-2.82 (m, 1H), 2.64-2.56 (m, 4H), 2.09 (dd, J=7.1, 12.3 Hz, 2H), 1.81-1.56 (m, 7H), 1.42 (d, J=6.6 Hz, 6H).

### Example CN: 4-((1R,3r,5S,6r)-6-(1-Isopropyl-3-(o-tolyl)-1H-pyrazol-5-yl)bicyclo[3.1.0]hexan-3-yl)-1,4-oxazepane and 4-((1R,3s,5S,6r)-6-(1-isopropyl-3-(o-tolyl)-1H-pyrazol-5-yl)bicyclo[3.1.0]hexan-3-yl)-1,4-oxazepane (Compounds 197* & 198*)

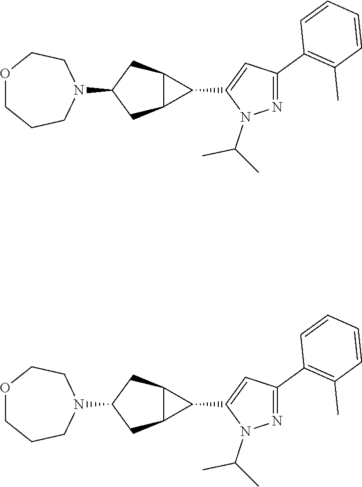 [SMILES: Cc1ccccc1-c1cc([C@H]2[C@@H]3C[C@H](N4CCCOCC4)C[C@@H]32)n(C(C)C)n1 ; Cc1ccccc1-c1cc([C@H]2[C@@H]3C[C@@H](N4CCCOCC4)C[C@@H]32)n(C(C)C)n1]

The titled compounds were synthesized following a procedure similar to synthesize compounds 175* and 176* using (3-fluorophenyl)boronic acid in step 2. The two diastereoisomers were purified by prep-HPLC (acetonitrile/water gradient with 0.1% NH₄OH) to provide compounds 197* and 198*. The relative stereochemistry was arbitrarily assigned. LCMS (ESI) [M+H]⁺=380.3.

Compound 197*: ¹H NMR (400 MHz, CDCl₃) δ 7.54-7.46 (m, 1H), 7.24-7.11 (m, 3H), 5.88 (s, 1H), 4.75-4.61 (m, 1H), 3.87-3.70 (m, 4H), 3.37 (m, 1H), 2.96-2.59 (m, 4H), 2.46 (s, 3H), 2.43-2.29 (m, 2H), 1.91 (m, 1H), 1.75-1.60 (m, 6H), 1.55 (d, J=6.6 Hz, 6H).

Compound 198*: ¹H NMR (400 MHz, DMSO-d₆) δ 7.56-7.41 (m, 1H), 7.25-7.12 (m, 3H), 6.08 (s, 1H), 4.71 (td, J=6.5, 13.0 Hz, 1H), 3.78-3.53 (m, 4H), 2.96 (br s, 1H), 2.67 (br s, 4H), 2.42 (s, 3H), 2.20-2.04 (m, 2H), 1.92-1.51 (m, 7H), 1.42 (d, J=6.5 Hz, 6H).

### Example CO: 4-((1R,3r,5S,6r)-6-(3-(3-(Difluoromethoxy)phenyl)-1-isopropyl-1H-pyrazol-5-yl)bicyclo[3.1.0]hexan-3-yl)-1,4-oxazepane and 4-((1R,3s,5S,6r)-6-(3-(3-(difluoromethoxy)phenyl)-1-isopropyl-1H-pyrazol-5-yl)bicyclo[3.1.0]hexan-3-yl)-1,4-oxazepane (Compounds 199* & 200*)

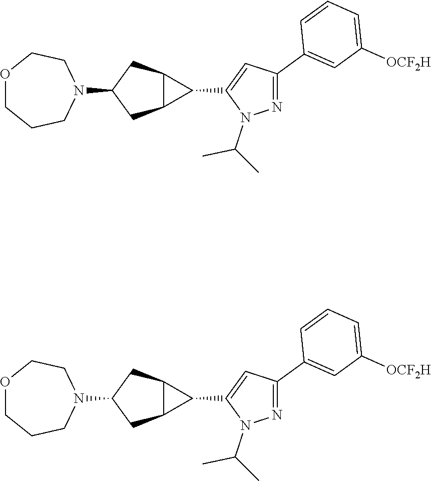

The titled compounds were synthesized following a procedure similar to synthesize compounds 175* and 176* using (3-(difluoromethoxy)phenyl)boronic acid in step 2. The two diastereoisomers were purified by prep-HPLC (acetonitrile/water gradient with 0.1% NH₄OH) to provide compounds 199* and 200*. The relative stereochemistry was arbitrarily assigned. LCMS (ESI) [M+H]⁺=432.2.

Compound 199*: ¹H NMR (400 MHz, DMSO-d₆) δ 7.60 (d, J=7.8 Hz, 1H), 7.50-7.38 (m, 2H), 7.27 (s, 1H), 7.11-7.02 (m, 1H), 6.34 (s, 1H), 4.83-4.63 (m, 1H), 3.72-3.52 (m, 4H), 3.30-3.21 (m, 1H), 2.65-2.58 (m, 4H), 2.23-2.13 (m, 2H), 1.90 (t, J=3.1 Hz, 1H), 1.76 (m, 2H), 1.65-1.56 (m, 4H), 1.45 (d, J=6.5 Hz, 6H).

Compound 200*: ¹H NMR (400 MHz, DMSO-d₆) δ 7.60 (d, J=7.8 Hz, 1H), 7.51-7.38 (m, 2H), 7.27 (s, 1H), 7.10-7.02 (m, 1H), 6.37 (s, 1H), 4.72 (m, 1H), 3.71-3.49 (m, 4H), 2.95-2.82 (m, 1H), 2.64-2.56 (m, 4H), 2.15-2.05 (m, 2H), 1.81-1.59 (m, 7H), 1.43 (d, J=6.8 Hz, 6H).

### Example CP: 4-((1R,3r,5S,6r)-6-(3-(4-(Difluoromethoxy)phenyl)-1-isopropyl-1H-pyrazol-5-yl)bicyclo[3.1.0]hexan-3-yl)-1,4-oxazepane (Compound 201*)

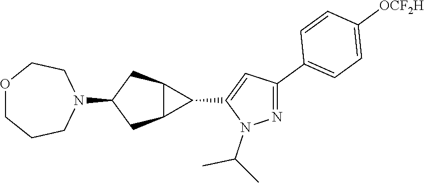

The titled compounds were synthesized following a procedure similar to synthesize compounds 175* and 176* using (4-(difluoromethoxy)phenyl)boronic acid in step 2. The two diastereoisomers were purified by prep-HPLC (acetonitrile/water gradient with 0.1% NH₄OH) to provide compound 201*. The relative stereochemistry was arbitrarily assigned and only one isomer was isolated. LCMS (ESI) [M+H]⁺=432.2.

Compound 201*: ¹H NMR (400 MHz, DMSO-d₆) δ 7.79-7.69 (m, 2H), 7.24 (s, 1H), 7.16 (d, J=8.7 Hz, 2H), 6.27 (s, 1H), 4.82-4.62 (m, 1H), 3.71-3.56 (m, 4H), 3.31-3.23 (m, 1H), 2.68-2.58 (m, 4H), 2.23-2.12 (m, 2H), 1.88 (t, J=3.1 Hz, 1H), 1.76 (m, 2H), 1.66-1.55 (m, 4H), 1.44 (d, J=6.6 Hz, 6H).

### Example CO: 4-((1R,3s,5S,6r)-6-(1-isopropyl-3-(5-(trifluoromethyl)pyridin-3-yl)-1H-pyrazol-5-yl)bicyclo[3.1.0]hexan-3-yl)morpholine (Compound 202)

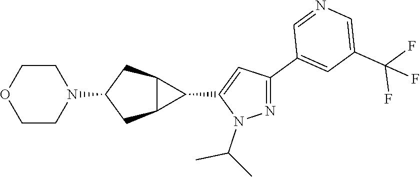 [SMILES: CC(C)n1nc(-c2cncc(C(F)(F)F)c2)cc1[C@H]1[C@@H]2C[C@@H](N3CCOCC3)C[C@@H]21]

The titled compound was synthesized following a procedure similar to synthesize compounds 175* and 176* using morpholine in step 1 and (5-(trifluoromethyl)pyridin-3-yl)boronic acid in step 2. The two diastereoisomers were purified by prep-HPLC (acetonitrile/water gradient with 0.1% NH₄OH) to provide compound 171 and 202). The relative stereochemistry was assigned based on ¹H NMR analysis. LCMS (ESI) [M+H]⁺=421.3.

Compound 202: ¹H NMR (400 MHz, DMSO-d₆) δ 9.22 (d, J=1.9 Hz, 1H), 8.84 (d, J=2.2 Hz, 1H), 8.36 (s, 1H), 6.64 (s, 1H), 4.82-4.71 (m, 1.H), 3.56 (t, J=4.6 Hz, 4H), 2.47-2.40 (m, 1H), 2.38-2.30 (m, 4H), 2.14 (dd, J=11.9, 6.9 Hz, 2H), 1.82 (t, J=3.0 Hz, 1H), 1.74-1.62 (m, 4H), 1.45 (d, J=6.5 Hz, 6H).

### Example CR: 4-((1R,3r,5S,6r)-6-(1-isopropyl-3-(2-(trifluoromethyl)pyrimidin-5-yl)-1H-pyrazol-5-yl)bicyclo[3.1.0]hexan-3-yl)morpholine (Compound 203)

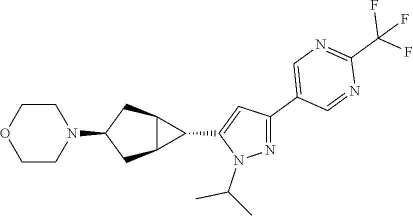 [SMILES: CC(C)n1nc(-c2cnc(C(F)(F)F)nc2)cc1[C@H]1[C@@H]2C[C@H](N3CCOCC3)C[C@@H]21]

The titled compound was synthesized following a procedure similar to synthesize compounds 175* and 176* using morpholine in step 1 and [2-(trifluoromethyl)pyrimidin-5-yl]boronic acid in step 2. The two diastereoisomers were purified by prep-HPLC (acetonitrile/water gradient with 0.1% NH₄OH) to provide compound 203. The relative stereochemistry was assigned based on ¹H NMR analysis and only one isomer was isolated. LCMS (ESI) [M+H]⁺=422.3.

Compound 203: ¹H NMR (400 MHz, DMSO-d₆) δ 9.31 (s, 2H), 6.64 (s, 1H), 4.85-4.70 (m, 1H), 3.56 (t, J=4.6 Hz, 4H), 2.85-2.74 (m, 1H), 2.39-2.32 (m, 4H), 2.17-2.04 (m, 3H), 1.76 (dd, J=13.7, 6.0 Hz, 2H), 1.68-1.61 (m, 2H), 1.48 (d, J=6.6 Hz, 6H).

### Example CS: (R)-4-((1R,3r,5S,6r)-6-(1-isopropyl-3-(2-(trifluoromethyl)pyrimidin-5-yl)-1H-1,2,4-triazol-5-yl)bicyclo[3.1.0]hexan-3-yl)-3-methylmorpholine (Compound 204)

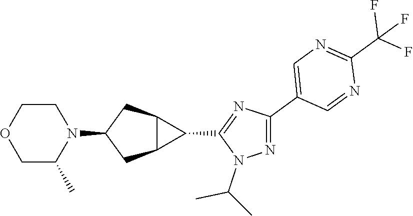 [SMILES: CC(C)n1nc(-c2cnc(C(F)(F)F)nc2)nc1[C@H]1[C@@H]2C[C@H](N3CCOC[C@H]3C)C[C@@H]21]

The titled compound was synthesized following a procedure similar to compound 51 using (1R,5S,6r)-6-(1-isopropyl-3-(5-(trifluoromethyl)pyridin-3-yl)-1H-1,2,4-triazol-5-yl)bicyclo[3.1.0]hexan-3-one and (2R)-2-methylmorpholine in final step. Purification of the crude mixture was performed by reverse phase chromatography (water (NH₃H₂O+NH₄HCO₃)-ACN, 20%-50%) to provide the title compound 205 (second peak on HPLC (basic), 27.1 mg, 30.8% yield). The relative stereochemistry was determined by 2D-NMR. LCMS (ESI) [M+H]⁺=437.5.

Compound 204: ¹H NMR (400 MHz, CD₃OD) δ 9.45 (s, 2H), 4.92-4.89 (m, 1H), 3.78-3.63 (m, 3H), 3.54-3.44 (m, 2H), 2.88-2.85 (m, 1H), 2.69-2.63 (m, 1H), 2.53-2.49 (m, 1H), 2.33-2.20 (m, 2H), 2.18 (t, J=3.2 Hz, 1H), 2.04 (br s, 2H), 1.86-1.81 (m, 1H), 1.78-1.73 (m, 1H), 1.58 (d, J=6.8 Hz, 6H), 1.09 (d, J=6.8 Hz, 3H).

### Biological Assay Examples

### Mouse OPC Preparation

To assess effects of treatments on OPCs, all treatments were assayed in two or more independent platings of epiblast stem cell-derived OPCs (EpiSC). EpiSC-derived OPCs were obtained using in vitro differentiation protocols and culture conditions described previously (Najm et al, 2011, Nature Methods). OPCs were expanded and frozen down in aliquots. OPCs were thawed into growth conditions for at least one passage before use in further assays.

### Determination of EC₅₀ Values of Compounds

### A: In Vitro Phenotypic Screening of OPCs

EpiSC-derived OPCs were grown and expanded in poly-L-ornithine (PO) and laminin-coated flasks in N2B27 media (DMEM/F12 (Gibco), N2-MAX (R&D Systems), B-27 (ThermoFisher), and GlutaMax (Gibco)) supplemented with FGF2 (10 μg/mL, R&D systems, 233-FB-025) and PDGF-AA (10 μg/mL, R&D systems, 233-AA-050) before harvesting for experiments. The cells were seeded onto poly-L-ornithine or poly-D-lysine coated CellCarrier Ultra plates (PerkinElmer) coated with laminin (Sigma, L2020) at a density of 150,000/cm² in N2B27 media without growth factors. For dose-response testing, a 1000× compound stock in dimethyl sulphoxide (DMSO) was added to assay plates, resulting in 8-point dose curves with final concentrations between 1000 nM and 0.5 nM. Positive controls and DMSO vehicle controls were included in each assay plate. Cells were incubated under standard conditions (37° C., 5% CO) for 3 days and fixed with 4% paraformaldehyde (PFA) in phosphate buffered saline (PBS) for 20 min. Fixed plates were washed with PBS, permeabilized with 0.1% Triton X-100, and blocked with 10% donkey serum (v/v) in PBS for 40 min. Then, cells were labelled with MBP antibodies (Abcam, ab7349; 1:200) for 2 h at room temperature, washed with PBS, and stained with Alexa Fluor conjugated secondary antibodies (1:500) for 45 min. Nuclei were visualized by DAPI staining (Sigma; 1 g/ml), followed by further PBS washes.

### B: High-Content Imaging and Analysis

Cells and cell culture plates were imaged on the Operetta High Content Imaging and Analysis system (PerkinElmer). Analysis (PerkinElmer Harmony and Columbus software) began by identifying intact nuclei stained by DAPI. The peri-nuclear region of each cell was then cross-referenced with the mature myelin protein (MBP) stain to identify oligodendrocyte nuclei, and from this the percentage of oligodendrocytes was calculated. EC₅₀ values were calculated using The Levenberg-Marquardt algorithm to fit a Hill equation to dose-response data (0.5 nM to 1000 nM). The results are provided in Table 3 (OPC EC₅₀).

### Determination of Potency and Enzyme Target

### GC/MS-Based Sterol Profiling

Sterols were monitored using a modified Folch wash protocol (Hubler et al, 2018, Nature). EpiSC-derived OPCs were plated at 100,000 cells per well in PO- and laminin-coated 96-well plates in N2B27 media without growth factors. After 24 hours, cells were rinsed with saline and plates were frozen. Cholesterol-d7 standard was then added to each well before drying under nitrogen stream and derivatization with 55 μl of bis(trimethylsilyl) trifluoroacetamide. After derivatization, 2 μl were analyzed by gas chromatography/mass spectrometry using an Agilent 5973 Network Mass Selective Detector equipped with a 6890 gas chromatograph system and a HP-5MS capillary column (30 m×0.25 mm×0.25 mm). Samples were analyzed in full scan mode using electron impact ionization; ion fragment peaks were integrated to calculate sterol abundance, and quantitation was relative to cholesterol-d7. The following ion fragments were used to quantitate each metabolite: cholesterol-d7 (465), FF-Mas (482), cholesterol (368), zymostenol (458), zymosterol (456), Desmosterol (456, 343), 7-dehydrocholesterol (456, 325), lanosterol (393), lathosterol (458), 14-dehydrozymostenol (456, 351). For reference, Table 2 shows sterol GC-MS analytes and their relationship with inhibitors of cholesterol biosynthesis. All standards were obtained from Avanti Polar Lipids unless otherwise indicated. Calibration curves were generated by injecting varying concentrations of sterol standards and maintaining a fixed amount of cholesterol-D7. For normalized zymostenol accumulation results, the total amount of zymostenol measured after drug treatment was divided by the total amount of zymostenol accumulated after 24 hr treatment with 100 nM positive control reference. EC₅₀ values were calculated using The Levenberg-Marquardt algorithm to fit a Hill equation to dose-response data (8 doses from 0.15 nM to 333 nM). EC₅₀ values for zymostenol (Zymostenol GCMS EC₅₀) are provided in Table 3.

TABLE 2

| Analyte increased | Cholesterol enzyme inhibited |
|---|---|
| Lanosterol | CYP51 |
| FF-MAS | TM7SF2 (Sterol 14 Reductase) |
| 14-dehydrozymostenol | TM7SF2 (Sterol 14 Reductase) |
| Zymosterol | EBP |
| Zymostenol | EBP |
| 7-dehydrocholesterol | DHCR7 |

Membrane preparation: To examine compound binding affinity to EBP, human EBP was overexpressed in human embryonic kidney 293 cells. Cell pellet was lysed in 10 times weight binding buffer (50 mM tris(hydroxymethyl)aminomethane (Tris, Alfa Aesar Cat #A18494), 5 mM MgCl₂ (Sigma Cat #M2670), 0.1 mM ethylenediaminetetraacetic acid tetrasodium salt hydrate (EDTA; Sigma Cat #E5391), 1× protease inhibitor cocktail, pH 7.5) on ice by using a dounce homogenizer. The solution was centrifuged at 25,000 g for 50 min at 4° C. The membrane pellet was re-suspended in binding buffer and run through a 25⅝ gauge needle. After checking the concentration by Bradford assay, the whole cell membrane solution was adjusted to 20 mg/mL and stored at −80° C.

Determination of equilibrium dissociation constant Kd of radioligand: Membrane prepared as described above was pre-incubated with PVT-WGA SPA beads (Perkinelmer Cat #RPNQ0003) at a ratio of 0.3 mg beads with 5 μg membrane per 25 μL binding buffer at 20° C. for 2 hours with gentle shaking. This binding solution was centrifuged at 400 g for 5 minutes to collect the bead/membrane mixture. After re-suspending the pellet in binding buffer at the same calculated volume with 0.01% (w/v) Bovine Serum Albumin (BSA) (Sigma Cat #A1933), the bead/membrane mixture was added in 384-well low-binding surface plate (PerkinElmer Cat #6057480) at 25 μl/well. Radioligand at different concentrations with and without the non-radio-labeled same ligand 5 μM (for nonspecific and total signal, respectively) was added to bring final volume to 50 μl/well with DMSO concentration at 0.1%. At equilibrium (3 hours after ligand addition), radiometric signal CPM was counted by using a Microbeta2 microplate counter (PerkinElmer). The Kd was determined by nonlinear regression fitting of specific signal plot against the concentration of radioligand [3H]-Ifenprodil (PerkinElmer Cat #NET1089250UC); K_{d}=15.86 nM at 10 nM concentration of radioligand used in assay.

Competition binding assay to determine compound affinity: The same conditions of the radioligand Kd study were used for compound single dose percentage inhibition and equilibrium dissociation constant Ki examinations, except 50 nL compound DMSO stock was pre-added in 384-well low-binding surface plate (PerkinElmer Cat #6057480) by Echo 550 (Labcyte) to reach the final concentration for single dose test at 1 μM, and dose response test from 0.06 nM to 5 μM (8 dose, 5 times dilution). A pre-incubated bead/membrane mixture was added in compound plate at 0.3 mg beads and 5 g membrane per well. Radioligand [3H]-Ifenprodil was added to reach optimized concentration [L] and bring assay volume to 50 μl. At equilibrium (3 hours after ligand addition), radiometric signal was counted as described above. The percentage inhibition of compound at each testing concentration was calculated by normalizing each condition's CPM readout to full block (5 μM non-radiolabeled ligand) and non-block (DMSO) control conditions. Compound binding inhibition IC₅₀ was determined by nonlinear regression fitting of percentage inhibition plot against compound concentration. Compound Ki was calculated from the equation Ki=IC₅₀/(1+[L]/Kd), which [L] was radioligand concentration used in assay. All tests had N bigger or equal to 2.

### Determination of Binding Affinity to EBP-7-Dehydrocholesterol Reductase

Membrane Preparation: human emopamil binding protein and human 7-dehydrocholesterol reductase co-expressing cells were generated by transient transfecting host human embryonic kidney (HEK) 293 cells with 2 DNA constructs containing each protein's coding sequence. Cells were suspension cultured at 37° C. with 5% CO₂ in FREESTYLE 293 Expression Medium (Thermofisher). Whole cell membrane was prepared by harvesting the cell pellet, adding cold membrane buffer (50 mM Tris, pH7.5, 1× Roche COMPLETE EDTA-free protease inhibitor cocktail) 10 times volume of the cell pellets weight, lysing cell pellet on ice by using Dounce homogenizer, spinning at 200 g 4° C. for 15 min, collecting supernatant and spinning again at 25000 g 4° C. for 50 min, transferring pellet to Dounce homogenizer, re-suspending pellet by homogenizing in membrane buffer on ice to reach ˜25 mg/mL, then keeping whole cell membrane aliquots at −80° C.

Compounds were prepared in a 96-well U bottom plate (Corning Cat #7007) using an Echo550 machine and 10 mM compound DMSO stock solution, followed by an 8-dose 5-fold serial dilutions protocol with final testing compound concentration ranging from 0.06 to 5000 nM, with DMSO back fill to 100 nL/well and n=2. DMSO and Ifenprodil (Sigma, Cat #12892) 5 μM wells were added in each plate as 0 and 100% inhibition reference controls with n=8 for each condition. The UniFilter-96 GF/B plates (PerkinElmer Cat #6005177) were pre-treated by adding 50 μl/well of 0.3% (v/v) Polyethylenimine (PEI) (branched, Sigma Cat #408727) to UniFilter-96 GF/B plates. The plates were sealed and incubated at 4° C. for 3 hrs. Then, the plates were washed with ice-cold assay buffer 3 times. The radioligand binding assay was prepared by adding assay buffer diluted hEBP-DHCR7 membrane at 66.7 g/ml×150 μl/well into the 96-well compound plate to reach 10 μg membrane per well. Then, the assay buffer diluted [3H]—(S)-6-(2-Methyl-3-(6-(trifluoromethyl)pyridin-3-yl)propyl)-2-thia-6-azaspiro[3.4]octane 2,2-dioxide (Moravek, Cat #MT-1003106) was added at 25 nM×50 μl/well. Following this, the plate was centrifuged at 1000 rpm for 30 secs. The plate was then sealed and agitated at 600 rpm at 22° C. for 5 min, and then incubated at 22° C. for 3 hrs. The incubation was stopped by transferring the binding solution to the pre-treated UniFilter-96 GF/B plate, vacuum filtrated, and then washed four times with ice-cold assay buffer. Following this, the plates were dried at 37° C. for 45 min. The plates were then sealed at the bottom. 40 l/well of scintillation cocktail was added to the plates. A MicroBeta2 microplate counter was then used to read the plate and analyze the data. For reference and test compounds, the results are expressed as % Inhibition, using the normalization equation: N=100−100×(U−C₂)/(C1−C2), where U is the unknown value, C1 is the average of high controls, and C2 is the average value of low controls. The IC₅₀ was determined by fitting percentage of inhibition as a function of compound concentrations with Hill equation using XLfit. Results are expressed as hEBP-DHCR7 Ki (μM) in Table 3. Ki was calculated as described above. The asterix (*) indicates an isolated isomer or isolated group of isomers, but that the stereochemistry has been arbitrarily assigned; ND=not determined.

TABLE 3

| | hEBP- | Mouse | |
|---|---|---|---|
| | DHCR7 | Zymostenol | OPC |
| Cmpd | Ki | GCMS | EC₅₀ |
| No. | (μM) | EC₅₀ (μM) | (μM) |
| 1* | 0.0389 | 0.0209 | 0.0806 |
| 2* | 0.0405 | 0.017 | 0.0975 |
| 3 | 0.0324 | 0.0395 | ND |
| 4 | 0.00828 | 0.0123 | 0.0387 |
| 5 | 0.0527 | 0.0442 | 0.196 |
| 6 | 0.0347 | 0.0105 | 0.0343 |
| 7* | 0.135 | 0.0263 | 0.158 |
| 8* | 0.0382 | 0.0448 | 0.280 |
| 9* | 0.113 | 0.0214 | 0.0734 |
| 10* | 0.0958 | 0.018 | 0.0542 |
| 11* | 0.134 | 0.0197 | 0.108 |
| 12* | 0.12 | 0.064 | 0.0638 |
| 13 | ND | 0.00148 | 0.00318 |
| 14 | ND | 0.0402 | 0.0813 |
| 15 | ND | 0.00598 | 0.0224 |
| 16 | ND | 0.203 | 0.333 |
| 17 | ND | 0.00174 | 0.00416 |
| 18 | ND | 0.0528 | 0.0785 |
| 19* | 0.00993 | 0.00792 | 0.140 |
| 20* | ND | 0.064 | 0.232 |
| 21* | 0.0271 | 0.00771 | 0.0517 |
| 22* | 0.0109 | 0.00133 | 0.00673 |
| 23* | 0.0155 | 0.0131 | 0.132 |
| 24* | 0.0089 | 0.00165 | 0.0113 |
| 25* | ND | 0.234 | 0.0655 |
| 26* | 0.0226 | 0.0117 | 0.085 |
| 27* | ND | 0.141 | ND |
| 28* | 0.0299 | 0.00811 | 0.0804 |
| 29* | 0.02 | 0.00319 | 0.0198 |
| 30* | 0.0325 | 0.0148 | 0.09 |
| 31 | 0.1 | 0.241 | 0.643 |
| 32 | 0.0345 | 0.052 | 0.354 |
| 33 | 0.0194 | 0.00533 | 0.0305 |
| 34 | 0.0345 | 0.052 | 0.354 |
| 35* | 0.112 | 0.03 | 0.175 |
| 36* | 0.211 | 0.23 | 0.144 |
| 37* | 0.033 | 0.018 | 0.098 |
| 38* | 0.0476 | 0.00932 | 0.071 |
| 39* | 0.771 | 0.255 | 0.523 |
| 40* | 0.111 | 0.078 | 0.325 |
| 41* | 0.0226 | 0.00286 | 0.00775 |
| 42* | 0.0236 | 0.009 | 0.0439 |
| 43* | 0.065 | 0.243 | ND |
| 44* | 0.065 | 0.062 | ND |
| 45* | 0.053 | 0.011 | 4.80E−02 |
| 46* | 0.0348 | 0.00142 | 9.07E−04 |
| 47 | 0.043 | 0.046 | 0.145 |
| 48 | 0.0184 | 0.00728 | 0.0116 |
| 49 | 0.053 | 0.042 | 0.075 |
| 50 | 0.021 | 0.00162 | 0.00772 |
| 51 | 0.358 | 0.077 | 0.0825 |
| 52 | 0.0181 | 0.0031 | 0.00629 |
| 53 | 0.0125 | 0.0138 | 0.075 |
| 54 | 0.0106 | 0.003 | 0.0124 |
| 58* | 0.0262 | 0.0251 | 0.138 |
| 59* | 0.0734 | 0.0192 | ND |
| 60* | 0.0192 | 0.00552 | 0.0362 |
| 61* | 0.0691 | 0.0219 | 0.102 |
| 62* | 0.0131 | 0.00173 | 0.0183 |
| 63* | 0.0144 | 0.00349 | 0.0218 |
| 64* | 0.0156 | 0.00275 | ND |
| 65* | 0.0282 | 0.0162 | 0.103 |
| 66 | 0.0502 | 0.00284 | 0.0149 |
| 67 | 0.05 | 0.0665 | 0.233 |
| 68 | 0.119 | 0.146 | 0.417 |
| 69 | 0.0279 | 0.00819 | 0.0273 |
| 70* | 0.00876 | 0.00643 | 6.50E−04 |
| 71* | 0.029 | 0.205 | 7.49E−01 |
| 72 | 1.17 | 0.333 | 9.80E−01 |
| 73 | 0.109 | 0.00395 | 0.00841 |
| 74 | 0.037 | 0.168 | ND |
| 75 | 0.012 | 0.00283 | ND |
| 76 | 0.015 | 0.00118 | 0.00156 |
| 77 | 0.129 | 0.191 | ND |
| 78* | ND | >0.333 | ND |
| 79* | ND | 0.0231 | ND |
| 80* | 0.528 | 0.112 | ND |
| 81* | ND | 0.0273 | ND |
| 82 | 0.087 | 0.28 | ND |
| 83 | 0.0109 | 0.0125 | 0.0784 |
| 84 | 0.0278 | 0.00349 | ND |
| 85 | 0.472 | 0.279 | ND |
| 86* | 0.0312 | 0.0287 | ND |
| 87* | 0.0262 | 0.0164 | 0.0489 |
| 88* | 0.0882 | 0.627 | ND |
| 89* | 0.0424 | 0.0219 | ND |
| 90* | 0.011 | 0.0337 | ND |
| 91* | 0.0128 | 0.0113 | 0.0676 |
| 92* | 0.0776 | 0.252 | ND |
| 93* | 0.0197 | 0.0225 | 0.125 |
| 94 | 0.243 | 0.187 | ND |
| 95 | 0.0333 | 0.0203 | 0.136 |
| 96 | 0.631 | 0.928 | ND |
| 97 | 0.0151 | 0.00387 | 0.0114 |
| 98 | 0.0117 | 0.00486 | 0.0308 |
| 99 | 0.0878 | 0.062 | ND |
| 100 | 0.011 | 0.00599 | ND |
| 101 | 0.00722 | 0.0011 | 0.00202 |
| 102 | 0.073 | 0.055 | ND |
| 103 | 0.0361 | 0.00934 | 0.0934 |
| 104 | 0.0192 | 0.052 | 0.433 |
| 105 | 0.00889 | 0.00315 | 0.0117 |
| 202 | 0.0177 | 0.00476 | 0.0216 |
| 106 | 0.0151 | 0.00387 | 0.0114 |
| 107 | 0.0117 | 0.00486 | 0.0308 |
| 108 | 0.0878 | 0.062 | ND |
| 109* | 0.164 | 0.044 | 0.285 |
| 110* | 0.0148 | 0.00248 | 0.0139 |
| 111* | 0.045 | 0.00164 | 0.00615 |
| 112* | 0.0908 | 0.0238 | 0.196 |
| 113* | 0.00973 | 0.00185 | 0.00481 |
| 114* | 0.0147 | 0.008 | 0.0257 |
| 115* | 0.035 | 0.0972 | ND |
| 116* | 0.169 | 0.049 | ND |
| 117* | 0.0346 | 0.0135 | ND |
| 118* | 0.0322 | 0.0115 | 0.0932 |
| 119 | 0.584 | 0.213 | ND |
| 120 | 0.016 | 0.00125 | 0.0028 |
| 121 | 0.564 | 0.193 | ND |
| 122 | 0.0266 | 0.00781 | ND |
| 123 | 0.0404 | 0.0106 | ND |
| 124 | 2.4 | 0.376 | ND |
| 125 | 0.245 | 0.716 | ND |
| 126 | 0.0308 | 0.00679 | ND |
| 127 | 0.0571 | 0.00132 | 0.006 |
| 128 | 0.118 | 0.0166 | ND |
| 129 | 0.0232 | 0.00279 | ND |
| 130* | 0.392 | 0.619 | ND |
| 131* | 0.106 | 0.168 | ND |
| 132* | 0.088 | 0.352 | ND |
| 133* | 0.0171 | 0.0212 | 0.274 |
| 144 | 0.325 | 0.0057 | 0.048 |
| 145 | 0.299 | 0.0835 | ND |
| 146 | 0.0113 | 9.40E−04 | ND |
| 147 | 1.74 | 0.0145 | ND |
| 148 | 0.0222 | 0.0134 | ND |
| 149 | 0.0683 | 0.129 | ND |
| 150 | 3.54 | 0.946 | ND |
| 151 | 0.505 | 0.129 | ND |
| 152 | 3.42 | 0.00214 | 0.017 |
| 153 | 0.0582 | 0.0134 | ND |
| 154 | 0.465 | 0.221 | ND |
| 155 | 0.465 | 0.305 | ND |
| 156 | 0.0438 | 0.00407 | ND |
| 157* | 0.051 | 0.0592 | ND |
| 158* | 0.0338 | 0.00538 | ND |
| 159* | 0.0122 | 0.00425 | ND |
| 160 | 0.0327 | 0.00103 | 0.0327 |
| 161 | 0.0893 | 0.024 | 0.0893 |
| 162 | 0.838 | 0.371 | 0.838 |
| 163 | 0.0328 | 0.00436 | 0.0328 |
| 164* | 1.64 | 0.143 | ND |
| 165* | 0.0404 | 0.00455 | 0.035 |
| 166* | 0.041 | 0.0305 | 0.108 |
| 167* | 0.0251 | 0.00358 | 0.029 |
| 168 | 0.388 | 0.229 | ND |
| 169 | 0.0302 | 0.0076 | 0.0062 |
| 170 | 0.00687 | 0.00298 | 0.00533 |
| 171 | ND | 0.00588 | 0.0166 |
| 202 | 0.188 | 0.236 | 0.723 |
| 172 | 0.0134 | 0.004 | 0.00827 |
| 173 | 0.00781 | 0.004 | 0.0223 |
| 174 | 0.0383 | 0.00751 | 0.035 |
| 175* | 0.0142 | 0.00546 | 0.0113 |
| 176* | 0.00864 | 0.00171 | 0.00182 |
| 177* | 0.0155 | 0.00416 | 0.00982 |
| 178* | 0.0358 | 0.0656 | 0.555 |
| 179* | 0.0109 | 0.000952 | 0.0264 |
| 180* | 0.0306 | 0.0245 | 0.124 |
| 181* | 0.0101 | 0.0070 | 0.0124 |
| 182* | 0.0158 | 0.0185 | 0.0369 |
| 183* | 0.0106 | 0.0086 | 0.0131 |
| 184* | 0.0213 | 0.0271 | 0.0559 |
| 185* | 0.012 | 0.00766 | 0.0113 |
| 186* | 0.0206 | 0.0567 | 0.431 |
| 187* | 0.0203 | 0.00775 | 0.0373 |
| 188* | 0.169 | 0.122 | 0.549 |
| 189* | 0.00914 | 0.00343 | 0.00332 |
| 190* | 0.00963 | 0.0109 | 0.0251 |
| 191* | 0.00804 | 0.00266 | 0.00852 |
| 192* | 0.0126 | 0.0227 | 0.0616 |
| 193* | 0.00729 | 0.00541 | 0.00712 |
| 194* | 0.0131 | 0.0423 | 0.131 |
| 195* | 0.0127 | 0.00242 | 0.00749 |
| 196* | 0.0127 | 0.0133 | 0.0462 |
| 197* | 0.00987 | 0.00546 | 0.0101 |
| 198* | 0.0202 | 0.0413 | 0.105 |
| 199* | 0.0082 | 0.00206 | 0.00401 |
| 200* | 0.0116 | 0.0137 | 0.0367 |
| 201* | 0.011 | 0.00823 | 0.0245 |
| 203 | 0.0275 | 0.0214 | 0.0973 |
| 204 | 0.016 | 0.00207 | 0.006 |

### Comparative Data

The compounds of Formula (I) as described herein do not comprise an amino group attached to Ring B. As shown in Table 4, presence of an amine group (—NH₂) leads to decreased inhibition of EBP and decreased activity as shown in the mouse zymostenol and OPC assays.

For example, presence of an amine group on Ring B in Compound A led to a 19.5-fold decrease in EBP inhibition, a 5.9-fold decrease in activity in the mouse zymostenol assay, and a 4.1-fold decrease in activity in the OPC assay as compared to compound 202.

Presence of an amine group on Ring B in Compound B led to a 41.7-fold decrease in activity in the OPC assay as compared to compound 171.

Presence of an amine group on Ring B in Compound C led to a 4.9-fold decrease in activity in the mouse zymostenol assay, and a 3.8-fold decrease in activity in the OPC assay as compared to compound 29*.

TABLE 4

| | hEBP-DHCR7 | Mouse | OPC |
|---|---|---|---|
| | Ki | Zymostenol | EC₅₀ |
| Compound | (μM) | GCMS EC₅₀ (μM) | (μM) |
| 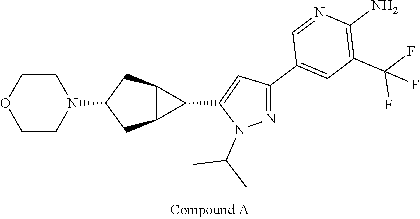 [SMILES: CC(C)n1nc(-c2cnc(N)c(C(F)(F)F)c2)cc1[C@H]1[C@@H]2C[C@@H](N3CCOCC3)C[C@@H]21] | 3.67 | 1.40 | 3.0 |
| 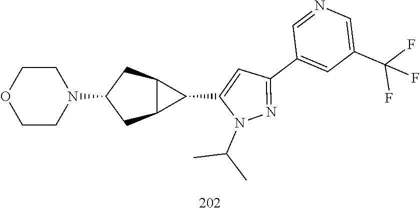 [SMILES: CC(C)n1nc(-c2cncc(C(F)(F)F)c2)cc1[C@H]1[C@@H]2C[C@@H](N3CCOCC3)C[C@@H]21] | 0.188 | 0.236 | 0.723 |
| 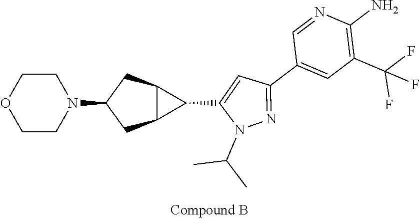 [SMILES: CC(C)n1nc(-c2cnc(N)c(C(F)(F)F)c2)cc1[C@H]1[C@@H]2C[C@H](N3CCOCC3)C[C@@H]21] | ND | ND | 0.692 |
| 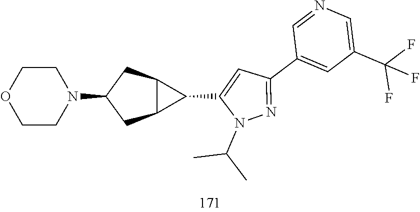 [SMILES: CC(C)n1nc(-c2cncc(C(F)(F)F)c2)cc1[C@H]1[C@@H]2C[C@H](N3CCOCC3)C[C@@H]21] | ND | 0.00588 | 0.0166 |
| 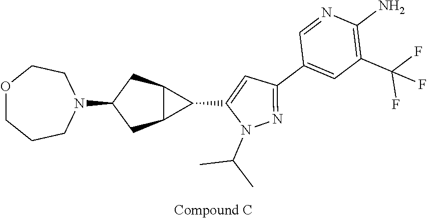 [SMILES: CC(C)n1nc(-c2cnc(N)c(C(F)(F)F)c2)cc1[C@H]1[C@@H]2C[C@H](N3CCCOCC3)C[C@@H]21] | ND | 0.0156 | 0.075 |
| 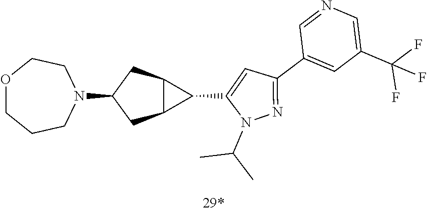 [SMILES: CC(C)n1nc(-c2cncc(C(F)(F)F)c2)cc1[C@H]1[C@@H]2C[C@H](N3CCCOCC3)C[C@@H]21] | 0.02 | 0.00319 | 0.0198 |

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A compound of Formula I: 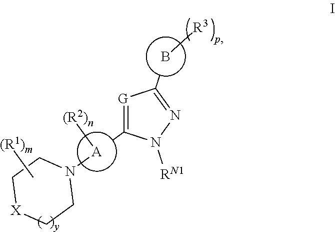
or a pharmaceutically acceptable salt thereof,
wherein:
n is 0 or 1;
p is 1 or 2;
Ring A is a saturated 5- or 6-membered monocyclic or bicyclic carbocyclyl;
Ring B is phenyl, 6-membered heteroaryl, or 6-membered saturated or partially saturated cycloalkyl, wherein the heteroaryl contains one or two heteroatoms;
R³, in each instance, is independently selected from the group consisting of C₁-C₆ alkyl, halo-C₁-C₆ alkyl, hydroxy, C₁-C₆ alkoxy, halo-C₁-C₆ alkoxy, halo, and cyano;
G is N or CR^{G}, wherein R^{G} is selected from the group consisting of hydrogen, C₁-C₆ alkyl, and halo-C₁-C₆ alkyl;
R^{N1} is selected from the group consisting of hydrogen, C₁-C₆ alkyl, halo-C₁-C₆ alkyl, and C₃-C₅ cycloalkyl;
R² is selected from the group consisting of C₁-C₆ alkyl, halo, and halo-C₁-C₆ alkyl;
y is 1 or 2;
m is 0, 1, 2, or 3;
X is O or NR^{H};
R^{H} is selected from the group consisting of hydrogen, C₁-C₆ alkyl, halo-C₁-C₆ alkyl, C₃-C₅ cycloalkyl, hydroxy-C₁-C₆ alkyl, and C₁-C₆ alkoxy-C₁-C₆ alkyl;
and,
R¹ in each instance, is selected from the group consisting of C₁-C₆ alkyl, halo-C₁-C₆ alkyl, C₁-C₆ alkoxy, hydroxy, NR^{E}R^{F}, halo-C₁-C₆ alkoxy, hydroxy-C₁-C₆ alkyl, and C₁-C₆ alkoxy-C₁-C₆ alkyl; or, two R¹ groups come together to form a —CH₂— or —CH₂CH₂— bridge;
R^{E} and R^{F} are each independently selected from the group consisting of hydrogen, C₁-C₆ alkyl, halo-C₁-C₆ alkyl, C₃-C₅ cycloalkyl, hydroxy-C₁-C₆ alkyl, and C₁-C₆ alkoxy-C₁-C₆ alkyl.

2. The compound of claim 1, wherein Ring A is selected from the group consisting of cyclopentyl, cyclohexyl, and bicyclo[3.1.0]hexanyl.

3. (canceled)

4. The compound of claim 2, wherein Ring A is cyclopentyl, and having the formula:

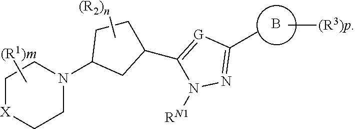

5.-7. (canceled)

8. The compound of claim 1, wherein Ring B is selected from the group consisting of cyclohexyl, phenyl, pyridinyl, pyrimidinyl, and pyrazinyl.

9.-10. (canceled)

11. The compound of claim 2, which is of formula:

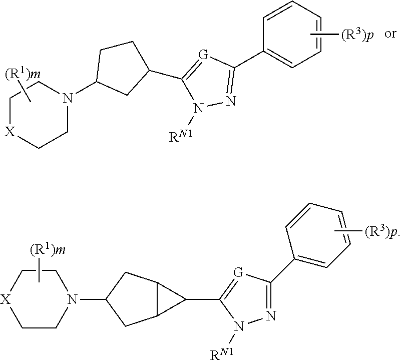

12.-13. (canceled)

14. The compound of claim 1, wherein the compound is of Formula Ia or Ia′: 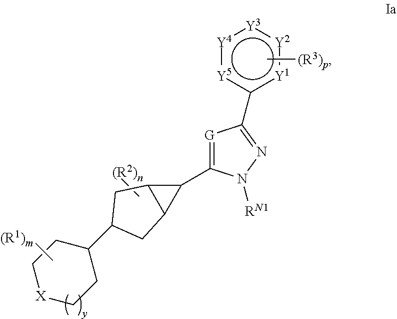
or a pharmaceutically acceptable salt thereof,
wherein,
Y¹, Y², Y³, Y⁴, and Y⁵ are each independently N, C or CH, provided that only one or two of Y¹, Y², Y³, Y⁴, and Y⁵ can be N; or, 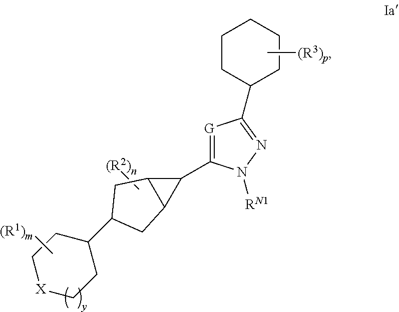
or a pharmaceutically acceptable salt thereof.

15. The compound of claim 1, wherein the compound is of Formula Ib: 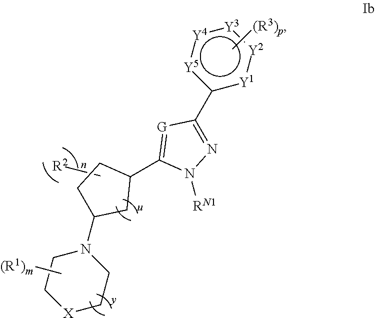
or a pharmaceutically acceptable salt thereof,
wherein:
Y¹, Y², Y³, Y⁴, and Y⁵ are each independently N, C or CH, provided that only one or two of Y¹, Y², Y³, Y⁴, and Y⁵ can be N; and
u is 1 or 2.

16. The compound of claim 14, wherein
(i) Y¹ is CH, Y² is CR³, Y³ is CH, Y⁴ is N, and Y⁵ is CH;
(ii) Y¹ is CH, Y² is N, Y³ is CR³, Y⁴ is CH, and Y⁵ is CH;
(iii) Y¹ is N, Y² is CR³, Y³ is CH, Y⁴ is CH, and Y⁵ is CH;
(iv) Y¹ is CH, Y² is CR³, Y³ is CH, Y⁴ is CH, and Y⁵ is CH;
(v) Y¹ is CH, Y² is CR³, Y³ is CH, Y⁴ is CH, and Y⁵ is N;
(vi) Y¹ is CH, Y² is N, Y³ is CR³, Y⁴ is N, and Y⁵ is CH;
(vii) Y¹ is N, Y² is CR³, Y³ is N, Y⁴ is CH, and Y⁵ is CH;
(viii) Y¹ is CH, Y² is CR³, Y³ is N, Y⁴ is CH, and Y⁵ is N;
(ix) Y¹ is N, Y² is CR³, Y³ is CH, Y⁴ is N, and Y⁵ is CH;
(x) Y¹ is CH, Y² is CR³, Y³ is CR³, Y⁴ is CH, and Y⁵ is CH;
(xi) Y¹ is CH, Y² is N, Y³ is CR³, Y⁴ is CR³, and Y⁵ is CH;
(xii) Y¹ is CH, Y² is CR³, Y³ is N, Y⁴ is CH, and Y⁵ is CH;
(xiii) Y¹ is CH, Y² is CH, Y³ is CR³, Y⁴ is CH, and Y⁵ is CH;
(xiv) Y¹ is N, Y² is CH, Y³ is CR³, Y⁴ is CH, and Y⁵ is CH; or,
(xv) Y¹ is CR³, Y² is CH, Y³ is CH, Y⁴ is CH, and Y⁵ is CH.

17. The compound of claim 15, wherein
(i) Y¹ is CH, Y² is CR³, Y³ is CH, Y⁴ is N, and Y⁵ is CH;
(ii) Y¹ is CH, Y² is N, Y³ is CR³, Y⁴ is CH, and Y⁵ is CH;
(iii) Y¹ is N, Y² is CR³, Y³ is CH, Y⁴ is CH, and Y⁵ is CH;
(iv) Y¹ is CH, Y² is CR³, Y³ is CH, Y⁴ is CH, and Y⁵ is CH;
(v) Y¹ is CH, Y² is CR³, Y³ is CH, Y⁴ is CH, and Y⁵ is N;
(vi) Y¹ is CH, Y² is N, Y³ is CR³, Y⁴ is N, and Y⁵ is CH;
(vii) Y¹ is N, Y² is CR³, Y³ is N, Y⁴ is CH, and Y⁵ is CH;
(viii) Y¹ is CH, Y² is CR³, Y³ is N, Y⁴ is CH, and Y⁵ is N;
(ix) Y¹ is N, Y² is CR³, Y³ is CH, Y⁴ is N, and Y⁵ is CH;
(x) Y¹ is CH, Y² is CR³, Y³ is CR³, Y⁴ is CH, and Y⁵ is CH;
(xi) Y¹ is CH, Y² is N, Y³ is CR³, Y⁴ is CR³, and Y⁵ is CH;
(xii) Y¹ is CH, Y² is CR³, Y³ is N, Y⁴ is CH, and Y⁵ is CH;
(xiii) Y¹ is CH, Y² is CH, Y³ is CR³, Y⁴ is CH, and Y⁵ is CH;
(xiv) Y¹ is N, Y² is CH, Y³ is CR³, Y⁴ is CH, and Y⁵ is CH; or,
(xv) Y¹ is CR³, Y² is CH, Y³ is CH, Y⁴ is CH, and Y⁵ is CH.

18.-30. (canceled)

31. The compound of claim 1, wherein the compound is of Formula Ib′: 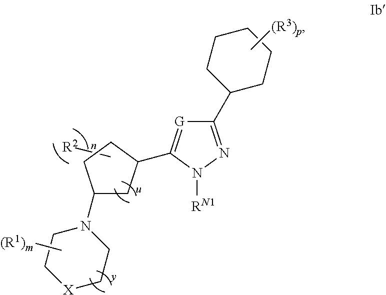
or a pharmaceutically acceptable salt thereof.

32.-40. (canceled)

41. The compound of claim 1, wherein G is N.

42. The compound of claim 41, having the formula:

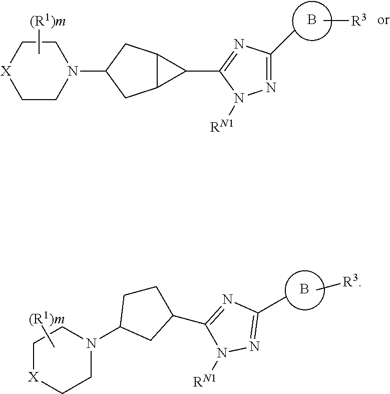

43. The compound of claim 42, wherein Ring B is cyclohexyl, phenyl, pyridinyl, pyrimidinyl, or pyrazinyl.

44. The compound of claim 1, wherein G is CH.

45. The compound of claim 44, having the formula:

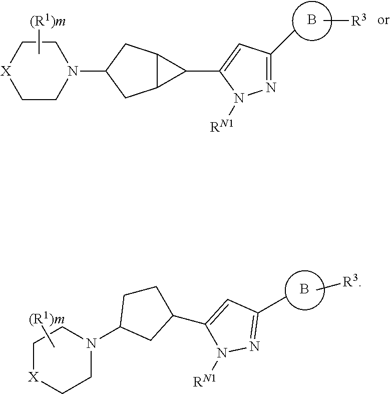

46.-50. (canceled)

51. The compound of claim 1, wherein X is O.

52.-60. (canceled)

61. The compound of claim 51, wherein X, R¹, m, and y in the ring form:

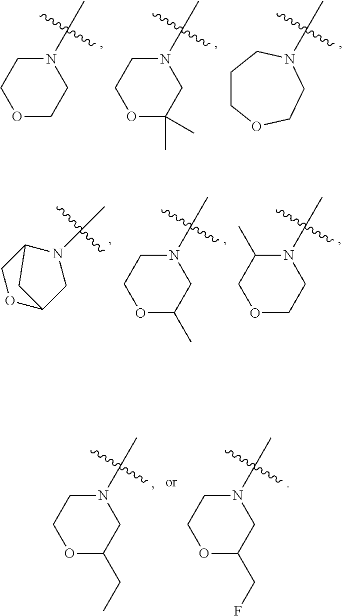

62. The compound of claim 1, wherein X is NR^{H}.

63.-65. (canceled)

66. The compound of claim 62, wherein X, R¹, m, and y in the ring form:

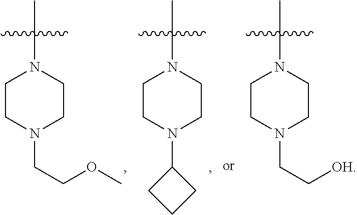

67. The compound of claim 1, wherein the compound is of Formula Ia1 or lbl: 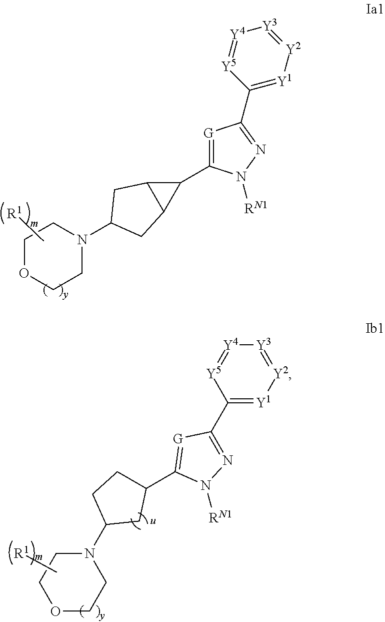
or a pharmaceutically acceptable salt thereof,
wherein:
u is 1 or 2;
m is 0, 1, or 2;
Y¹, Y², Y³, Y⁴, and Y⁵ are each independently CH, CR³, or N, provided that only one or two of Y¹, Y², Y³, Y⁴, and Y⁵ can be N;
G is N or CH; and
R¹, if present, in each instance is independently selected from the group consisting of C₁-C₆ alkyl and halo-C₁-C₆ alkyl; or wherein two R¹ groups come together to form a —CH₂— or —CH₂CH₂— bridge.

68.-78. (canceled)

79. The compound of claim 67, wherein:
Y¹ is CH, Y² is CR³, Y³ is CH, Y⁴ is N, and Y⁵ is CH;
Y¹ is CH, Y² is N, Y³ is CR³, Y⁴ is CH, and Y⁵ is CH;
Y¹ is N, Y² is CR³, Y³ is CH, Y⁴ is CH, and Y⁵ is CH;
Y¹ is CH, Y² is CR³, Y³ is CH, Y⁴ is CH, and Y⁵ is CH;
Y¹ is CH, Y² is CR³, Y³ is CH, Y⁴ is CH, and Y⁵ is N;
Y¹ is CH, Y² is N, Y³ is CR³, Y⁴ is N, and Y⁵ is CH;
Y¹ is N, Y² is CR³, Y³ is N, Y⁴ is CH, and Y⁵ is CH;
Y¹ is CH, Y² is CR³, Y³ is N, Y⁴ is CH, and Y⁵ is N;
Y¹ is N, Y² is CR³, Y³ is CH, Y⁴ is N, and Y⁵ is CH;
Y¹ is CH, Y² is CR³, Y³ is CR³, Y⁴ is CH, and Y⁵ is CH;
Y¹ is CH, Y² is N, Y³ is CR³, Y⁴ is CR³, and Y⁵ is CH;
Y¹ is CH, Y² is CR³, Y³ is N, Y⁴ is CH, and Y⁵ is CH;
Y¹ is CH, Y² is CH, Y³ is CR³, Y⁴ is CH, and Y⁵ is CH;
Y¹ is N, Y² is CH, Y³ is CR³, Y⁴ is CH, and Y⁵ is CH; or,
Y¹ is CR³, Y² is CH, Y³ is CH, Y⁴ is CH, and Y⁵ is CH.

80.-84. (canceled)

85. The compound of claim 1, wherein the compound is of Formula Ia2 or Ib2: 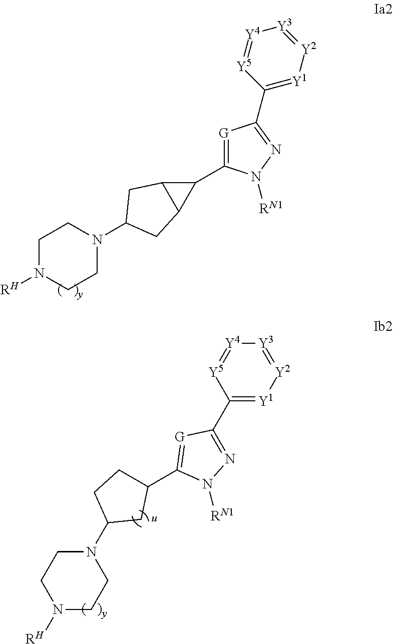
or a pharmaceutically acceptable salt thereof,
wherein:
u is 1 or 2;
Y¹, Y², Y³, Y⁴, and Y⁵ are each independently CH, CR³, or N, provided that only one or two of Y¹, Y², Y³, Y⁴, and Y⁵ can be N; and
G is N or CH.

86.-96. (canceled)

97. The compound of claim 1, wherein Ring A is a bicyclo[3.1.0]hexanyl having the following structure: 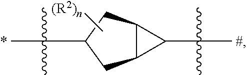
wherein * indicates attachment to the N-atom and # indicates attachment to C-atom on the triazole or pyrazole moiety.

98.-106. (canceled)

107. The compound of any one of claims 1, 4, 11, 15, 31, 42, 45, 67, or 85, wherein Ring A is a cyclopentyl having the following structure:

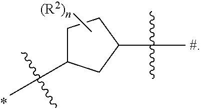

108.-112. (canceled)

113. The compound of claim 1, or a pharmaceutically acceptable salt thereof, selected from Table 1.

114. A pharmaceutical composition comprising a compound according to claim 1, or a pharmaceutically acceptable salt thereof, and a pharmaceutically acceptable excipient.

115. A method of treating a disorder in a subject in need thereof, the method comprising administering to the subject in need thereof a therapeutically effective amount of a compound according to claim 1, or a pharmaceutically acceptable salt thereof.

116.-117. (canceled)

118. The method of claim 115, wherein the disorder is a myelin-related disorder, optionally wherein the myelin-related disorder is multiple sclerosis (MS), neuromyelitis optica (NMO), optic neuritis, pediatric leukodystrophy, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy, or radiation-induced demyelination.

119. (canceled)

120. A method of promoting myelination in a subject in need thereof, comprising administering to the subject a therapeutically effective amount of a compound according to claim 1, or a pharmaceutically acceptable salt thereof.

121.-122. (canceled)

123. The method of claim 120, wherein the subject has a myelin-related disorder.

124.-126. (canceled)
